(19) United States
(12) Patent Application Publication
(10) Pub. No.: US 2010/0267697 A1
(43) Pub. Date: Oct. 21, 2010

(54) ION CHANNEL MODULATORS

(75) Inventors: Vincent P. Galullo, South Grafton, MA (US); Robert Zelle, Stow, MA (US); Danielle Soenen, Irvine, CA (US); Christopher Todd Baker, Bedford, MA (US); Paul Will, Sudbury, MA (US); Hormoz Mazdiyasni, Marlborough, MA (US); Jinsong Guo, Beijing (CN); Andrew Fensome, Wayne, PA (US); Jeffrey Curtis Kern, Gilbertsville, PA (US); William Jay Moore, Collegeville, PA (US); Edward George Melenski, Collegeville, PA (US); Justin Kaplan, Philadelphia, PA (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/518,342

(22) PCT Filed: Dec. 11, 2007
(86) PCT No.: PCT/US2007/087060

§ 371 Date: Dec. 14, 2009

Related U.S. Application Data
(60) Provisional application No. 60/874,102, filed on Dec. 11, 2006.

(51) Int. Cl. A61K 31/5513; A61K 31/55; A61K 31/54; A61K 31/535; A61K 31/5355; A61K 31/4995; A61K 31/501; A61K 31/497; A61K 31/506; A61K 31/44; A61K 31/4725; A61K 31/426; A61K 31/4709; A61K 31/4458; A61K 31/4453; A61K 31/4402; A61K 31/445; A61K 31/166; C07D 403/04; C07D 401/04; C07D 413/04; C07D 417/04; C07D 413/12; C07D 403/12; C07D 241/36; C07D 497/10; C07D 217/00; C07D 215/00; C07D 401/12; C07D 211/98; C07D 213/04; C07D 405/12; C07D 213/73; C07D 277/593; C07C 233/42

(52) U.S. Cl. 514/217.04; 514/218; 514/227.8; 514/236.8; 514/237.2; 514/249; 514/252.03; 514/253.01; 514/253.1; 514/275; 514/278; 514/307; 514/370; 514/314; 514/318; 514/326; 514/329; 514/332; 514/336; 514/352; 514/629; 540/480; 540/597; 540/598; 544/60; 544/131; 544/133; 544/238; 544/331; 544/349; 544/360; 544/369; 546/19; 546/146; 546/164; 546/167; 546/194; 546/208; 546/209; 546/244; 546/265; 546/283.4; 546/311; 548/194; 564/220

(57) ABSTRACT

The present teachings provide compounds of Formula (I)

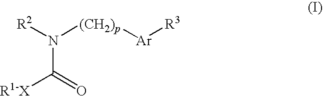

and pharmaceutically acceptable salts, hydrates, and esters thereof, wherein Ar, R¹, R², R³, p, and X are defined herein. The present teachings also provide processes for producing said compounds and their pharmaceutically acceptable salts, hydrates and esters, and methods of treating a pathological condition or disorder, or alleviating a symptom thereof, using said compounds including their pharmaceutically acceptable salts, hydrates and esters. The compounds can be useful in modulating ion channel activity including treating a variety of conditions associated with the abnormal modulation of one or more voltage-gated calcium channels.

This application claims the benefit of U.S. Provisional Application Ser. No. 60/874,102, filed Dec. 11, 2006.

### FIELD OF THE INVENTION

The present teachings relate to certain substituted aliphatic amides and related derivatives, processes for their preparation, and their use in therapeutic treatments.

### BACKGROUND OF THE INVENTION

All cells rely on the regulated movement of inorganic ions across cell membranes to perform essential physiological functions. Electrical excitability, synaptic plasticity, and signal transduction are examples of processes in which changes in ion concentration play a critical role. In general, the ion channels that permit these changes are proteinaceous pores consisting of one or multiple subunits, each containing two or more membrane-spanning domains. Most ion channels have selectivity for specific ions, primarily Na⁺, K⁺, Ca²⁺, or Cl⁻, by virtue of physical preferences for size and charge. Electrochemical forces, rather than active transport, drive ions across membranes, thus a single channel may allow the passage of millions of ions per second. Channel opening, or “gating” is tightly controlled by changes in voltage or by ligand binding, depending on the subclass of channel. Ion channels are attractive therapeutic targets due to their involvement in so many physiological processes, yet the generation of drugs with specificity for particular channels in particular tissue types remains a major challenge.

Voltage-gated ion channels open in response to changes in membrane potential. For example, depolarization of excitable cells such as neurons results in a transient influx of Na⁺ ions, which propagates nerve impulses. This change in membrane potential is sensed by voltage-gated K⁺ channels, which then allow an efflux of K⁺ ions. The efflux of K⁺ ions repolarizes the membrane. Other cell types rely on voltage-gated Ca²⁺ channels to generate action potentials. Voltage-gated ion channels also perform important functions in non-excitable cells, such as the regulation of secretory, homeostatic, and mitogenic processes. Ligand-gated ion channels can be opened by extracellular stimuli such as neurotransmitters (e.g., glutamate, serotonin, and acetylcholine), or intracellular stimuli (e.g., cAMP, Ca²⁺, and phosphorylation).

The Caᵥ2 family of voltage-gated calcium channels consists of 3 main subtypes Caᵥ2.1 (P or Q-type calcium currents), Caᵥ2.2 (N-type calcium currents), and Caᵥ2.3 (R-type calcium currents). These currents are found almost exclusively in the central nervous system (CNS), peripheral nervous system (PNS) and neuroendocrine cells, and constitute the predominant forms of presynaptic voltage-gated calcium current. Presynaptic calcium entry is modulated by many types of G-protein coupled receptors (GPCRs) and modulation of Caᵥ2 channels is a widespread and highly efficacious means of regulating neurotransmission. The subunit composition of the Caᵥ2 channels is defined by their α₁ subunit, which forms the pore and contains the voltage-sensing gates (α₁2.1, α₁2.2, and α₁2.3, also known as α_{1A}, α_{1B}, and α_{1E}, respectively) and the β and α₂ subunits.

Genetic or pharmacological perturbations in ion channel function can have dramatic clinical consequences. Long QT syndrome, epilepsy, cystic fibrosis, and episodic ataxia are a few examples of heritable diseases resulting from mutations in ion channel subunits. Toxic side effects such as arrhythmia and seizure, which can be triggered by certain drugs, can be due to interference with ion channel function (Sirois, J. E. and Atchison, W. D. (1996), Neurotoxicology, 17(1): 63-84; Keating, M. T. (1996), Science, 272: 681-685). Drugs are useful for the therapeutic modulation of ion channel activity, and have applications in treatment of many pathological conditions, including hypertension, angina pectoris, myocardial ischemia, asthma, bladder overactivity, alopecia, pain, heart failure, dysmenorrhea, type II diabetes, arrhythmia, graft rejection, seizure, convulsions, epilepsy, stroke, gastric hypermotility, psychoses, cancer, muscular dystrophy, and narcolepsy (Coghlan, M. J. et al. (2001), J. Med. Chem., 44: 1627-1653; Ackerman, M. J. and Clapham, D. E. (1997), N. Eng. J. Med., 336: 1575-1586). The growing number of identified ion channels and understanding of their complexity will assist in future efforts at therapies, that can modify ion channel function.

Therapeutic modulation of Caᵥ2 channel activity has applications in treatment of many pathological conditions. All primary sensory afferents provide input to neurons in the dorsal horns of the spinal cord and in dorsal root ganglia neurons in the dorsal horn, and calcium influx through Caᵥ2.2 channels triggers the release of neurotransmitters from presynaptic nerve terminals in the spinal cord. Hence, blockade of Caᵥ2.2 channels is expected to be broadly efficacious because these channels are in a common pathway downstream from the wide variety of receptors that mediate pain (Julius, D. and Basbaum, A. I. (2001), Nature, 413: 203-216). Indeed, intrathecal injection of the Caᵥ2.2-selective conotoxin ziconitide (SNX-111) has been shown to be effective against both neuropathic pain and inflammatory pain in animals and man (Bowersox, S. S. et al. (1996), J. Pharmacol. Exp. Ther., 279: 1243-1249). Ziconotide has also been shown to be effective as a neuroprotective agent in rat models of global or focal ischemia (Colburne, F. et al. (1999), Stroke, 30: 662-668). Thus, it is reasonable to conclude that modulation of Caᵥ2.2 can have implications in the treatment of neuroprotection and/or stroke.

Caᵥ2.2 channels are found in the periphery and mediate catecholamine release from sympathetic neurons and adrenal chroffin cells. Some forms of hypertension result from elevated sympathetic tone. Caᵥ2.2 modulators could be particularly effective in treating this disorder. Although complete block of Caᵥ2.2 channels can cause hypotension or impair baroreceptor reflexes, partial inhibition by Caᵥ2.2 modulators might reduce hypertension with minimal reflex tachycardia (Uneyama, O. D. (1999), Int. J. Mol. Med., 3: 455-466).

Overactive bladder (OAB) is characterized by storage symptoms such as urgency, frequency, and nocturia, with or without urge incontinence, resulting from the overactivity of the detrusor muscle in the bladder. OAB can lead to urge incontinence. The etiology of OAB and painful bladder syndrome is unknown, although disturbances in nerves, smooth muscle and urothelium can cause OAB (Steers, W., Rev. Urol., 4: S7-S18). There is evidence to suggest that reduction of bladder hyperactivity may be indirectly effected by inhibition of Caᵥ2.2 and/or Caᵥ1 channels.

The localization of Caᵥ2.1 channels in the superficial laminae of the dorsal horn of the spinal cord suggests involvement of these channels in the perception and maintenance of certain forms of pain (Vanegas, H. and Schaible, H. (2000), Pain, 85: 9-18). Complete elimination of Caᵥ2.1 calcium currents alters synaptic transmission, resulting in severe ataxia. Gabapentin has been used clinically for many years as an add-on therapy for the treatment of epilepsy. In recent years, it has emerged as a leading treatment of neuropathic pain. Clinical trials have shown gabapentin to be effective for the treatment of post-herpetic neuralgia, diabetic neuropathy, trigeminal neuralgia, migrane and fibromyalgia (Mellegers, P. G. et al. (2001), Clin. J. Pain, 17: 284-295). Gabapentin was designed as a metabologically stable GABA mimetic, but most studies find no effect on the GABA receptors. The α₂δ subunit of voltage-gated calcium channels has been identified as a high affinity binding site for gabapentin in the CNS. There is evidence that suggests that gabapentin could inhibit neurotransmission in the spinal cord by interfering with the function of the α₂δ subunits, thereby inhibiting presynaptic calcium currents.

### SUMMARY OF THE INVENTION

The present teachings relate to compounds of formula (I):

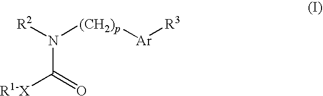

and pharmaceutically acceptable salts, hydrates, and esters thereof, wherein Ar, R¹, R², R³, p, and X are defined as described herein. It should be understood that when reference is made to “compounds” described herein, pharmaceutically acceptable salts, hydrates, and esters thereof are included within that reference.

The present teachings also provide methods of making the compounds of formula (I) and pharmaceutically acceptable salts, hydrates, and esters thereof, and methods of using the compounds of formula (I) and pharmaceutically acceptable salts, hydrates, and esters thereof for the therapeutic modulation of ion channel function, and treatment of one or more conditions, particularly those mediated by certain calcium channel subtype targets. The methods of using the compounds and pharmaceutically acceptable salts, hydrates, and esters thereof generally include administering a therapeutically effective amount of a compound of formula (I) or a pharmaceutically acceptable salt, hydrate, or ester thereof, to a mammal.

### DETAILED DESCRIPTION OF THE INVENTION

Embodiments of the present invention provide compounds that can modulate the activity of ion channels in a mammal, for example, Caᵥ2.2 voltage-gated calcium channels, and can treat a variety of pathological conditions, states, disorders or diseases.

Unless otherwise indicated, the following terms are held to have the following meanings as used herein.

The term “mammal” refers to any warm blooded species, such as a human. The term “ion channel” includes at least voltage-gated calcium channels and voltage-gated sodium channels such as, without limitation, Caᵥ1.1, Caᵥ1.2, Caᵥ1.3, Caᵥ2.1, Caᵥ2.2, Caᵥ2.3, Caᵥ3.1, Caᵥ3.2, Naᵥ1.1, Naᵥ1.2, Naᵥ1.3, Naᵥ1.7, Naᵥ1.8, and Naᵥ1.9. As used herein, “Caᵥ2.2 voltage-gated calcium channel” refers to a voltage-gated calcium channel containing at least one Caᵥ2.2α₁ subunit. The phrase “ion channel mediated condition” refers to any condition or pathological state of a mammal or any disease present in a mammal that can be treated, or the symptoms of which can be alleviated, by modulation of the activity of one or more ion channels such as Caᵥ2.2 voltage-gated calcium channels.

As used herein, “halo” or “halogen” refers to fluoro, chloro, bromo, and iodo.

As used herein, “oxo” refers to a double-bonded oxygen (i.e., ═O).

As used herein, “alkyl” refers to a straight-chain or branched saturated hydrocarbon group. Examples of alkyl groups include methyl (Me), ethyl (Et), propyl (e.g., n-propyl and isopropyl), butyl (e.g., n-butyl, isobutyl, s-butyl, t-butyl), pentyl groups (e.g., n-pentyl, isopentyl, neopentyl), and the like. A lower alkyl group typically has up to 6 carbon atoms. In various embodiments, an alkyl group has 1-6 carbon atoms, and is referred to as a “C₁₋₆alkyl group.” Examples of C₁₋₆alkyl groups include, but are not limited to, methyl, ethyl, propyl (e.g., n-propyl and isopropyl), and butyl groups (e.g., n-butyl, isobutyl, s-butyl, t-butyl). A branched alkyl group has at least 3 carbon atoms (e.g., an isopropyl group) and up to 6 carbon atoms, e.g. it is a C₃₋₆alkyl group, i.e., a branched lower alkyl group. Examples of branched lower alkyl groups include, but are not limited to:

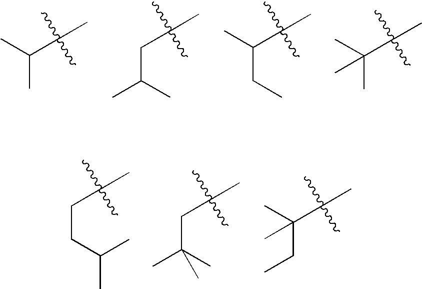

isopropyl, isobutyl, sec-butyl, tert-butyl, isopentyl, neopentyl, and tert-pentyl.

As used here, a divalent C₁₋₆alkyl group can be a straight chain or branched alkyl group, which as a linking group is capable of forming a covalent bond with two other moieties. Examples of a divalent C₁₋₆alkyl group include, for example, a methylene group, an ethylene group, an ethylidene group, an n-propylene group, an isopropylene group, an isobutylene group, a s-butylene group, an n-butylene group, and a t-butylene group.

As used herein, “alkenyl” refers to a straight-chain or branched alkyl group having one or more carbon-carbon double bonds. Examples of alkenyl groups include, but are not limited to, ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene). A branched alkenyl group has at least 3 carbon atoms, and in various embodiments, has up to 6 carbon atoms, e.g. it is a C₃₋₆alkenyl group.

The term “alkynyl” refers to a straight-chain or branched alkyl group having one or more carbon-carbon triple bonds. Examples of alkynyl groups include, but are not limited to, ethynyl, propynyl, butynyl, pentynyl, and the like. The one or more carbon-carbon triple bonds can be internal (such as in 2-butyne) or terminal (such as in 1-butyne). The alkynyl group is suitably a C₃₋₆ alkynyl group.

As used herein, “alkoxy” refers to an —O-alkyl group wherein the alkyl group may be a straight or branched chain. Examples of alkoxy groups include, but are not limited to, methoxy, ethoxy, propoxy (e.g., n-propoxy and isopropoxy), t-butoxy groups, and the like.

A divalent alkoxy group means an alkoxy group which, as a linking group, is capable of forming a covalent bond with two other moieties (—O-alkyl-).

As used herein, “haloalkyl” refers to an alkyl group having one or more halogen substituents. Examples of haloalkyl groups include, but are not limited to, —CF₃, —C₂F₅, —CHF₂, —CH₂F, —CCl₃, —CHCl₂, —CH₂Cl, —C₂Cl₅, and the like. Perhaloalkyl groups, i.e., alkyl groups wherein all of the hydrogen atoms are replaced with halogen atoms (e.g., CF₃ and C₂F₅), are included within the definition of “haloalkyl.”

As used herein, “haloalkoxy” refers to an alkoxy group having one or more halogen substituents. Examples of haloalkoxy groups include, but are not limited to, —OCF₃, —OC₂F₅, —OCHF₂, and the like.

As used herein, “cycloalkyl” refers to a non-aromatic carbocyclic group including cyclized alkyl, alkenyl, and alkynyl groups. A cycloalkyl group can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. In various embodiments, a cycloalkyl group has 3-6 carbon atoms, and is referred to as a “C₃₋₆cycloalkyl group.” Examples of C₃₋₆ cycloalkyl groups include, but are not limited to, cyclopropyl, cyclopropylmethyl, cyclopropylethyl, cyclopropylpropyl, cyclobutyl, cyclobutylmethyl, cyclobutylethyl, cyclopentyl, cyclopentylmethyl, cyclohexyl, cyclopentenyl, cyclohexenyl, and cyclohexadienyl groups, as well as their homologs, isomers, and the like.

As used herein, “heteroatom” refers to an atom of any element other than carbon or hydrogen and includes, for example, nitrogen, oxygen, sulfur, phosphorus, and selenium.

As used herein, “cycloheteroalkyl” refers to a non-aromatic cycloalkyl group having 5-10 ring atoms, among which 1 to 3 ring atoms are heteroatoms independently selected from oxygen (O), nitrogen (N) and sulfur (S), and optionally contains one or more, e.g., two, double or triple bonds. One or more N or S atoms in a cycloheteroalkyl ring can be oxidized (e.g., morpholine N-oxide, thiomorpholine S-oxide, thiomorpholine S,S-dioxide). Cycloheteroalkyl groups can also contain one or more oxo groups, such as piperidone, oxazolidinone, pyrimidine-2,4(1H,3H)-dione, pyridin-2(1H)-one, and the like. Examples of cycloheteroalkyl groups include, among others, morpholine, thiomorpholine, pyran, imidazolidine, imidazoline, oxazolidine, pyrazolidine, pyrazoline, pyrrolidine, pyrroline, tetrahydrofuran, tetrahydrothiophene, tetrahydroisoquinoline, piperidine, piperazine, and the like. A cycloheteroalkyl group can be optionally substituted. For example, in some embodiments, one or more carbon ring atoms of a cycloheteroalkyl group can bear a substituent independently selected from a halogen, a C₁₋₆alkyl group, —C(O)—NR_{d}Rₑ, —Y—OR_{c}, —Y—NR_{d}Rₑ, a —Y-phenyl group, a —Y-(5-7 cycloheteroalkyl) group, a —Y-(5-9 membered heteroaryl) group, or a —Y—O-(5-7 membered heteroaryl) group, and/or one or more nitrogen ring atoms of a cycloheteroalkyl group can bear a substituent independently selected from a halogen, a C₁₋₆alkyl group, —C(O)R_{c}, —C₂₋₆alkyl-OR_{c}, —C₂₋₆alkyl-NR_{d}Rₑ, —Y—C(O)NR_{d}Rₑ, an —S(O)₂—C₁₋₆alkyl group, a —C₂₋₆alkyl-(5-7 membered cycloheteroalkyl) group, or a 5-7 membered heteroaryl group, wherein Y, R_{c}, R_{d}, and Rₑ are as defined hereinbelow. Further, each of the phenyl substituents immediately above can be optionally substituted with 1 to 3 substituents independently selected from a halogen, a C₁₋₆alkyl group, a C₁₋₆haloalkyl group, and a C₁₋₆alkoxy group, and each of the 5-7 membered cycloheteroalkyl substituents, the 5-7 membered heteroaryl substituents, and the 5-9 membered heteroaryl substituents immediately above can be optionally substituted with 1 to 3 substituents independently selected from a halogen and a C₁₋₆alkyl group.

As used herein, “aryl” refers to an aromatic monocyclic hydrocarbon ring system or a polycyclic ring system in which two or more aromatic hydrocarbon rings are fused (i.e., having a bond in common with) together or at least one aromatic monocyclic hydrocarbon ring is fused to one or more cycloalkyl and/or cycloheteroalkyl rings. An aryl group can have from 6 to 14 carbon atoms in its ring system, which can include multiple fused rings. In some embodiments, a polycyclic aryl group can have from 7 to 14 carbon atoms. Any suitable ring position of the aryl group can be covalently linked to the defined chemical structure. Examples of aryl groups having only aromatic carbocyclic ring(s) include, but are not limited to, phenyl, 1-naphthyl (bicyclic), 2-naphthyl (bicyclic), anthracenyl (tricyclic), phenanthrenyl (tricyclic) and like groups. Examples of polycyclic ring systems in which at least one aromatic carbocyclic ring is fused to one or more cycloalkyl and/or cycloheteroalkyl rings include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include, but are not limited to, benzodioxanyl, benzodioxolyl, chromanyl, indolinyl groups, and the like. In some embodiments, aryl groups optionally contain up to three independently selected substitution groups. For example, a phenyl group, in some embodiments, can be optionally substituted with 1 to 3 substituents independently selected from a halogen, CN, —C(O)OR_{c}, —NR_{d}Rₑ, a C₁₋₆alkyl group, a C₁₋₆haloalkyl group, and a C₁₋₆alkoxy group, wherein R_{c}, R_{d}, and Rₑ are as defined hereinbelow.

As used herein, “heteroaryl” refers to an aromatic monocyclic ring system or a polycyclic ring system where at least one of the rings present in the ring system is aromatic, containing 5-7 or 5-9 ring atoms, among which 1 to 3 ring atoms are heteroatoms independently selected from oxygen (O), nitrogen (N) and sulfur (S). Polycyclic heteroaryl groups include two or more heteroaryl rings fused together, and monocyclic heteroaryl rings fused to one or more aromatic carbocyclic rings, non-aromatic carbocyclic rings, and/or non-aromatic cycloheteroalkyl rings. The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of heteroaryl groups include, for example, the 5-membered monocyclic and 5-6 bicyclic ring systems shown below:

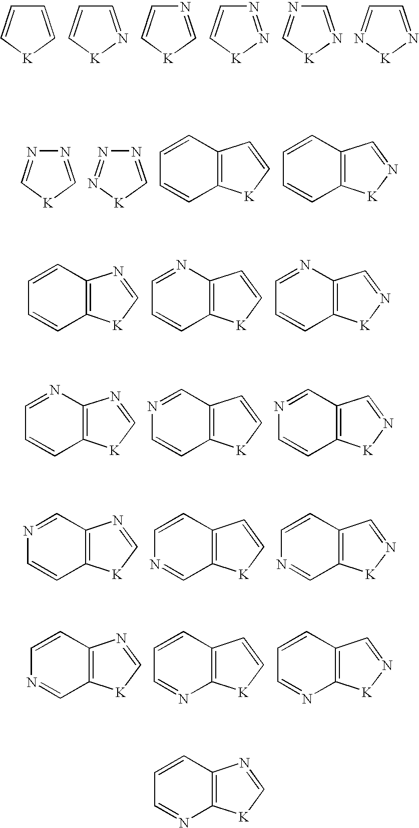

where K is O, S, NH, or NR′; and R′ can be selected from a halogen, a C₁₋₆alkyl group, a C(O)R_{c} group, a C₂₋₆alkyl-OR_{c} group, a C₂₋₆alkyl-NR_{d}Rₑ group, a —Y—C(O)NR_{d}Rₑ group, an S(O)₂—C₁₋₆alkyl group, a 5-7 membered heteroaryl group, and a C₂₋₆alkyl-(5-7 membered cycloheteroalkyl) group, where Y, R_{c}, R_{d} and Rₑ are as defined hereinbelow. Examples of such heteroaryl rings include, but are not limited to, pyrrole, furan, thiophene, pyridine, pyrimidine, pyridazine, pyrazine, triazole, tetrazole, pyrazole, imidazole, isothiazole, thiazole, thiadiazole, isoxazole, oxazole, oxadiazole, indole, isoindole, benzofuran, benzothiophene, quinoline, 2-methylquinoline, isoquinoline, quinoxaline, quinazoline, benzotriazole, benzimidazole, benzothiazole, benzisothiazole, benzisoxazole, benzoxadiazole, benzoxazole, cinnoline, 1H-indazole, 2H-indazole, indolizine, isobenzofuran, naphthyridine, phthalazine, pteridine, purine, oxazolopyridine, thiazolopyridine, imidazopyridine, furopyridine, thienopyridine, pyridopyrimidine, pyridopyrazine, pyridopyridazine, thienothiazole, thienoxazole, and thienoimidazole. Further examples of heteroaryl groups include, but are not limited to, 4,5,6,7-tetrahydroindole, tetrahydroquinoline, benzothienopyridine, benzofuropyridine, and the like. In some embodiments, heteroaryl groups can be substituted with up to three independently selected substitution groups. For example, in some embodiments, one or more nitrogen atoms can be substituted with independently selected R′ groups as defined above, and/or one or more carbon ring atoms of a cycloheteroalkyl group can bear a substituent independently selected from a halogen, a C₁₋₆alkyl group, —C(O)—NR_{d}Rₑ, —Y—OR_{c}, —Y—NR_{d}Rₑ, a —Y-phenyl group, a —Y-(5-7 cycloheteroalkyl) group, a —Y-(5-9 membered heteroaryl) group, or a —Y—O-(5-7 membered heteroaryl) group, wherein Y, R_{c}, R_{d}, and Rₑ are as defined hereinbelow. Further, each of the phenyl substituents immediately above can be optionally substituted with 1 to 3 substituents independently selected from a halogen, a C₁₋₆alkyl group, a C₁₋₆haloalkyl group, and a C₁₋₆alkoxy group, and each of the 5-7 membered cycloheteroalkyl substituents, the 5-7 membered heteroaryl substituents, and the 5-9 membered heteroaryl substituents immediately above can be optionally substituted with 1 to 3 substituents independently selected from a halogen and a C₁₋₆alkyl group.

Aa “divalent group” is defined herein as a linking group capable of forming a covalent bond with two other moieties. As used herein, a “leaving group” (“LG”) refers to a charged or uncharged atom (or group of atoms) that can be displaced as a stable species as a result of, for example, a substitution or elimination reaction. Examples of leaving groups include, but are not limited to, halide (e.g., Cl, Br, I), tosylate (toluenesulfonyl group, TsO), mesylate (methanesulfonyl group, MsO), brosylate (p-bromobenzenesulfonyl group, BsO), nosylate (4-nitrobenzenesulfonyl group, NsO), water (H₂O), ammonia (NH₃), and triflate (trifluoromethanesulfonyl group, OTf).

As used herein, a “protecting group” (“PtG”) refers to modification of a functional group that reduces the reactivity of the functional group in an unwanted reaction. Examples of protecting groups for amines include, but are not limited to, tert-butyloxycarbonyl (t-BOC), benzyl (Bn), and carbobenzyloxy (Cbz) groups. Examples of protecting groups for carbonyls include, but are not limited to, acetals and ketals. Examples of protecting groups for carboxylic acids include, but are not limited to, methyl esters, benzyl esters, tert-butyl esters, and silyl esters. See Greene, et al., Protective Groups in Organic Synthesis, 2d. Ed., Wiley & Sons, 1991, the entire disclosure of which is incorporated by reference herein for all purposes.

At various places in the present specification, substituents of compounds are disclosed in groups or in ranges. It is specifically intended that the description include each and every individual subcombination of the members of such groups and ranges. For example, the term “C₁₋₆alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆alkyl. By way of another example, the term “5-9 membered heteroaryl group” is specifically intended to individually disclose a heteroaryl group having 5, 6, 7, 8, 9, 5-9, 5-8, 5-7, 5-6, 6-9, 6-8, 6-7, 7-9, 7-8, and 8-9 ring atoms.

The present teachings provide compounds of formula (I):

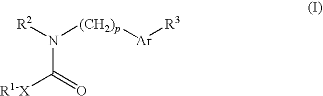

and pharmaceutically acceptable salts, hydrates, and esters thereof, wherein:
X is —NR_{c}—, —O—, or a covalent bond;

R¹ is a C₁₋₁₀alkylgroup, a branched C₃₋₁₀alkyl group, a branched C₃₋₁₀alkenyl group, a C₃₋₈cycloalkyl group, or a 5-10 membered cycloheteroalkyl group, wherein:
the C₁₋₁₀alkylgroup, the branched C₃₋₁₀alkyl group and the branched C₃₋₁₀alkenyl group are optionally substituted with 1 to 3 substituents independently selected from a halogen and a 5-7 membered heteroaryl group, wherein the 5-7 membered heteroaryl group is optionally substituted with 1 to 3 substitutents independently selected from —C(O)OR_{c}, a —Y—NR_{d}Rₑ group, and a —Y-phenyl group; and
the 5-10 membered cycloheteroalkyl group is optionally substituted with 1 to 3 substituents independently selected from a C₁₋₆alkyl group, an oxo group, and a —Y-phenyl group, wherein the phenyl group is optionally substituted with 1 to 3 substituted independently selected from a halogen and a C₁₋₆alkoxy group;
the C₃₋₈cycloalkyl group is optionally substituted with 1 to 3 substituents independently selected from a C₁₋₆alkyl group and a —Y-phenyl group, wherein the —Y-phenyl group is bonded to a carbon atom which is not bonded to X and is optionally substituted with 1 to 3 substituents independently selected from a halogen and a C₁₋₆alkoxy group;
R² is C₃₋₆cycloalkyl, 5,6,7,8-tetrahydronaphthalen-1-yl, indole, benzyl, or phenyl, wherein
   phenyl and benzyl are each optionally substituted with 1 to 3 substituents independently selected from halogen, C₁₋₆alkyl, C₁₋₆alkoxy, C₁₋₆haloalkyl, C₁₋₆haloalkoxy, CN, —C(O)OR_{c}, and —NR_{d}Rₑ; and the —C₃₋₆cycloalkyl is optionally substituted with 1 to 3 C₁₋₆alkyl groups;
Ar—R³ is selected from:

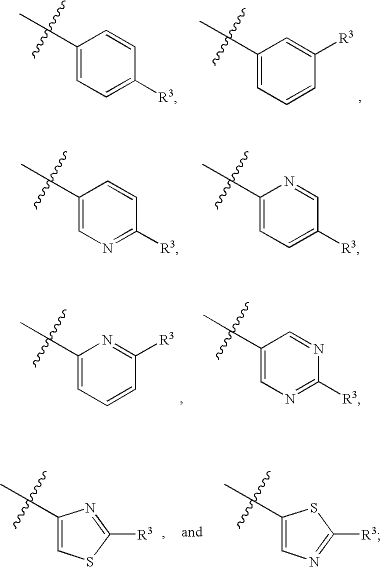

R³ is selected from halogen, C₁₋₁₀alkyl, C₁₋₁₀alkoxy, C₁₋₁₀haloalkyl, C₁₋₁₀ haloalkoxy, C(O)R_{c}, piperidin-4-yl, C₃₋₆cycloalkyl, phenyl, 2-quinolin-3-yl, and —Y—NR_{f}R_{g}; wherein the phenyl is optionally substituted with 1 to 3 substituents independently selected from halogen, C₁₋₆haloalkyl, —OR_{c}, and —C(O)OR_{c}; and the C₁₋₁₀alkyl and the C₁₋₁₀alkoxy are optionally substituted with 1-3 substitutents selected from halogen, phenyl, and —OH;
Y, at each occurrence, is independently a divalent C₁₋₆alkyl group or a covalent bond;
R_{c}, R_{d} and Rₑ, at each occurrence, independently are H or a C₁₋₆alkyl group;
R_{f} and R_{g}, at each occurrence, independently are selected from H, —C(O) R_{c}, —C₂₋₆alkyl-OR_{c}, —C₂₋₆alkyl-NR_{d}Rₑ, a C₁₋₆alkyl group, a C₃₋₆cycloalkyl group, a —Y-phenyl group, a —C(O)-phenyl group, a —Y-(5-7 membered cycloheteroalkyl) group, a —Y-(5-7 membered heteroaryl) group, and a —C₂₋₆alkyl-O—Y-(5-7 membered heteroaryl) group, or
alternatively, R_{f} and R_{g} taken together with the nitrogen atom to which they are bonded form a 5-8 membered cycloheteroalkyl group or a 5-7 membered heteroaryl group, the 5-7 membered cycloheteroalkyl group and the 5-7 membered heteroaryl group containing up to two ring heteroatoms independently selected from nitrogen, oxygen, and sulfur, wherein
   any sulfur atom in the ring optionally is substituted with 1 or 2 oxo groups;
   one or more nitrogen atoms in the ring optionally are independently substituted with —C(O)R_{c}, —C₂₋₆alkyl-OR_{c}, —C₂₋₆alkyl-NR_{d}Rₑ, —Y—C(O)NR_{d}Rₑ, an —S(O)₂—C₁₋₆alkyl group, a —C₂₋₆alkyl-(5-7 membered cycloheteroalkyl) group, a C₁₋₆alkyl group, a —Y-(phenyl)_{q} group, a —C(O)O—C₁₋₆alkyl group, or a 5-7 membered heteroaryl group,
   one or more carbon atoms in the ring optionally are independently substituted with CN, —C(O)—NR_{d}Rₑ, —Y—OR_{c}, —Y—NR_{d}Rₑ, a —Y-phenyl group, a —Y-(5-7 cycloheteroalkyl) group, a —Y-(5-9 membered heteroaryl) group, a C₁₋₆alkyl group, or a —Y—O-(5-7 membered heteroaryl) group;
   each of the phenyl groups appearing anywhere in said R_{f} and R_{g} is optionally substituted with 1 to 3 substituents independently selected from a halogen, a C₁₋₆alkyl group, a C₁₋₆haloalkyl group, and a C₁₋₆alkoxy group; and
   each of the 5-7 membered cycloheteroalkyl groups, the 5-7 membered heteroaryl groups, and the 5-9 membered heteroaryl groups appearing anywhere in said R_{f} and R_{g} is optionally substituted with 1 to 3 substituents independently selected from a halogen and a C₁₋₆alkyl group;
p is 1, 2, 3, or 4; and
q is 1, 2, or 3.

In some embodiments, X can be —NH—, —O—, or a covalent bond. In particular embodiments, X can be a covalent bond.

In accordance with some embodiments, R¹ is a methyl group.

In certain embodiments, R¹ can be a branched lower alkyl group, for example,

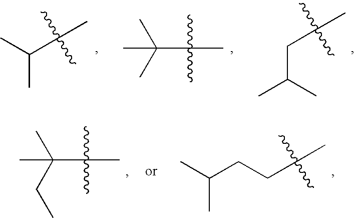

wherein each branched lower alkyl group optionally can be substituted with 1 to 3 substituents independently selected from a halogen, and a 5-7 membered heteroaryl group, where the 5-7 membered heteroaryl group optionally can be substituted with 1 to 3 substitutents independently selected from —C(O)OR_{c}, a —Y—NR_{d}Rₑ group, and a —Y-phenyl group, where Y, R_{c}, R_{d} and Rₑ are as defined herein. For example, R¹ can be tert-butyl optionally substituted with a 5-7 membered heteroaryl group, where the 5-7 membered heteroaryl group can be optionally substituted as described above.

In other embodiments, R¹ can be a C₃₋₆cycloalkyl group. For example, R¹ can be cyclopropyl, cyclobutyl, cyclopentyl, or cyclohexyl.

In certain embodiments, R¹ can be a 5-10 membered cycloheteroalkyl group optionally substituted with a C₁₋₆alkyl group or a benzyl group. For example, R¹ can be an oxygen-containing cycloheteroalkyl group, such as tetrahydrofuran-2-yl, tetrahydrofuran-3-yl, or tetrahydropyran-4-yl; or a nitrogen-containing cycloheteroalkyl group, such as piperidin-4-yl, pyrrolidin-2-yl, pyrrolidin-3-yl, or isoquinolin-3-yl, each of which optionally can include an nitrogen ring atom substituted with a methyl group or a benzyl group.

In some embodiments, R² can be a phenyl group optionally substituted with 1-2 substituents independently selected from a halogen, a C₁₋₆alkyl group, CN, —C(O)OR_{c}, and —NR_{d}Rₑ, wherein R_{c}, R_{d} and Rₑ are as defined above. For example, R² can be a 4-fluorophenyl group, a 4-chloro-phenyl group, a 4-methyl-phenyl group, a 3-methyl-phenyl group, a 2-methyl-phenyl group, a 4-fluoro-2-methyl-phenyl group, a 3,5-dimethylphenyl group, a 2,6-dimethylphenyl group, a 2-isopropylphenyl group, a 2-fluorophenyl group, a 3-fluorophenyl group, or a 3-isopropylphenyl group.

In other embodiments, R² can be selected from cyclopropyl, cyclobutyl, and cyclopentyl.

In certain embodiments, Ar—R³ can be

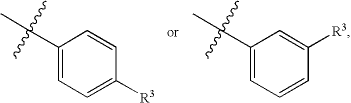

wherein R³ is as defined above.

In some embodiments, Ar—R³ can be

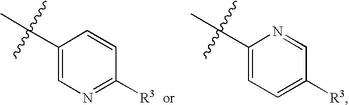

wherein R³ is as defined above.

In some embodiments, Ar—R³ can be

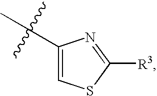

wherein R³ is as defined above.

In some embodiments, Ar—R³ can be

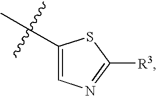

wherein R³ is as defined above.

In some embodiments, R³ can be NR_{f}R_{g}, wherein R_{f} and R_{g} are as defined above. In particular embodiments, R³ can be selected from NH₂, an NH—C₁₋₆alkyl group, an N(C₁₋₆alkyl)₂ group wherein the C₁₋₆alkyl groups do not need to be the same, an NH—C₃₋₆cycloalkyl group, an N(C₁₋₆alkyl)-C₃₋₆cycloalkyl group, an N(C₁₋₆alkyl)-C₂₋₆ alkyl-OR_{c} group, an N(C₁₋₆alkyl)-Y-(5-7 membered cycloheteroalkyl) group, an N(C₁₋₆alkyl)-phenyl group, an N(C₁₋₆alkyl)-Y-(5-7 membered heteroaryl) group, and an N(C₁₋₆alkyl)-C₂₋₆alkyl-O—Y-(5-7 membered heteroaryl) group, wherein each of the phenyl group, the 5-7 membered cycloheteroalkyl group, and the 5-7 membered heteroaryl group immediately above is optionally substituted with 1 to 3 substituents independently selected from a halogen and a C₁₋₆alkyl group, and Y and R_{c} are as defined above. For example, R³ can be a diethylamino group, a diphenylamino group or a cyclopropyl(ethyl)amino group.

In other embodiments, R³ can be an optionally substituted 5-7 membered cycloheteroalkyl group or an optionally substituted 5-7 membered heteroaryl group as described herein. In certain embodiments, R³ can be selected from a diazepanyl group, an imidazolyl group, a morpholinyl group, a piperidinyl group, a piperazinyl group, a pyridyl group, a pyrrolidyl group, an azepanyl group, an azocanyl group, an azepanyl group, and a thiomorpholinyl group, wherein each of these groups can include a nitrogen ring atom optionally substituted with —C(O)R_{c}, —C₂₋₆alkyl-OR_{c}, —C₂₋₆alkyl-NR_{d}Rₑ, —Y—C(O)NR_{d}Rₑ, an —S(O)₂—C₁₋₆alkyl group, a —C₂₋₆alkyl-(5-7 membered cycloheteroalkyl) group, a C₁₋₆alkyl group, or a 5-7 membered heteroaryl group, a carbon ring atom optionally substituted with —C(O)—NR_{d}Rₑ, —Y—OR_{c}, —Y—NR_{d}Rₑ, a —Y-phenyl group, a —Y-(5-7 cycloheteroalkyl) group, a —Y-(5-9 membered heteroaryl) group, or a —Y—O-(5-7 membered heteroaryl) group, and/or a sulfur ring atom optionally substituted with 1 or 2 oxo groups, wherein each of the phenyl groups immediately above is optionally substituted with 1 to 3 substituents independently selected from a halogen, a C₁₋₆alkyl group, a C₁₋₆haloalkyl group, and a C₁₋₆alkoxy group, and each of the 5-7 membered cycloheteroalkyl groups, the 5-7 membered heteroaryl groups, and the 5-9 membered heteroaryl groups immediately above is optionally substituted with 1 to 3 substituents independently selected from a halogen and a C₁₋₆alkyl group, wherein Y, R_{c}, R_{d} and Rₑ are as defined above.

In particular embodiments, R³ can be selected from a 1-[1,4]diazepanyl group, a 1-imidazolyl group, a 4-morpholinyl group, a 1-piperidinyl group, a 1-piperazinyl group, a 4-pyridyl group, a 1-pyrrolidyl group, and a 4-thiomorpholinyl group, wherein each of these groups can be optionally substituted as described above. For example, R³ can be a 1,4-dioxa-8-azaspiro[4.5]dec-8-yl group, a 4-(hydroxymethyl)piperidin-1-yl group, a 3-hydroxypiperidin-1-yl group, a 4-hydroxypiperidin-1-yl group, or a 3-(hydroxymethyl)piperidin-1-yl group.

In some embodiments, R³ can be a 1-piperazinyl group having a nitrogen atom in the ring optionally substituted with —C(O)R_{c}, —C₂₋₆alkyl-OR_{c}, —C₂₋₆alkyl-NR_{d}Rₑ, —C₁₋₆alkyl-C(O)NR_{d}Rₑ, an S(O)₂—C₁₋₆alkyl group, a —C₂₋₆alkyl-(5-7 membered cycloheteroalkyl) group, a C₁₋₆alkyl group, or a 5-7 membered heteroaryl group. For example, R³ can be a 4-methyl piperazin-1-yl group.

In other embodiments, R³ can be a 1-piperidinyl group having a carbon atom in the ring optionally substituted with —NR_{d}Rₑ, —C(O)—NR_{d}Rₑ, —Y—OR_{c}, a 5-7 cycloheteroalkyl group, a 5-9 membered heteroaryl group, or a —Y—O-(5-7 membered heteroaryl) group.

In other embodiments, R³ can be a halogen or a C₁₋₆haloalkyl group. For example, R³ can be a chloro group, an iodo group, a bromo group or a trifluoromethyl group.

In accordance with some embodiments, R³ can be a phenyl group optionally substituted with 1 to 2 substituents independently selected from a halogen, a C₁₋₆haloalkyl group, —OR_{c}, and —C(O)OR_{c}. For example, R³ can be a 4-trifluoromethylphenyl group, a 3-trifluoromethylphenyl group, a 2-trifluoromethylphenyl group, a 2-hydroxylphenyl group, a 3-hydroxylphenyl group, a 4-hydroxylphenyl group, a 2-fluorophenyl group, a 3-fluorophenyl group, or a 4-fluorophenyl group.

In accordance with some embodiments, p is 1.

In some embodiments, p is 2.

In certain embodiments, p is 3.

In certain preferred embodiments, Ar—R³ can be

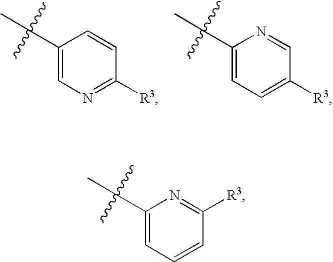

wherein R³ is as defined above.

In accordance with some preferred embodiments, Ar—R³ can be

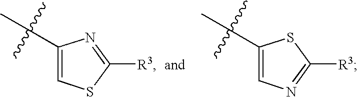

wherein R³ is as defined above.

Representative compounds of formula (I) in accordance with embodiments of the present invention include, but are not limited to, the compounds presented in Table 1 below.

TABLE 1

| No. | Structure | Chemical Name |
|---|---|---|
| 1 | 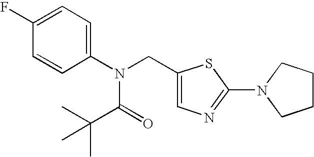 | N-(4-Fluorophenyl)-2,2-dimethyl-N-(2- pyrrolidin-1-yl-thiazol-5-ylmethyl)- propionamide |
| 2 | 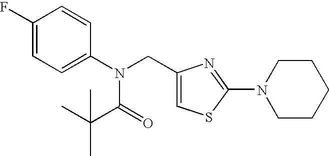 | N-(4-Fluorophenyl)-2,2-dimethyl-N-(2- piperidin-1-yl-thiazol-4-ylmethyl)-propionamide |
| 3 | 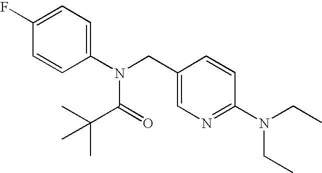 | N-(6-Diethylamino-pyridin-3-ylmethyl)-N-(4- fluorophenyl)-2,2-dimethyl-propionamide |
| 4 | 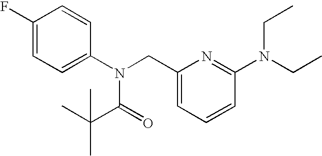 | N-(6-Diethylamino-pyridin-2-ylmethyl)-N-(4- fluorophenyl)-2,2-dimethyl-propionamide |
| 5 | 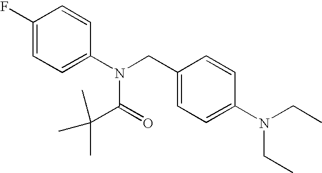 | N-(4-Diethylaminobenzyl)-N-(4-fluorophenyl)- 2,2-dimethyl-propionamide |
| 6 | 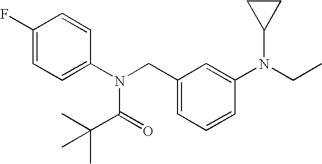 | N-[3-(Cyclopropyl(ethyl)amino)-benzyl]-N-(4- fluorophenyl)-2,2-dimethyl-propionamide |
| 7 | 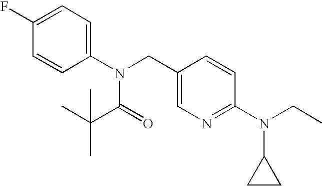 | N-[6-(Cyclopropyl(ethyl)amino)-pyridin-3- ylmethyl]-N-(4-fluorophenyl)-2,2-dimethyl- propionamide |
| 8 | 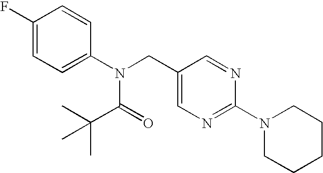 | N-(4-Fluorophenyl)-2,2-dimethyl-N-(2- piperidin-1-yl-pyrimidin-5-ylmethyl)- propionamide |
| 11 | 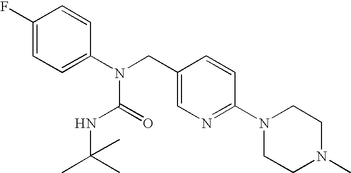 | 3-tert-Butyl-1-(4-fluorophenyl)-1-[6-(4-methyl- piperazin-1-yl)-pyridin-3-ylmethyl]-urea |
| 12 | 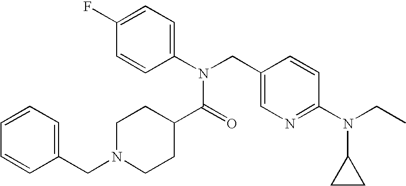 | 1-Benzyl-piperidine-4-carboxylic acid [6- (Cyclopropyl(ethyl)amino)-pyridin-3-ylmethyl]- (4-fluorophenyl)-amide |
| 13 | 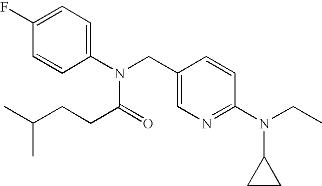 | 4-Methyl-pentanoic acid [6- (Cyclopropyl(ethyl)amino)-pyridin-3-ylmethyl]- (4-fluorophenyl)-amide |
| 14 | 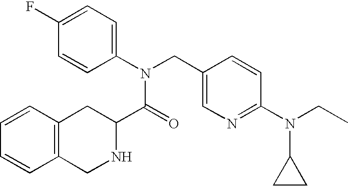 | 1,2,3,4-Tetrahydro-isoquinoline-3-carboxylic acid [6-(Cyclopropyl(ethyl)amino)-pyridin-3- ylmethyl]-(4-fluorophenyl)-amide |
| 15 | 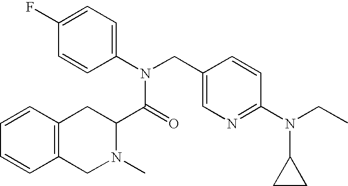 | 2-Methyl-1,2,3,4-tetrahydro-isoquinoline-3- carboxylic acid [6-(Cyclopropyl(ethyl)amino)- pyridin-3-ylmethyl]-(4-fluorophenyl)-amide |
| 16 | 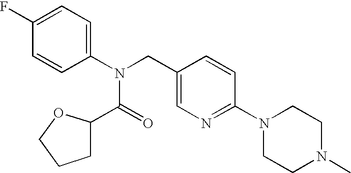 | Tetrahydro-furan-2-carboxylic acid (4- fluorophenyl)-[6-(4-methyl-piperazin-1-yl)- pyridin-3-ylmethyl]-amide |
| 17 | 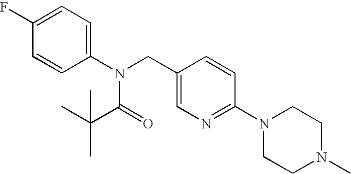 | N-(4-Fluorophenyl)-N-[6-(4-methyl-piperazin-1- yl)-pyridin-3-ylmethyl)-2,2-dimethyl- propionamide |
| 18 | 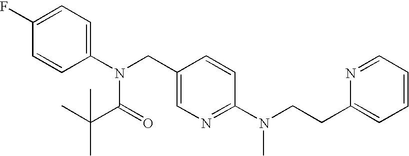 | N-(4-Fluorophenyl)-2,2-dimethyl-N-{6-[methyl- (2-pyridin-2-yl-ethyl)-amino]-pyridin-3- ylmethyl}-propionamide |
| 20 | 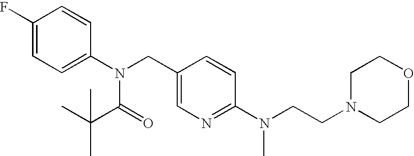 | N-(4-Fluorophenyl)-2,2-dimethyl-N-{6-[methyl- (2-morpholin-4-yl-ethyl)-amino]-pyridin-3- ylmethyl}-propionamide |
| 21 | 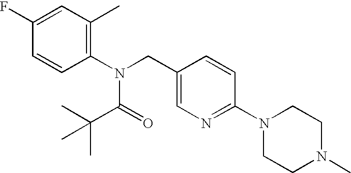 | N-(4-Fluoro-2-methyl-phenyl)-2,2-dimethyl-N- [6-(4-methyl-piperazin-1-yl)-pyridin-3-ylmethyl]- propionamide |
| 22 | 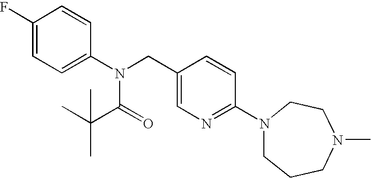 | N-(4-Fluorophenyl)-2,2-dimethyl-N-[6-(4- methyl-[1,4]diazepan-1-yl)-pyridin-3-ylmethyl]- propionamide |
| 23 | 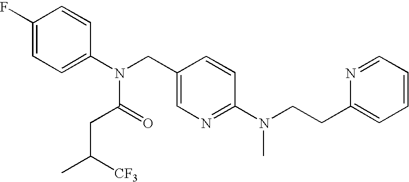 | 4,4,4-Trifluoro-N-(4-fluorophenyl)-3-methyl-N- {6-[methyl-(2-pyridin-2-yl-ethyl)-amino]-pyridin- 3-ylmethyl}-butyramide |
| 24 | 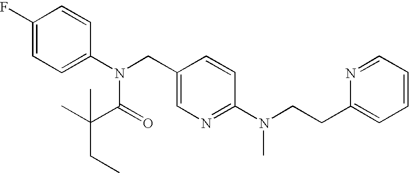 | N-(4-Fluorophenyl)-2,2-dimethyl-N-{6-[methyl- (2-pyridin-2-yl-ethyl)-amino]-pyridin-3- ylmethyl}-butyramide |
| 25 | 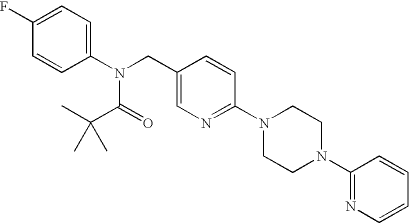 | N-(4-Fluorophenyl)-2,2-dimethyl-N-[6-(4- pyridin-2-yl-piperazin-1-yl)-pyridin-3-ylmethyl]- propionamide |
| 26 | 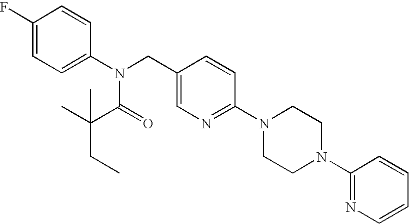 | N-(4-Fluorophenyl)-2,2-dimethyl-N-[6-(4- pyridin-2-yl-piperazin-1-yl)-pyridin-3-ylmethyl]- butyamide |
| 27 | 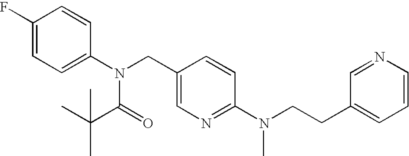 | N-(4-Fluorophenyl)-2,2-dimethyl-N-{6-[methyl- (2-pyridin-3-yl-ethyl)-amino]-pyridin-3- ylmethyl}-propionamide |
| 29 | 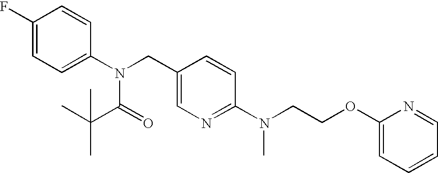 | N-(4-Fluorophenyl)-2,2-dimethyl-N-(6-{methyl- [2-(pyridin-2-yloxy)-ethyl]-amino}-pyridin-3- ylmethyl)-propanamide |
| 30 | 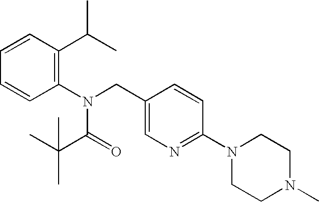 | N-(2-Isopropyl-phenyl)-2,2-dimethyl-N-[6-(4- methyl-piperazin-1-yl)-pyridin-3-ylmethyl]- propionamide |
| 31 | 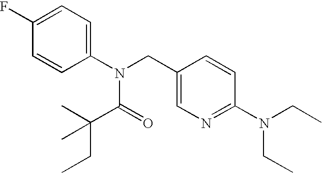 | N-(6-Diethylamino-pyridin-3-ylmethyl)-N-(4- fluorophenyl)-2,2-dimethyl-butyramide |
| 32 | 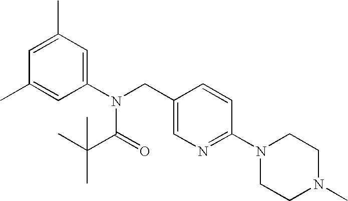 | N-(3,5-dimethylphenyl)-2,2-dimethyl-N-[6-(4- methyl-piperazin-1-yl)-pyridin-3-ylmethyl]- propionamide |
| 33 | 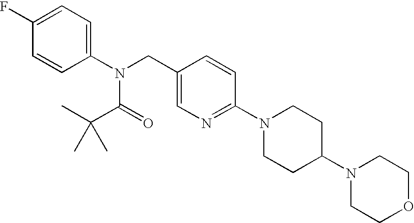 | N-(4-Fluorophenyl)-2,2-dimethyl-N-(4- morpholin-4-yl-3,4,5,6-tetrahydro-2H- [1,2′]bipyridinyl-5′-ylmethyl)-propionamide |
| 34 | 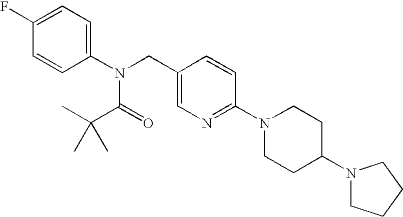 | N-(4-Fluorophenyl)-2,2-dimethyl-N-(4- pyrrolidin-1-yl-3,4,5,6-tetrahydro-2H- [1,2′]bipyridinyl-5′-ylmethyl)-propionamide |
| 35 | 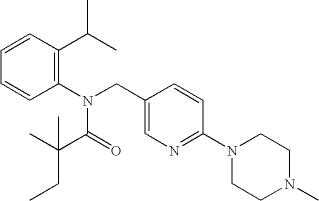 | N-(2-Isopropyl-phenyl)-2,2-dimethyl-N-[6-(4- methyl-piperazin-1-yl)-pyridin-3-ylmethyl]- butyramide |
| 36 | 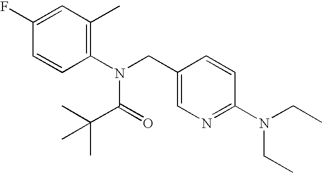 | N-(6-Diethylamino-pyridin-3-ylmethyl)-N-(4- fluoro-2-methyl-phenyl)-2,2-dimethyl- propionamide |
| 37 | 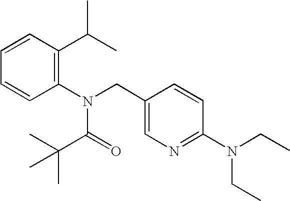 | N-(6-Diethylamino-pyridin-3-ylmethyl)-N-(2- isopropyl-phenyl)-2,2-dimethyl-propionamide |
| 38 | 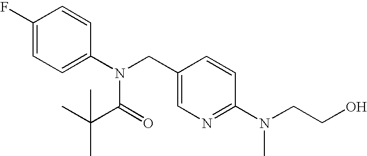 | N-(4-Fluorophenyl)-N-{6-[(2-hydroxy-ethyl)- methyl-amino]-pyridin-3-ylmethyl}-2,2- dimethyl-propionamide |
| 39 | 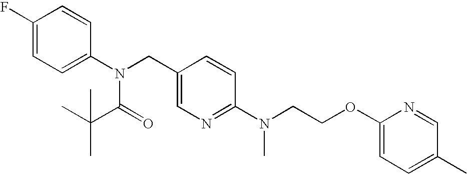 | N-(4-Fluorophenyl)-2,2-dimethyl-N-(6-{methyl- [2-(5-methyl-pyridin-2-yloxy)-ethyl]-amino}- pyridin-3-ylmethyl)-propionamide |
| 40 | 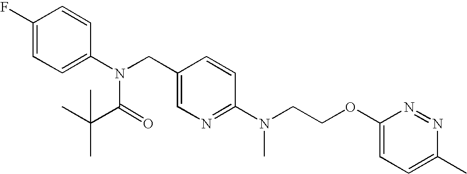 | N-(4-Fluorophenyl)-2,2-dimethyl-N-(6-{methyl- [2-(6-methyl-pyridazin-3-yloxy)-ethyl]-amino}- pyridin-3-ylmethyl)-propionamide |
| 41 | 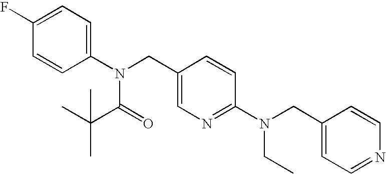 | N-[6-(Ethyl-pyridin-4-ylmethyl-amino)-pyridin- 3-ylmethyl]-N-(4-fluorophenyl)-2,2-dimethyl- propionamide |
| 42 | 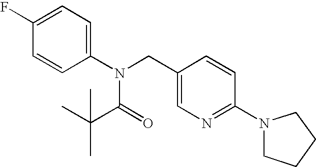 | N-(4-Fluorophenyl)-2,2-dimethyl-N-(6- pyrrolidin-1-yl-pyridin-3-ylmethyl)- propionamide |
| 43 | 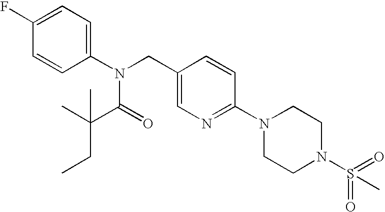 | N-(4-Fluorophenyl)-N-[6-(4-methanesulfonyl- piperazin-1-yl)-pyridin-3-ylmethyl]-2,2- dimethyl-butyramide |
| 44 | 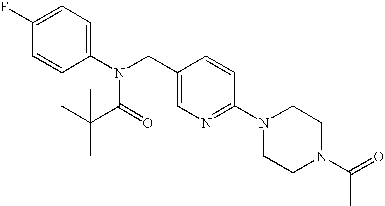 | N-[6-(4-Acetyl-piperazin-1-yl)-pyridin-3- ylmethyl]-N-(4-fluorophenyl)-2,2-dimethyl- propionamide |
| 45 | 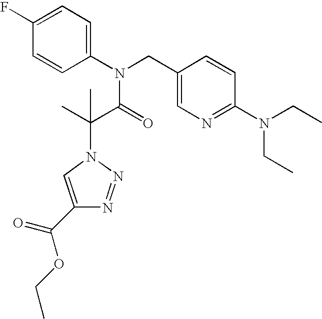 | 1-{1-[(6-Diethylamino-pyridin-3-ylmethyl)-(4- fluorophenyl)-carbamoyl]-1-methyl-ethyl}-1H- [1,2,3]triazole-4-carboxylic acid ethyl ester |
| 47 | 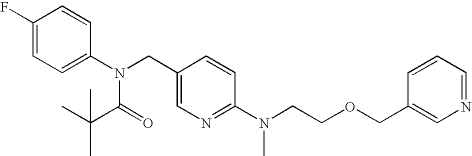 | N-(4-Fluorophenyl)-2,2-dimethyl-N-(6-{methyl- [2-(pyridin-3-ylmethoxy)-ethyl]-amino}-pyridin- 3-ylmethyl)-propionamide |
| 48 | 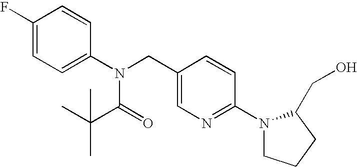 | N-(4-Fluorophenyl)-N-[6-(2-hydroxymethyl- pyrrolidin-1-yl)-pyridin-3-ylmethyl]-2,2- dimethyl-propionamide |
| 49 | 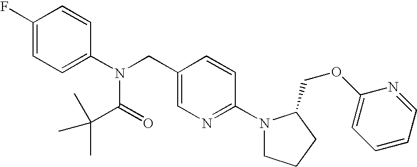 | N-(4-Fluorophenyl)-2,2-dimethyl-N-{6-[2- (pyridin-2-yloxymethyl)-pyrrolidin-1-yl]-pyridin- 3-ylmethyl}-propionamide |
| 50 | 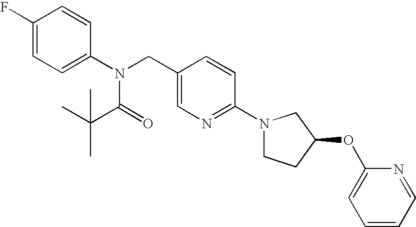 | N-(4-Fluorophenyl)-2,2-dimethyl-N-{6-[3- (pyridin-2-yloxy)-pyrrolidin-1-yl]-pyridin-3- ylmethyl}-propionamide |
| 51 | 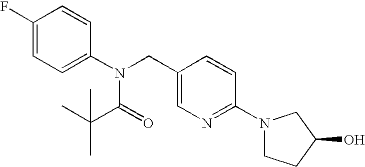 | N-(4-Fluorophenyl)-N-[6-(3-hydroxy-pyrrolidin- 1-yl)-pyridin-3-ylmethyl]-2,2-dimethyl- propionamide |
| 52 | 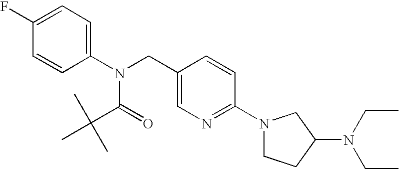 | N-[6-(3-Diethylamino-pyrrolidin-1-yl)-pyridin-3- ylmethyl]-N-(4-fluorophenyl)-2,2-dimethyl- propionamide |
| 53 | 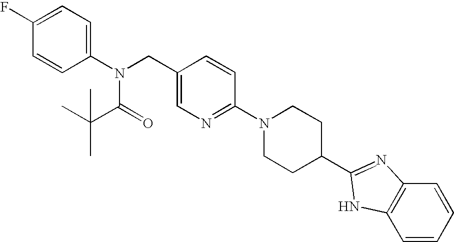 | N-[4-(1H-Benzoimidazol-2-yl)-3,4,5,6- tetrahydro-2H-[1,2′]bipyridinyl-5′-ylmethyl]-N- (4-fluorophenyl)-2,2-dimethyl-propionamide |
| 54 | 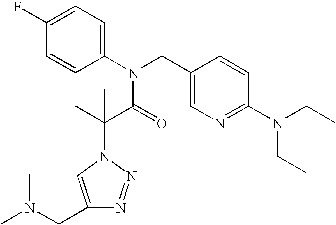 | N-(6-Diethylamino-pyridin-3-ylmethyl)-2-(4- dimethylaminomethyl-[1,2,3]triazol-1-yl)-N-(4- fluorophenyl)-isobutyramide |
| 55 | 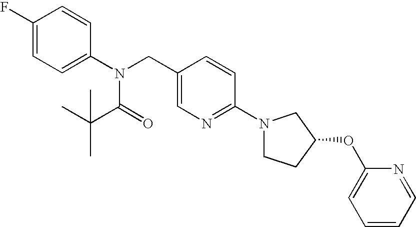 | N-(4-Fluorophenyl)-2,2-dimethyl-N-{6-[3- (pyridin-2-yloxy)-pyrroldiin-1-yl]-pyridin-3- ylmethyl}-propionamide |
| 56 | 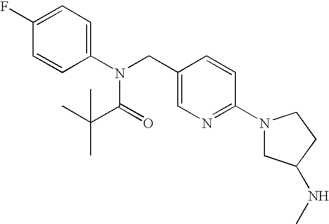 | N-(4-Fluorophenyl)-2,2-dimethyl-N-[6-(3- methylamino-pyrrolidin-1-yl)-pyridin-3- ylmethyl]-propionamide |
| 57 | 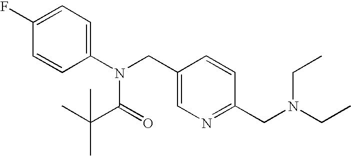 | N-(6-Diethylaminomethyl-pyridin-3-ylmethyl)- N-(4-fluorophenyl)-2,2-dimethyl-propionamide |
| 58 | 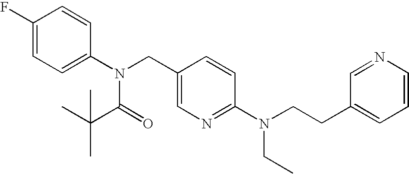 | N-{6-[Ethyl-(2-pyridin-3-yl-ethyl)-amino]- pyridin-3-ylmethyl}-N-(4-fluorophenyl)-2,2- dimethyl-propionamide |
| 59 | 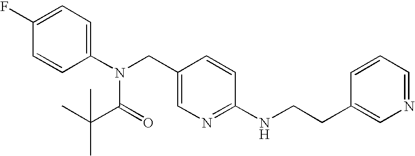 | N-(4-Fluorophenyl)-2,2-dimethyl-N-[6-(2- pyridin-3-yl-ethylamino)-pyridin-3-ylmethyl]- propionamide |
| 60 | 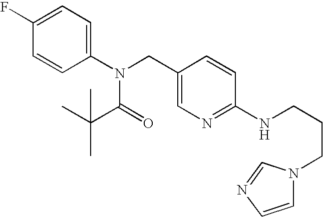 | N-(4-Fluorophenyl)-N-[6-(3-imidazol-1-yl- propylamino)-pyridin-3-ylmethyl]-2,2-dimethyl- propionamide |
| 61 | 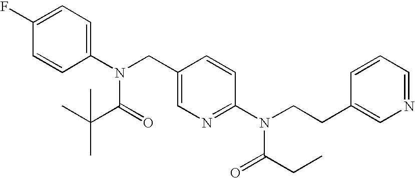 | N-(4-Fluorophenyl)-2,2-dimethyl-N-{6- [propionyl-(2-pyridin-3-yl-ethyl)-amino]-pyridin- 3-ylmethyl}-propionamide |
| 62 | 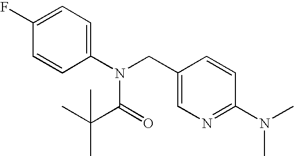 | N-(6-Dimethylamino-pyridin-3-ylmethyl)-N-(4- fluorophenyl)-2,2-dimethyl-propionamide |
| 63 | 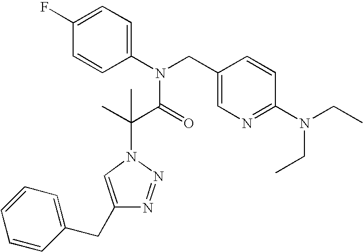 | 2-(4-Benzyl-[1,2,3]triazol-1-yl)-N-(6- diethylamino-pyridin-3-ylmethyl)-N-(4- fluorophenyl)-isobutyramide |
| 64 | 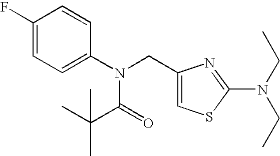 | N-(2-Diethylamino-thiazol-4-ylmethyl)-N-(4- fluorophenyl)-2,2-dimethyl-propionamide |
| 65 | 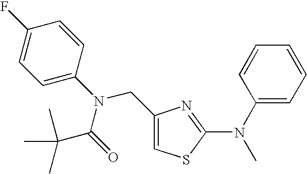 | N-(4-Fluorophenyl)-2,2-dimethyl-N-[2-(methyl- phenyl-amino)-thiazol-4-ylmethyl]- propionamide |
| 66 | 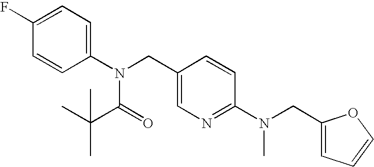 | N-(4-Fluorophenyl)-N-[6-(furan-2-ylmethyl- methyl-amino)-pyridin-3-ylmethyl]-2,2- dimethyl-propionamide |
| 67 | 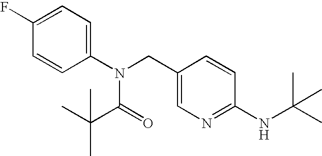 | N-(6-tert-Butylamino-pyridin-3-ylmethyl)-N-(4- fluorophenyl)-2,2-dimethyl-propionamide |
| 68 | 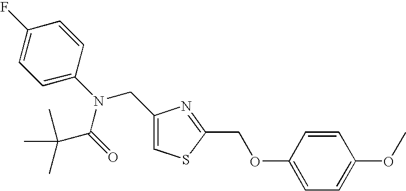 | N-(4-Fluorophenyl)-N-[2-(4-methoxy- phenoxymethyl)-thiazol-4-ylmethyl]-2,2- dimethyl-propionamide |
| 69 | 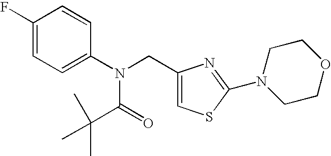 | N-(4-Fluorophenyl)-2,2-dimethyl-N-(2- morpholin-4-yl-thiazol-4-ylmethyl)- propionamide |
| 70 | 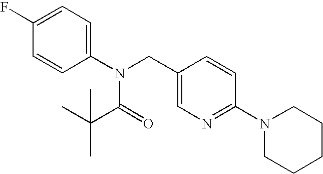 | N-(4-Fluorophenyl)-2,2-dimethyl-N-(3,4,5,6- tetrahydro-2H-[1,2′]bipyridinyl-5′-ylmethyl)- propionamide |
| 71 | 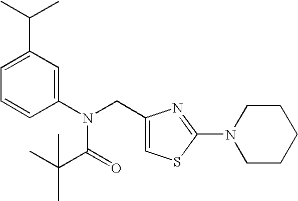 | N-(3-Isopropyl-phenyl)-2,2-dimethyl-N-(2- piperidin-1-yl-thiazol-4-ylmethyl)-propionamide |
| 72 | 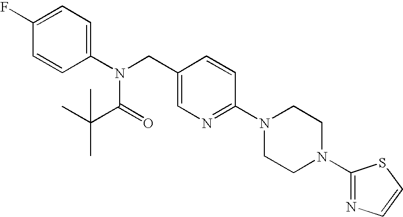 | N-(4-Fluorophenyl)-2,2-dimethyl-N-[6-(4- thiazol-2-yl-piperazin-1-yl)-pyridin-3-ylmethyl]- propionamide |
| 73 | 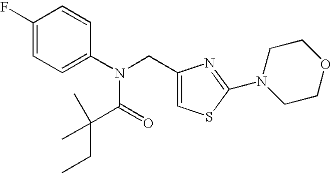 | N-(4-Fluorophenyl)-2,2-dimethyl-N-(2- morpholin-4-yl-thiazol-4-ylmethyl)-butyramide |
| 74 | 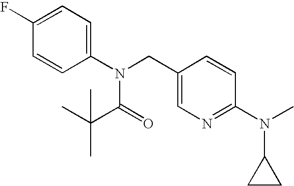 | N-[6-(Cyclopropyl-methyl-amino)-pyridin-3- ylmethyl]-N-(4-fluorophenyl)-2,2-dimethyl- propionamide |
| 75 | 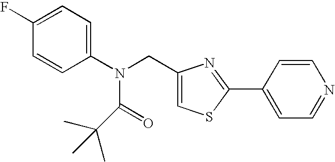 | N-(4-Fluorophenyl)-2,2-dimethyl-N-(2-pyridin- 4-yl-thiazol-4-ylmethyl)-propionamide |
| 76 | 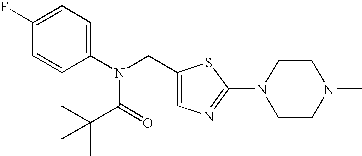 | N-(4-Fluorophenyl)-2,2-dimethyl-N-[2-(4- methyl-piperazin-1-yl)-thiazol-5-ylmethyl]- propionamide |
| 77 | 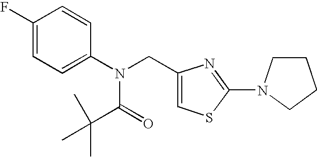 | N-(4-Fluorophenyl)-2,2-dimethyl-N-(2- pyrrolidin-1-yl-thiazol-4-ylmethyl)- propionamide |
| 78 | 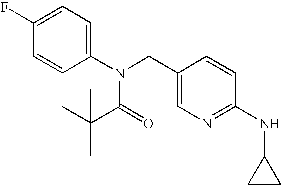 | N-(6-Cyclopropylamino-pyridin-3-ylmethyl)-N- (4-fluorophenyl)-2,2-dimethyl-propionamide |
| 79 | 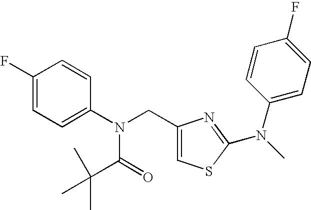 | N-(4-Fluorophenyl)-N-{2-[(4-fluorophenyl)- methyl-amino]-thiazol-4-ylmethyl}-2,2- dimethyl-propionamide |
| 80 | 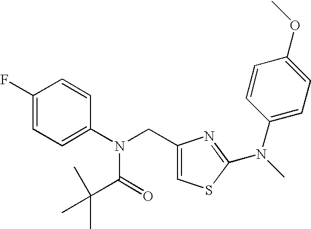 | N-(4-Fluorophenyl)-N-{2-[(4-methoxy-phenyl)- methyl-amino]-thiazol-4-ylmethyl}-2,2- dimethyl-propionamide |
| 81 | 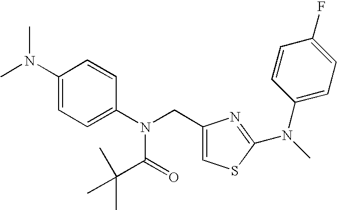 | N-(4-Dimethylamino-phenyl)-N-{2-[(4- fluorophenyl)-methyl-amino]-thiazol-4- ylmethyl}-2,2-dimethyl-propionamide |
| 82 | 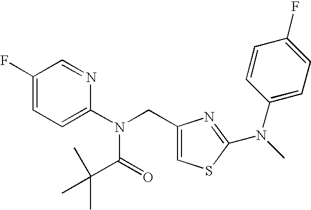 | N-{2-[(4-Fluorophenyl)-methyl-amino]-thiazol- 4-ylmethyl}-N-(5-fluoro-pyridin-2-yl)-2,2- dimethyl-propionamide |
| 83 | 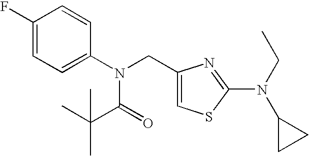 | N-[2-(Cyclopropyl(ethyl)amino)-thiazol-4- ylmethyl]-N-(4-fluorophenyl)-2,2-dimethyl- propionamide |
| 84 | 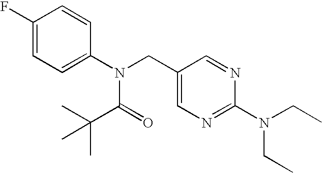 | N-(2-Diethylamino-pyrimidin-5-ylmethyl)-N-(4- fluorophenyl)-2,2-dimethyl-propionamide |
| 85 | 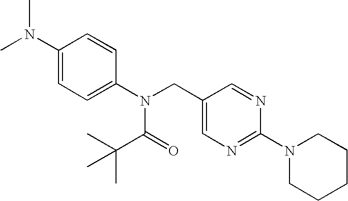 | N-(4-Dimethylamino-phenyl)-2,2-dimethyl-N- (2-piperidin-1-yl-pyrimidin-5-ylmethyl)- propionamide |
| 86 | 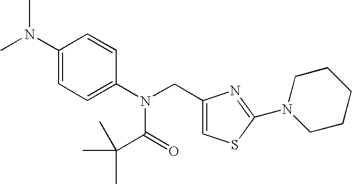 | N-(4-Dimethylamino-phenyl)-2,2-dimethyl-N- (2-piperidin-1-yl-thiazol-4-ylmethyl)- propionamide |
| 87 | 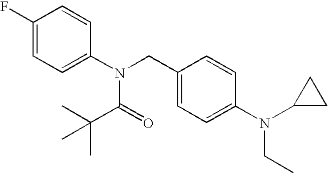 | N-[4-(Cyclopropyl(ethyl)amino)-benzyl]-N-(4- fluorophenyl)-2,2-dimethyl-propionamide |
| 88 | 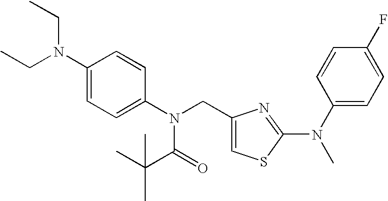 | N-(4-Diethylamino-phenyl)-N-{2-[(4- fluorophenyl)-methyl-amino]-thiazol-4- ylmethyl}-2,2-dimethyl-propionamide |
| 89 | 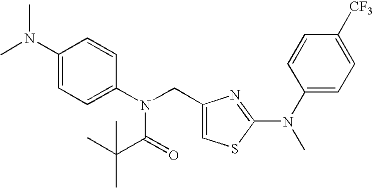 | N-(4-Dimethylamino-phenyl)-2,2-dimethyl-N- {2-[methyl-(4-trifluoromethyl-phenyl)-amino]- thiazol-4-ylmethyl}-propionamide |
| 90 | 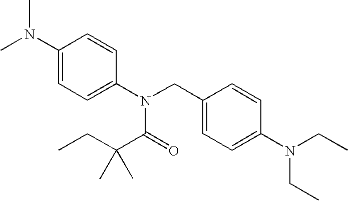 | N-(4-Diethylaminobenzyl)-N-(4-dimethylamino- phenyl)-2,2-dimethyl-butyramide |
| 91 | 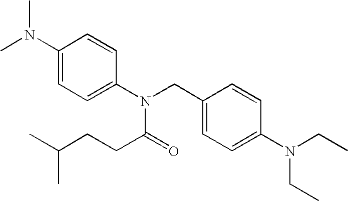 | 4-Methyl-pentanoic acid (4- Diethylaminobenzyl)-(4-dimethylamino- phenyl)-amide |
| 92 | 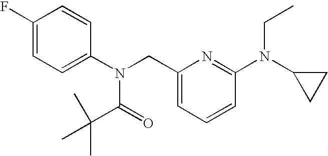 | N-[6-(Cyclopropyl(ethyl)amino)-pyridin-2- ylmethyl]-N-(4-fluorophenyl)-2,2-dimethyl- propionamide |
| 93 | 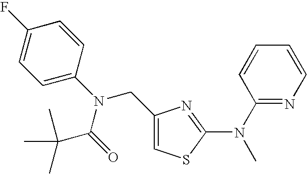 | N-(4-Fluorophenyl)-2,2-dimethyl-N-[2-(methyl- pyridin-2-yl-amino)-thiazol-4-ylmethyl]- propionamide |
| 94 | 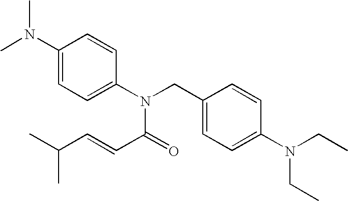 | 4-Methyl-pent-2-enoic acid (4- Diethylaminobenzyl)-(4-dimethylamino- phenyl)-amide |
| 95 | 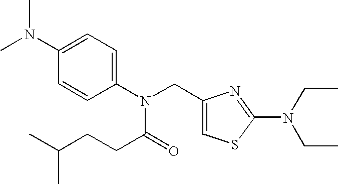 | 4-Methyl-pentanoic acid (2-diethylamino- thiazol-4-ylmethyl)-(4-dimethylamino-phenyl)- amide |
| 96 | 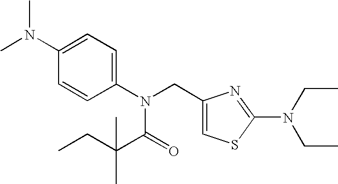 | N-(2-Diethylamino-thiazol-4-ylmethyl)-N-(4- dimethyalmino-phenyl)-2,2-dimethyl- butyramide |
| 97 | 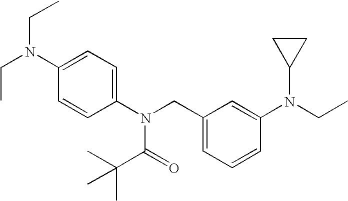 | N-[3-(Cyclopropyl(ethyl)amino)-benzyl]-N-(4- diethylamino-phenyl)-2,2-dimethyl- propionamide |
| 98 | 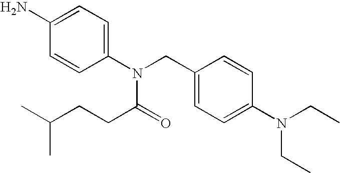 | 4-Methyl-pentanoic acid (4-aminophenyl)-(4- Diethylaminobenzyl)-amide |
| 99 | 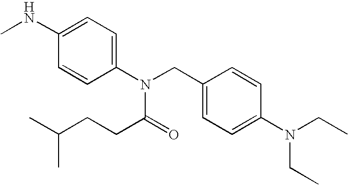 | 4-Methyl-pentanoic acid (4- Diethylaminobenzyl)-(4-methylamino-phenyl)- amide |
| 100 | 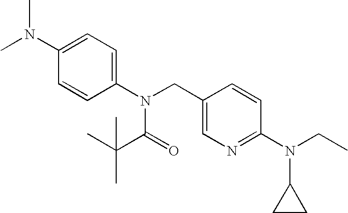 | N-[6-(Cycloproyl(ethyl)amino)-pyridin-3- ylmethyl]-N-(4-dimethylamino-phenyl)-2,2- dimethyl-propionamide |
| 101 | 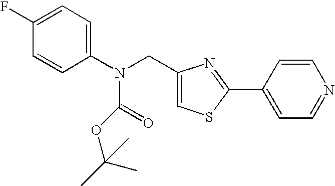 | (4-Fluorophenyl)-(2-pyridin-4-yl-thiazol-4- ylmethyl)-carabamic acid tert-butyl ester |
| 102 | 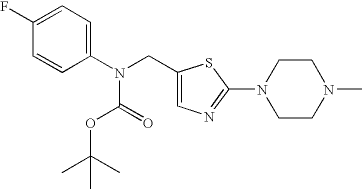 | (4-Fluorophenyl)-[2-(4-methyl-piperazin-1-yl)- thiazol-5-ylmethyl]-carbamic acid tert-butyl ester |
| 103 | 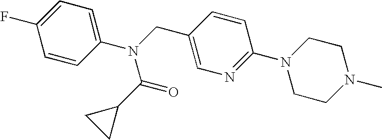 | Cyclopropanecarboxylic acid (4-fluorophenyl)- [6-(4-methyl-piperaizn-1-yl)-pyridin-3-ylmethyl]- amide |
| 104 | 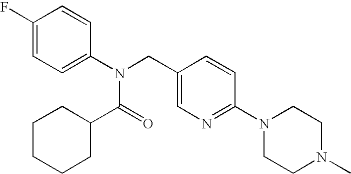 | Cyclohexanecarboxylic acid (4-fluorophenyl)- [6-(4-methyl-piperazin-1-yl)-pyridin-3-ylmethyl]- amide |
| 106 | 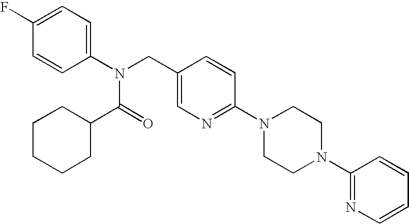 | Cyclohexanecarboxylic acid (4-fluorophenyl)- [6-(4-pyridin-2-yl-piperazin-1-yl)-pyridin-3- ylmethyl]-amide |
| 107 | 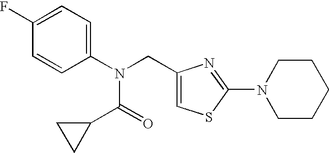 | Cyclopropanecarboxylic acid (4-fluorophenyl)- (2-piperidin-1-yl-thiazol-4-ylmethyl)-amide |
| 108 | 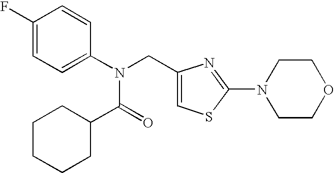 | Cyclohexanecarboxylic acid (4-fluorophenyl)- (2-morpholin-4-yl-thiazol-4-ylmethyl)-amide |
| 109 | 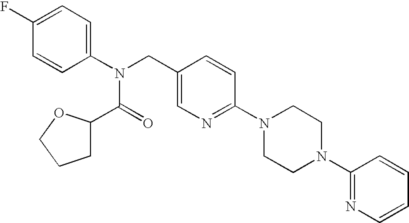 | Tetrahydro-furan-2-carboxylic acid (4- fluorophenyl)-[6-(4-pyridin-2-yl-piperazin-1-yl)- pyridin-3-ylmethyl]-amide |
| 110 | 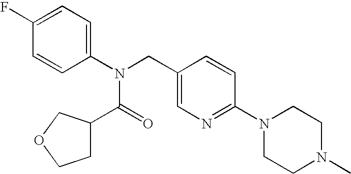 | N-(4-fluorophenyl)-N-{[6-(4-methylpiperazin-1- yl)pyridin-3-yl]methyl}tetrahydrofuran-2- carboxamide |
| 111 | 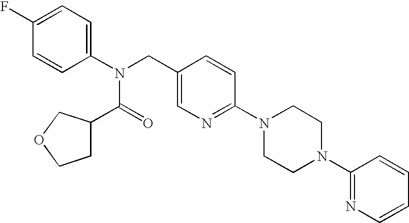 | Tetrahydro-furan-3-carboxylic acid (4- fluorophenyl)-[6-(4-pyridin-2-yl-piperazin-1-yl)- pyridin-3-ylmethyl]-amide |
| 112 | 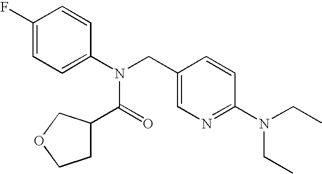 | Tetrahydro-furan-3-carboxylic acid (6- diethylamino-pyridin-3-ylmethyl)-(4- fluorophenyl)-amide |
| 113 | 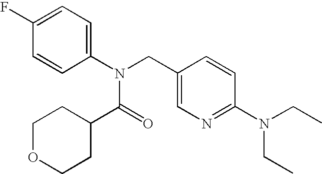 | Tetrahydro-pyran-4-carboxylic acid (6- diethylamino-pyridin-3-ylmethyl)-(4- fluorophenyl)-amide |
| 114 | 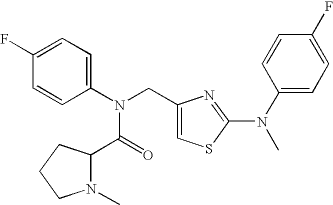 | 1-Methyl-pyrrolidine-2-carboxylic acid (4- fluroophenyl)-{2-[(4-fluorophenyl)-methyl- amino]-thiazol-4-ylmethyl]-amide |
| 115 | 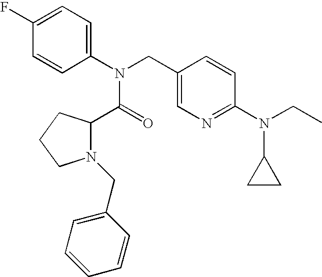 | 1-Benzyl-pyrrolidine-2-carboxylic acid [6- (Cyclopropyl(ethyl)amino)-pyridin-3-ylmethyl]- (4-fluorophenyl)-amide |
| 116 | 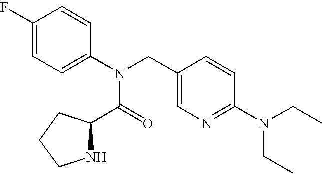 | N-{[6-(diethylamino)pyridin-3-yl]methyl}-N-(4- fluroophenyl)-L-prolinamide |
| 117 | 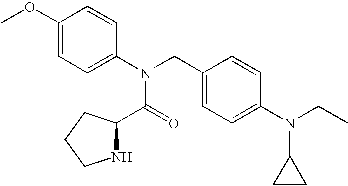 | N-{4-[cyclopropyl(ethyl)amino]benzyl}-N-(4- methoxyphenyl)-L-prolinamide |
| 118 | 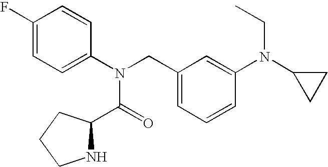 | N-{3-[cyclopropyl(ethyl)amino]benzyl}-N-(4- fluorophenyl)-L-prolinamide |
| 119 | 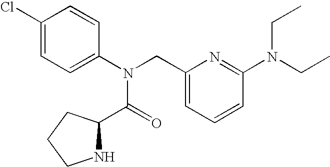 | N-(4-chlorophenyl)-N-{[6-(diethylamino)pyridin-2- yl]methyl}-L-prolinamide |
| 120 | 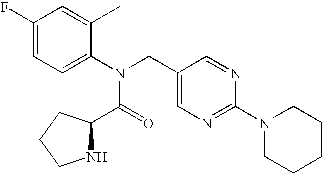 | N-(4-fluoro-2-methylphenyl)-N-[(2-piperidin-1- ylpyrimidin-5-yl)methyl]-L-prolinamide |
| 121 | 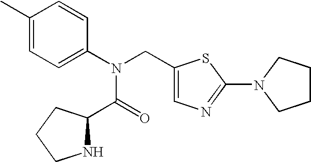 | N-(4-methylphenyl)-N-[(2-pyrrolidin-1-yl-1,3- thiazol-5-yl)methyl]-L-prolinamide |
| 122 | 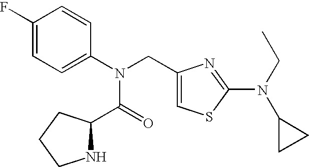 | N-({2-[cyclopropyl(ethyl)amino]-1,3-thiazol-4- yl}methyl)-N-(4-fluorophenyl)-L-prolinamide |
| 123 | 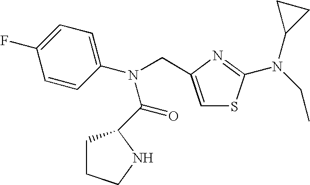 | N-({2-[cyclopropyl(ethyl)amino]-1,3-thiazol-4- yl}methyl)-N-(4-fluorophenyl)-D-prolinamide |
| 124 | 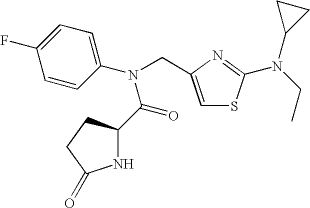 | N-({2-[cyclopropyl(ethyl)amino]-1,3-thiazol-4- yl}methyl)-N-(4-fluorophenyl)-5-oxo-L- prolinamide |
| 125 | 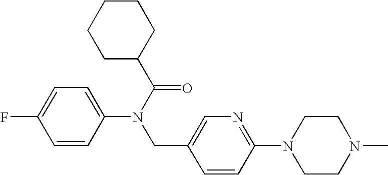 | N-(4-fluorophenyl)-N-{[6-(4-methylpiperazin-1- yl)pyridin-3-yl]methyl}cyclohexanecarboxamide |
| 126 | 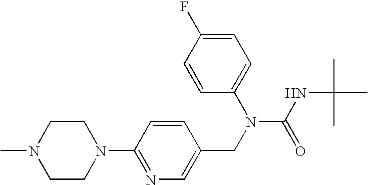 | N′-(tert-butyl)-N-(4-fluorophenyl)-N-{[6-(4- methylpiperazin-1-yl)pyridin-3-yl]methyl}urea |
| 127 | 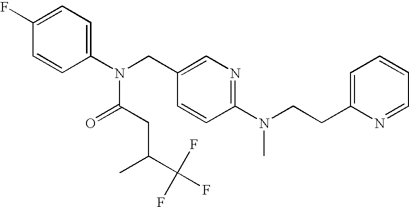 | 4,4,4-trifluoro-N-(4-fluorophenyl)-3-methyl-N-({(6- [methyl(2-pyridin-2-ylethyl)amino]pyridin-3- yl}methyl)butanamide |
| 128 | 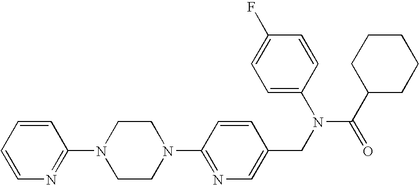 | N-(4-fluorophenyl)-N-{[6-(4-pyridin-2-ylpiperazin- 1-yl)pyridin-3-yl]methyl}cyclohexanecarboxamide |
| 129 | 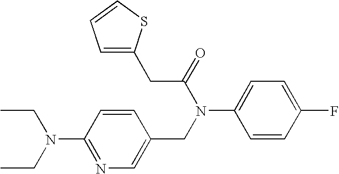 | N-{[(6-(diethylamino)pyridin-3-yl]methyl}-N-(4- fluorophenyl)-2-(2-thienyl)acetamide |
| 130 | 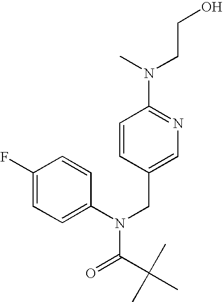 | N-(4-fluorophenyl)-N-({6-[(2- hydroxyethyl)(methyl)amino]pyridin-3-yl}methyl)- 2,2-dimethylpropanamide |
| 131 | 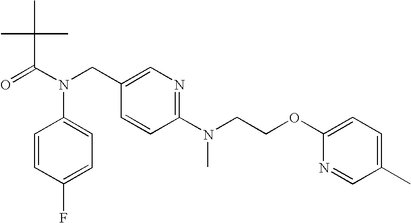 | N-(4-fluorophenyl)-2,2-dimethyl-N-{[6-(methyl{2- [(5-methylpyridin-2-yl)oxy]ethyl}amino)pyridin-3- yl]methyl}propanamide |
| 132 | 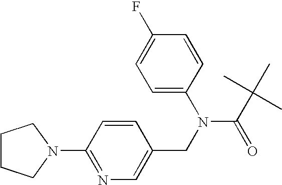 | N-(4-fluorophenyl)-2,2-dimethyl-N-[(6-pyrrolidin- 1-ylpyridin-3-yl)methyl]propanamide |
| 133 | 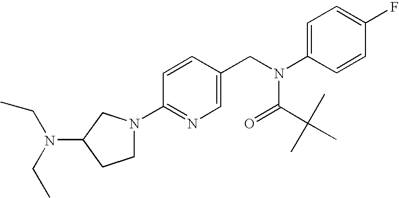 | N-({6-[3-(diethylamino)pyrrolidin-1-yl]pyridin-3- yl}methyl)-N-(4-fluorophenyl)-2,2- dimethylpropanamide |
| 134 | 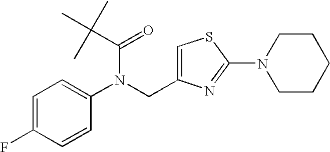 | N-(4-fluorophenyl)-2,2-dimethyl-N-[(2-piperidin-1- yl-1,3-thiazol-4-yl)methyl]propanamide |
| 135 | 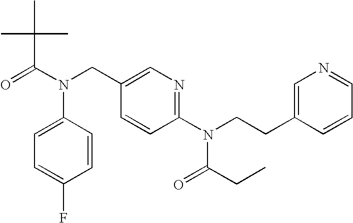 | N-(4-fluorophenyl)-2,2-dimethyl-N-({6- [propionyl)2-pyridin-3-ylethyl)amino]pyridin-3- yl}methyl)propanamide |
| 136 | 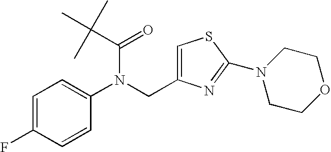 | N-(4-fluorophenyl)-2,2-dimethyl-N-[(2-morpholin- 4-yl-1,3-thiazol-4-yl)methyl]propanamide |
| 137 | 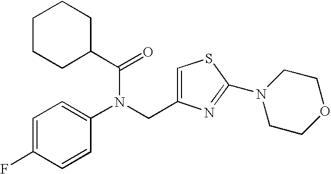 | N-(4-fluorophenyl)-N-[(2-morpholin-4-yl-1,3- thiazol-4-yl)methyl]cyclohexanecarboxamide |
| 138 | 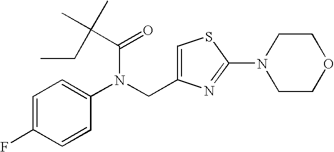 | N-(4-fluorophenyl)-2,2-dimethyl-N-[(2-morpholin- 4-yl-1,3-thiazol-4-yl)methyl]butanamide |
| 139 | 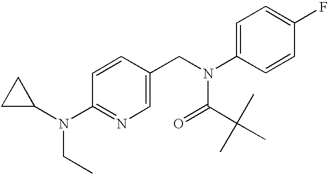 | N-({6-[cyclopropyl(ethyl)amino]pyridin-3- yl}methyl)-N-(4-fluorophenyl)-2,2- dimethylpropanamide |
| 140 | 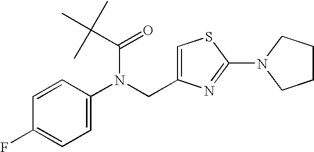 | N-(4-fluorophenyl)-2,2-dimethyl-N-[(2-pyrrolidin- 1-yl-1,3-thiazol-4-yl)methyl]propanamide |
| 141 | 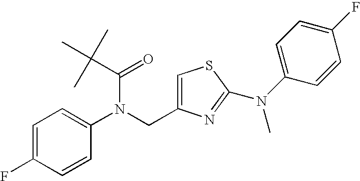 | N-(4-fluorophenyl)-N-({2-[(4- fluorophenyl)(methyl)amino]-1,3-thiazol-4- yl}methyl)-2,2-dimethylpropanamide |
| 142 | 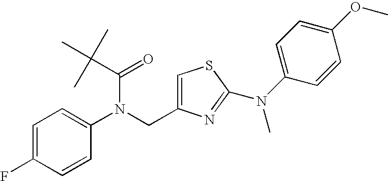 | N-(4-fluroophenyl)-N-({2-[(4- methoxyphenyl)(methyl)amino]-1,3-thiazol-4- yl}methyl)-2,2-dimethylpropanamide |
| 143 | 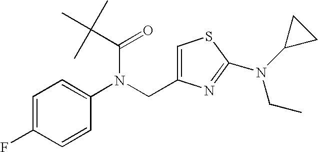 | N-({2-[cyclopropyl(ethyl)amino]-1,3-thiazol-4- yl}methyl)-N-(4-fluorophenyl)-2,2- dimethylpropanamide |
| 144 | 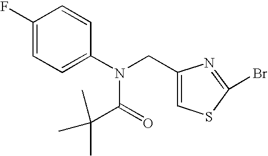 | N-[(2-bromo-1,3-thiazol-4-yl)methyl]-N-(4- fluorophenyl)-2,2-dimethylpropanamide |
| 145 | 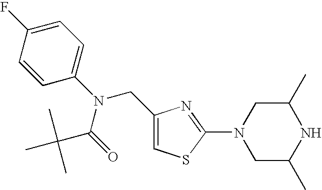 | N-{[2-(3,5-dimethylpiperazin-1-yl)-1,3-thiazol-4- yl]methyl}-N-(4-fluorophenyl)-2,2- dimethylpropanamide |
| 146 | 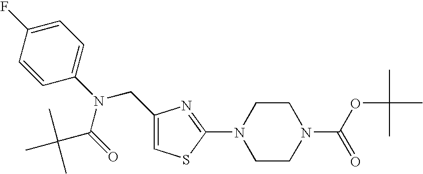 | tert-butyl 4-(4-{[(2,2-dimethylpropanoyl)(4- fluorophenyl)amino]methyl}--1,3-thiazol-2- yl)piperazine-1-carboxylate |
| 147 | 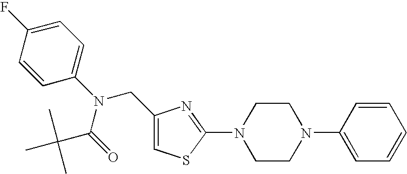 | N-(4-fluorophenyl)-2,2-dimethyl-N-{[2-(4- phenylpiperazin-1-yl)-1,3-thiazol-4- yl]methyl}propanamide |
| 148 | 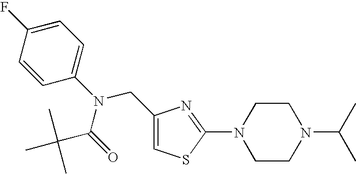 | N-(4-fluroophenyl)-N-{[2-(4-isopropylpiperazin-1- yl)-1,3-thiazol-4-yl]methyl}-2,2- dimethylpropanamide |
| 149 | 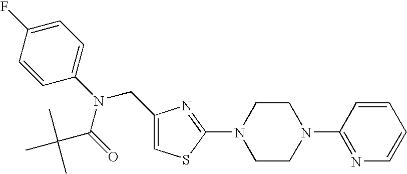 | N-(4-fluorophenyl)-2,2-dimethyl-N-{[2-(4-pyridin- 2-ylpiperazin-1-yl)--1,3-thiazol-4- yl]methyl}propanamide |
| 150 | 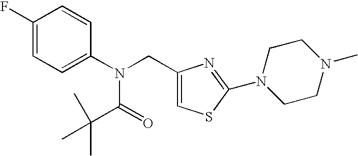 | N-(4-fluorophenyl)-2,2-dimethyl-N-{[2-(4- methylpiperazin-1-yl)-1,3-thiazol-4- yl]methyl}propanamide |
| 151 | 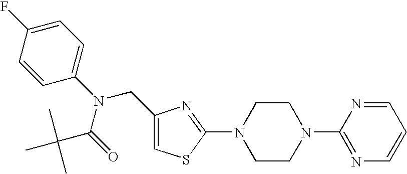 | N-(4-fluorophenyl)-2,2-dimethyl-N-{[2-(4- pyrimidin-2-ylpiperazin-1-yl)--1,3-thiazol-4- yl]methyl}propanamide |
| 152 | 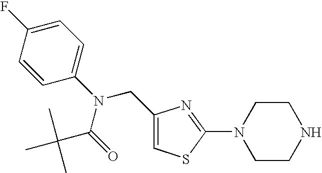 | N-(4-fluorophenyl)-2,2-dimethyl-N-[(2-piperazin- 1-yl-1,3-thiazol-4-yl)methyl]propanamide |
| 153 | 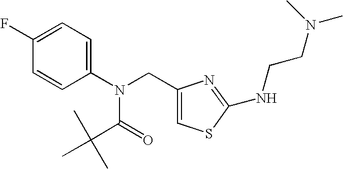 | N-[(2-{[2-(dimethylamino)ethyl]amino}-1,3- thiazol-4-yl)methyl]-N-(4-fluorophenyl)-2,2- dimethylpropanamide |
| 154 | 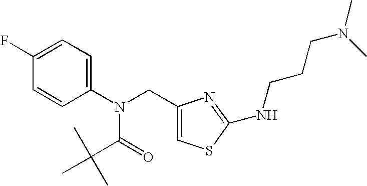 | N-[2-{[3-(dimethylamino)propyl]amino}-1,3- thiazol-4-yl)methyl]-N-(4-fluorophenyl)-2,2- dimethylpropanamide |
| 155 | 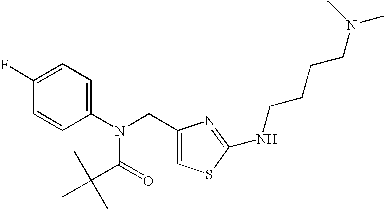 | N-[(2-{[4-(dimethylamino)butyl]amino}-1,3- thiazol-4-yl)methyl]-N-(4-fluorophenyl)-2,2- dimethylpropanamide |
| 156 | 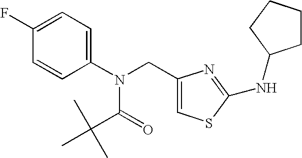 | N-{[2-(cyclopentylamino)-1,3-thiazol-4-yl]methyl}- N-(4-fluorophenyl)-2,2-dimethylpropanamide |
| 157 | 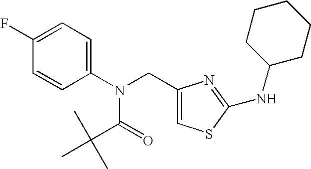 | N-{[2-(cyclohexylamino)-1,3-thiazol-4-yl]methyl}- N-(4-fluorophenyl)-2,2-dimethylpropanamide |
| 158 | 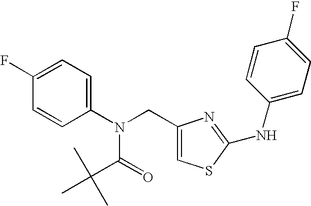 | N-(4-fluroophenyl)-N-({2-[(4-fluorophenyl)amino]- 1,3-thiazol-4-yl}methyl)-2,2- dimethylpropanamide |
| 159 | 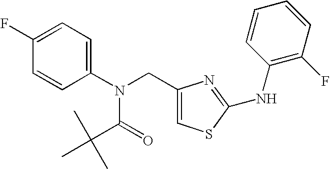 | N-(4-fluorophenyl)-N-({2-[(2-fluorophenyl)amino]- 1,3-thiazol-4-yl}methyl)-2,2- dimethylpropanamide |
| 160 | 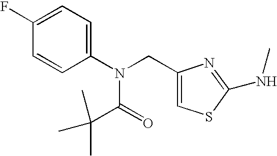 | N-(4-fluorophenyl)-2,2-dimethyl-N-{[2- (methylamino)-1,3-thiazol-4- yl]methyl}propanamide |
| 161 | 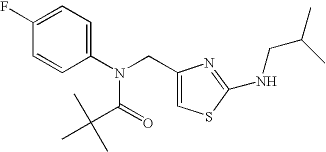 | N-(4-fluorophenyl)-N-{[2-(isobutylamino)-1,3- thiazol-4-yl]methyl}-2,2-dimethylpropanamide |
| 162 | 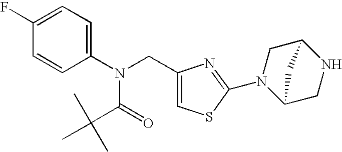 | N-({2-[(1S,4S)-2,5-diazabicycl[2.2.1]hept-2-yl]- 1,3-thiazol-4-yl}methyl)-N-(4-fluorophenyl)-2,2- dimethylpropanamide |
| 163 | 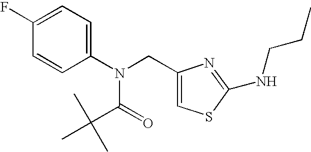 | N-(4-fluorophenyl)-2,2-dimethyl-N-{[2- (propylamino)-1,3-thiazol-4- yl]methyl}propanamide |
| 164 | 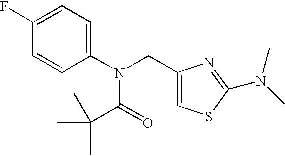 | N-{[2-(dimethylamino)-1,3-thiazol-4-yl]methyl}-N- (4-fluorophenyl)-2,2-dimethylpropanamide |
| 165 | 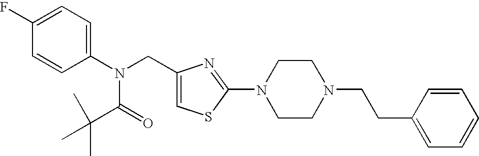 | N-(4-fluroophenyl)-2,2-dimethyl-N-({2-[4-(2- phenylethyl)piperazin-1-yl]--1,3-thiazol-4- yl}methyl)propanamide |
| 166 | 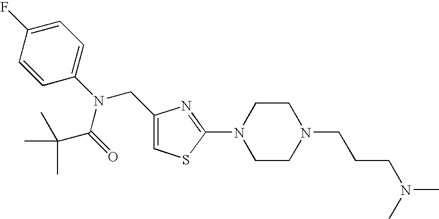 | N-[(2-{4-[3-(dimethylamino)propyl]piperazin-1-yl}- 1,3-thiazol-4-yl)methyl]-N-(4-fluorophenyl)-2,2- dimethylpropanamide |
| 167 | 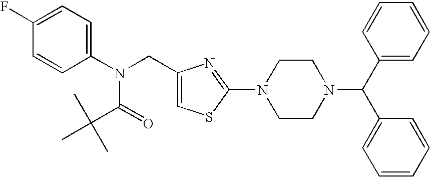 | N-({2-[4-(diphenylmethyl)piperazin-1-yl]-1,3- thiazol-4-yl}methyl)-N-(4-fluroophenyl)-2,2- dimethylpropanamide |
| 168 | 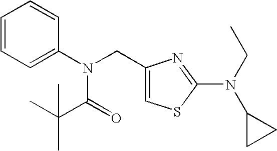 | N-({2-[cyclopropyl(ethyl)amino]-1,3-thiazol-4- yl}methyl)-2,2-dimethyl-N-phenylpropanamide |
| 169 | 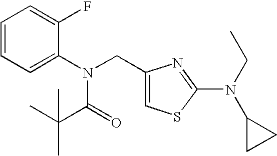 | N-({2-[cyclopropyl(ethyl)amino]-1,3-thiazol-4- yl}methyl)-N-(2-fluorophenyl)-2,2- dimethylpropanamide |
| 170 | 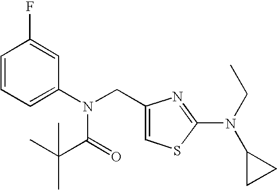 | N-({2-[cyclopropyl(ethyl)amino]-1,3-thiazol-4- yl}methyl)-N-(3-fluorophenyl)-2,2- dimethylpropanamide |
| 171 | 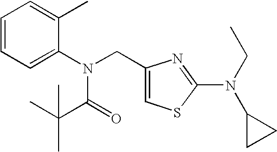 | N-({2-[cyclopropyl(ethyl)amino]-1,3-thiazol-4- yl}methyl)-2,2-dimethyl-N-(2- methylphenyl)propanamide |
| 172 | 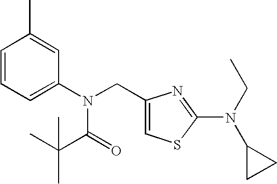 | N-({2-[cyclopropyl(ethyl)amino]-1,3-thiazol-4- yl}methyl)-2,2-dimethyl-N-(3- methylphenyl)propanamide |
| 173 | 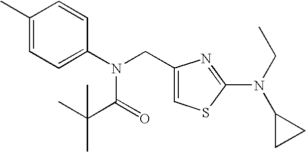 | N-({2-[cyclopropyl(ethyl)amino]-1,3-thiazol-4- yl}methyl)-2,2-dimethyl-N-(4- methylphenyl)propanamide |
| 174 | 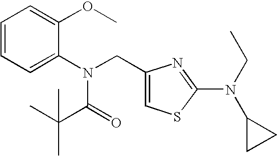 | N-({2-[cyclopropyl(ethyl)amino]-1,3-thiazol-4- yl}methyl)-N-(2-methoxyphenyl)-2,2- dimethylpropanamide |
| 175 | 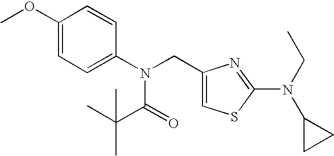 | N-({2-[cyclopropyl(ethyl)amino]-1,3-thiazol-4- yl}methyl)-N-(4-methoxyphenyl)-2,2- dimethylpropanamide |
| 176 | 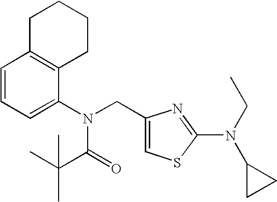 | N-({2-[cyclopropyl(ethyl)amino]-1,3-thiazol-4- yl}methyl)-2,2-dimethyl-N-(5,6,7,8- tetrahydronaphthalen-1-yl)propanamide |
| 177 | 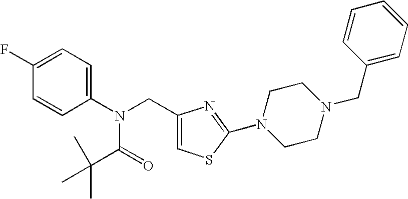 | N-{[2-(4-benzylpiperazin-1-yl)-1,3-thiazol-4- yl]methyl}-N-(4-fluorophenyl)-2,2- dimethylpropanamide |
| 178 | 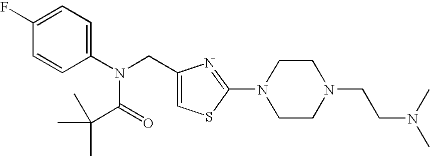 | N-[(2-{4-[2-(dimethylamino)ethyl]piperazin-1-yl}- 1,3-thiazol-4-yl)methyl]-N-(4-fluorophenyl)-2,2- dimethylpropanamide |
| 179 | 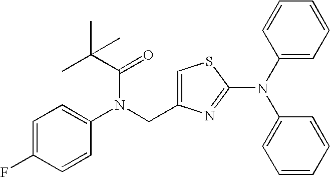 | N-{[2-(diphenylamino)-1,3-thiazol-4-yl]methyl}-N- (4-fluorophenyl)-2,2-dimethylpropanamide |
| 180 | 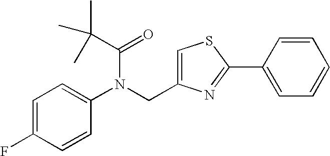 | N-(4-fluorophenyl)-2,2-dimethyl-N-[(2-phenyl-1,3- thiazol-4-yl)methyl]propanamide |
| 181 | 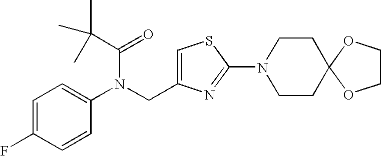 | N-{[2-(1,4-dioxa-8-azaspiro[4.5]dec-8-yl)-1,3- thiazol-4-yl]methyl}-N-(4-fluorophenyl)-2,2- dimethylpropanamide |
| 182 | 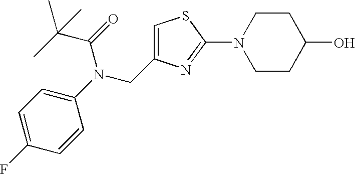 | N-(4-fluorophenyl)-N-{[2-(4-hydroxypiperidin-1- yl)-1,3-thiazol-4-yl]methyl}-2,2- dimethylpropanamide |
| 183 | 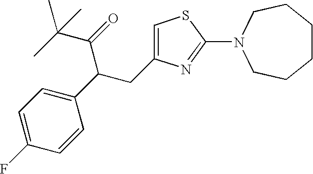 | N-[(2-azepan-1-yl-1,3-thiazol-4-yl)methyl]-N-(4- fluorophenyl)-2,2-dimethylpropanamide |
| 184 | 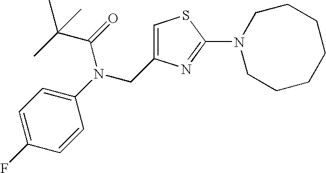 | N-[(2-azocan-1-yl-1,3-thiazol-4-yl)methyl]-N-(4- fluorophenyl)-2,2-dimethylpropanamide |
| 185 | 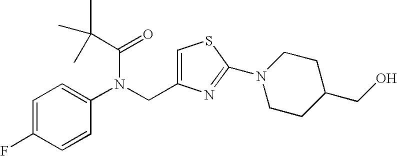 | N-(4-fluorophenyl)-N-({2-[4- (hydroxymethyl)piperidin-1-yl]-1,3-thiazol-4- yl}methyl)-2,2-dimethylpropanamide |
| 186 | 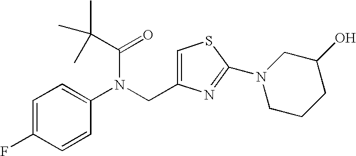 | N-(4-fluroophenyl)-N-{[2-(3-hydroxypiperidin-1- yl)-1,3-thiazol-4-yl]methyl}-2,2- dimethylpropanamide |
| 187 | 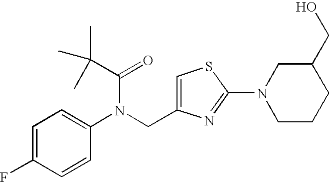 | N-(4-fluorophenyl)-N-({2-[3- (hydroxymethyl)piperidin-1-yl]-1,3-thiazol-4- yl}methyl)-2,2-dimethylpropanamide |
| 188 | 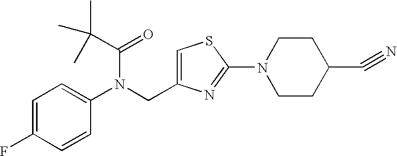 | N-{[2-(4-cyanopiperidin-1-yl)-1,3-thiazol-4- yl]methyl}-N-(4-fluorophenyl)-2,2- dimethylpropanamide |
| 189 | 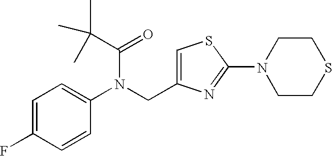 | N-(4-fluorophenyl)-2,2-dimethyl-N-[(2- thiomorpholin-4-yl-1,3-thiazol-4- yl)methyl]propanamide |
| 190 | 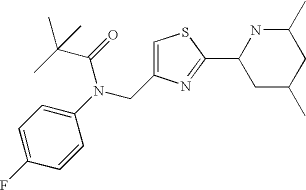 | N-{[2-(3,5-dimethylpiperidin-1-yl)-1,3-thiazol-4- yl]methyl}-N-(4-fluorophenyl)-2,2- dimethylpropanamide |
| 191 | 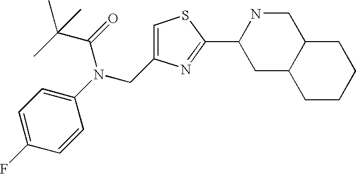 | N-(4-fluorophenyl)-2,2-dimethyl-N-{[2- (octahydroisoquinolin-2(1H)-yl)--1,3-thiazol-4- yl]methyl}propanamide |
| 192 | 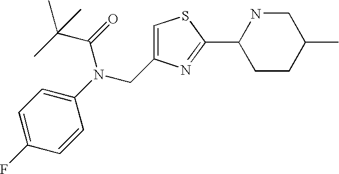 | N-(4-fluorophenyl)-2,2-dimethyl-N-{[2-(4- methylpiperidin-1-yl)-1,3-thiazol-4- yl]methyl}propanamide |
| 193 | 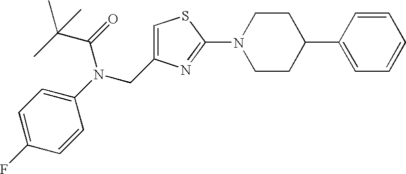 | N-(4-fluorophenyl)-2,2-dimethyl-N-{[2-(4- phenylpiperidin-1-yl)-1,3-thiazol-4- yl]methyl}propanamide |
| 194 | 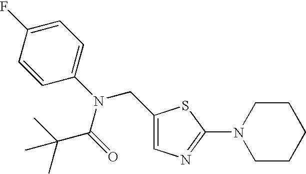 | N-(4-fluorophenyl)-2,2-dimethyl-N-[(2-piperidin-1- yl-1,3-thiazol-5-yl)methyl]propanamide |
| 195 | 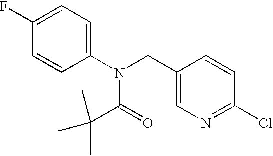 | N-[(6-chloropyridin-3-yl)methyl]-N-(4- fluorophenyl)-2,2-dimethylpropanamide |
| 196 | 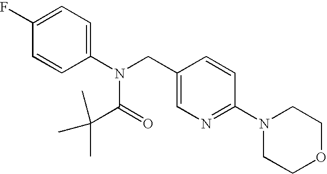 | N-(4-fluorophenyl)-2,2-dimethyl-N-[(6-morpholin- 4-ylpyridin-3-yl)methyl]propanamide |
| 197 | 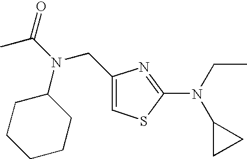 | N-cyclohexyl-N-({2-[cyclopropyl(ethyl)amino]-1,3- thiaozl-4-yl}methyl)acetamide |
| 198 | 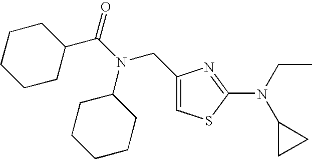 | N-cyclohexyl-N-({2-[cyclopropyl(ethyl)amino]-1,3- thiazol-4-yl}methyl)cyclohexanecarboxamide |
| 199 | 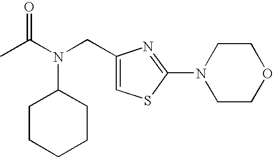 | N-cyclohexyl-N-[(2-morpholin-4-yl-1,3-thiazol-4- yl)methyl]acetamide |
| 200 | 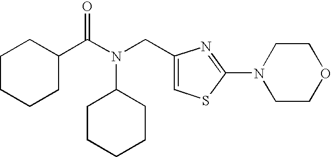 | N-cyclohexyl-N-[(2-morpholin-4-yl-1,3-thiazol-4- yl)methyl]cyclohexanecarboxamide |
| 201 | 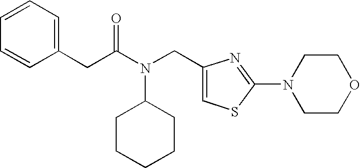 | N-cyclohexyl-N-[(2-morpholin-4-yl-1,3-thiazol-4- yl)methyl]-2-phenylacetamide |
| 202 | 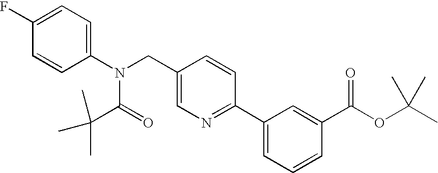 | tert-butyl 3-(5-{[(2,2-dimethylpropanoyl)(4- fluorophenyl)amino]methyl}pyridin-2-yl)benzoate |
| 203 | 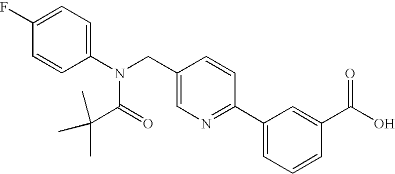 | 3-(5-{[(2,2-dimethylpropanoyl)(4- fluorophenyl)amino]methyl}pyridin-2-yl)benzoic acid |
| 204 | 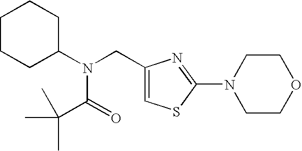 | N-cyclohexyl-2,2-dimethyl-N-[(2-morpholin-4-yl- 1,3-thiazol-4-yl)methyl]propanamide |
| 205 | 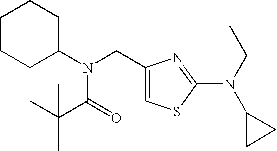 | N-cyclohexyl-N-({2-[cyclopropyl(ethyl)amino]-1,3- thiazol-4-yl}methyl)-2,2-dimethylpropanamide |
| 206 | 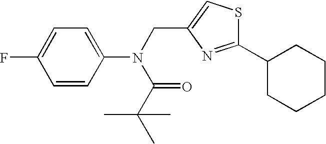 | N-[(2-cyclohexyl-1,3-thiazol-4-yl)methyl]-N-(4- fluorophenyl)-2,2-dimethylpropanamide |
| 207 | 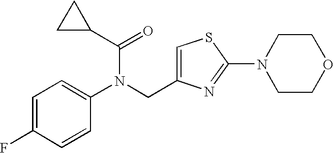 | N-(4-fluorophenyl)-N-[(2-morpholin-4-yl-1,3- thiazol-4-yl)methyl]cyclopropanecarboxamide |
| 208 | 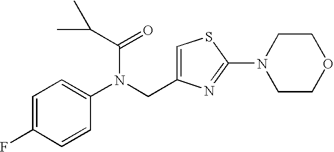 | N-(4-fluorophenyl)-2-methyl-N-[(2-morpholin-4- yl-1,3-thiazol-4-yl)methyl]propanamide |
| 209 | 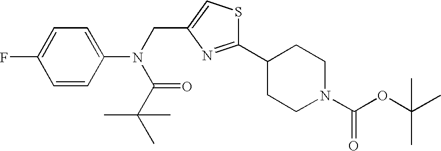 | tert-butyl 4-(4-{[(2,2-dimethylpropanoyl)(4- fluorophenyl)amino]methyl}--1,3-thiazol-2- yl)piperidine-1-carboxylate |
| 210 | 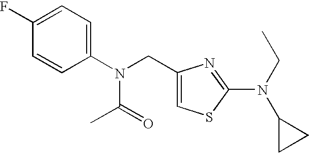 | N-({2-[cyclopropyl(ethyl)amino]-1,3-thiazol-4- yl}methyl)-N-(4-fluorophenyl)acetamide |
| 211 | 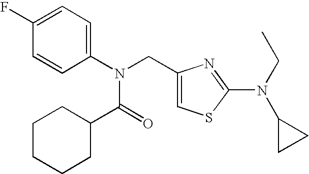 | N-({2-[cyclopropyl(ethyl)amino]-1,3-thiazol-4- yl}methyl)-N-(4- fluorophenyl)cyclohexanecarboxamide |
| 212 | 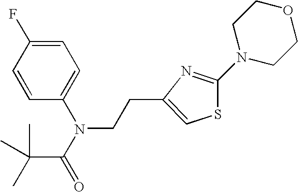 | N-(4-flurophenyl)-2,2-dimethyl-N-[2-(2- morpholin-4-yl-1,3-thiazol-4- yl)ethyl]propanamide |
| 213 | 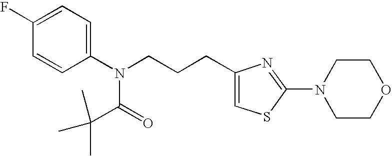 | N-(4-fluorophenyl)-2,2-dimethyl-N-[3-(2- morpholin-4-yl-1,3-thiazol-4- yl)propyl]propanamide |
| 214 | 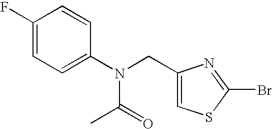 | N-[(2-bromo-1,3-thiazol-4-yl)methyl]-N-(4- fluorophenyl)acetamide |
| 215 | 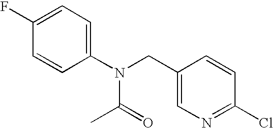 | N-[(6-chloropyridin-3-yl)methyl]-N-(4- fluorophenyl)acetamide |
| 216 | 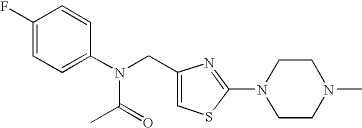 | N-(4-fluorophenyl)-N-{[2-(4-methylpiperazin-1- yl)-1,3-thiazol-4-yl]methyl}acetamide |
| 217 | 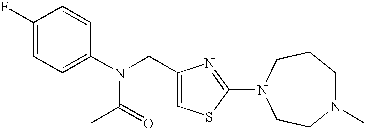 | N-(4-fluorophenyl)-N-{[2-(4-methyl-1,4-diazepan- 1-yl)-1,3-thiazol-4-yl]methyl}acetamide |
| 218 | 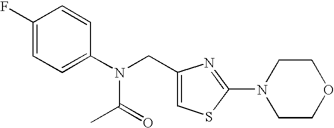 | N-(4-fluorophenyl)-N-[(2-morpholin-4-yl-1,3- thiaozl-4-yl)methyl]acetamide |
| 219 | 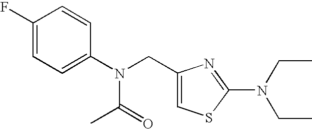 | N-{[2-(diethylamino)-1,3-thiaozl-4-yl]methyl}-N- (4-fluorophenyl)acetamide |
| 220 | 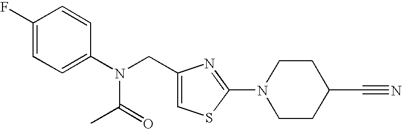 | N-{[2-(4-cyanopiperidin-1-yl)-1,3-thiazol-4- yl]methyl}-N-(4-fluorophenyl)acetamide |
| 221 | 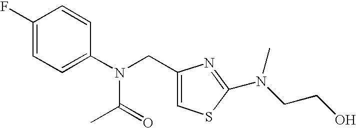 | N-(4-fluorophenyl)-N-({2-[(2- hydroxyethyl)(methyl)amino]-1,3-thiazol-4- yl}methyl)acetamide |
| 222 | 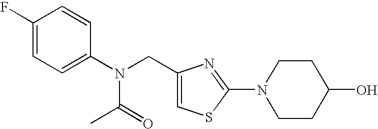 | N-(4-fluorophenyl)-N-{[2-(4-hydroxypiperidin-1- yl)-1,3-thiazol-4-yl]methyl}acetamide |
| 223 | 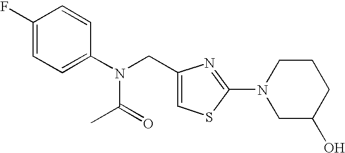 | N-(4-fluorophenyl)-N-{[2-(3-hydroxypiperidin-1- yl)-1,3-thiazol-4-yl]methyl}acetamide |
| 224 | 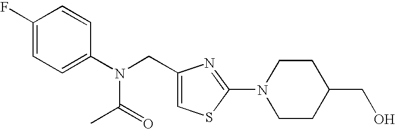 | N-(4-fluorophenyl)-N-({2-[4- (hydroxymethyl)piperidin-1-yl]-1,3-thiazol-4- yl}methyl)acetamide |
| 225 | 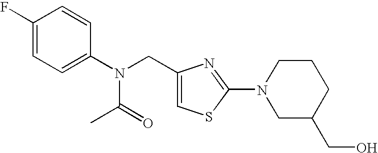 | N-(4-fluorophenyl)-N-({2-[3- (hydroxymethyl)piperidin-1-yl]-1,3-thiazol-4- yl}methyl)acetamide |
| 226 | 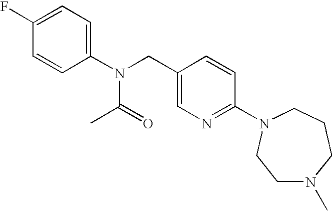 | N-(4-fluorophenyl)-N-{[6-(4-methyl-1,4-diazepan- 1-yl)pyridin-3-yl]methyl}acetamide |
| 227 | 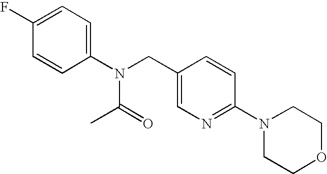 | N-(4-fluorophenyl)-N-[(6-morpholin-4-ylpyridin-3- yl)methyl]acetamide |
| 228 | 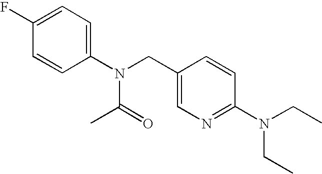 | N-{[6-(diethylamino)pyridin-3-yl]methyl}-N-(4- fluorophenyl)acetamide |
| 229 | 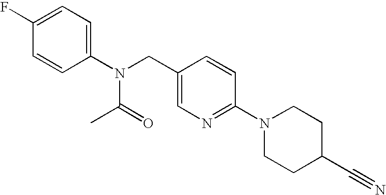 | N-{[6-(4-cyanopiperidin-1-yl)pyridin-3-yl]methyl}- N-(4-fluorophenyl)acetamide |
| 230 | 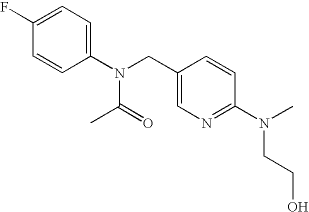 | N-(4-fluorophenyl)-N-({6-[(2- hydroxyethyl)(methyl)amino]pyridin-3- yl}methyl)acetamide |
| 231 | 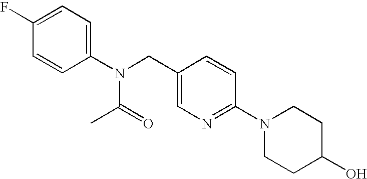 | N-(4-fluorophenyl)-N-{[6-(4-hydroxypiperidin-1- yl)pyridin-3-yl]methyl}acetamide |
| 232 | 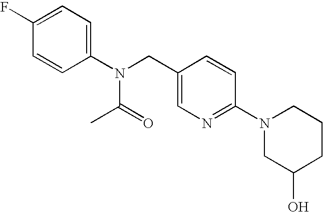 | N-(4-fluorophenyl)-N-{[6-(3-hydroxypiperidin-1- yl)pyridin-3-yl]methyl}acetamide |
| 233 | 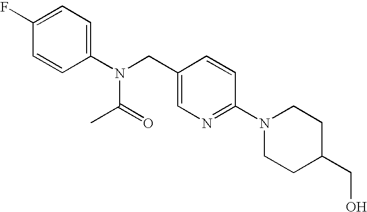 | N-(4-fluorophenyl)-N-({6-[4- (hydroxymethyl)piperidin-1-yl]pyridin-3- yl}methyl)acetamide |
| 234 | 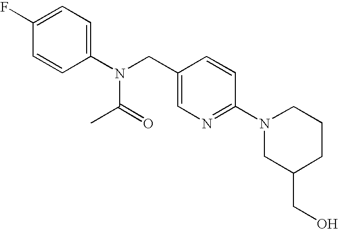 | N-(4-fluorophenyl)-N-({6-[3- (hydroxymethyl)piperidin-1-yl]pyridin-3- yl}methyl)acetamide |
| 235 | 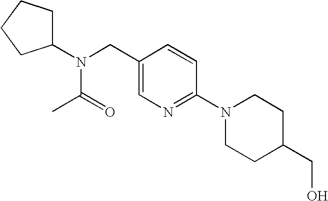 | N-cyclopentyl-N-({6-[4-(hydroxymethyl)piperidin- 1-yl]pyridin-3-yl}methyl)acetamide |
| 236 | 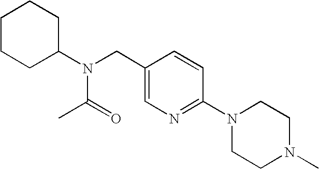 | N-cyclohexyl-N-{[6-(4-methylpiperzin-1- yl)pyridin-3-yl]methyl}acetamide |
| 237 | 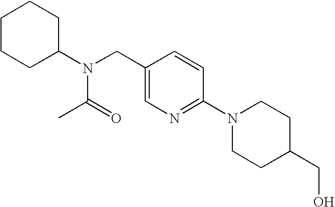 | N-cyclohexyl-N-({6-[4-(hydroxymethyl)piperidin- 1-yl]pyridin-3-yl}methyl)acetamide |
| 238 | 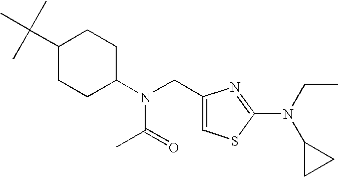 | N-(4-tert-butylcyclohexyl)-N-({2- [cyclopropyl(ethyl)amino]-1,3-thiazol-4- yl}methyl)acetamide |
| 239 | 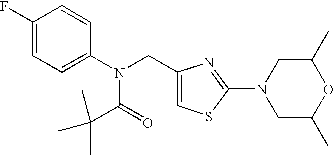 | 2,2-dimethylpropanamide N-{[2-(2,6-dimethylmorpholin-4-yl)-1,3-thiazol-4- yl]methyl}-N-(4-fluorophenyl)- |
| 240 | 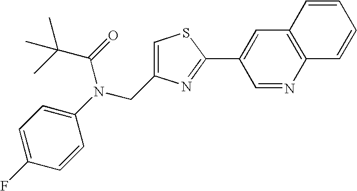 | N-(4-fluroophenyl)-2,2-dimethyl-N-[(2-quinolin-3- yl-1,3-thiazol-4-yl)methyl]propanamide |
| 241 | 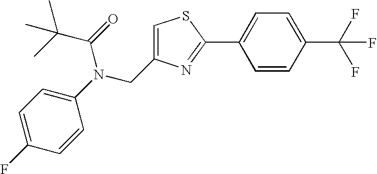 | N-(4-fluorophenyl)-2,2-dimethyl-N-({2-[4- (trifluoromethyl)phenyl]-1,3-thiazol-4- yl}methyl)propanamide |
| 242 | 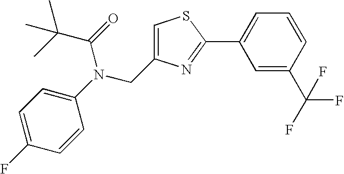 | N-(4-fluorophenyl)-2,2-dimethyl-N-({2-[3- (trifluoromethyl)phenyl]-1,3-thiazol-4- yl]methyl)propanamide |
| 243 | 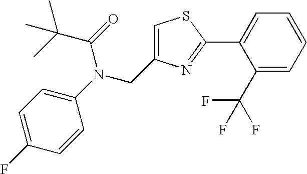 | N-(4-fluorophenyl)-2,2-dimethyl-N-({2-[2- (trifluoromethyl)phenyl]-1,3-thiazol-4- yl}methyl)propanamide |
| 244 | 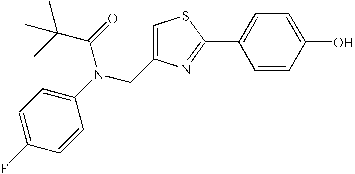 | N-(4-fluorophenyl)-N-{[2-(4-hydroxyphenyl)-1,3- thiazol-4-yl]methyl}-2,2-dimethylpropanamide |
| 245 | 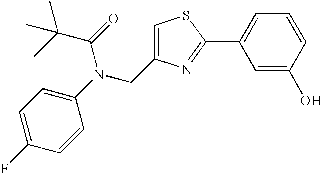 | N-(4-fluorophenyl)-N-{[2-(3-hydroxyphenyl)-1,3- thiazol-4-yl]methyl}-2,2-dimethylpropanamide |
| 246 | 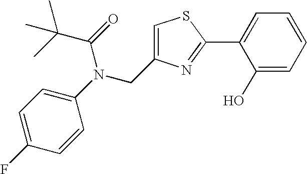 | N-(4-fluorophenyl)-N-{[2-(2-hydroxyphenyl)-1,3- thiazol-4-yl]methyl}-2,2-dimethylpropanamide |
| 247 | 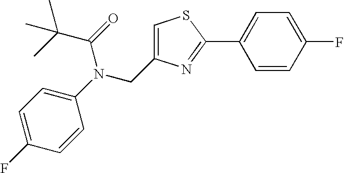 | N-(4-fluorophenyl)-N-{[2-(4-fluorophenyl)-1,3- thiazol-4-yl]methyl}-2,2-dimethylpropanamide |
| 248 | 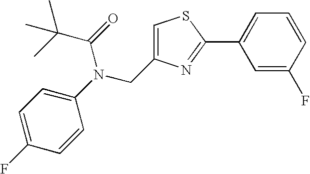 | N-(4-fluorophenyl)-N-{[2-(3-fluorophenyl)-1,3- thiazol-4-yl]methyl}-2,2-dimethylpropanamide |
| 249 | 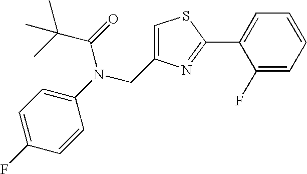 | N-(4-fluorophenyl)-N-{[2-(2-fluorophenyl)-1,3- thiazol-4-yl]methyl}-2,2-dimethylpropanamide |
| 250 | 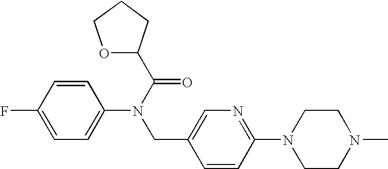 | N-(4-fluorophenyl)-N-{[6-(4-methylpiperazin-1- yl)pyridin-3-yl]methyl}tetrahydrofuran-2- carboxamide |
| 251 | 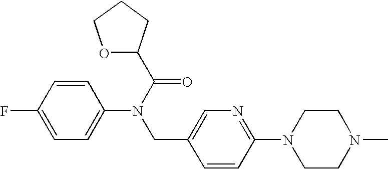 | N-(4-fluorophenyl)-N-{[6-(4-methylpiperazin-1- yl)pyridin-3-yl]methyl}tetrahydrofuran-3- carboxamide |
| 252 | 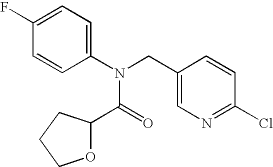 | N-[(6-chloropyridin-3-yl)methyl]-N-(4- fluuorophenyl)tetrahydrofuran-2-carboxamide |
| 253 | 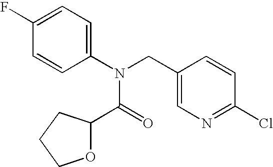 | N-[(6-chlroopyridin-3-yl)methyl]-N-(4- fluorophenyl)tetrahydrofuran-3-carboxamide |
| 254 | 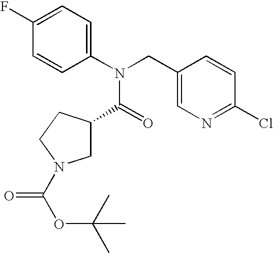 | tert-butyl (3S)-3-{[(6-chloropyridin-3-yl)methyl](4- fluorophenyl)carbamoyl}pyrrolidine-1-carboxylate |
| 255 | 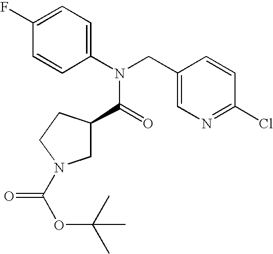 | tert-butyl (3R)-3-{[(6-chloropyridin-3-yl)methyl](4- fluorophenyl)carbamoyl}pyrrolidine-1-carboxylate |
| 256 | 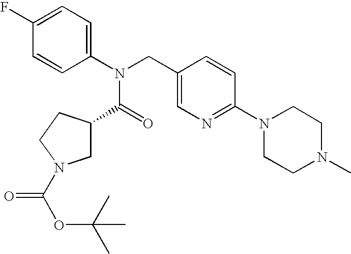 | tert-butyl (3S)-3-[(4-fluorophenyl){[6-(4- methylpiperazin-1-yl)pyridin-3- yl]methyl}carbamoyl]pyrrolidine-1-carboxylate |
| 257 | 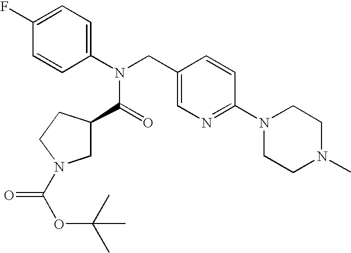 | tert-butyl (3R)-3-[(4-fluorophenyl){[6-(4- methylpiperazin-1-yl)pyridin-3- yl]methyl}carbamoyl]pyrrolidine-1-carboxylate |
| 258 | 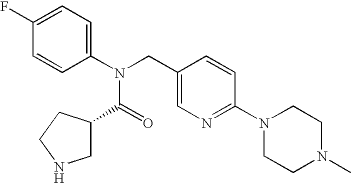 | (3S)-N-(4-fluorophenyl)-N-{[6-(4-methylpiperazin- 1-yl)pyridin-3-yl]methyl}pyrrolidine-3- carboxamide |
| 259 | 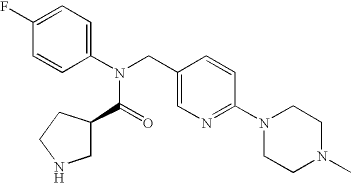 | (3R)-N-(4-fluorophenyl)-N-{[6-(4- methylpiperazin-1-yl)pyridin-3- yl]methyl}pyrrolidine-3-carboxamide |
| 260 | 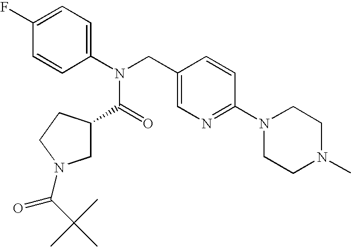 | (3S)-1-(2,2-dimethylpropanoyl)-N-(4- fluorophenyl)-N-{[6-(4-methylpiperazin-1- yl)pyridin-3-yl]methyl}pyrrolidine-3-carboxamide |
| 261 | 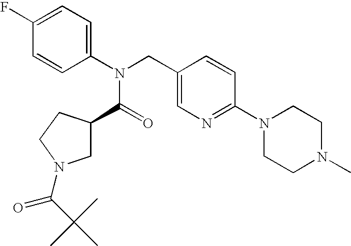 | (3R)-1-(2,2-dimethylpropanoyl)-N-(4- fluorophenyl)-N-{[6-(4-methylpiperazin-1- yl)pyridin-3-yl]methyl}pyrrolidine-3-carboxamide |

Pharmaceutically acceptable salts of the compounds of formula (I), which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, benzenesulfonic, benzoic, camphorsulfonic, citric, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and as well as other known pharmaceutically acceptable acids.

Pharmaceutically acceptable esters in the present invention refer to non-toxic esters of the compounds of formula (I), preferably the alkyl esters such as methyl, ethyl, propyl, isopropyl, butyl, isobutyl or pentyl esters, of which the methyl ester is preferred. However, other esters such as phenyl-C₁₋₅alkyl may be employed if desired. Examples of pharmaceutically acceptable esters include, but are not limited to, C₂-C₆alkyl esters such as methyl esters and ethyl esters. Pharmaceutically acceptable esters include esters made with aliphatic carboxylic acids, preferably those with a linear chain of between two and six carbon atoms, preferably acetic acid, and made with aromatic carboxylic acids, e.g. C₇₋₁₂ acids such as benzoic acid. The aliphatic and aromatic acids may optionally be substituted by one or more C₁₋₄alkyl groups.

Also provided in accordance with the present teachings are prodrugs of the compounds disclosed herein. As used herein, “prodrug” refers to a moiety that produces, generates or releases a compound of the present teachings when administered to a mammalian subject. Prodrugs can be prepared by modifying functional groups present in the compounds in such a way that the modifications are cleaved, either by routine manipulation or in vivo, from the parent compounds. Examples of prodrugs include compounds as described herein that contain one or more molecular moieties appended to a hydroxyl, amino, sulfhydryl, or carboxyl group of the compound, and that when administered to a mammalian subject, is cleaved in vivo to form the free hydroxyl, amino, sulfhydryl, or carboxyl group, respectively. Examples of prodrugs can include, but are not limited to, acetate, formate and benzoate derivatives of alcohol and amine functional groups in the compounds of the present teachings. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, the entire disclosures of which are incorporated by reference herein for all purposes.

Carboxylic acid amide compounds of formula (I) in accordance with the present invention can be prepared as outlined in the schemes below and as illustrated in the examples, from (a) commercially available starting materials, (b) compounds known in the literature, or readily prepared intermediates using literature procedures, or (c) new intermediates described in the schemes and experimental procedures herein.

Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but one skilled in the art can determine such conditions by routine optimization procedures. Those skilled in the art of organic synthesis will recognize that the nature and order of the synthetic steps presented may be varied for the purpose of optimizing the formation of the compounds described herein.

Reactions are performed in a solvent appropriate to the reagents and materials employed and suitable for the transformation being effected. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected. One skilled in the art of organic synthesis can readily selected suitable solvents.

It is understood by those skilled in the art of organic synthesis that the various functionalities present on the molecule must be consistent with the chemical transformation proposed. This may necessitate routine judgment as to the order of synthetic steps, and the need for protecting groups for remote functionalities. One skilled in the art can readily determine the need for protection and deprotection and select appropriate protecting groups. The chemistry of protecting groups can be found, for example, in Greene, et al., Protective Groups in Organic Synthesis, 2d. Ed., Wiley & Sons, 1991, the entire disclosure of which is herein incorporated by reference.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatograpy (HPLC) or thin layer chromatography.

In the schemes provided herein, unless expressed to the contrary, variables in chemical formulae are as defined in other formulae herein. For example, Ar, R¹, R², R³, and X in the schemes are defined as in any of the formulae herein, except where defined otherwise in the schemes.

One method for preparing compounds of formula (I) where X is a covalent bond involves the coupling of an aliphatic acid or acid derivative (II) with an appropriate amine (III) as shown in Scheme 1 below:

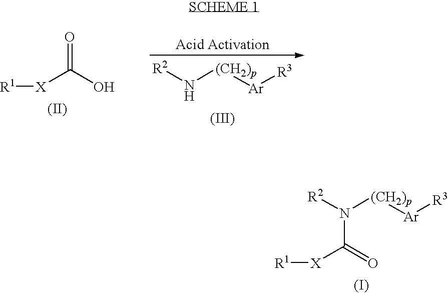

An aliphatic acid (II), or alternatively an activated acid derivative, is coupled with the desired amine (III) to provide a compound of Formula (I). Many aliphatic acids and their derivatives are commercially available or can otherwise be prepared by literature methods.

Examples of activated acid derivatives include, for example, acid chlorides, esters, acylimidazoles, anhydrides; these activated acid derivatives can be generated in situ or as isolated compounds. Representative activating agents include, but are not limited to, sulfuryl chloride, thionyl chloride, 2-chloro-4,6-dimethoxy-1,3,5-triazine, and carbodiimides such as 1-[3-(dimethylamino)propyl]-3-ethyl-carbodiimide and dicyclohexyl carbodiimide; for examples of amide bond formation and acid activation, see Montalbetti C. A. G. N. and Falque, V. (2005), Tetrahedron, 61(46): 10827-10852, the entire disclosure of which is herein incorporated by reference.

Scheme 2 illustrates a method for preparing compounds of formula (I) where X is —NR_{c}—, by coupling an appropriate isocyanate (IV) with the desired amine (III).

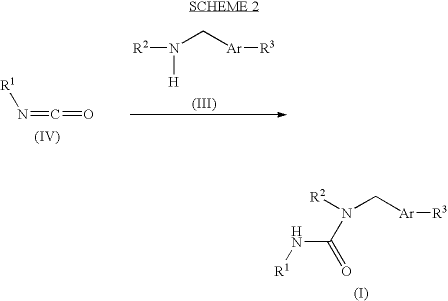

Scheme 3 illustrates a method for preparing compounds of formula (I) where X is —O—, by coupling a appropriate formate (V) with the desired amine (III).

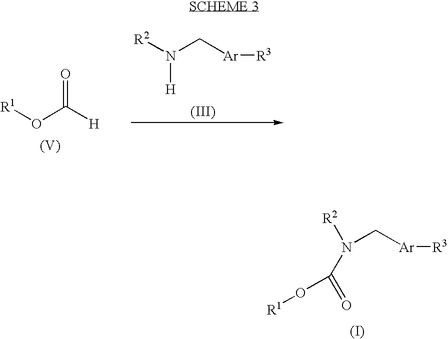

Alternatively the R³ group can be incorporated in the last step of the synthesis, as illustrated in Scheme 4 below.

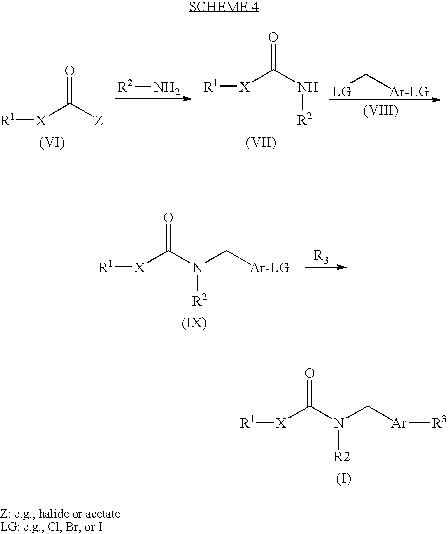

In this scheme, an acid halide, anhydride or activated acid derivative (VI) is reacted with the appropriate amine (R²—NH₂) to provide the amide (VII). Alkylation of the resulting amide (VII) with a compound of formula (VIII) provides the substituted amide (IX). Compounds of formula (VIII) are either commercially available or can otherwise be readily synthesized. Displacement of the leaving group on the substituted amide (IX) with the desired R³ group provides a compound of Formula (I).

The amine (III) can be synthesized as described in Scheme 5 below.

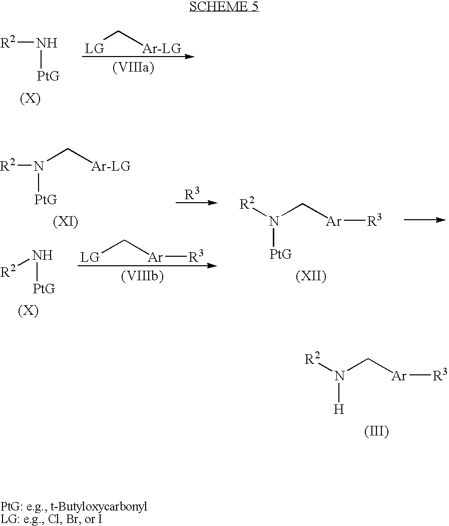

In this scheme, alkylation of a protected amine (X) with a compound of formula (VIIIa) provides the protected alkylated amine (XI). Displacement of the leaving group on compound (XI) with the appropriate amine (R³, wherein R³ is NR_{f}R_{g}) provides the amine-substituted aryl/heteroaryl derivative (XII). Alternatively, alkylation of the protected amine (X) with a compound of formula (VIIIb) provides the amine-substituted aryl/heteroaryl derivative (XII) directly. Removal of the protecting group (PtG) under standard conditions provides the desired amine (III).

Alternatively, the amine (III) can be synthesized from commercially available substituted acid halides, anhydrides or other activated carboxylic acid derivatives (VIIIa or VIIIb), as illustrated in Scheme 6 below.

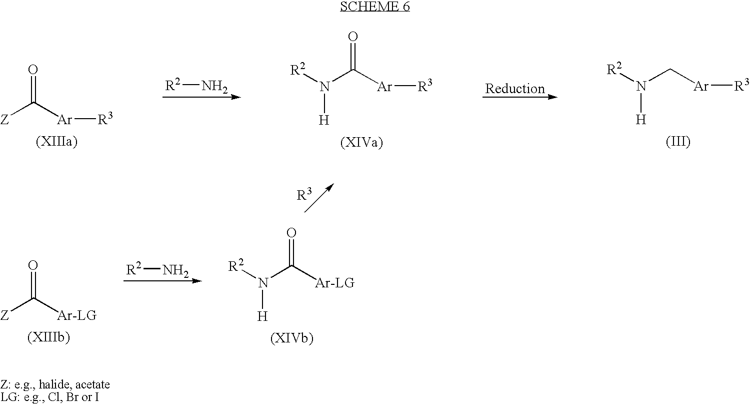

In this scheme, a substituted acid halide, anhydride or activated carboxylic acid derivative (XIIIa or XIIIb) is reacted with the appropriate amine R²—NH₂ to provide the amide (XIVa or XIVb). In the case of amide (XIVb), displacement of the leaving group (LG) with the appropriate amine (R³, wherein R³ is NR_{f}R_{g}) provides amide (XIVa). Finally, the amide (XIVa) is reduced under standard conditions to provide the desired amine (III).

A third approach commences with a substituted aryl/heteroaryl compound (XV), as illustrated in Scheme 7 below.

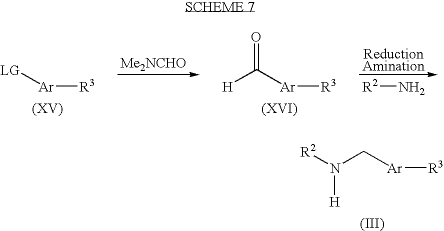

More specifically, conversion of a compound of formula (XV) to the corresponding organometallic derivative and treatment with dimethylformamide (Me₂NCHO) provides the aryl/heteroaryl aldehyde (XVI). Reductive amination with the appropriate amine (R²—NH₂) provides the desired amine (III).

Evaluation of representative compounds according to embodiments of this invention indicated that the compounds of the present teachings can modulate the activity of ion channels in a mammal, for example, Caᵥ2.2 voltage-gated calcium channels.

A variety of pathological conditions, states, disorders or diseases can be treated by modulating the activity of certain ion channels. As used herein, “ion channel mediated condition” refers to any condition or pathological state of a mammal or any disease present in a mammal that can be treated, or the symptoms of which can be alleviated, by modulation of the activity of one or more ion channels such as Caᵥ2.2 voltage-gated calcium channels. An ion channel mediated condition can be attributed to the abnormal functioning of one or more ion channels. An ion channel can be functioning abnormally when, for example, the ion channel exhibits abnormally increased or decreased activation.

By way of non-limiting examples, ion channel mediated conditions include conditions associated with neuronal hyperexcitability, conditions associated with abnormal glutamate regulation, pain, convulsions, epilepsy, stroke, anxiety disorders, neuronal disorders, traumatic brain injury, angina, hypertension, congestive heart failure, myocardial ischemia, arrhythmia, diabetes, urinary incontinence, hot flush, thermal disregulation, and combinations thereof.

Examples of conditions associated with neuronal hyperexcitability include, but are not limited to, convulsions, including neonatal convulsions, epilepsy, episodic ataxia, myokymia, cerebral ischemia, cerebral palsy, stroke, traumatic brain injury, traumatic spinal cord injury, asphyxia, anoxia, prolonged cardiac surgery, and combinations thereof.

Examples of conditions associated with the abnormal regulation of glutamate include, but are not limited to, hypoglycemia or diseases associated with abnormal glutamate regulation such as, without limitation, Parkinson's disease, Huntingdon's disease, Alzheimer's disease, amyotrophic lateral sclerosis, AIDS-related dementia, and combinations thereof.

Examples of anxiety disorders include, but are not limited to, agoraphobia, panic disorder, specific phobia, social phobia, obsessive compulsive disorder, posttraumatic stress disorder, acute stress disorder, generalized anxiety disorder, separation anxiety disorder, substance-induced anxiety disorder, and anxiety disorder not otherwise specified.

Examples of pain include, but are not limited to various types of nociceptic or neuropathic pain, such as, without limitation, inflammatory pain, musculoskeletal pain, bony pain, lumbosacral pain, neck or upper back pain, visceral pain, somatic pain, pain associated with diabetic neuropathy, cancer pain, pain caused by injury or surgery such as burn pain, headaches such as migraines or tension headaches, and combinations of these pains. One skilled in the art will recognize that these pain types can overlap one another. For example, a pain caused by inflammation can also be visceral or musculoskeletal in nature. Other examples of pain include those related to conditions of hyperalgesia, allodynia, or both. The types of pain listed above can be acute (short duration) or chronic (regularly reoccurring or persistent), centralized or peripheral, and can be with or without peripheral or central sensitization.

Accordingly, the compounds of the present teachings can be useful for the treatment of a pathological condition, disorder or disease, and the alleviation of a symptom thereof, in a mammal, for example, a human. The pathological condition, disorder or disease, or a symptom thereof, can be, but is not limited to, one of the various ion channel mediated conditions described above. In some embodiments, the compounds of the present teachings can be used for pain therapy, including treating, by way of non-limiting examples, the various types of pain described above. As used herein, “treating” refers to partially or completely alleviating, inhibiting, preventing and/or ameliorating the condition. The present teachings therefore include use of the compounds disclosed herein as active therapeutic substances for the treatment of a variety of ion channel mediated conditions as well as for pain therapy.

For example, the compounds disclosed herein can be useful for treating the various conditions associated with neuronal hyperexcitability, the various conditions associated with abnormal glutamate regulation, the various anxiety and neuronal disorders, angina, hypertension, congestive heart failure, myocardial ischemia, arrhythmia, diabetes, urinary incontinence, and combinations thereof, as described above.

The compounds disclosed herein also can be useful for treating pain, including chronic pain that is neuropathic pain associated with damage to or pathological changes in the peripheral nervous system or the central nervous system; visceral pain associated with, by way of non-limiting examples, the abdominal, pelvic, and/or perineal regions or pancreatitis; musculoskeletal pain; bony pain associated with, by way of non-limiting examples, bone or joint degenerating disorders such as osteoarthritis, rheumatoid arthritis, or spinal stenosis; cancer pain; musculoskeletal pain associated with, by way of non-limiting examples, the lower or upper back, spine, fibromylagia, temporomandibular joint, or myofascial pain syndrome; headaches such migraine or tension headaches; pain associated with infections such as HIV or shingles, sickle cell anemia, autoimmune disorders, multiple sclerosis, and inflammation in accordance with the methods described herein.

Inflammatory pain can be associated with a variety of medical conditions such as osteoarthritis, rheumatoid arthritis, surgery, or injury. Neuropathic pain may be associated with, for example, diabetic neuropathy, peripheral neuropathy, post-herpetic neuralgia, trigeminal neuralgia, lumbar or cervical radiculopathies, fibromyalgia, glossopharyngeal neuralgia, reflex sympathetic dystrophy, causalgia, thalamic syndrome, nerve root avulsion, or nerve damage cause by injury resulting in peripheral and/or central sensitization such as phantom limb pain, reflex sympathetic dystrophy or postthoracotomy pain, cancer, chemical injury, toxins, nutritional deficiencies, or viral or bacterial infections such as shingles or HIV, or combinations thereof. The methods of use for compounds of this invention further include treatments in which the neuropathic pain is a condition secondary to metastatic infiltration, adiposis dolorosa, burns, or central pain conditions related to thalamic conditions.

Chronic pain may be associated with diabetes, post traumatic pain of amputation, lower back pain, spinal cord damage, cancer, chemical injury, chemotherapy induced peripheral neuropathy, toxins, major surgery, peripheral nerve damage due to traumatic injury, post-herpetic neuralgia, trigeminal neuralgia, lumbar or cervical radiculopathies, fibromyalgia, glossopharyngeal neuralgia, reflex sympathetic dystrophy, causalgia, thalamic syndrome, nerve root avulsion, reflex sympathetic dystrophy or post thoracotomy pain, nutritional deficiencies, viral infection, bacterial infection, metastatic infiltration, adiposis dolorosa, burns, central pain conditions related to thalamic conditions; and any combination thereof.

As used herein, the term “chronic pain” refers to centralized or peripheral pain that is intense, localized, sharp, or stinging, and/or dull, aching, diffuse, or burning in nature and that occurs for extended periods of time (i.e., persistent and/or regularly reoccurring), including, for the purpose of the present invention, neuropathic pain and cancer pain. Chronic pain includes neuropathic pain, hyperalgesia, and/or allodynia.

One skilled in the art will also recognize that at least some of the types of pain described above can be attributed to a condition associated with the abnormal activity of one or more ion channels such as, but not limited to, the abnormal regulation of glutamate.

The present teachings therefore include methods of administering to a mammal a therapeutically effective amount of a compound disclosed herein. As used herein, “administer” or “administering” refers to either directly administering a compound of the present teachings or a pharmaceutical composition containing the compound, or administering the compound or pharmaceutical composition indirectly via a prodrug derivative or analog which will form an equivalent amount of the active compound or substance within the body. The methods also can include identifying a mammal in need of such treatment, and administering a therapeutically effective amount of a compound disclosed herein to the mammal in need thereof. As used herein, “therapeutically effective” refers to a substance or an amount that elicits a desirable biological activity or effect.

In some embodiments, the method includes administering to a mammal a pharmaceutical composition that comprises a compound disclosed herein in combination or association with a pharmaceutically acceptable carrier. The compound of the present teachings can be administered alone or in combination with other therapeutically effective compounds or therapies for the treatment of such condition(s). For example, the other therapeutically effective compounds can include a cardiovascular disease agent and/or a nervous system disease agent. A nervous system disease agent can be a peripheral nervous system (PNS) disease agent and/or a central nervous (CNS) disease agent.

The present teachings also relate to in vitro or in vivo methods of modulating the activity of ion channels including, but not limited to, Caᵥ2.2 voltage-gated calcium channels. In some embodiments, such methods include contacting a Caᵥ2.2 voltage-gated calcium channel with a compound disclosed herein. In certain embodiments, the methods include monitoring the activity of ion channels. In various embodiments, the present teachings relate to methods of modulating the activity of an ion channel such as a Caᵥ2.2 voltage-gated calcium channel that include in vitro or in vivo administration of a pharmaceutically effective amount of one or more compounds of formula (I). As used herein, “pharmaceutically effective” refers to an amount that can elicit an intended biological activity or effect.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to treat the symptoms of the disease and its complications. The dosage to be used in the treatment of a specific individual typically must be subjectively determined by the attending physician. The variables involved include the specific condition and its state as well as the size, age and response pattern of the patient.

The present teachings also provide pharmaceutical compositions comprising at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions. Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known antiinflammatory agents. Oral formulations containing an active compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided active compound. In tablets, an active compound can be mixed with a carrier having the necessary compression properties in suitable proportions and compacted in the shape and size desired. The powders and tablets can contain up to about 99% or greater of the active compound.

Capsules can contain mixtures of active compound(s) with inert filler(s) and/or diluent(s) such as the pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents can include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidol silicon dioxide, phosphates, sodium dodecylsulfate, magnesium aluminum silicate, and triethanolamine. Oral formulations herein can utilize standard delay or time-release formulations to alter the absorption of the active compound(s). The oral formulation can also consist of administering an active compound in water or fruit juice, containing appropriate solubilizers or emulisifiers as needed.

Liquid carriers can be used in preparing solutions, suspensions, emulsions, syrups, and elixirs. An active compound described herein can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described above, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intrathecal, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the active compound. The unit dosage forms can be packaged compositions, for example, packeted powders, vials, ampoules, prefilled syringes or sachets containing liquids. Alternatively, the unit dosage form can be a capsule or tablet itself, or it can comprise the appropriate number of any such compositions in package form. Such unit dosage form may contain from about 1 mg/kg of active compound to about 500 mg/kg of active compound, and can be given in a single dose or in two or more doses. Such doses can be administered in any manner useful in directing the active compound(s) to the recipient's bloodstream, including orally, via implants, parenterally (including intravenous, intraperitoneal and subcutaneous injections), rectally, vaginally, and transdermally. Such administrations can be carried out using the compounds of the present teachings including pharmaceutically acceptable salts thereof, in lotions, creams, foams, patches, suspensions, solutions, and suppositories (e.g., rectal and vaginal).

In some cases, it may be desirable to administer a compound directly to the airways of the patient in the form of a dry powder or an aerosol. For administration by intranasal or intrabronchial inhalation, the compounds of the present teachings can be formulated, for example, into an aqueous or partially aqueous solution.

Compounds described herein can be administered enterally or parenterally (such as, without limitation, interperitoneal, intramuscular, intravascular, intrathecal, intra-articular or subcuteaneous injection or infusion). Solutions or suspensions of these active compounds or pharmaceutically acceptable salts thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In preferred embodiments, the form is sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts thereof, in lotions, creams, foams, patches, suspensions, solutions, and suppositories (e.g., rectal and vaginal). Topical formulations that deliver active compound(s) through the epidermis can be useful for localized treatment of inflammation and arthritis.

Transdermal administration can be accomplished through the use of a transdermal patch containing an active compound and a carrier that can be inert to the active compound, can be non-toxic to the skin, and can allow delivery of the active compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active compound can also be suitable. A variety of occlusive devices can be used to release the active compound into the blood stream, such as a semi-permeable membrane covering a reservoir containing the active compound with or without a carrier, or a matrix containing the active compound. Other occlusive devices are known in the literature.

Compounds described herein can be administered into a body cavity, (e.g., rectally or vaginally) in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art. For example, the compounds described herein can be administered in the form of liposomes. As is known in the art, liposomes are generally derived from phospholipids or other lipid substances, and are formed by mono or multilamellar hydrated liquid crystals that are dispersed in an aqueous medium. Any nontoxic, pharmacologically acceptable lipid capable of forming liposomes can be used.

To increase the effectiveness of compounds of the present teachings, it can be desirable to combine a compound with other agents effective in the treatment of the target disease. For inflammatory diseases, other active compounds (i.e., other active ingredients or agents) effective in their treatment, and particularly in the treatment of asthma and arthritis, can be administered with active compounds of the present teachings. The other agents can be administered at the same time or at different times than the compounds disclosed herein.

Throughout the description, where compositions are described as having, including, or comprising specific components, or where processes are described as having, including, or comprising specific process steps, it is contemplated that compositions of the present teachings also can consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such optical isomers (enantiomers) and diastereomers (geometric isomers), as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Throughout the specification, structures may or may not be presented with chemical names. Where any question arises as to nomenclature, the structure prevails.

Aspects of the present teachings can be further understood in light of the following examples, which should not be construed as limiting the scope of the present teachings in any way.

More specifically, the following examples illustrate various synthetic routes that can be used to prepare reagents and intermediates, including appropriate amines and carboxylic acids, that can be used to prepare compounds of formula (I).

### EXAMPLES

Amines of formula R²NH(CH₂)ₚArR³, including those provided in the following examples and others commercially available or prepared according to procedures known in the art, can be coupled with various carboxylic acids and acid derivatives to provide compounds of formula (I). Useful carboxylic acids and activated derivatives include those provided in the following examples as well as those that are commercially available or prepared according to procedures known in the art.

Compound Numbers 1-261 were prepared in accordance with Representative Schemes 1-20 and the following specific examples of analogous compounds using the appropriate starting materials. Selected compounds are shown in Table 2 below.

It is understood by those skilled in the art of organic synthesis that the substitution patterns of the starting materials determines the substitution patterns of the products, and the skilled practitioner will be able to exercise routine judgment for the selection of suitable starting materials in order to prepare specific products, the order of synthetic steps, and the need for protecting groups for remote functionalities.

While certain acyl chlorides are illustrated in the representative schemes as examples of activated acid derivatives useful for acylation of amines, other reagents for amide bond formation as known in the art can be utilized in the preparation of compounds of formula (I) in accordance with the teachings herein.

In some cases, the compounds were isolated as hydrochloride salts prepared via standard protocols using anhydrous hydrogen chloride as a gas, or as a solution in dioxane or diethyl ether. Those skilled in the art will also appreciate that the protonation state of the test compound is in accordance with the pH of the assay conditions, typically buffered as specified in the assay protocols, and not of the salt form or free base of the compound as synthesized.

When reference is made to HPLC retention time, the following HPLC conditions were used:
HPLC A: Waters Xterra RP18, 3.5 u, 150×4.6 mm; Temperature 40° C.; Flow Rate 1.2 mL/min; Mobile Phase Comp. 85/15-5/95 (Ammon. Form. Buff. Ph=3.5/ACN+MeOH) for 10 min, hold 4 min; Injection Volume 5 L; Detector Wavelength 210-370 nM.
HPLC C: Mobile phase gradient=5% acetonitrile/95% ammonium acetate (10 mM) to 95% acetonitrile/5% ammonium acetate (10 mM) over 2.5 min, hold for 1.5 min, then re-equilibrate. Column=Keystone Aquasil™ C18 column (2×50 mm, 5 mM). Detection=214 nm and 254 nm.

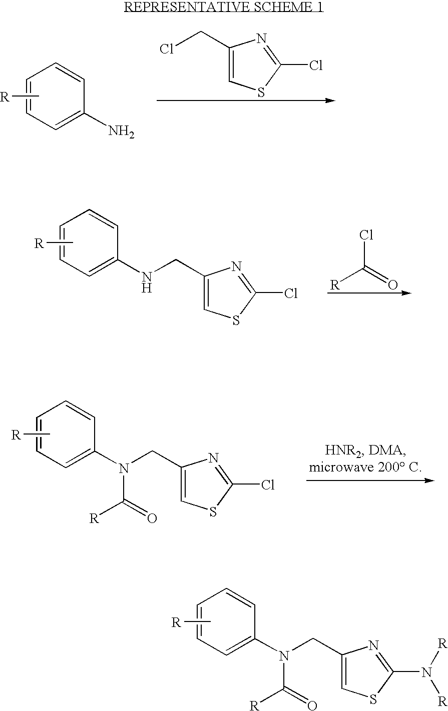

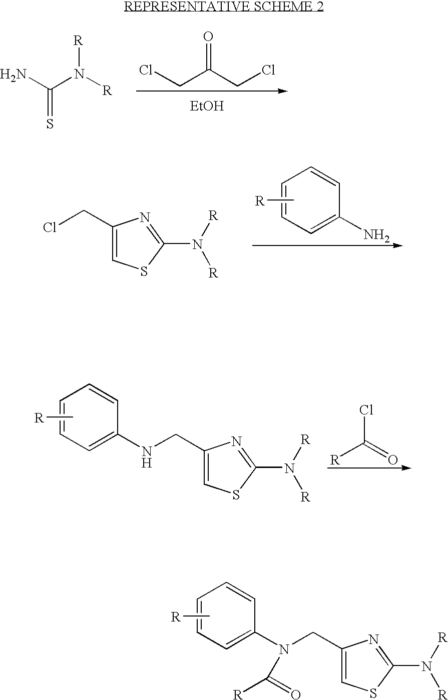

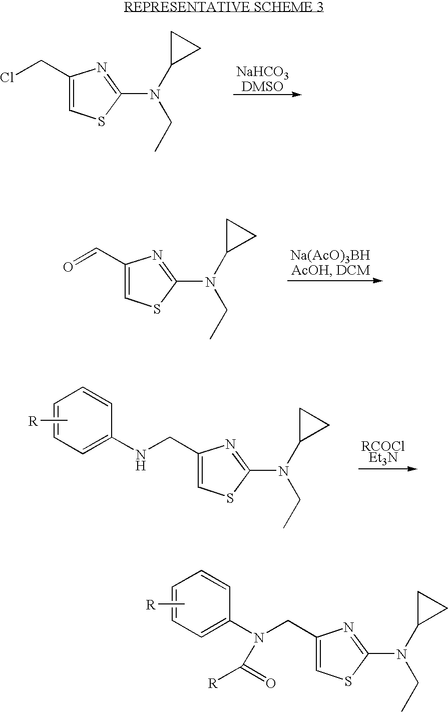

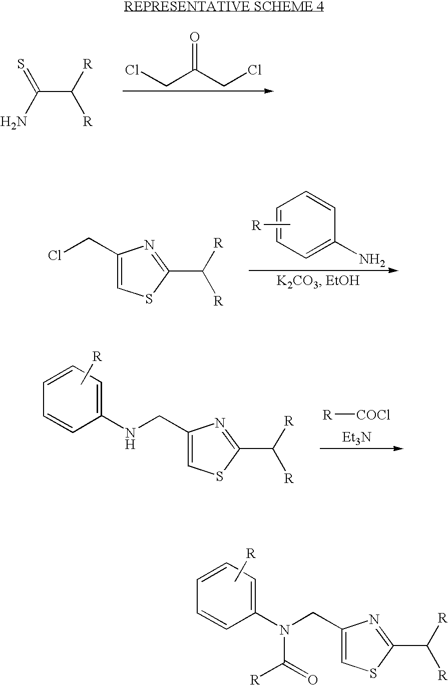

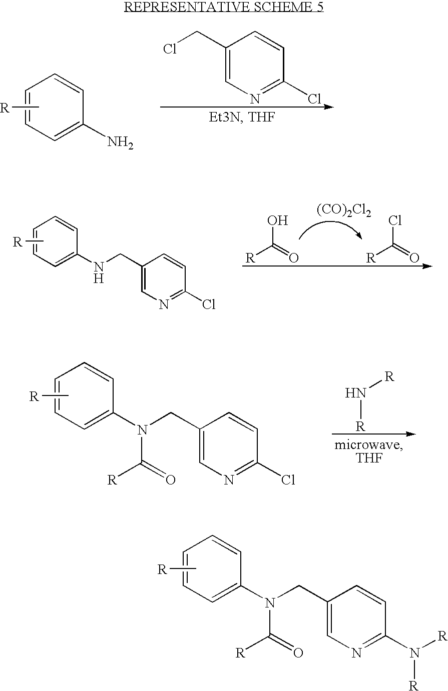

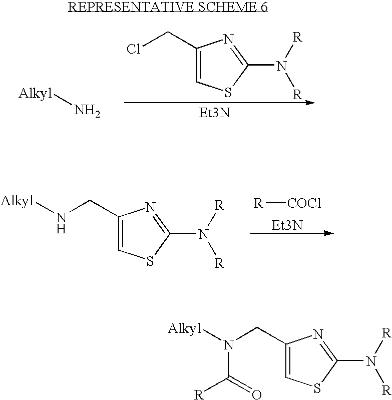

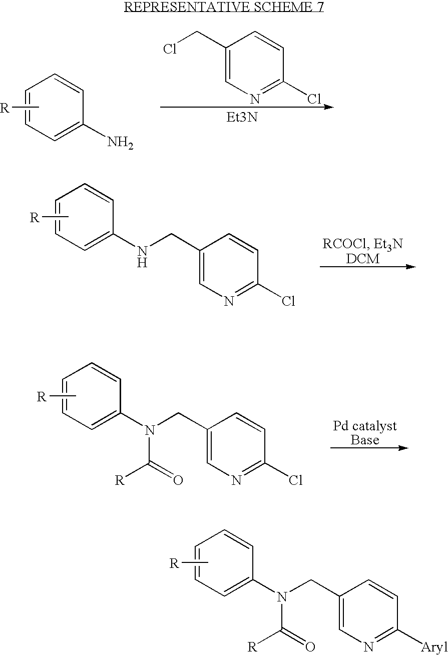

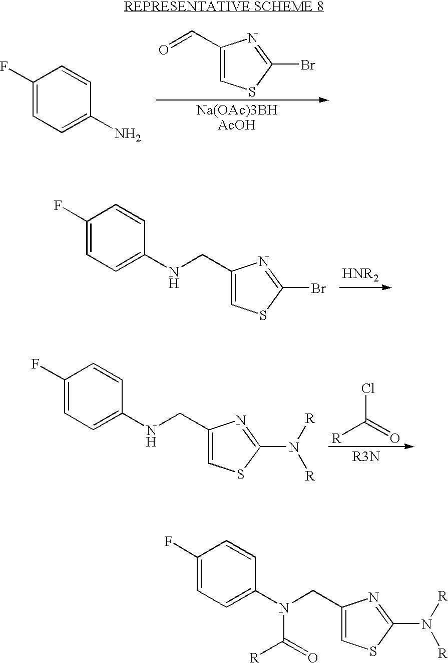

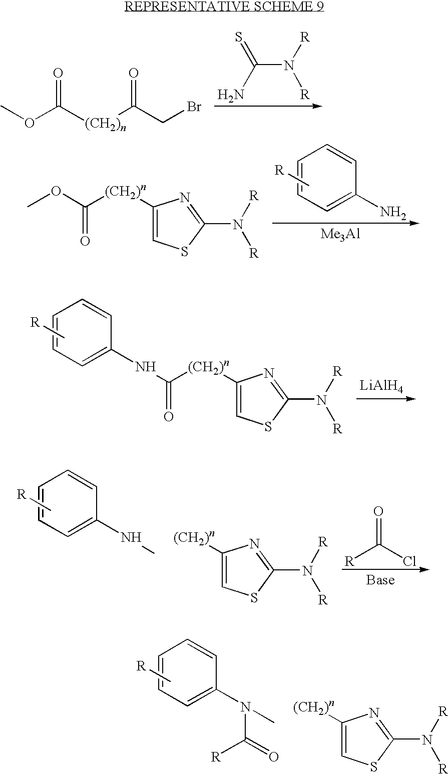

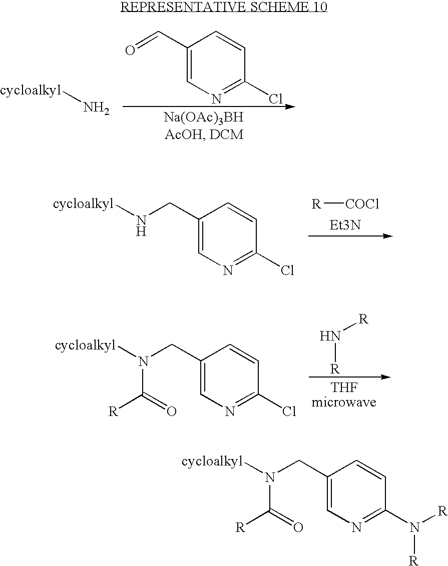

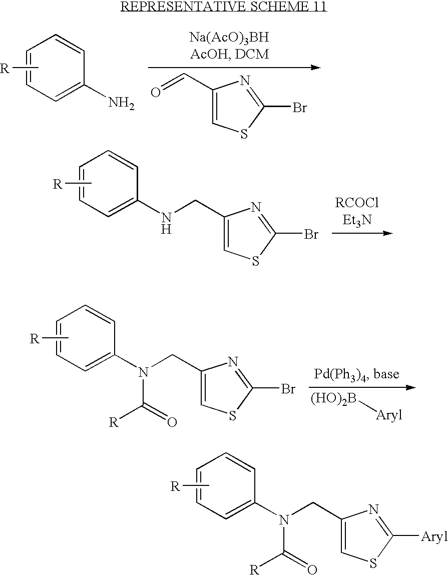

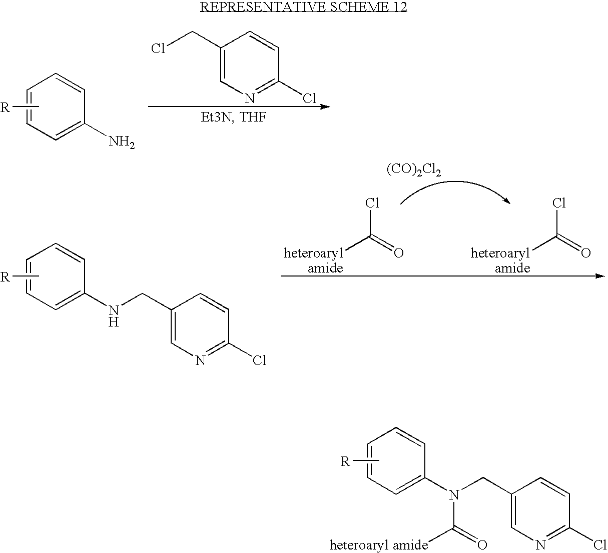

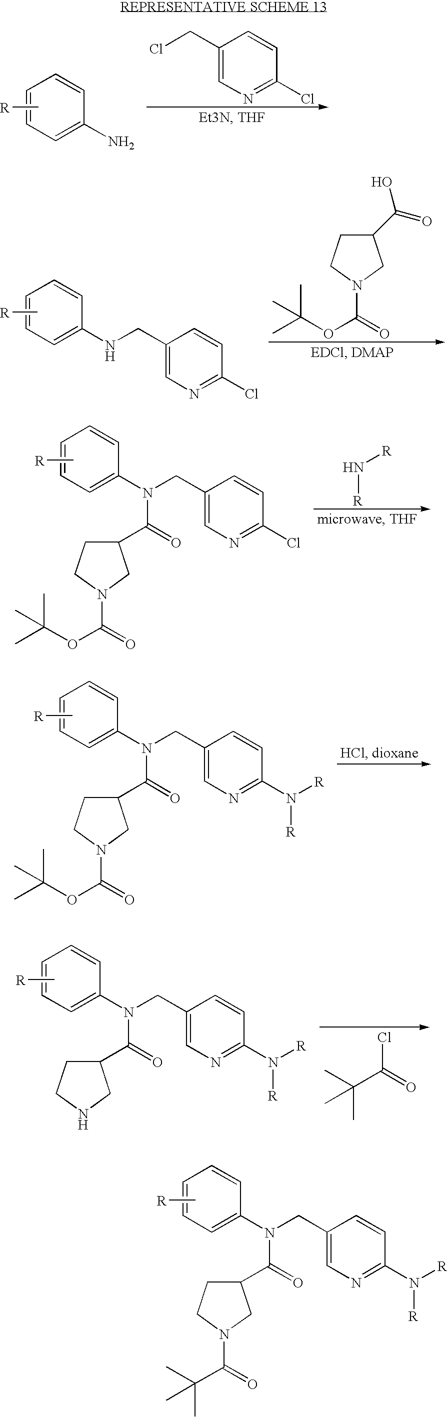

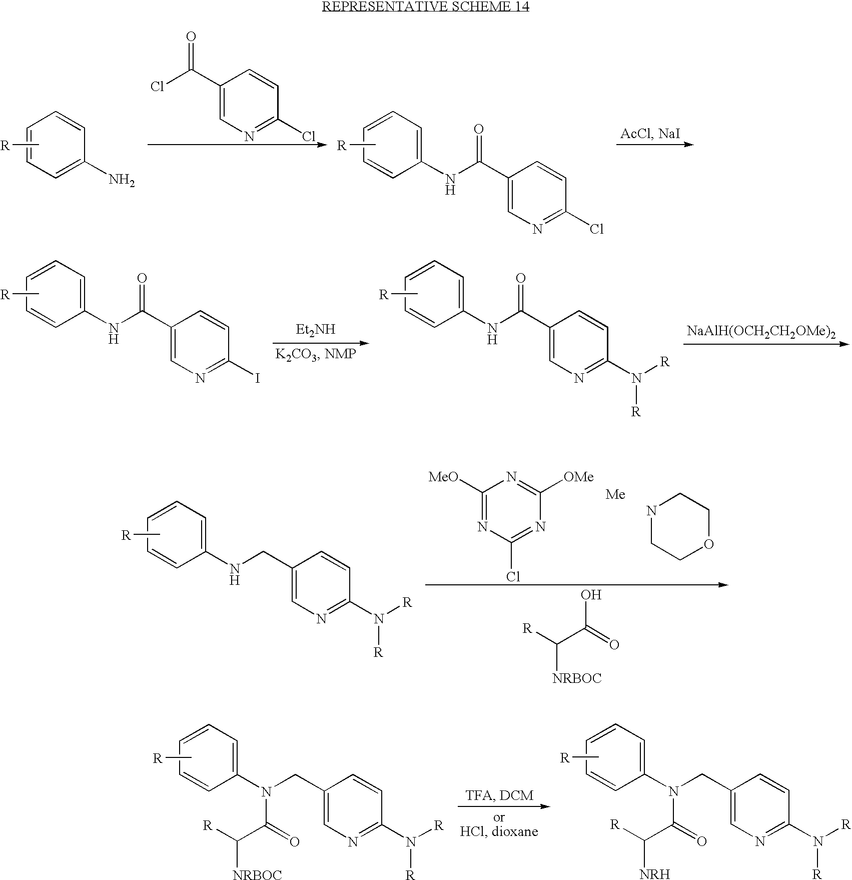

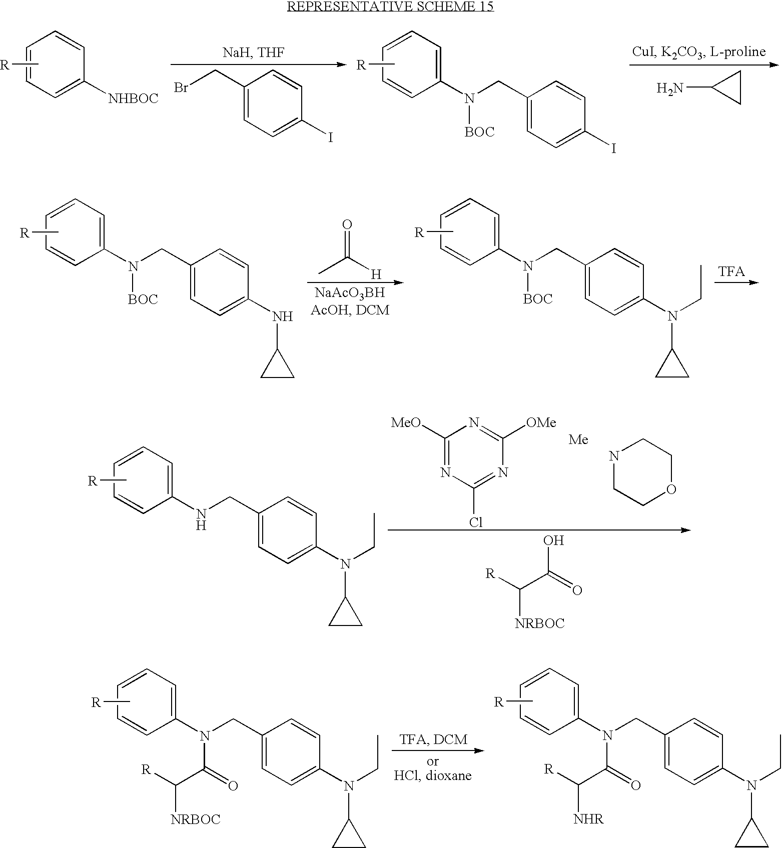

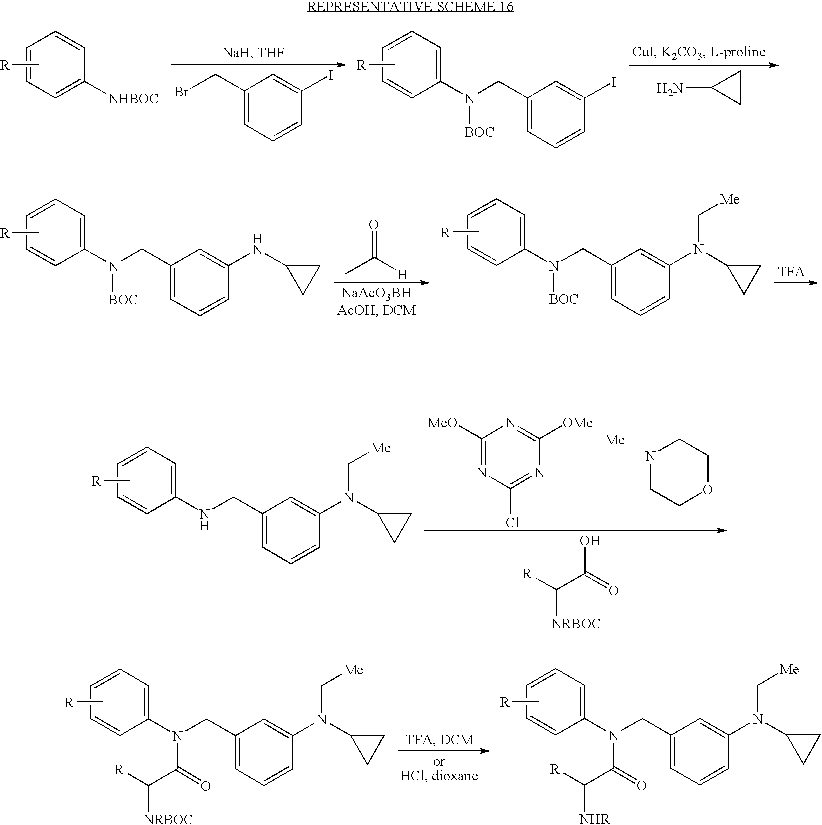

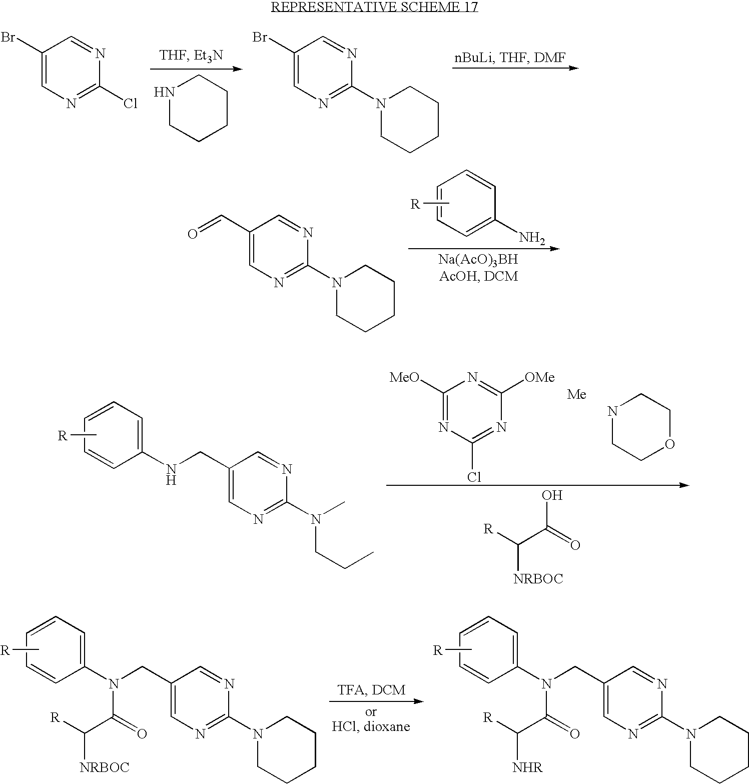

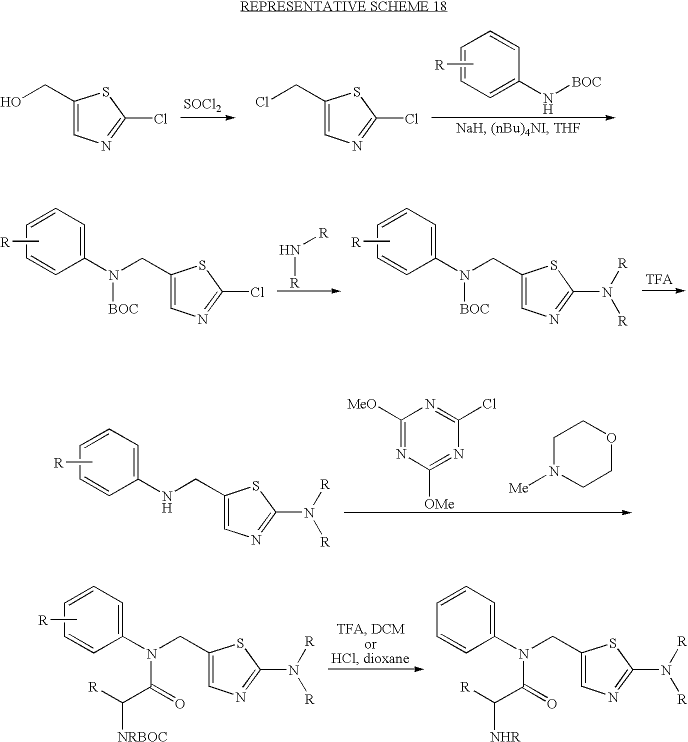

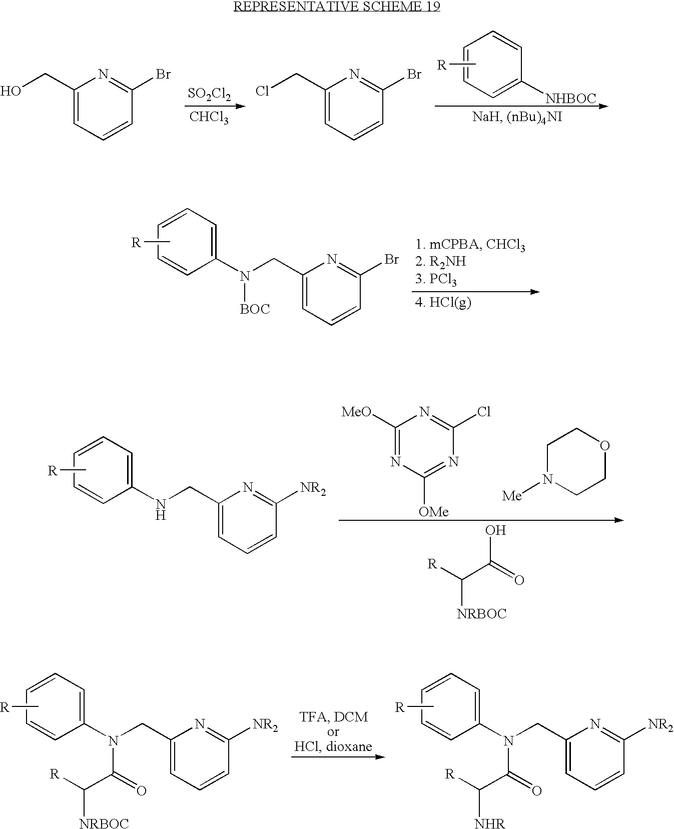

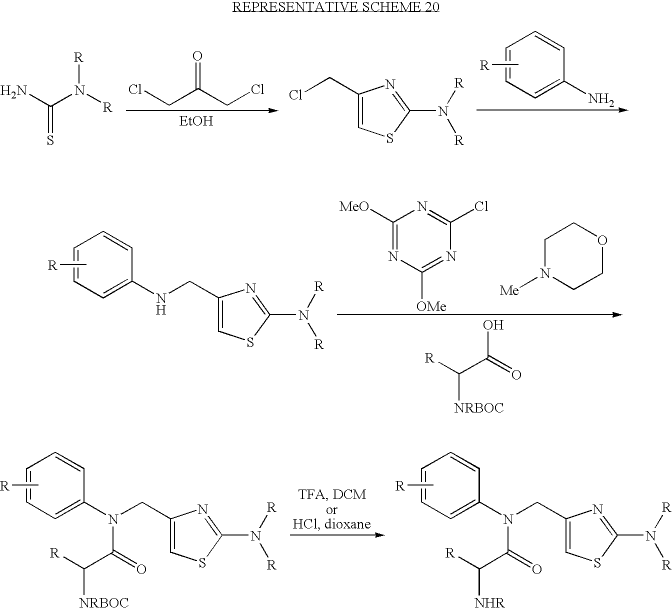

### Example 1A

### PREPARATION OF {5-[(4-FLUORO-PHENYLAMINO)-METHYL]-PYRIDIN-2-YL}-DIETHYLAMINE

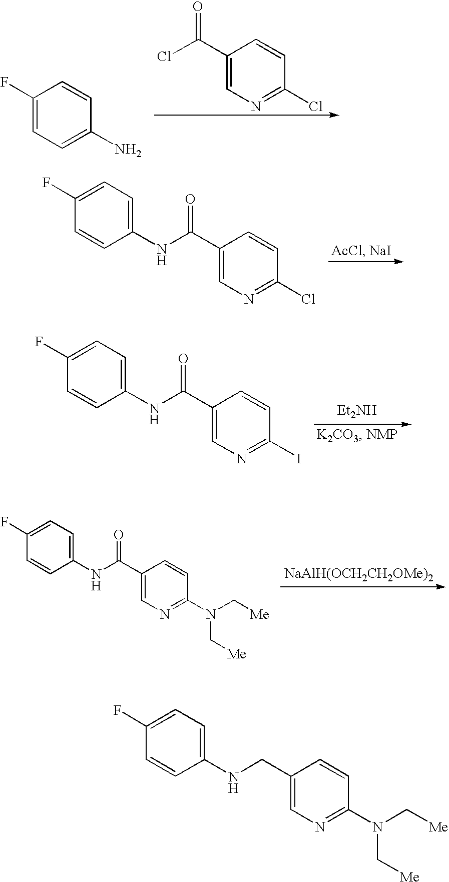

### Part I: Preparation of 6-chloro-N-(4-fluoro-phenyl)-nicotinamide

To a solution of 4-fluoroaniline (15.8 g, 142 mmol) in dichloromethane (450 mL) at 0° C. was slowly added a solution of 6-chloro-nicotinoyl chloride (25 g, 142 mmol) in dichloromethane (50 mL), followed by triethylamine (23.7 mL, 170 mmol). After the addition was complete, the reaction was stirred at 0° C. for 30 minutes, followed by warming to room temperature. After stirring for 30 minutes, the resulting solid was filtered, washed with water and dried under reduced pressure to provide 6-chloro-N-(4-fluoro-phenyl)-nicotinamide (35 g, 139.6 mmol) as a white solid.

### Part II: Preparation of N-(4-fluoro-phenyl)-6-iodo-nicotinamide

To a solution of 6-chloro-N-(4-fluoro-phenyl)-nicotinamide (6.4 g, 25.5 mmol) in acetone (130 mL) was added sodium iodide (38.2 g, 255.3 mmol) followed by the dropwise addition of acetyl chloride (7.3 mL, 102 mmol). The yellow mixture was heated at reflux for 1 hour. The reaction mixture was cooled to room temperature and concentrated to dryness under reduced pressure. The residue was partitioned between ethyl acetate (10 mL) and 1N sodium hydroxide (10 mL). The organic phase was washed with brine, dried over anhydrous sodium sulfate, filtered and concentrated under reduced pressure to provide N-(4-fluoro-phenyl)-6-iodo-nicotinamide (6.76 g, 19.8 mmol) as a white solid.

### Part III: Preparation of 6-diethylamino-N-(4-fluoro-phenyl)-nicotinamide

A mixture of N-(4-fluoro-phenyl)-6-iodo-nicotinamide (684 mg, 2 mmol), diethylamine hydrochloride (0.326 g, 4 mmol), and potassium carbonate (911 mg, 6.6 mmol) in 1-methyl-2-pyrrolidinone (2 mL) was heated at 140° C. in a sealed tube for 65 hours. After cooling to room temperature, a saturated aqueous sodium bicarbonate solution (5 mL) was added, followed by extraction into ethyl acetate (5 mL). The organic phase was dried over anhydrous sodium sulfate, filtered and concentrated under reduced pressure to provide 6-diethylamino-N-(4-fluoro-phenyl)-nicotinamide as a solid which was used directly in the next reaction without further purification.

### Part IV: Preparation of {5-[(4-fluoro-phenylamino)-methyl]-pyridin-2-yl}-diethyl-amine

The 6-diethylamino-N-(4-fluoro-phenyl)-nicotinamide was suspended in a mixture of toluene (5 mL) and tetrahydrofuran (10 mL) and stirred at 0° C. To the reaction was slowly added sodium bis(2-methoxyethoxy)aluminum hydride (65 wt. % in toluene, 1.8 mL). The reaction was allowed to warm to room temperature and stirred for 15 minutes followed by heating at 50° C. for 1 hour. The mixture was cooled to room temperature and quenched by the slow addition of an aqueous saturated sodium bicarbonate solution (10 mL) and 6N sodium hydroxide (10 mL) followed by extraction into ethyl acetate (30 mL). The organic phase was dried over anhydrous sodium sulfate, filtered and concentrated under reduced pressure to provide {5-[(4-fluoro-phenylamino)-methyl]-pyridin-2-yl}diethyl-amine (300 mg, 1.2 mmol) as an oil.

### Example 1B

### ALTERNATIVE PREPARATION OF {5-[(4-FLUORO-PHENYLAMINO)-METHYL]-PYRIDIN-2-YL}-DIETHYL-AMINE DIHYDROCHLORIDE

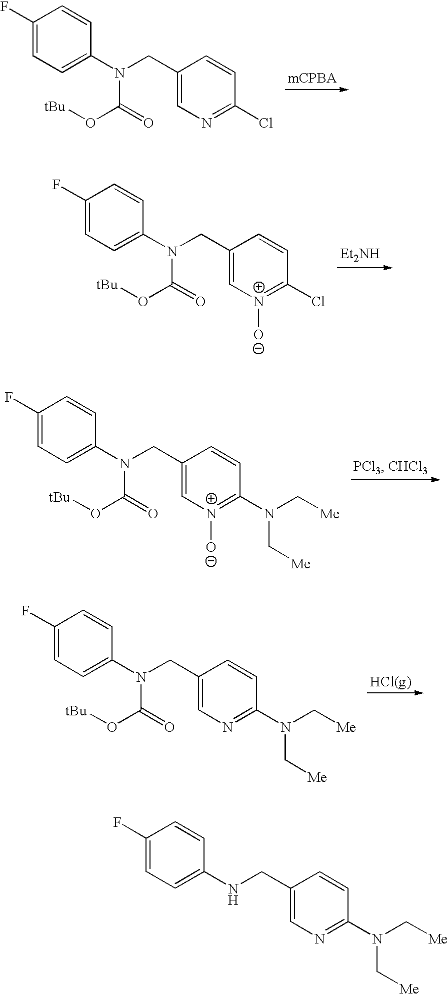

### Part I: Preparation of (6-chloro-1-oxy-pyridin-3-ylmethyl)-(4-fluoro-phenyl)-carbamic acid tert-butyl ester

To a solution of (6-chloro-pyridin-3-ylmethyl)-(4-fluoro-phenyl)-carbamic acid tert-butyl ester (1 g, 2.97 mmol) in chloroform (10 mL) was added m-chloroperbenzoic acid (1 g, 4.5 mmol) and the reaction heated at 50° C. for 6 hours. (6-Chloro-pyridin-3-ylmethyl)-(4-fluoro-phenyl)-carbamic acid tert-butyl ester can be prepared analogously to (6-bromo-pyridin-2-ylmethyl)-(4-fluoro-phenyl)-carbamic acid tert-butyl ester following the procedures described in Example 3 infra. The reaction was cooled to room temperature, diluted with dichloromethane (6 mL), and washed with 3N sodium hydroxide (6 mL). The organic layer was dried over anhydrous sodium sulfate, filtered and evaporated to dryness under reduced pressure. Purification by chromatography (silica gel; 3:7 ethyl acetate:hexane) provided (6-chloro-1-oxy-pyridin-3-ylmethyl)-(4-fluoro-phenyl)-carbamic acid tert-butyl ester (1 g, 2.8 mmol) as a colorless oil.

### Part II: Preparation of (6-diethylamino-1-oxy-pyridin-3-ylmethyl)-(4-fluoro-phenyl)-carbamic acid tert-butyl ester

(6-Chloro-1-oxy-pyridin-3-ylmethyl)-(4-fluoro-phenyl)-carbamic acid tert-butyl ester (1 g, 2.8 mmol) and diethyl amine (2.9 mL, 28.4 mmol) were combined in a sealed tube. The reaction was heated at 130° C. overnight. The reaction was cooled to room temperature and concentrated under reduced pressure. Purification by chromatography (silica gel; ethyl acetate) provided (6-diethylamino-1-oxy-pyridin-3-ylmethyl)-(4-fluoro-phenyl)-(4-fluoro-phenyl)-carbamic acid tert-butyl ester (1 g, 2.5 mmol) as a brown oil.

### Part III: Preparation of (6-diethylamino-pyridin-3-ylmethyl)-(4-fluoro-phenyl)-carbamic acid tert-butyl ester

(6-Diethylamino-1-oxy-pyridin-3-ylmethyl)-(4-fluoro-phenyl)-(4-fluoro-phenyl)-carbamic acid tert-butyl ester (1 g, 2.5 mmol) was dissolved in chloroform (10 mL). Phosphorous trichloride (336 μL, 3.85 mmol) was added, and the reaction was stirred at room temperature for 45 minutes. The reaction mixture was diluted with dichloromethane (10 mL) and washed with 3N sodium hydroxide (20 mL). The organic layer was dried over anhydrous sodium sulfate, filtered and evaporated to dryness under reduced pressure. Purification by chromatography (silica gel; 1:9 ethyl acetate:hexane) provided (6-diethylamino-pyridin-3-ylmethyl)-(4-fluoro-phenyl)-carbamic acid tert-butyl ester (920 mg, 2.5 mmol) as a colorless oil.

### Part IV: Preparation of {5-[(4-fluoro-phenylamino)-methyl]-pyridin-2-yl}-diethyl-amine dihydrochloride

To a solution of (6-diethylamino-pyridin-3-ylmethyl)-(4-fluoro-phenyl)-carbamic acid tert-butyl ester (900 mg, 2.4 mmol) in methanol (10 mL) was added gaseous hydrochloric acid at 0° C. The reaction was allowed to warm to room temperature and stirred for 30 minutes. The reaction was concentrated to provide {5-[(4-fluoro-phenylamino)-methyl]-pyridin-2-yl}-diethyl-amine dihydrochloride (665 mg, 2.4 mmol) as a white solid.

### Example 2

### PREPARATION OF CYCLOPROPYL-{5-[(4-FLUORO-PHENYLAMINO)-METHYL]-PYRIDIN-2-YL}-ETHYL-AMINE DIHYDROCHLORIDE

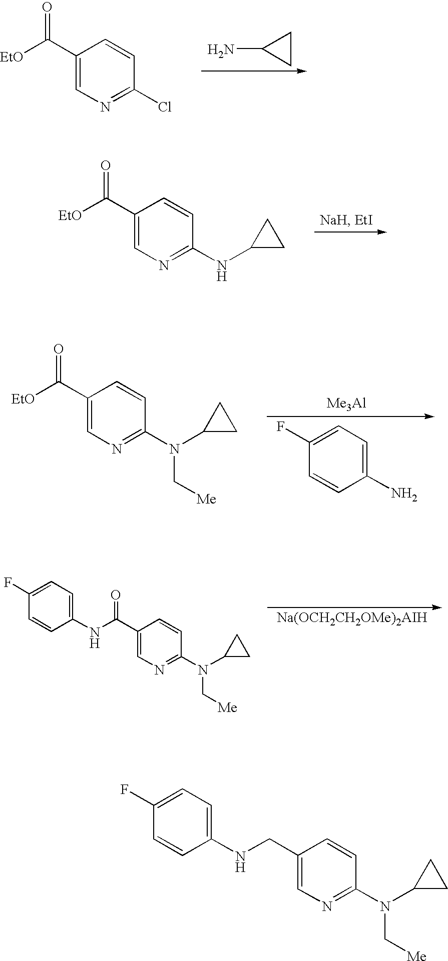

### Part I: Preparation of 6-cyclopropylamino-nicotinic acid ethyl ester

A mixture of ethyl-6-chloro-nicotinate (10 g, 53.9 mmol) in cyclopropyl amine (10 mL) was heated in a sealed tube at 80° C. for 12 hours. The reaction was cooled and the mixture purified by chromatography (silica gel; ethyl acetate:hexane gradient elution) to provide 6-cyclopropylamino-nicotinic acid ethyl ester (6.9 g, 32.2 mmol) as an oil.

### Part II: Preparation of 6-(cyclopropyl-ethyl-amino)-nicotinic acid ethyl ester

To a solution of 6-cyclopropylamino-nicotinic acid ethyl ester (6.9 g, 32.2 mmol) in anhydrous tetrahydrofuran (80 mL) containing dimethyl formamide (50 μL) at 0° C. was added sodium hydride (60% dispersion in mineral oil, 1.85 g, 48.3 mmol). The reaction was allowed to warm to room temperature and stirred for 30 minutes. The reaction was treated with ethyl iodide (3.0 mL, 48.3 mmol) and the reaction allowed to stir overnight. The reaction was quenched by the addition of water (10 mL), followed by extraction with ethyl acetate (2×50 mL). The organic phases were combined, washed with saturated sodium bicarbonate, dried over anhydrous sodium sulfate, filtered and concentrated under reduced pressure. Purification by chromatography (silica gel; ethyl acetate:hexane gradient elution) provided 6-(cyclopropyl-ethyl-amino)-nicotinic acid ethyl ester (6.45 g, 27.5 mmol) as an oil.

### Part III: Preparation of 6-(cyclopropyl-ethyl-amino)-N-(4-fluoro-phenyl)-nicotinamide

To a solution of 4-fluoroaniline (4.65 mL, 35.8 mmol) in toluene (50 mL) was slowly added 2M trimethylaluminum in toluene (16.5 mL, 33 mmol) and the reaction allowed to stir for 1 hour. A solution of 6-(cyclopropyl-ethyl-amino)-nicotinic acid ethyl ester (6.45 g, 27.5 mmol) in toluene (25 mL) was added and the reaction heated to 60° C. After 12 hours, the reaction was cooled to room temperature and quenched by the dropwise addition of methanol. The reaction was concentrated under reduced pressure and the residue taken up into ethyl acetate (100 mL). The organic phase was washed with saturated sodium bicarbonate (25 mL), saturated potassium-sodium tartrate (25 mL), dried over anhydrous sodium sulfate, filtered and concentrated under reduced pressure. Purification by chromatography (silica gel; ethyl acetate:hexane gradient elution) provided 6-(cyclopropyl-ethyl-amino)-N-(4-fluoro-phenyl)-nicotinamide (7.25 g, 24.1 mmol).

### Part IV: Preparation of cyclopropyl-{5-[(4-fluoro-phenylamino)-methyl]-pyridin-2-yl}-ethyl-amine dihydrochloride

To a mixture of 6-(cyclopropyl-ethyl-amino)-N-(4-fluoro-phenyl)-nicotinamide (7.25 g, 24.1 mmol) in toluene (20 mL) and anhydrous tetrahydrofuran (40 mL) at 0° C. was slowly added sodium bis(2-methoxyethoxy)aluminum hydride (65 wt. % in toluene, 16 mL). The reaction was allowed to warm to room temperature and stirred for 15 minutes followed by heating at 50° C. for 1 hour. The mixture was cooled to room temperature and quenched by the slow addition of an aqueous saturated sodium bicarbonate solution (20 mL) and 6N sodium hydroxide (20 mL) followed by extraction into ethyl acetate (60 mL). The organic phase was dried over anhydrous sodium sulfate, filtered and concentrated under reduced pressure. Purification by chromatography (silica gel; ethyl acetate:hexane gradient elution) provided cyclopropyl-{5-[(4-fluoro-phenylamino)-methyl]-pyridin-2-yl}-ethyl-amine (8.0 g, 23.1 mmol).

The free base was treated with ethereal hydrochloric acid to provide cyclopropyl-{5-[(4-fluoro-phenylamino)-methyl]-pyridin-2-yl}-ethyl-amine dihydrochloride (6.8 g, 23.1 mmol) as a white solid.

### Example 3

### PREPARATION OF DIETHYL-{6-[(4-FLUORO-PHENYLAMINO)-METHYL]-PYRIDIN-2-Yl}-AMINE DIHYDROCHLORIDE

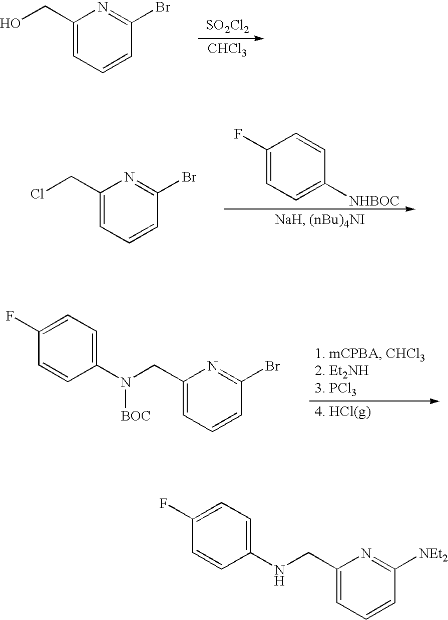

### Part I: Preparation of 2-bromo-6-chloromethyl-pyridine

To a solution of (6-bromo-pyridin-2-yl)-methanol (1.5 g, 8.0 mmol) in chloroform (10 mL) was added dropwise sulfuryl chloride (1.29 mL, 16 mmol) and the reaction stirred overnight. The reaction was concentrated under reduced pressure to provide a yellow semi-solid. The material was triturated with diethyl ether/hexanes and the solid collected to provide 2-bromo-6-chloromethyl-pyridine (900 mg, 4.37 mmol) as a sticky white solid.

### Part II: Preparation of (6-bromo-pyridin-2-ylmethyl)-(4-fluoro-phenyl)-carbamic acid tert-butyl ester

To a solution of (4-fluoro-phenyl)-carbamic acid tert-butyl ester (60144-53-8, 750 mg, 3.55 mmol) in tetrahydrofuran (10 mL) was added sodium hydride (60% dispersion in mineral oil, 150 mg, 3.9 mmol). After 30 minutes, tetra-n-butylammonium iodide (51 mg, 0.36 mmol) and 2-bromo-6-chloromethyl-pyridine (804 mg, 3.9 mmol) was added to the reaction and the mixture was heated to 70° C. After 1 hour, the reaction was cooled to room temperature, quenched with saturated sodium bicarbonate (10 mL) and extracted with ethyl acetate (2×15 mL). The organic phases were combined, dried over anhydrous sodium sulfate, filtered and concentrated under reduced pressure. Flash chromatography (silica gel; 10% ethyl acetate in hexanes) provided (6-bromo-pyridin-2-ylmethyl)-(4-fluoro-phenyl)-carbamic acid tert-butyl ester (700 mg, 1.84 mmol) as an oil which solidified upon standing.

### Part III: Preparation of (6-bromo-1-oxy-pyridin-2-ylmethyl)-(4-fluoro-phenyl)-carbamic acid tert-butyl ester

To a solution of (6-bromo-pyridin-2-ylmethyl)-(4-fluoro-phenyl)-carbamic acid tert-butyl ester (700 mg, 1.84 mmol) in chloroform (8 mL) was added m-chloroperbenzoic acid (477 mg, 2.76 mmol) and the reaction heated to 50° C. After stirring overnight, the reaction was cooled to room temperature, diluted with chloroform (10 mL) and washed with 3N sodium hydroxide (5 mL). The layers were separated and the organic phase was dried over anhydrous sodium sulfate, filtered and concentrated under reduced pressure to provide a yellow solid upon standing. Purification by chromatography (silica gel; 10-20% ethyl acetate in chloroform) provided (6-bromo-1-oxy-pyridin-2-ylmethyl)-(4-fluoro-phenyl)-carbamic acid tert-butyl ester (400 mg, 1.0 mmol) as a white solid.

### Part IV: Preparation of (6-diethylamino-1-oxy-pyridin-2-ylmethyl)-(4-fluoro-phenyl)-carbamic acid tert-butyl ester

A suspension of (6-bromo-1-oxy-pyridin-2-ylmethyl)-(4-fluoro-phenyl)-carbamic acid tert-butyl ester (400 mg, 1.0 mmol) in diethylamine (7 mL) was heated to 130° C. in a sealed tube. After stirring overnight, the reaction was cooled to room temperature and partitioned between brine (10 mL) and ethyl acetate (15 mL). The layers were separated and the organic phase was dried over anhydrous sodium sulfate, filtered and concentrated under reduced pressure to provide a dark liquid. Flash chromatography (silica gel; 30-75% ethyl acetate in chloroform) provided (6-diethylamino-1-oxy-pyridin-2-ylmethyl)-(4-fluoro-phenyl)-carbamic acid tert-butyl ester (230 mg, 0.59 mmol) as a light yellow oil.

### Part V: Preparation of diethyl-{6-[(4-fluoro-phenylamino)-methyl]-pyridin-2-yl}-amine dihydrochloride

To a solution of (6-diethylamino-1-oxy-pyridin-2-ylmethyl)-(4-fluoro-phenyl)-carbamic acid tert-butyl ester (230 mg, 0.59 mmol) in chloroform (2 mL) was added phosphorous trichloride (121 mg, 0.89 mmol). After stirring for 1 hour, the reaction was diluted with chloroform (10 mL) and washed with 3N sodium hydroxide (5 mL). The organic phase was dried over anhydrous sodium sulfate, filtered and concentrated under reduced pressure to provide a yellow oil. The oil was dissolved in chloroform (2 mL) and treated with trifluoroacetic acid (1 mL) and allowed to stir for 1 hour. The reaction was concentrated under reduced pressure and the residue treated with ethereal hydrochloric acid. The resulting solid was collected to provide diethyl-{6-[(4-fluoro-phenylamino)-methyl]-pyridin-2-yl}-amine dihydrochloride (182 mg, 0.59 mmol) as a white solid.

### Example 4

### PREPARATION OF ETHYL-CYCLOPROPYL-{[4-(4-FLUORO-PHENYLAMINO)-METHYL]-PHENYL}-AMINE

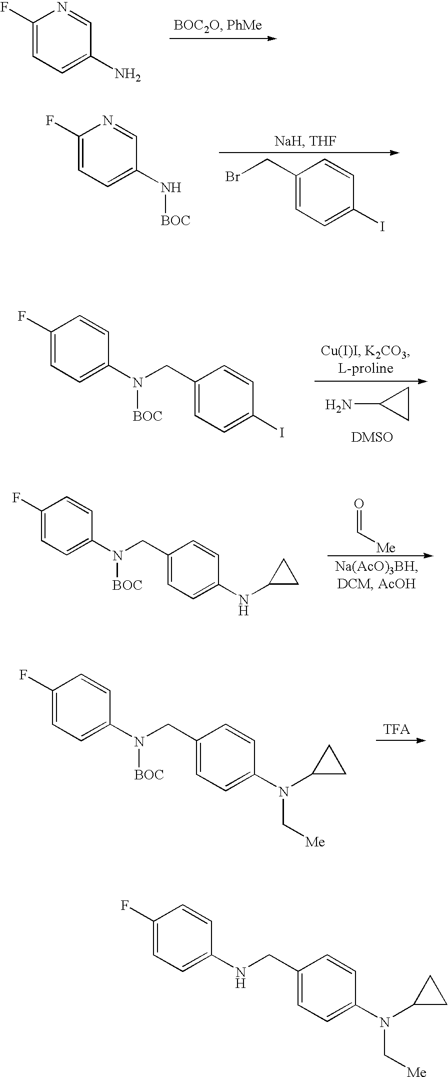

### Part I: Preparation of (4-fluoro-phenyl)-carbamic acid tert-butyl ester

A mixture of 4-fluoroaniline (4.2 mL, 44.1 mmol) and carbonic acid di-tert-butyl ester (11.55 g, 52.9 mmol) in toluene (100 mL) was heated at reflux overnight. The reaction was cooled to room temperature and the solvent was removed under reduced pressure. The residue was triturated with hexanes to provide (4-fluoro-phenyl)-carbamic acid tert-butyl ester (8.4 g, 39.8 mmol) as an off-white solid.

### Part II: Preparation of (4-fluoro-phenyl)-(4-iodo-benzyl)-carbamic acid tert-butyl ester

A solution of (4-fluoro-phenyl)-carbamic acid tert-butyl ester (9.98 g, 47.3 mmol) in anhydrous tetrahydrofuran (150 mL) was cooled to 0° C. and treated with sodium hydride (60% dispersion in mineral oil, 2.3 g, 56.8 mmol). The mixture was warmed to room temperature and stirred for 30 minutes. To the reaction was added 1-bromomethyl-4-iodo-benzene (14.0 g, 47.3 mmol) and the mixture was allowed to stir at room temperature overnight. The reaction was diluted with water (50 mL) and extracted with ethyl acetate (3×50 mL). The organic phases were combined, washed with brine (50 mL), dried over anhydrous sodium sulfate, filtered and concentrated under reduced pressure. Purification by chromatography (silica gel; 5% ethyl acetate in hexanes) provided (4-fluoro-phenyl)-(4-iodo-benzyl)-carbamic acid tert-butyl ester (18 g, 42.1 mmol) as a colorless oil.

### Part III: Preparation of (4-cyclopropylamino-benzyl)-(4-fluoro-phenyl)-carbamic acid tert-butyl ester

A mixture of (4-fluoro-phenyl)-(4-iodo-benzyl)-carbamic acid tert-butyl ester (10 g, 23.4 mmol), cyclopropylamine (4.86 mL, 70.2 mmol), copper (I) iodide (445 mg, 2.34 mmol), potassium carbonate (6.5 g, 46.8 mmol), and L-proline (540 mg, 4.68 mmol) were combined in dimethylsulfoxide (100 mL) and heated at 80° C. for 5 hours. The reaction mixture was cooled, diluted with water (50 mL), and extracted with ethyl acetate (2×100 mL). The combined organic layers were washed with brine, dried over anhydrous sodium sulfate, filtered and evaporated to dryness under reduced pressure. The (4-cyclopropylamino-benzyl)-(4-fluoro-phenyl)-carbamic acid tert-butyl ester was used in the next step without further purification.

### Part IV: Preparation of [4-(cyclopropyl-ethyl-amino)-benzyl]-(4-fluoro-phenyl)-carbamic acid tert-butyl ester

To the (4-cyclopropylamino-benzyl)-(4-fluoro-phenyl)-carbamic acid tert-butyl ester from the previous step in dichloromethane (100 mL) was added acetaldehyde (1.44 mL, 25.7 mmol) and acetic acid (1.6 mL, 28.1 mmol). The solution was stirred at room temperature for 30 minutes, followed by the addition of sodium triacetoxyborohydride (2.0 g, 9.4 mmol). After 30 minutes, another portion of sodium triacetoxyborohydride (2.0 g, 9.4 mmol) was added. A third portion of sodium triacetoxyborohydride (2.0 g, 9.4 mmol) was added and the reaction stirred for 30 minutes. The reaction mixture was basified with 1N sodium hydroxide to pH 10 and extracted with dichloromethane (2×50 mL). The organic phases were combined, dried over anhydrous sodium sulfate, filtered and the solvent removed under reduced pressure to provide a yellow oil. Flash chromatography (silica gel; 10% ethyl acetate in hexanes) provided [4-(cyclopropyl-ethyl-amino)-benzyl]-(4-fluoro-phenyl)-carbamic acid tert-butyl ester a yellow oil, which was used directly in the next reaction.

### Part V: Preparation of ethyl-cyclopropyl-{[4-(4-fluoro-phenylamino)-methyl]-phenyl}-amine

To a solution of [4-(cyclopropyl-ethyl-amino)-benzyl]-(4-fluoro-phenyl)-carbamic acid tert-butyl ester from the previous step in dichloromethane (20 mL) was added trifluoroacetic acid (20 mL) at 0° C. The solution was warmed up to room temperature and stirred for 30 minutes. The reaction was concentrated to dryness under reduced pressure and the residue was dissolved in dichloromethane (20 mL). The organic layer was washed with 3N sodium hydroxide (10 mL), dried over anhydrous sodium sulfate, filtered and evaporated to dryness under reduced pressure. Flash chromatography (silica gel; 10% ethyl acetate in hexanes) provided ethyl-cyclopropyl-{[4-(4-fluoro-phenylamino)-methyl]-phenyl}-amine (6 g, 21.1 mmol) as a yellow oil.

### Example 5

### PREPARATION OF DIETHYL-{[4-(4-FLUORO-PHENYLAMINO)-METHYL]-PHENYL}-AMINE DIHYDROCHLORIDE

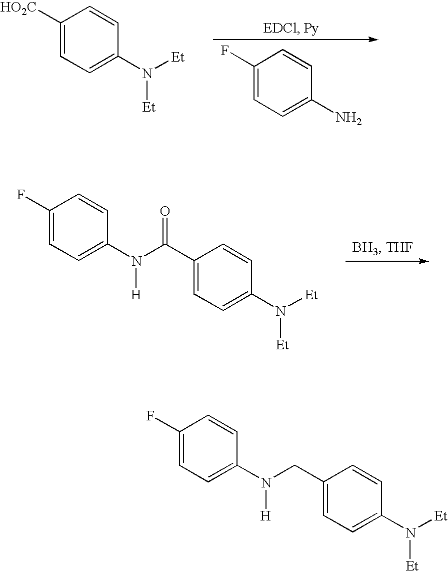

### Part I: Preparation of 4-diethylamino-N-(4-fluoro-phenyl)-benzamide

To a solution of 4-diethylamino-benzoic acid (1.0 g, 5.2 mmol) and N-(3-dimethylaminopropyl)-N′-ethylcarbodiimide (1.4 g, 9.4 mmol) in pyridine (10 mL) was added 4-fluoroaniline (446 μL, 4.7 mmol) and the reaction stirred overnight. The reaction was concentrated under reduced pressure to provide a red oil, which was partitioned between saturated sodium bicarbonate, and hexanes and flash ethyl acetate. The resulting precipitate was collected and dried under reduced pressure to provide 4-diethylamino-N-(4-fluoro-phenyl)-benzamide (1.2 g, 4.2 mmol) as a white solid.

### Part II: Preparation of diethyl-{[4-(4-fluoro-phenylamino)-methyl]-phenyl}-amine dihydrochloride

To a solution of 4-diethylamino-N-(4-fluoro-phenyl)-benzamide (600 mg, 2.1 mmol) in anhydrous tetrahydrofuran (10 mL) was added dropwise 1M borane tetrahydrofuran complex (6.3 mL, 6.3 mmol). The reaction was heated to reflux and stirred for 3 hours. The reaction was cooled to room temperature and treated with saturated hydrochloric acid in methanol (6 mL) and heated to reflux for 3 hours. The reaction was cooled to room temperature and the resulting precipitate filtered and dried to provide diethyl-{[4-(4-fluoro-phenylamino)-methyl]-phenyl}-amine dihydrochloride (622 mg, 1.8 mmol) as a white solid.

### Example 6

### PREPARATION OF ETHYL-CYCLOPROPYL-{[3-(4-FLUORO-PHENYLAMINO)-METHYL]-PHENYL)}AMINE

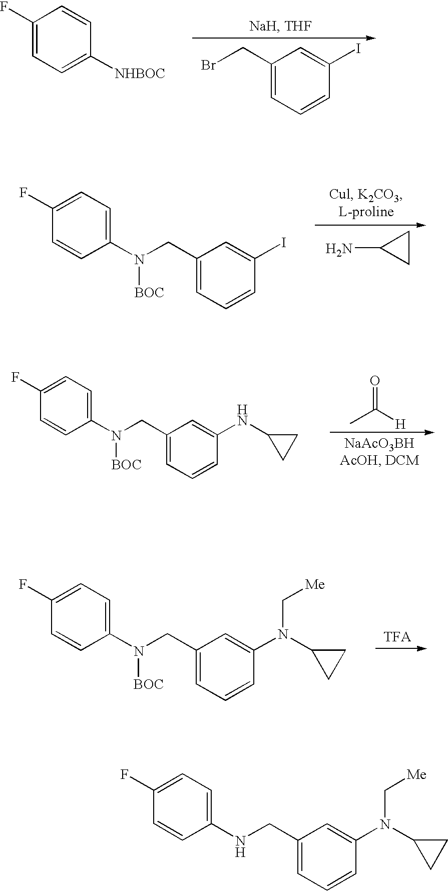

### Part I: Preparation of (4-fluoro-phenyl)-(3-iodo-benzyl)-carbamic acid tert-butyl ester

A solution of (4-fluoro-phenyl)-carbamic acid tert-butyl ester (5 g, 47.3 mmol) in anhydrous tetrahydrofuran (80 mL) was cooled to 0° C. and treated with sodium hydride (60% dispersion in mineral oil, 1.1 g, 28.4 mmol). The mixture was warmed to room temperature and stirred for 30 minutes. To the reaction was added 1-bromomethyl-3-iodo-benzene (7.0 g, 23.7 mmol) and the mixture was allowed to stir at room temperature overnight. The reaction was diluted with water (50 mL) and extracted with ethyl acetate (3×50 mL). The organic phases were combined, washed with brine (50 mL), dried over anhydrous sodium sulfate, filtered and concentrated under reduced pressure. Purification by chromatography (silica gel; 5% ethyl acetate in hexanes) provided (4-fluoro-phenyl)-(3-iodo-benzyl)-carbamic acid tert-butyl ester (9 g, 21.1 mmol) as a colorless oil, which was contaminated with residual (4-fluoro-phenyl)-carbamic acid tert-butyl ester.

### Part II: Preparation of (3-cyclopropylamino-benzyl)-(4-fluoro-phenyl)-carbamic acid tert-butyl ester

A mixture of (4-fluoro-phenyl)-(3-iodo-benzyl)-carbamic acid tert-butyl ester (5.8 g, 13.6 mmol), cyclopropylamine (3.8 mL, 54.4 mmol), copper (I) iodide (260 mg, 1.36 mmol), potassium carbonate (7.5 g, 54.4 mmol), and L-proline (313 mg, 2.72 mmol) were combined in dimethylsulfoxide (60 mL) and heated at 80° C. for 4 hours. The reaction mixture was cooled, diluted with water (50 mL), and extracted with ethyl acetate (2×100 mL). The combined organic layers were washed with brine, dried over anhydrous sodium sulfate, filtered and evaporated to dryness under reduced pressure. The (3-cyclopropylamino-benzyl)-(4-fluoro-phenyl)-carbamic acid tert-butyl ester was used in the next step without further purification.

### Step III: Preparation of [3-(cyclopropyl-ethyl-amino)-benzyl]-(4-fluoro-phenyl)-carbamic acid tert-butyl ester

To the (3-cyclopropylamino-benzyl)-(4-fluoro-phenyl)-carbamic acid tert-butyl ester from the previous step in dichloromethane (30 mL) was added acetaldehyde (840 μL, 15 mmol) and acetic acid (933 μL, 16.3 mmol). The solution was stirred at room temperature for 30 minutes, followed by the addition of sodium triacetoxyborohydride (3.5 g, 16.32 mmol). After 30 minutes, another portion of sodium triacetoxyborohydride (3.5 g, 16.32 mmol) was added. A third portion of sodium triacetoxyborohydride (3.5 g, 16.32 mmol) was added and the reaction stirred for 30 minutes. The reaction mixture was basified with 1N sodium hydroxide to pH 10 and extracted with dichloromethane (2×50 mL). The organic phases were combined, dried over anhydrous sodium sulfate, filtered and the solvent removed under reduced pressure to provide a yellow oil. Flash chromatography (silica gel; 10% ethyl acetate in hexanes) provided (3-(cyclopropyl-ethyl-amino)-benzyl)-(4-fluoro-phenyl)-carbamic acid tert-butyl ester a yellow oil, which was used directly in the next reaction.

### Part IV: Preparation of ethyl-cyclopropyl-{[3-(4-fluoro-phenylamino)-methyl]-phenyl}-amine

To a solution of [3-(cyclopropyl-ethyl-amino)-benzyl]-(4-fluoro-phenyl)-carbamic acid tert-butyl ester from the previous step in dichloromethane (20 mL) was added trifluoroacetic acid (20 mL) at 0° C. The solution was warmed to room temperature and stirred for 30 minutes. The reaction was concentrated to dryness under reduced pressure and the residue was dissolved in dichloromethane (20 mL). The organic layer was washed with 3N sodium hydroxide (10 mL), dried over anhydrous sodium sulfate, filtered and evaporated to dryness under reduced pressure. Flash chromatography (silica gel; 10% ethyl acetate in hexanes) provided ethyl-cyclopropyl-{[3-(4-fluoro-phenylamino)-methyl]-phenyl}-amine (3.4 g, 12 mmol) as a yellow oil.

### Example 7

### PREPARATION OF (4-FLUORO-PHENYL)-[4-(4-METHYL-PIPERAZIN-1-YL)-BENZYL]-AMINE

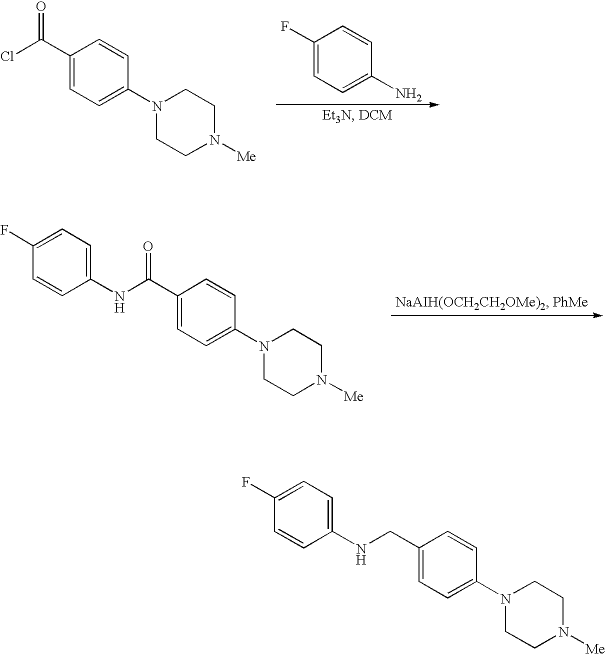

### Part I: Preparation of N-(4-fluoro-phenyl)-4-(4-methyl-piperazin-1-yl)-benzamide

To a solution of 4-fluoroaniline (15.8 g, 142 mmol) in dichloromethane (200 mL) at 0° C. was added dropwise a solution of 4-(4-methylpiperazin-1-yl)-benzoyl chloride (25 g, 142 mmol). As a precipitate formed, the reaction was slowly diluted with additional dichloromethane (300 mL). Triethylamine (23.7 mL, 170 mmol) was added and the reaction was stirred for 30 minutes. The reaction was then warmed to room temperature and stirred for 30 minutes. The resulting precipitate was filtered and washed with water. The filtrate was treated with water, upon which additional precipitates formed. The precipitate was collected and combined with the previously obtained precipitate. The material was dried under reduced pressure overnight to provide N-(4-fluoro-phenyl)-4-(4-methyl-piperazin-1-yl)-benzamide (35 g, 140 mmol) as a white solid.

### Part II: Preparation of (4-fluoro-phenyl)-[4-(4-methyl-piperazin-1-yl)-benzyl]amine

To a solution of N-(4-fluoro-phenyl)-4-(4-methyl-piperazin-1-yl)-benzamide (7.6 g, 24.2 mmol) in anhydrous toluene (50 mL) and anhydrous terahydrofuran (25 mL) at 0° C. was added dropwise sodium bis(2-methoxyethoxy)aluminum hydride (65 wt. % in toluene, 22 mL). After the addition was complete the reaction was heated to reflux and stirred for 1 hour. The reaction was cooled to 0° C. and treated by dropwise addition of 6N sodium hydroxide (50 mL). The reaction was warmed to room temperature, diluted with toluene (50 mL) and stirred for 2 hours. The layers were separated and the aqueous phase washed with toluene (50 mL). The organic phases were combined, washed with saturated sodium bicarbonate (30 mL), water (30 mL), and brine (30 mL). The organic phase was filtered through a pad of Celite® and the Celite® pad washed with ethyl acetate. The organic filtrates were combined and concentrated under reduced pressure to provide a yellow solid. The material was treated with dichloromethane and hexanes to provide (4-fluoro-phenyl)-[4-(4-methyl-piperazin-1-yl)-benzyl]-amine (5.7 g, 19 mmol) as a white solid.

### Example 8

### PREPARATION OF (4-FLUORO-PHENYL)-(2-PIPERIDIN-1-YL-PYRIMIDIN-5-YLMETHYL)-AMINE

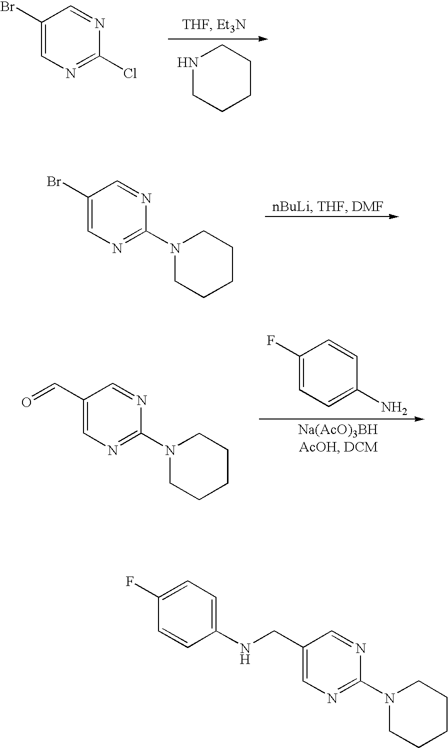

### Part I: Preparation of 5-bromo-2-piperidin-1-yl-pyrimidine

To solution of 5-bromo-2-chloro-pyrimidine (3.0 g, 15.5 mmol) in dichloromethane (30 mL) at room temperature was added piperidine (1.53 mL, 15.5 mmol) followed by the dropwise addition of triethylamine (3.23 mL, 23.3 mmol). The reaction was stirred at room temperature overnight. The reaction was diluted with dichloromethane (20 mL), washed with a saturated aqueous sodium bicarbonate solution (50 mL), followed by brine (50 mL). The organic phase was dried with anhydrous sodium sulfate, filtered and concentrated under reduced pressure. Flash chromatography (silica gel; 5% ethyl acetate in hexanes) provided 5-bromo-2-piperidin-1-yl-pyrimidine as a white solid (3.74 g, 15.5 mmol).

### Part II: Preparation of 2-piperidin-1-yl-pyrimidine-5-carbaldehyde

To a solution of 5-bromo-2-piperidin-1-yl-pyrimidine (1.3 g, 5.4 mmol) in anhydrous tetrahydrofuran (30 mL) at −78° C. was added 1.6 M n-butyl lithium in hexanes (3.7 mL, 5.93 mmol). The mixture was stirred at a temperature below −70° C. for 1 hour. Dimethylformamide (4.2 mL, 53.9 mmol) was added dropwise and the reaction was stirred at a temperature below −70° C. for 1 hour. The reaction was quenched with saturated ammonium chloride (10 mL), diluted with water (20 mL) and extracted with ethyl acetate (2×20 mL). The organic phases were combined, dried over anhydrous sodium sulfate, filtered and the solvent removed under reduced pressure to provide a viscous brown oil. Flash chromatography (silica gel; 5-30% ethyl acetate in hexanes) provided 2-piperidin-1-yl-pyrimidine-5-carbaldehyde (0.76 g, 4.0 mmol) as a white solid.

### Part III: Preparation of (4-fluoro-phenyl)-(2-piperidin-1-yl-pyrimidin-5-ylmethyl)-amine

2-Piperidin-1-yl-pyrimidine-5-carbaldehyde (0.76 g, 4.0 mmol), 4-fluoroaniline (0.76 mL, 8.0 mmol), and acetic acid (0.25 mL, 4.4 mmol) were combined in dichloromethane (8 mL). The solution was stirred at room temperature for 30 minutes, followed by the addition of sodium triacetoxyborohydride (0.28 mg, 1.32 mmol). After 30 minutes, another portion of sodium triacetoxyborohydride (0.28 mg, 1.32 mmol) was added. A third portion of sodium triacetoxyborohydride (0.28 mg, 1.32 mmol) was added and the reaction stirred for 30 minutes. The reaction mixture was basified with 1N sodium hydroxide to pH 10 and extracted with dichloromethane (2×50 mL). The organic phases were combined, dried over anhydrous sodium sulfate, filtered and the solvent removed under reduced pressure to provide a yellow oil. Flash chromatography (silica gel; 5-30% ethyl acetate in hexanes) provided (4-fluoro-phenyl)-(2-piperidin-1-yl-pyrimidin-5-ylmethyl)-amine (0.93 g, 3.25 mmol) as a yellow oil.

### Example 9

### PREPARATION OF (4-FLUORO-PHENYL)-(2-PYRROLIDIN-1-YL-THIAZOL-5-YLMETHYL)-AMINE DIHYDROCHLORIDE

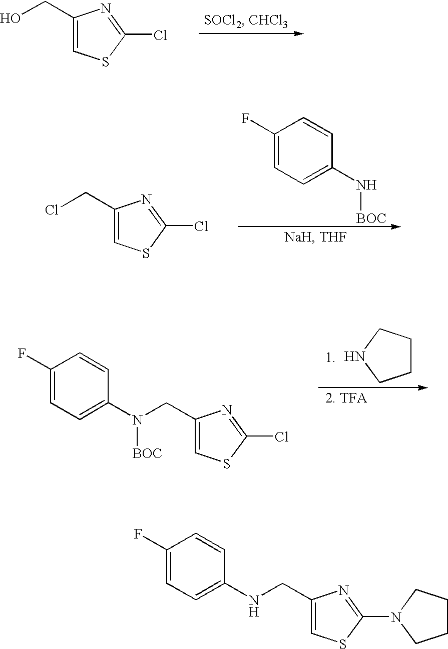

### Part I: Preparation of 2-chloro-5-chloromethyl thiazole

To a solution of 2-chloro-thiazol-5-yl-methanol (1 g, 6.7 mmol) in chloroform (10 mL) was added thionyl chloride (1.6 g, 13.4 mmol), and the reaction was allowed to stir at room temperature overnight. The solvent was removed under reduced pressure to afford a cloudy oil (1.1 g, 6.5 mmol) which was used directly in the next reaction without further purification.

### Part II: Preparation of 2-chloro-thiazol-5-ylmethyl-4-fluorophenylcarbamic acid tert-butyl ester

To a solution of 4-fluorophenylcarbamic acid tert-butyl ester (1.3 g, 6.2 mmol) in anhydrous tetrahydrofuran (15 mL) was added sodium hydride (60% dispersion in mineral oil, 261 mg, 6.8 mmol). After the initial gas evolution had ceased, the reaction was allowed to stir for 15 minutes. Tetra-n-butylammonium iodide (227 mg, 0.6 mmol) was then added followed by addition of the 2-chloro-5-chloromethyl thiazole prepared above. The mixture was heated to reflux for 1 hour. After cooling, the reaction was carefully neutralized with cold saturated sodium bicarbonate (10 mL) and extracted with ethyl acetate (2×20 mL). The organic layers were combined, dried over anhydrous sodium sulfate, filtered and the solvent removed under reduced pressure to provide a dark oil. Flash chromatography (silica gel; 5%-10% ethyl acetate in hexanes) provided 2-chloro-thiazol-5-ylmethyl-4-fluorophenylcarbamic acid tert-butyl ester (1.5 g, 4.4 mmol) as a yellow oil.

### Part III: Preparation of 4-fluorophenyl-2-pyrrolidin-1-yl-thiazol-5-ylmethylcarbamic acid tert-butyl ester

A solution of 2-chloro-thiazol-5-ylmethyl-4-fluorophenylcarbamic acid tert-butyl ester (1.5 g, 4.4 mmol) in pyrrolidine (1.6 mL, 22 mmol) was heated in a sealed tube to 130° C. and stirred overnight. After cooling, the reaction was partitioned between water and ethyl acetate. The organic layer was separated, dried over anhydrous sodium sulfate, filtered and the solvent removed under reduced pressure to provide 4-fluorophenyl-2-pyrrolidin-1-yl-thiazol-5-ylmethylcarbamic acid tert-butyl ester as a yellow oil.

### Part IV: Preparation of (4-fluoro-phenyl)-(2-pyrrolidin-1-yl-thiazol-5-ylmethyl)-amine dihydrochloride

To the free base of 4-fluorophenyl-2-pyrrolidin-1-yl-thiazol-5-ylmethylcarbamic acid tert-butyl ester in dichloromethane (10 mL) was added trifluoroacetic acid (4 mL) and the reaction was stirred at room temperature for 3 hours. After removing the solvent under reduced pressure, the resulting oil was dissolved in diethyl ether and treated with excess ethereal hydrochloric acid. The resulting solid was collected by filtration and dried to provide (4-fluorophenyl)-(2-pyrrolidin-1-yl-thiazol-5-ylmethyl)-amine dihydrochloride (274 mg, 1.2 mmol) as a white solid.

### Example 10

### PREPARATION OF (4-FLUORO-PHENYL)-(2-PIPERIDIN-1-YL-THIAZOL-4-YLMETHYL)-AMINE DIHYDROCHLORIDE

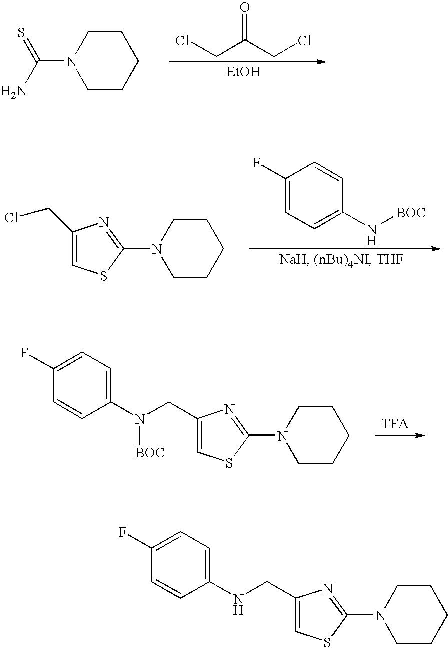

### Part I: Preparation of 1-(4-chloromethyl-thiazol-2-yl)-piperidine

A suspension of piperidine-1-carbothioamide (1.0 g, 6.9 mmol) and 1,3-dichloro-propan-2-one (876 mg, 6.9 mmol) in ethanol (10 mL) was heated to 80° C. and the reaction monitored by liquid chromatography (LC)/mass spectrometry (MS). After 1 hour, the reaction was cooled and the solvent was removed under reduced pressure to provide a pinkish-violet liquid. The liquid was dissolved into ice water (10 mL) and slowly treated with solid sodium bicarbonate, upon which a white precipitate formed. The solid was collected by filtration and dried under reduced pressure. The solid was triturated with hexane and filtered. The filtrate was collected, dried over anhydrous sodium sulfate, filtered and the solvent removed under reduced pressure to afford 1-(4-chloromethyl-thiazol-2-yl)-piperidine (1.1 g, 5.1 mmol) as an off-white solid.

### Part II: Preparation of (4-fluoro-phenyl)-(2-piperidin-1-yl-thiazol-4-ylmethyl)-carbamic acid tert-butyl ester

To a solution of 4-fluorophenylcarbamic acid tert-butyl ester (1.02 g, 4.6 mmol) in tetrahydrofuran (15 mL) was added sodium hydride (60% dispersion in mineral oil, 206 mg, 5.1 mmol). After the initial gas evolution had ceased, the reaction was allowed to stir for 15 minutes. Tetra-n-butylammonium iodide (189 mg, 0.5 mmol) was then added followed by the addition of 1-(4-chloromethyl-thiazol-2-yl)-piperidine (1.1 g, 5.1 mmol) prepared above. The mixture was heated to reflux for 1 hour. After cooling, the reaction was carefully neutralized with cold saturated sodium bicarbonate (10 mL) and extracted with ethyl acetate (2×20 mL). The organic layers were combined, dried over anhydrous sodium sulfate, filtered and the solvent removed under reduced pressure to provide an oil. Flash chromatography (silica gel; 10% ethyl acetate in hexanes) provided (4-fluoro-phenyl)-(2-piperidin-1-yl-thiazol-4-ylmethyl)-carbamic acid tert-butyl ester (1.25 g, 3.2 mmol) as a white solid.

### Part III: Preparation of (4-fluoro-phenyl)-(2-piperidin-1-yl-thiazol-4-ylmethyl)-amine dihydrochloride

To a solution of (4-fluoro-phenyl)-(2-piperidin-1-yl-thiazol-4-ylmethyl)-carbamic acid tert-butyl ester (1.25 g, 3.2 mmol) in dichloromethane (15 mL) was added trifluoroacetic acid (4 mL) and the reaction was stirred at room temperature for 1.5 hours. After removing the solvent under reduced pressure, the resulting oil was dissolved in diethyl ether and treated with excess ethereal hydrochloric acid. The resulting solid was collected by filtration and dried to provide (4-fluoro-phenyl)-(2-piperidin-1-yl-thiazol-4-ylmethyl)-amine dihydrochloride (1.05 g, 3.2 mmol) as a white solid.

### Example 11

### PREPARATION OF (4-FLUORO-PHENYL)-(2-PIPERIDIN-1-YL-THIAZOL-5-YLMETHYL)-AMINE DIHYDROCHLORIDE

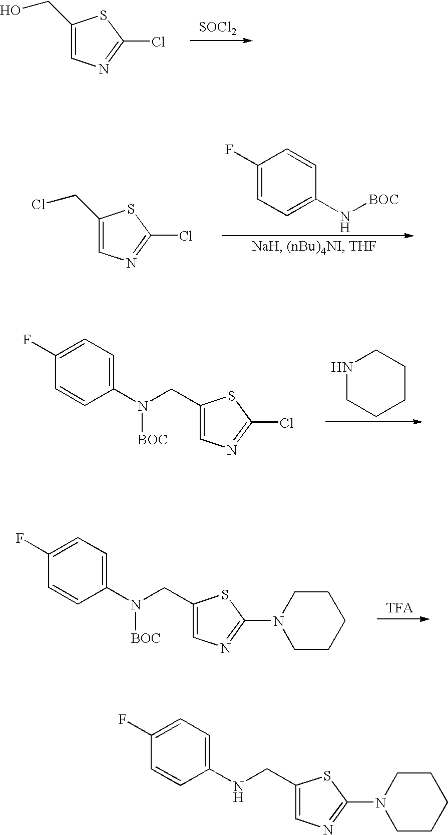

### Part I: Preparation of (2-chloro-thiazol-5-ylmethyl)-(4-fluoro-phenyl)-carbamic acid tert-butyl ester

To a solution of 2-chloro-thiazol-5-yl-methanol (1 g, 6.7 mmol) in chloroform (10 mL) was added thionyl chloride (1.6 g, 13.4 mmol), and the reaction was allowed to stir at room temperature overnight. The solvent was removed under reduced pressure to afford a cloudy oil (1.1 g, 6.5 mmol) which was used directly in the next reaction without further purification.

To a solution of (4-fluorophenyl)-carbamic acid tert-butyl ester (1.3 g, 6.2 mmol) in anhydrous tetrahydrofuran (15 mL) was added sodium hydride (60% dispersion in mineral oil, 261 mg, 6.8 mmol). After the initial gas evolution had ceased, the reaction was allowed to stir for 15 minutes. Tetra-n-butylammonium iodide (227 mg, 0.6 mmol) was then added followed by addition of 2-chloro-5-chloromethyl thiazole prepared above. The mixture was heated to reflux for 1 hour. After cooling, the reaction was carefully neutralized with cold saturated sodium bicarbonate (10 mL) and extracted with ethyl acetate (2×20 mL). The organic layers were combined, dried over anhydrous sodium sulfate, filtered and the solvent removed under reduced pressure to provide a dark oil. Flash chromatography (silica gel; 5%-10% ethyl acetate in hexanes) provided (2-chloro-thiazol-5-ylmethyl)-(4-fluorophenyl)-carbamic acid tert-butyl ester as a yellow oil (1.5 g, 4.4 mmol).

### Part II: Preparation of (4-fluoro-phenyl)-(2-piperidin-1-yl-thiazol-5-ylmethyl)-carbamic acid tert-butyl ester

A solution of (2-chloro-thiazol-5-ylmethyl)-(4-fluorophenyl)-carbamic acid tert-butyl ester (1.5 g, 4.4 mmol) in piperidine (10 mL) was heated in a sealed tube to 130° C. and stirred overnight. After cooling, the reaction was partitioned between water and ethyl acetate. The organic layer was separated, dried over anhydrous sodium sulfate, filtered and the solvent removed under reduced pressure to obtain a yellow oil. Flash chromatography (silica gel; 10%-20% ethyl acetate in hexanes) provided (4-fluorophenyl)-(2-piperidin-1-yl-thiazol-5-ylmethyl)-carbamic acid tert-butyl ester as a light yellow oil (981 mg, 2.6 mmol).

### Part III: Preparation of (4-fluoro-phenyl)-(2-piperidin-1-yl-thiazol-5-ylmethyl)-amine dihydrochloride

To a solution of (4-fluorophenyl)-(2-piperidin-1-yl-thiazol-5-ylmethyl)-carbamic acid tert-butyl ester (981 mg, 2.6 mmol) in dichloromethane (10 mL) was added trifluoroacetic acid (4 mL) and the reaction was stirred at room temperature for 3 hours. After removing the solvent under reduced pressure, the resulting oil was dissolved in diethyl ether and treated with excess ethereal hydrochloric acid. The resulting solid was collected by filtration and dried to provide (4-fluorophenyl)-(2-piperidin-1-yl-thiazol-5-ylmethyl)-amine dihydrochloride as a white solid (472 mg, 1.7 mmol).

### Example 12

### PREPARATION OF 2 CHLORO-4-CHLOROMETHYL-THIAZOLE HYDROCHLORIDE

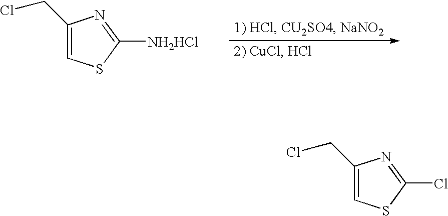

4-chloromethyl-thiazol-2-ylamine hydrochloride (3.0 g, 16.2 mmol) was dissolved in 25 mL HCl. Copper sulfate (90 mg, 0.5 mmol) was added and the reaction mixture was cooled to 0 [symbol] C. Sodium nitrite (2.29 g, 33 mmol) was dissolved in 15 mL of water and added over 15 minutes. The reaction proceeds for 1 hour, warming to room temperature. Copper chloride (1.6 g, 16.2 mmol) was dissolved in 5 mL HCl and added portionwise over 5 minutes. The reaction mixture was extracted 2 times with 100 mL diethyl ether. The organic layers were combined and washed successively with 150 mL water, 150 mL 10% ammonium hydroxide, and 150 mL saturated sodium chloride. The organic layer was dried over magnesium sulfate and solvent was removed under vacuum to yield 2 chloro-4-chloromethyl-thiazole (1.67 g, 9.9 mmol) as an orange oil. This oil was used without further purification.

### Example 13

### PREPARATION OF N-(4-FLUOROPHENYL)-2,2-DIMETHYL-N-(2-PYRROLIDIN-1-YL-THIAZOL-5-YLMETHYL)-PROPIONAMIDE HYDROCHLORIDE (COMPOUND NO. 1)

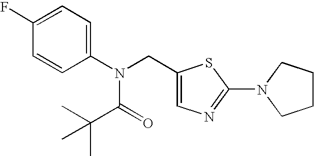

To a solution of (4-fluorophenyl)-(2-pyrrolidin-1-yl-thiazol-5-ylmethyl)-amine (180 mg, 0.65 mmol, Example 9) and triethyl amine (270 μL, 1.95 mmol) in chloroform (4 mL) was added dropwise trimethylacetyl chloride (120 μL, 0.975 mmol). After stirring for 1 hour, the reaction was diluted with dichloromethane (10 mL) and washed with saturated sodium bicarbonate (5 mL). The organic phase was dried over anhydrous sodium sulfate, filtered and evaporated to dryness under reduced pressure. Reverse phase chromatography and isolation of the free base provided N-(4-fluorophenyl)-2,2-dimethyl-N-(2-pyrrolidin-1-yl-thiazol-5-ylmethyl)-propionamide as an off-white solid.

Treatment of the free base with ethereal hydrochloric acid provided N-(4-fluorophenyl)-2,2-dimethyl-N-(2-pyrrolidin-1-yl-thiazol-5-ylmethyl)-propionamide hydrochloride (150 mg, 0.42 mmol) as a white solid.

### Example 14

### PREPARATION OF N-(4-FLUOROPHENYL)-2,2-DIMETHYL-N-(2-PIPERIDIN-1-YL-THIAZOL-4-YLMETHYL)-PROPIONAMIDE HYDROCHLORIDE (COMPOUND NO. 2)

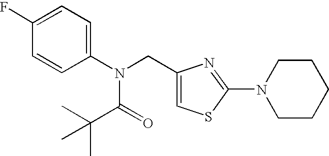

A solution of N-(4-fluorophenyl)-2,2-dimethyl-propionamide (335 mg, 1.72 mmol) in anhydrous tetrahydrofuran (6 mL) was cooled to 0° C. and treated with sodium hydride (60% dispersion in mineral oil, 32 mg, 2.1 mmol). The mixture was warmed to room temperature and stirred for 15 minutes. To the reaction were added tetra-n-butylammonium iodide (125 mg, 0.34 mmol) and (4-fluorophenyl)-(2-piperidin-1-yl-thiazol-4-ylmethyl)-amine (456 mg, 2.1 mmol, Example 10) and the reaction heated to 80° C. After 1 hour, the reaction was cooled to room temperature, and quenched with saturated sodium bicarbonate (10 mL). The reaction was extracted with ethyl acetate (2×10 mL). The organic phases were combined, dried over anhydrous sodium sulfate, filtered, and concentrated under reduced pressure. The resulting semi-solid was triturated with ethyl acetate/hexanes and water to provide a white solid after filtration. MS (base): m/z 376 [M+H]; HPLC (base): tᵣ=3.4 min.

The free base was suspended in methanol (2 mL) and treated with ethereal hydrochloric acid. The solvent was removed to provide N-(4-fluorophenyl)-2,2-dimethyl-N-(2-piperidin-1-yl-thiazol-4-ylmethyl)-propionamide hydrochloride (493 mg, 1.20 mmol).

### Example 15

### PREPARATION OF N-(6-DIETHYLAMINO-PYRIDIN-3-YLMETHYL)-N-(4-FLUOROPHENYL)-2,2-DIMETHYL-PROPIONAMIDE TRIFLUOROACETATE (COMPOUND NO. 3)

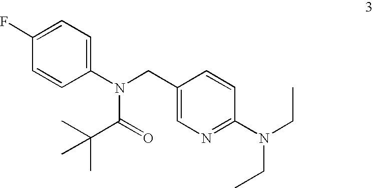

To a solution of {5-[(4-fluorophenylamino)-methyl]-pyridin-2-yl}-diethylamine (232 mg, 0.85 mmol) and triethyl amine (142 μL, 1.0 mmol, Example 1) in anhydrous tetrhydrofuran (4.2 mL) at 0° C. was added dropwise trimethyl acetyl chloride (125 μL, 1.0 mmol). The reaction was warmed to room temperature and allowed to stir overnight. The reaction was diluted with saturated sodium bicarbonate (10 mL) and extracted with ethyl acetate (2×15 mL). The organic phases were combined, dried over anhydrous sodium sulfate, filtered, and concentrated under reduced pressure. Purification by chromatography (silica gel: 10% ethyl acetate:hexanes) provided N-(6-diethylamino-pyridin-3-ylmethyl)-N-(4-fluorophenyl)-2,2-dimethyl-propionamide (320 mg, 0.9 mmol).

The free base was treated with 1% aqueous trifluoroacetic acid, and lypholized to provide N-(6-diethylamino-pyridin-3-ylmethyl)-N-(4-fluorophenyl)-2,2-dimethyl-propionamide trifluoroacetate (423 mg, 0.9 mmol) as a colorless gummy solid.

### Example 16

### PREPARATION OF N-(6-DIETHYLAMINO-PYRIDIN-2-YLMETHYL)-N-(4-FLUOROPHENYL)-2,2-DIMETHYL-PROPIONAMIDE HYDROCHLORIDE (COMPOUND NO. 4)

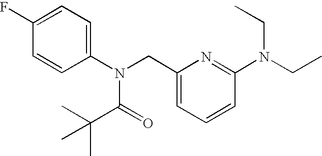

To a suspension of diethyl-{6-[(4-fluorophenylamino)-methyl]-pyridin-2-yl}-amine (195 mg, 0.56 mmol, Example 3) in anhydrous tetrahydrofuran (5 mL) was added triethylamine (124 μL, 2.24 mmol) and trimethylacetyl chloride (224 μL, 1.82 mmol) and the mixture was heated to reflux. After 2 hours, the reaction was cooled to room temperature, quenched with saturated sodium bicarbonate (10 mL) and extracted with ethyl acetate (2×10 mL). The organic phases were combined, dried over anhydrous sodium sulfate, filtered, and concentrated under reduced pressure. Purification by chromatography (silica gel: 10-15% ethyl acetate:hexanes) provided N-(6-diethylamino-pyridin-2-ylmethyl)-N-(4-fluorophenyl)-2,2-dimethyl-propionamide as a white solid.

Treatment of the free base with ethereal hydrochloric acid provided N-(6-diethylamino-pyridin-2-ylmethyl)-N-(4-fluorophenyl)-2,2-dimethyl-propionamide hydrochloride (170 mg, 0.43 mmol) as a gummy foam.

### Example 17

### PREPARATION OF N-(4-DIETHYLAMINOBENZYL)-N-(4-FLUOROPHENYL)-2,2-DIMETHYL-PROPIONAMIDE HYDROCHLORIDE (COMPOUND NO. 5)

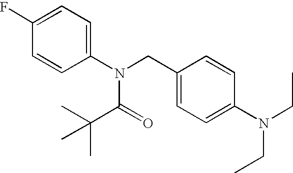

To a suspension of diethyl-{[4-(4-fluorophenylamino)-methyl]-phenyl}-amine (450 mg, 1.65 mmol, Example 5) in anhydrous tetrahydrofuran (10 mL) were added trimethylacetyl chloride (224 μL, 1.82 mmol) and triethyl amine (101 μL, 7.26 mmol) and the mixture heated to reflux. After 2 hours, the reaction was cooled to room temperature, quenched with saturated sodium bicarbonate (10 mL) and extracted with ethyl acetate (2×10 mL). The organic phases were combined, dried over anhydrous sodium sulfate, filtered, and concentrated under reduced pressure. Purification by chromatography (silica gel: 10% ethyl acetate:hexanes) provided N-(4-Diethylaminobenzyl)-N-(4-fluorophenyl)-2,2-dimethyl-propionamide.

The free base was treated with methanolic hydrochloric acid followed by concentration under reduced pressure to provide N-(4-Diethylaminobenzyl)-N-(4-fluorophenyl)-2,2-dimethyl-propionamide hydrochloride (500 mg, 1.28 mmol) as a white foam.

### Example 18

### PREPARATION OF N-(3-(CYCLOPROPYL(ETHYL)AMINO)-BENZYL)-N-(4-FLUOROPHENYL)-2,2-DIMETHYL-PROPIONAMIDE HYDROCHLORIDE (COMPOUND NO. 6)

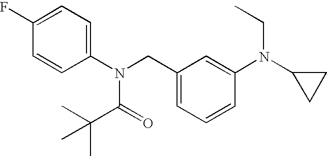

To a solution of ethyl-cyclopropyl-{[3-(4-fluorophenylamino)-methyl]-phenyl)}amine (300 mg, 1.05 mmol, Example 6) and triethyl amine (220 μL, 1.58 mmol) in dichloromethane (6 mL) at 0° C. was added trimethylacetyl chloride (155 μL, 1.58 mmol) and the reaction was warmed to room temperature. After stirring for 30 minutes, the reaction was quenched with saturated sodium bicarbonate (10 mL) and extracted with ethyl acetate (2×10 mL). The organic phases were combined, dried over anhydrous sodium sulfate, filtered, and concentrated under reduced pressure. Purification by reverse phase chromatography and isolation of the free base provided N-(3-(Cyclopropyl(ethyl)amino)-benzyl)-N-(4-fluorophenyl)-2,2-dimethyl-propionamide (272 mg, 0.74 mmol) as a white solid.

Treatment of the free base with ethereal hydrochloric acid provided N-(3-(Cyclopropyl(ethyl)amino)-benzyl)-N-(4-fluorophenyl)-2,2-dimethyl-propionamide hydrochloride (300 mg, 0.74 mmol) as a white solid.

### Example 19

### PREPARATION OF N-[6-(CYCLOPROPYL(ETHYL)AMINO)-PYRIDIN-3-YLMETHYL]-N-(4-FLUOROPHENYL)-2,2-DIMETHYL-PROPIONAMIDE HYDROCHLORIDE (COMPOUND NO. 7)

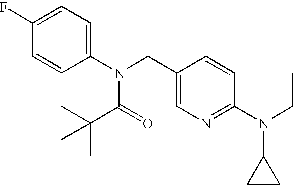

To a solution of ethyl-cyclopropyl-{[4-(4-fluorophenylamino)-methyl]-phenyl}-amine (200 mg, 0.56 mmol, Example 4) in dichloromethane (4 mL) were added trimethylacetyl chloride (83 μL, 0.67 mmol) and triethyl amine (312 μL, 2.24 mmol). After stirring for 1 hour, the reaction was quenched with saturated sodium bicarbonate (10 mL) and extracted with ethyl acetate (2×10 mL). The organic phases were combined, dried over anhydrous sodium sulfate, filtered, and concentrated under reduced pressure. Purification by reverse phase chromatography and isolation of the free base provided N-(6-(Cyclopropyl(ethyl)amino)-pyridin-3-ylmethyl)-N-(4-fluorophenyl)-2,2-dimethyl-propionamide.

Treatment of the free base with ethereal hydrochloric acid provided N-[6-(Cyclopropyl(ethyl)amino)-pyridin-3-ylmethyl)-N-(4-fluorophenyl)-2,2-dimethyl-propionamide hydrochloride (180 mg, 0.45 mmol) as a white solid.

### Example 20

### PREPARATION OF N-(4-FLUOROPHENYL)-2,2-DIMETHYL-N-(2-PIPERIDIN-1-YL-PYRIMIDIN-5-YLMETHYL)-PROPIONAMIDE HYDROCHLORIDE (COMPOUND NO. 8)

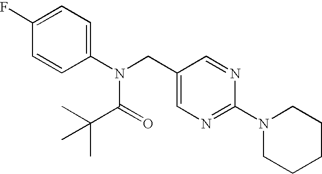

To a solution of (4-fluorophenyl)-(2-piperidin-1-yl-pyrimidin-5-ylmethyl)-amine (50 mg, 0.175 mmol, Example 8) and triethyl amine (37 μL, 0.26 mmol) in dichloromethane (4 mL) at 0° C. was added trimethylacetyl chloride (24 μL, 0.19 mmol) and the reaction was warmed to room temperature. After stirring for 1 hour, the reaction was quenched with saturated sodium bicarbonate (10 mL) and extracted with ethyl acetate (2×10 mL). The organic phases were combined, dried over anhydrous sodium sulfate, filtered, and concentrated under reduced pressure. Purification by reverse phase liquid chromatography provided N-(4-fluorophenyl)-2,2-dimethyl-N-(2-piperidin-1-yl-pyrimidin-5-ylmethyl)-propionamide (50 mg, 0.14 mmol) as a white solid.

Treatment of the free base with ethereal hydrochloric acid provided N-(4-fluorophenyl)-2,2-dimethyl-N-(2-piperidin-1-yl-pyrimidin-5-ylmethyl)-propionamide hydrochloride (57 mg, 0.14 mmol) as a white solid.

### Example 21

### PREPARATION OF 3-TERT-BUTYL-1-(4-FLUOROPHENYL)-1-(6-(4-METHYL-PIPERAZIN-1-YL)-PYRIDIN-3-YLMETHYL)-UREA HYDROCHLORIDE (COMPOUND NO. 11)

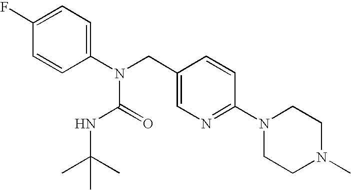

To a suspension of (4-fluorophenyl)-[6-(4-methyl-piperazin-1-yl)-pyridin-3-ylmethyl]-amine (150 mg, 0.5 mmol) in acetonitrile (2 mL) was added tert-butyl isocyanate (171 μL, 1.5 mmol) and the reaction was heated to 90° C. After 2 days, the reaction was cooled to room temperature and the solvent removed under reduced pressure. The residue was treated with ethereal hydrochloric acid to provide 3-tert-butyl-1-(4-fluorophenyl)-1-(6-(4-methyl-piperazin-1-yl)-pyridin-3-ylmethyl)-urea hydrochloride (160 mg, 0.37 mmol). MS (base): m/z 400 [M+H]; HPLC (base): tᵣ=4.4 min.

### Example 22

### PREPARATION OF 1-BENZYL-PIPERIDINE-4-CARBOXYLIC ACID [6-(CYCLOPROPYL(ETHYL)AMINO)-PYRIDIN-3-YLMETHYL]-(4-FLUOROPHENYL)-AMIDE DIHYDROCHLORIDE (COMPOUND NO. 12)

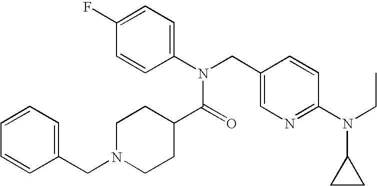

To a suspension of cyclopropyl-{5-[(4-fluorophenylamino)-methyl]-pyridin-2-yl}-ethyl-amine (358 mg, 1 mmol, Example 2) and 1-tert-butoxy carbonyl piperidine-4-carboxylic acid (252 mg, 1.1 mmol) in pyridine (10 mL) was added N-(3-dimethylaminopropyl)-N′-ethylcarbodiimide (233 mg, 1.5 mmol) and a seed crystal of 4-dimethyl aminopyridine. After stirring overnight, the reaction was diluted with ethyl acetate (40 mL), and washed with water (2×20 mL), saturated sodium bicarbonate (20 mL), and brine (20 mL). The organic phase was dried over anhydrous magnesium sulfate, filtered and concentrated under reduced pressure to provide 4-{([6-(Cyclopropyl(ethyl)amino)-pyridin-3-ylmethyl]-(4-fluorophenyl)-carbamoyl}-piperidine-1-carboxylic acid tert-butyl ester.

To a solution of 4-{[6-(Cyclopropyl(ethyl)amino)-pyridin-3-ylmethyl]-(4-fluorophenyl)-carbamoyl}-piperidine-1-carboxylic acid tert-butyl ester in dichloromethane (10 mL) was added trifluoroacetic acid (5 mL). After stirring for 2 hours, the reaction was concentrated under reduced pressure to provide piperidine-4-carboxylic acid [6-(Cyclopropyl(ethyl)amino)-pyridin-3-ylmethyl]-(4-fluorophenyl)-amide (447 mg, 0.9 mmol).

To a solution of piperidine-4-carboxylic acid [6-(Cyclopropyl(ethyl)amino)-pyridin-3-ylmethyl]-(4-fluorophenyl)-amide (447 mg, 0.9 mmol) in methanol (10 mL) was added (108 μL, 0.9 mmol) of benzyl bromide and potassium carbonate (373 mg, 2.7 mmol) and the reaction heated to reflux. After stirring overnight, the reaction was cooled to room temperature and the solvent removed under reduced pressure. The residue was taken up into ethyl acetate (20 mL), washed with saturate sodium bicarbonate, dried over anhydrous magnesium sulfate, filtered and concentrated under reduced pressure to provided 1-benzyl-piperidine-4-carboxylic acid [6-(Cyclopropyl(ethyl)amino)-pyridin-3-ylmethyl]-(4-fluorophenyl)-amide. MS (base): m/z 487 [M+H]; HPLC (base): tᵣ=3.8 min.

Treatment of the free base with ethereal hydrochloric acid provided 1-benzyl-piperidine-4-carboxylic acid [6-(Cyclopropyl(ethyl)amino)-pyridin-3-ylmethyl]-(4-fluorophenyl)-amide dihydrochloride (448 mg, 0.8 mmol) as a white solid.

### Example 23

### PREPARATION OF 4-METHYL-PENTANOIC ACID [6-(CYCLOPROPYL(ETHYL)AMINO)-PYRIDIN-3-YLMETHYL]-(4-FLUOROPHENYL)-AMIDE HYDROCHLORIDE (COMPOUND NO. 13)

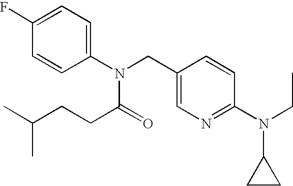

To a solution of cyclopropyl-{5-[(4-fluorophenylamino)-methyl]-pyridin-2-yl}-ethyl-amine (259 mg, 0.72 mmol, Example 2) and N-(3-dimethylaminopropyl)-N′-ethylcarbodiimide (167 mg, 1.1 mmol) in pyridine (4 mL) was added 4-methylvaleric acid (139 mg, 1.2 mmol) and a seed crystal of 4-dimethyl aminopyridine. After stirring overnight, the reaction was diluted with ethyl acetate (40 mL) washed with water (2×20 mL), saturated sodium bicarbonate (20 mL), and brine (20 mL). The organic phase was dried over anhydrous magnesium sulfate, filtered and concentrated under reduced pressure to provide 4-methyl-pentanoic acid [6-(Cyclopropyl(ethyl)amino)-pyridin-3-ylmethyl]-(4-fluorophenyl)-amide.

Treatment of the free base with ethereal hydrochloric acid provided 4-methyl-pentanoic acid [6-(Cyclopropyl(ethyl)amino)-pyridin-3-ylmethyl]-(4-fluorophenyl)-amide hydrochloride (231 mg, 0.55 mmol) as a gummy solid.

### Example 24

### PREPARATION OF 1,2,3,4-TETRAHYDRO-ISOQUINOLINE-3-CARBOXYLIC ACID [6-(CYCLOPROPYL(ETHYL)AMINO)-PYRIDIN-3-YLMETHYL]-(4-FLUOROPHENYL)-AMIDE DIHYDROCHLORIDE (COMPOUND NO. 14)

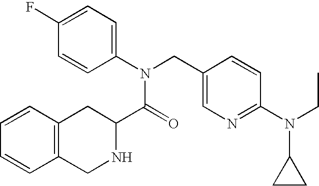

To a solution of cyclopropyl-{5-[(4-fluorophenylamino)-methyl]-pyridin-2-yl}-ethyl-amine (358 mg, 1.0 mmol, Example 2) and N-(3-dimethylaminopropyl)-N′-ethylcarbodiimide (233 mg, 1.5 mmol) in pyridine (4 mL) was added 3,4-dihydro-1H-isoquinoline-2,3-dicarboxylic acid 2-benzyl ester (342 mg, 1.1 mmol) and a seed crystal of 4-dimethyl aminopyridine. After stirring overnight, the reaction was diluted with ethyl acetate (50 mL), and washed with water (2×20 mL), saturated sodium bicarbonate (20 mL), and brine (20 mL). The organic phase was dried over anhydrous magnesium sulfate, filtered and concentrated under reduced pressure to provide 3-{[6-(Cyclopropyl(ethyl)amino)-pyridin-3-ylmethyl]-(4-fluorophenyl)-carbamoyl}-3,4-dihydro-1H-isoquinoline-2-carboxylic acid benzyl ester (434 mg, 0.75 mmol).

To a solution of 3-{[6-(Cyclopropyl(ethyl)amino)-pyridin-3-ylmethyl]-(4-fluorophenyl)-carbamoyl}-3,4-dihydro-1H-isoquinoline-2-carboxylic acid benzyl ester (434 mg, 0.75 mmol) in ethyl acetate was added 5% palladium on carbon and the reaction was stirred overnight under a hydrogen atmosphere at 30 psi. The hydrogen atmosphere was replaced with nitrogen and the reaction was filtered through Celite®. The filtrate was concentrated under reduced pressure. The residue was purified by reverse phase chromatography and isolation of the free base provided 1,2,3,4-tetrahydro-isoquinoline-3-carboxylic acid [6-(Cyclopropyl(ethyl)amino)-pyridin-3-ylmethyl]-(4-fluorophenyl)-amide. MS (base): m/z 445 [M+H]; HPLC (base): tᵣ=3.6 min.

Treatment of the free base with ethereal hydrochloric acid provided 1,2,3,4-tetrahydro-isoquinoline-3-carboxylic acid [6-(Cyclopropyl(ethyl)amino)-pyridin-3-ylmethyl]-(4-fluorophenyl)-amide dihydrochloride (245 mg, 0.55 mmol) as a white solid.

### Example 25

### PREPARATION OF 2-METHYL-1,2,3,4-TETRAHYDRO-ISOQUINOLINE-3-CARBOXYLIC ACID [6-(CYCLOPROPYL(ETHYL)AMINO)-PYRIDIN-3-YLMETHYL]-(4-FLUOROPHENYL)-AMIDE DIHYDROCHLORIDE (COMPOUND NO. 15)

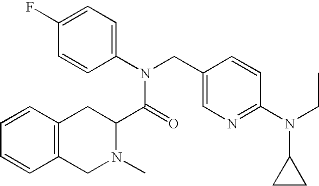

To a solution of the free base of 1,2,3,4-tetrahydro-isoquinoline-3-carboxylic acid [6-(Cyclopropyl(ethyl)amino)-pyridin-3-ylmethyl]-(4-fluorophenyl)-amide (322 mg, 0.67 mmol, Example 25) in dichloromethane (8 mL) and aqueous formaldehyde (37%, 60 μL) was added sodium triacetoxy borohydride (636 mg, 3.0 mmol) and the reaction allowed to stir overnight. The reaction was carefully neutralized with cold saturated sodium bicarbonate (10 mL) and extracted with ethyl acetate (2×20 mL). The organic layers were combined, dried over anhydrous sodium sulfate, filtered and the solvent removed under reduced pressure to provide 2-methyl-1,2,3,4-tetrahydro-isoquinoline-3-carboxylic acid [6-(Cyclopropyl(ethyl)amino)-pyridin-3-ylmethyl]-(4-fluorophenyl)-amide. MS (base): m/z 459 [M+H]; HPLC (base): tᵣ=3.5 min.

Treatment of the free base with ethereal hydrochloric acid provided 2-methyl-1,2,3,4-tetrahydro-isoquinoline-3-carboxylic acid [6-(Cyclopropyl(ethyl)amino)-pyridin-3-ylmethyl]-(4-fluorophenyl)-amide dihydrochloride (320 mg, 0.6 mmol) as a white solid.

### Example 26

### PREPARATION OF TETRAHYDRO-FURAN-2-CARBOXYLIC ACID (4-FLUOROPHENYL)-(6-(4-METHYL-PIPERAZIN-1-YL)-PYRIDIN-3-YLMETHYL)-AMIDE TRIFLUOROACETATE (COMPOUND NO. 16)

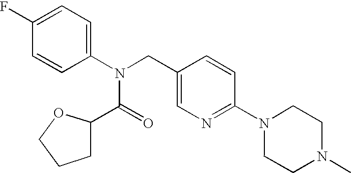

To a mixture of 2-tetrahydrofuroic acid (288 mL, 3 mmol) in chloroform (5 mL) was added thionyl chloride (660 μL, 9 mmol) and the reaction heated to reflux for 1 hour. The reaction was cooled and concentrated under reduced pressure to provide tetrahydro-furan-2-carbonyl chloride (405 mg, 3 mmol).

To a solution of tetrahydro-furan-2-carbonyl chloride (135 mg, 1 mmol) in dichloromethane (5 mL) and triethyl amine (278 μL, 2 mmol) was added (4-fluorophenyl)-[6-(4-methyl-piperazin-1-yl)-pyridin-3-ylmethyl]-amine (300 mg, 1.0 mmol). After stirring for 30 minutes, the reaction was quenched with saturated sodium bicarbonate (10 mL) and extracted with ethyl acetate (2×10 mL). The organic phases were combined, dried over anhydrous sodium sulfate, filtered, and concentrated under reduced pressure. Purification by reverse phase chromatography and isolation of the free base provided 2-(4-chloro-phenyl)-N-(4-fluorophenyl)-N-(6-(4-methyl-piperazin-1-yl)-pyridin-3-ylmethyl)-isobutyramide trifluoroacetate. MS (base): m/z 399 [M+H]; HPLC (base): tᵣ=3.7 min.

### Example 27

### PREPARATION OF N-(4-FLUOROPHENYL)-2,2-DIMETHYL-N-[(2-PHENYL-1,3-THIAZOL-4-YL)METHYL]PROPANAMIDE (COMPOUND NO. 180)

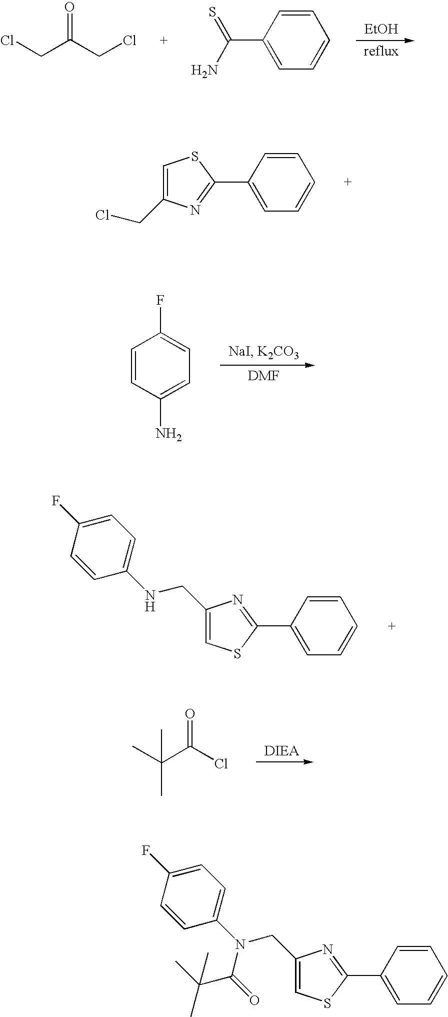

### Part 1: Preparation of 4-Chloromethyl-2-phenylthiazole

To a solution of thiobenzamide (3.0 g, 21.9 mmol) in 100 mL ethanol 40 mL tetrahydrofuran at 65 [symbol] C was added dichloroacetone (3.05 g, 24 mmol). The reaction proceeded at reflux for 5 hours. Solvent was removed under vacuum and quenched with sodium bicarbonate, then extracted with ethyl acetate. Ethyl acetate fractions were combined and washed with brine, then dried over anhydrous magnesium sulfate. The organic layer was then dried onto silica and chromatographed (silica gel; 0 to 100% ethyl acetate in hexane) providing 4-Chloromethyl-2-phenylthiazole (2.1 g, 10 mmol) as an orange oil.

### Part II: Preparation of (4-fluoro-phenyl)-(2-phenylthiazole-4-ylmethyl)-amine

The compound 4-Chloromethyl-2-phenylthiazole (2.1 g, 10 mmol) was dissolved in dimethyl formamide (10 mL). 4-fluoroanaline (4.8 mL, 50 mmol), sodium iodide (1.5 g, 10 mmol) and potassium carbonate (2.76 g, 20 mmol) were added and heated to 65 [symbol] C for two days. The reaction mixture was diluted with water and extracted with diethyl ether. The organic fractions were combined and dried over magnesium sulfate, then evaporated onto silica gel. The reaction mixture was chromatographed (silica gel; 0 to 100% ethyl acetate in hexane) yielding a dark oil. Excess 4-fluoroanaline was then removed under reduced pressure. The resulting oil was dissolved in diethyl ether and was washed with 2 N HCl. The organic layer was then washed with 2 N NaOH. The organic layer was dried over magnesium sulfate then the solvent removed under vacuum providing (4-fluoro-phenyl)-(2-phenylthiazole-4-ylmethyl)-amine (1.42 g, 5 mmol) as brown powder.

### Part III: Preparation of N-(4-fluorophenyl)-2,2-dimethyl-N-[(2-phenyl-1,3-thiazol-4-yl)methyl]propanamide

The compound (4-fluoro-phenyl)-(2-phenylthiazole-4-ylmethyl)-amine (200 mg, 0.7 mmol) was dissolved in tetrahydrofuran (5 mL). Trimethyl acetyl chloride (0.104 mL, 0.84 mmol) was added followed by diisopropylethylamine (0.146 ml, 0.84 mmol). The reaction was stirred overnight at room temperature. The reaction was diluted with water and extracted with ethyl acetate. The organic layers were combined and dried over magnesium sulfate. The organic layer was evaporated onto silica gel and chromatographed (silica gel; 0 to 30% ethyl acetate in hexane). The solvent was removed under vacuum resulting in an off white solid, N-(4-fluorophenyl)-2,2-dimethyl-N-[(2-phenyl-1,3-thiazol-4-yl)methyl]propanamide (76.3 mg, 0.2 mmol).

### Example 28

### PREPARATION OF N-{[2-(1,4-DIOXA-8-AZASPIRO[4.5]DEC-8-YL)-1,3-THIAZOL-4-YL]METHYL}-N-(4-FLUOROPHENYL)-2,2-DIMETHYLPROPANAMIDE (COMPOUND NO. 181)

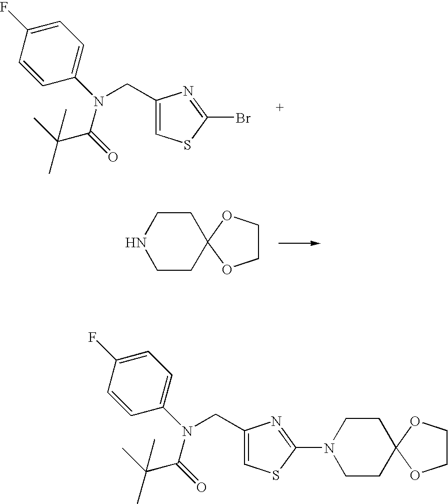

N-[(2-bromo-1,3-thiazol-4-yl)methyl]-N-(4-fluorophenyl)-2,2-dimethylpropanamide (415 mg, 1.1 mmol) was dissolved in 1,4dioxane-8-azaspiro[4.5]-decane (1.08 mL, 8.4 mmol). The reaction was heated to 80 [symbol] C overnight. The reaction mixture was partitioned between 20 mL ethyl acetate and 20 mL saturated sodium bicarbonate. The organic layers were combined and dried over magnesium sulfate and then evaporated. The resulting white solid was dried under reduced pressure providing N-{[2-(1,4-dioxa-8-azaspiro[4.5]dec-8-yl)-1,3-thiazol-4-yl]methyl}-N-(4-fluorophenyl)-2,2-dimethylpropanamide (450 mg, 0.104 mmol).

### Example 29

### PREPARATION OF N-(4-FLUOROPHENYL)-N-({2-[3-(HYDROXYMETHYL)PIPERIDIN-1-YL]-1,3-THIAZOL-4-YL}METHYL)-2,2-DIMETHYLPROPANAMIDE (COMPOUND NO. 187)

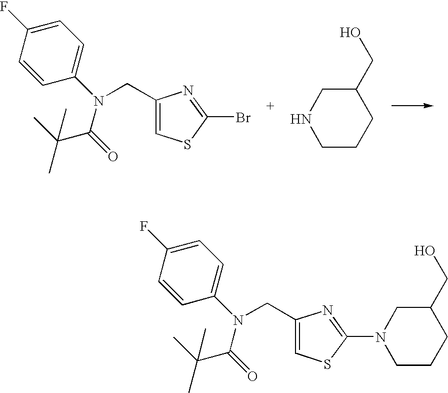

N-[(2-bromo-1,3-thiazol-4-yl)methyl]-N-(4-fluorophenyl)-2,2-dimethylpropanamide (200 mg, 0.54 mmol) was dissolved in 1 mL dimethylsulfoxide. 3-piperidinemethanol (435 mg, 3.78 mmol) was added, the reaction proceeded overnight at 80 [symbol] C. The reaction mixture was partitioned between 20 mL ethyl acetate and 2 mL saturated sodium bicarbonate. The organic layers were combined, dried over magnesium sulfate, evaporated on to silica and chromatographed (silica gel; 0-100% ethyl actetate in hexane). The solvent was removed under vacuum affording N-(4-fluorophenyl)-N-({2-[3-(hydroxymethyl)piperidin-1-yl]-1,3-thiazol-4-yl}methyl)-2,2-dimethylpropanamide (110 mg, 0.27 mmol) as an off white solid.

### Example 30

### PREPARATION OF N-(4-FLUOROPHENYL)-2,2-DIMETHYL-N-[(2-QUINOLIN-3-YL-1,3-THIAZOL-4-YL)METHYL]PROPANAMIDE (COMPOUND NO. 240)

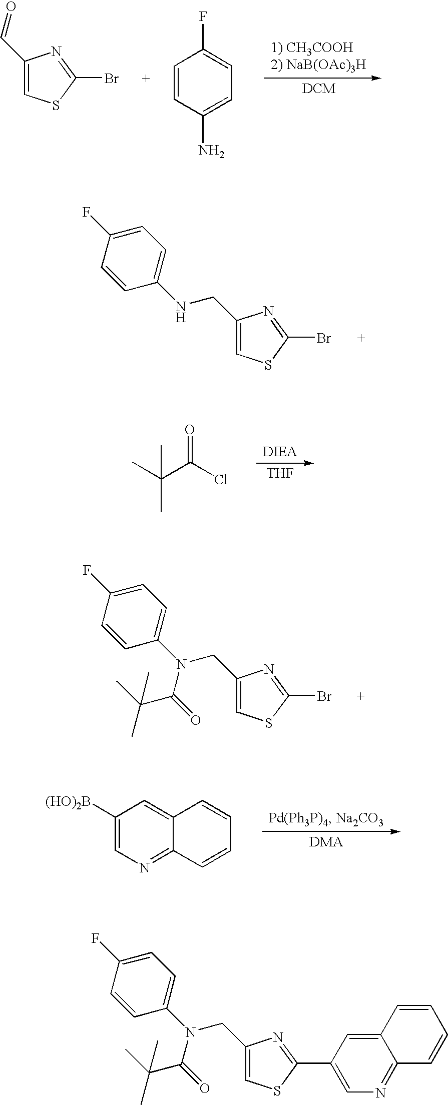

### Part I: Preparation of (2-bromo-thiazol-4-ylmethyl)-(4-fluoro-phenyl)-amine

2-bromothiazole-4-carbaldehyde (5.0 g, 26 mmol) was dissolved in 500 mL dichloromethane. 4 fluoroanaline (4.99 mL, 52 mmol) was added, followed by 2.5 mL acetic acid. The reaction was stirred 20 minutes under at room temperature. Sodium triacetoxyborohydride (7.2 g, 34 mmol) was added over a period of 45 minutes to the reaction mixture while under nitrogen. The reaction proceeded overnight at room temperature. The reaction was washed 3 times with 500 mL saturated sodium bicarbonate. The organic layer was dried over magnesium sulfate. The reaction mixture was evaporated on to silica and chromatographed (silica gel; 0-30% ethyl actetate in hexane). The solvent was removed under vacuum affording yielding a clear oil that was recrystallized from hexanes to yield a white solid. Upon filtering, the solid was dried under reduced pressure providing (2-bromo-thiazol-4-ylmethyl)-(4-fluoro-phenyl)-amine (4.23 g, 14.7 mmol).

### Part II: Preparation of N-[(2-bromo-1,3-thiazol-4-yl)methyl]-N-(4-fluorophenyl)-2,2-dimethylpropanamide

(2-bromo-thiazol-4-ylmethyl)-(4-fluoro-phenyl)-amine (4.23 g, 14.7 mmol) was dissolved in 200 mL tetrahydrofuran. Trimethylacetyl chloride (2.19 mL, 17.8 mmol) was added, followed by the addition diisopropylethylamine (3.1 ml, 17.8 mmol). The reaction proceeded at room temperature overnight. The reaction mixture was partitioned between 250 mL ethyl acetate and 250 mL water. The aqueous layer was extracted 3 times with 250 mL ethyl acetate the organic layers were dried over magnesium sulfate. The reaction mixture was evaporated onto silica and chromatographed (silica gel; 0-20% ethyl actetate in hexane). The solvent was removed under vacuum affording yielding a clear oil that was recrystallized from cold hexanes. The resulting white powder was filtered and the solid was dried under reduced pressure providing N-[(2-bromo-1,3-thiazol-4-yl)methyl]-N-(4-fluorophenyl)-2,2-dimethylpropanamide (3.4 g, 9.2 mmol).

### Part III: Preparation of N-(4-fluorophenyl)-2,2-dimethyl-N-[(2-quinolin-3-yl-1,3-thiazol-4-yl)methyl]propanamide

N-[(2-bromo-1,3-thiazol-4-yl)methyl]-N-(4-fluorophenyl)-2,2-dimethylpropanamide (100 mg, 0.27 mmol) was dissolved in 2 mL dimethylacetamide in a microwave reaction vessel. Quinoline-3-boronic acid (69.2 mg, 0.4 mmol) was added followed by tetrakis palladium (16 mg, 5 mol %), and sodium carbonate. The reaction proceeds for 15 minutes at 125 [symbol] C in a microwave. The reaction mixture was partitioned between 20 mL ethyl acetate and 20 mL saturated sodium bicarbonate. The organic layers were combined and dried over magnesium sulfate. The solvent was removed under vacuum and the reaction mixture was evaporated onto silica gel and chromatographed (silica gel; 0-100% ethyl actetate in hexane). The solvent was then removed under vacuum affording N-(4-fluorophenyl)-2,2-dimethyl-N-[(2-quinolin-3-yl-1,3-thiazol-4-yl)methyl]propanamide (44.9 mg, 0.11 mmol) as an off white solid.

### Example 31

### PREPARATION OF N-(4-FLUOROPHENYL)-2,2-DIMETHYL-N-{[2-(4-PYRIMIDIN-2-YLPIPERAZIN-1-YL)-1,3-THIAZOL-4-YL]METHYL}PROPANAMIDE (COMPOUND NO. 151) AND N-(4-FLUOROPHENYL)-2,2-DIMETHYL-N-{[2-(4-METHYLPIPERAZIN-1-YL)-1,3-THIAZOL-4-YL]METHYL}PROPANAMIDE (COMPOUND NO. 150)

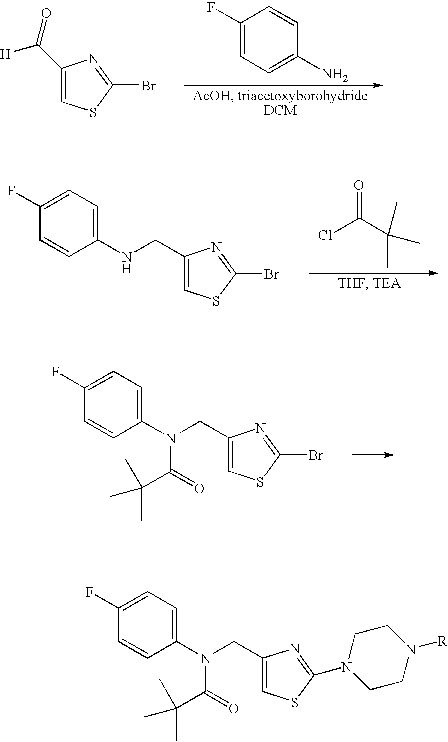

### Part I: N-[(2-bromo-1,3-thiazol-4-yl)methyl]-4-fluoroaniline

2-bromothiazole-4-carbaldehyde (7.5 g, 39 mmol), 4-fluoroaniline (7.5 mL, 78 mmol), and acetic acid (3 mL) were combined in dichloromethane (750 mL). The mixture was stirred 20 minutes, and sodium triacetoxyborohydride (10.8 g, 51 mmol) was added portionwise over a 45 minute period The mixture was partitioned between saturated sodium bicarbonate/ethyl acetate, the organic layer was separated, dried over magnesium sulfate, and concentrated. The residue was purified by chromatography (silica gel; 10% to 30% ethylacetate/hexane, gradient elution) to provide N-[(2-bromo-1,3-thiazol-4-yl)methyl]-4-fluoroaniline (7 g, 62%).

### Part II: N-[(2-bromo-1,3-thiazol-4-yl)methyl]-N-(4-fluorophenyl)-2,2-dimethylpropanamide

N-[(2-bromo-1,3-thiazol-4-yl)methyl]-4-fluoroaniline (0.900 g, 3.14 mmol) and diisopropylethylamine (0.6 mL, 3.5 mmol) were combined in tetrahydrofuran (25 mL) followed by trimethylacetylchloride (0.43 mL, 3.5 mmol) and stirred 16 hours at room temperature. The mixture was partitioned between water/ethylacetate, the organic layer was separated, and concentrated. The residue was purified by chromatography (silica gel; 0% to 100% ethylacetate/hexane, gradient elution) to provide N-[(2-bromo-1,3-thiazol-4-yl)methyl]-N-(4-fluorophenyl)-2,2-dimethylpropanamide (0.800 g, 69%).

### Part III: N-(4-fluorophenyl)-2,2-dimethyl-N-{[2-(4-pyridin-2-ylpiperazin-1-yl)-1,3-thiazol-4-yl]methyl}propanamide

A mixture of N-[(2-bromo-1,3-thiazol-4-yl)methyl]-N-(4-fluorophenyl)-2,2-dimethylpropanamide (0.240 g, 0.640 mmol) and 1-(2-pyridyl)piperazine (0.88 mL, 6.4 mmol) were combined and heated to 50° C. for 16 hours. The mixture was partitioned between saturated sodium bicarbonate/ethylacetate, the organic layer was separated, and concentrated. The residue was purified by chromatography (silica gel; 20% to 50% ethylacetate/hexane, gradient elution) to provide N-(4-fluorophenyl)-2,2-dimethyl-N-{[2-(4-pyridin-2-ylpiperazin-1-yl)-1,3-thiazol-4-yl]methyl}propanamide (0.150 g, 51%).

The resulting oil was dissolved in diethylether and 4N HCl/dioxane was added until precipitation stopped. The solvent was removed under reduced pressure to obtain N-(4-fluorophenyl)-2,2-dimethyl-N-{[2-(4-pyridin-2-ylpiperazin-1-yl)-1,3-thiazol-4-yl]methyl}propanamide trihydrochloride (0.150 g, 41%).

### Part IV: N-(4-fluorophenyl)-2,2-dimethyl-N-{[2-(4-methylpiperazin-1-yl)-1,3-thiazol-4-yl]methyl}propanamide

Follow procedure from part III. Changes: 1-methypiperazine (0.64 mL, 6.4 mmol). The residue was purified by chromatography (silica gel; 2% 7N ammonia/MeOH/dichloromethane to provide N-(4-fluorophenyl)-2,2-dimethyl-N-{[2-(4-pyridin-2-ylpiperazin-1-yl)-1,3-thiazol-4-yl]methyl}propanamide as an impure oil (0.130 g). 0.130 g of N-(4-fluorophenyl)-2,2-dimethyl-N-{[2-(4-methylpiperazin-1-yl)-1,3-thiazol-4-yl]methyl}propanamide (in 4 mL of MeOH) was injected onto a Supercritical Fluid Chromatography (SFC) instrument were collected using a Berger MultiGram Prep SFC (Berger Instruments, Inc. Newark, Del.) under the following conditions: Achiral prep SFC column (5, 250 mm L×20 mm ID), 35° C. column temperature, 20% MeOH w/0.2% DMEA as CO₂ modifier, 50 mL/min flow rate, 100 bar outlet pressure, 220 nm UV detection to provide N-(4-fluorophenyl)-2,2-dimethyl-N-{[2-(4-pyridin-2-ylpiperazin-1-yl)-1,3-thiazol-4-yl]methyl}propanamide (0.100 g, 40%). The resulting oil was dissolved in diethylether (10 mL) and 4N HCl/dioxane was added until precipitation stopped. To the suspension was added MeOH (enough to dissolve precipitate), then ether was added until slightly turbid, the flask scratched, and the crystallized N-(4-fluorophenyl)-2,2-dimethyl-N-{[2-(4-methylpiperazin-1-yl)-1,3-thiazol-4-yl]methyl}propanamide was collected and dried to provide (0.035 g, 12%).

### Part V: N-(4-fluorophenyl)-2,2-dimethyl-N-{[2-(4-pyrimidin-2-ylpiperazin-1-yl)-1,3-thiazol-4-yl]methyl}propanamide

A mixture of N-[(2-bromo-1,3-thiazol-4-yl)methyl]-N-(4-fluorophenyl)-2,2-dimethylpropanamide (0.200 g, 0.54 mmol) and 1-(2-pyrimidyl)piperazine (0.600 mL, 3.6 mmol) were combined in dimethylsulfoxide (1 mL) and heated to 75° C. for 16 hours. The mixture was partitioned between saturated sodium bicarbonate/ethylacetate, the organic layer was separated, and concentrated. The residue was purified by chromatography (silica gel; 20% to 50% ethylacetate/hexane, gradient elution) to provide N-(4-fluorophenyl)-2,2-dimethyl-N-{[2-(4-pyrimidin-2-ylpiperazin-1-yl)-1,3-thiazol-4-yl]methyl}propanamide (0.090 g, 36%).

The resulting oil was dissolved in diethylether (10 mL) and 0.7 mL of 4N HCl/dioxane was added. To the suspension was added MeOH (enough to dissolve precipitate), then ether was added until slightly turbid, the flask scratched and the crystallized N-(4-fluorophenyl)-2,2-dimethyl-N-{[2-(4-pyrimidin-2-ylpiperazin-1-yl)-1,3-thiazol-4-yl]methyl}propanamide dihydrochloride was collected and dried to provide (0.100 g, 35%).

### Example 32

### N-({2-[CYCLOPROPYL(ETHYL)AMINO]-1,3-THIAZOL-4-YL}METHYL)-2,2-DIMETHYL-N-PHENYLPROPANAMIDE (COMPOUND 168)

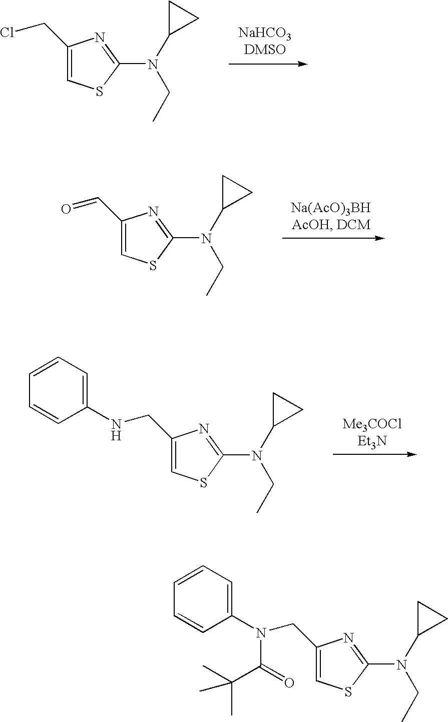

### Step I. Preparation of 2-(cyclopropyl(ethyl)amino)thiazole-4-carbaldehyde

A solution of 4-(chloromethyl)-N-cyclopropyl-N-ethylthiazol-2-amine (4.59 g, 21.17 mmol) in DMSO (100 mL) was treated with sodium hydrogen carbonate (7.1 g, 85 mmol) and heated to 100° C. under nitrogen. After 16 h, the reaction was cooled, partitioned between water and diethyl ether; the organic layer was separated, dried (MgSO₄) and evaporated. The residue was purified by column chromatography (SiO₂, hexanes:ethyl acetate 1:0 to 1:1) to afford the product (2.81 g, 14.3 mmol).

### Step II. Preparation of N-({2-[cyclopropyl(ethyl)amino]-1,3-thiazol-4-yl}methyl)-2,2-dimethyl-N-phenylpropanamide

To a solution of aniline (0.082 mL, 0.9 mmol) in dichloromethane (5 mL) containing acetic acid (0.1 mL) was added 2-(cyclopropyl(ethyl)amino)thiazole-4-carbaldehyde (0.20 g, 1.01 mmol) and sodium triacetoxyborohydride (0.425 g, 2 mmol). After 2 h. at room temperature, the mixture was treated with trimethylacetyl chloride (0.24 mL, 2 mmol) and pyridine (0.17 mL, 2.1 mmol). After 16 h. at room temperature the mixture was concentrated and purified by column chromatography (SiO2, hexanes:ethyl acetate, gradient elution) to afford the title compound as the free base. To the free base of the product was added hydrogen chloride (4N in dioxane). The mixture was evaporated, then co-evaporated with ethanol to afford the title compound as the hydrochloride salt (92.2 mg).

### Example 33

### TERT-BUTYL (3S)-3-{[(6-CHLOROPYRIDIN-3-YL)METHYL](4-FLUOROPHENYL)CARBAMOYL}PYRROLIDINE-1-CARBOXYLATE (COMPOUND NO. 254)

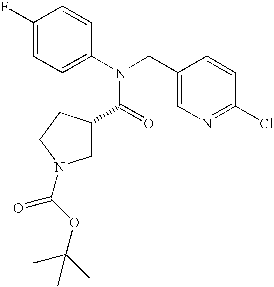

To a stirred solution of S-1-Boc-pyrrolidine-3-carboxylic acid (0.22 g, 1.0 mmol) and DMAP (0.12 g, 1.0 mmol) in dichloromethane (4 mL) was added EDC (0.21 g, 1.09 mmol) and the resulting solution was stirred room temperature. After 20 minutes, a solution of N-[(6-chloropyridin-3-yl)methyl]-4-fluoroaniline (0.20 g, 0.84 mmol) in dichloromethane (3 mL) was added and the combined solution was stirred overnight at room temperature. The mixture was then washed with water and concentrated. Flash column separation using 0-50% ethyl acetate/hexane gave tert-butyl (3S)-3-{[(6-chloropyridin-3-yl)methyl](4-fluorophenyl)carbamoyl}pyrrolidine-1-carboxylate as an oil. (0.24 g, 65%).

### Example 34

### (3S)—N-(4-FLUOROPHENYL)-N-{[6-(4-METHYLPIPERAZIN-1-YL)PYRIDIN-3-YL]METHYL}PYRROLIDINE-3-CARBOXAMIDE (COMPOUND NO. 259)

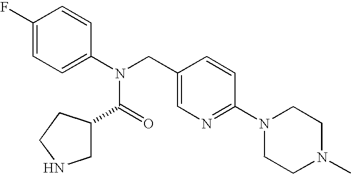

### Part 1: To a stirred solution of tert-butyl (3S)-3-{[(6-chloropyridin-3-ylmethyl](4-fluorophenyl)carbamoyl}pyrrolidine-1-carboxylate (0.21 g, 0.49 mmol) THF (1 mL) was added N-methylpiperizine (0.3 mL, 2.7 mmol) and the resulting solution was microwave irradiated to 180° C. for 60 minutes. The resulting mixture was concentrated and flash column separation using 0-8% methanol/methylene chloride gave tert-butyl (3S)-3-[(4-fluorophenyl){[6-(4-methylpiperazin-1-yl)pyridin-3-yl]methyl}carbamoyl]pyrrolidine-1-carboxylate as a white solid.

### Part 2: To a stirred solution of tert-butyl (3S)-3-[(4-fluorophenyl){[6-(4-methylpiperazin-1-yl)pyridin-3-yl]methyl}carbamoyl]pyrrolidine-1-carboxylate in ethyl acetate was added 4N HCl 1,4-dioxane solution (2 mL) and the resulting mixture was stirred overnight at room temperature. Concentration and trituration using ethyl ether gave (3S)—N-(4-fluorophenyl)-N-{[6-(4-methylpiperazin-1-yl)pyridin-3-yl]methyl}pyrrolidine-3-carboxamide as a white solid. (0.03 g, 15% overall).

### Example 35

### (3S)-1-(2,2-DIMETHYLPROPANOYL)-N-(4-FLUOROPHENYL)-N-{[6-(4-METHYLPIPERAZIN-1-YL)PYRIDIN-3-YL]METHYL}PYRROLIDINE-3-CARBOXAMIDE (COMPOUND NO. 260)

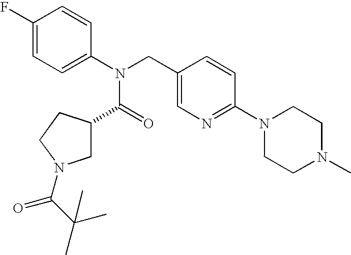

To a stirred solution of (3S)—N-(4-fluorophenyl)-N-{[6-(4-methylpiperazin-1-yl)pyridin-3-yl]methyl}pyrrolidine-3-carboxamide (0.11 g, 0.22 mmol) and triethylamine (0.15 mL, 1.0 mmol) in dichloromethane (2 mL) was added pivaloyl chloride (0.027 g, 0.22 mmol) and the resulting solution was stirred several hours at room temperature. The reaction as washed with 1N NaOH and concentrated. Flash column separation using 0-7% methanol/methylene chloride gave (3S)-1-(2,2-dimethylpropanoyl)-N-(4-fluorophenyl)-N-{[6-(4-methylpiperazin-1-yl)pyridin-3-yl]methyl}pyrrolidine-3-carboxamide as a white solid. (0.033 g, 27%)

### Example 36

### N-[(6-CHLOROPYRIDIN-3-YL)METHYL]-N-(4-FLUOROPHENYL)TETRAHYDROFURAN-2-CARBOXAMIDE (COMPOUND NO. 252)

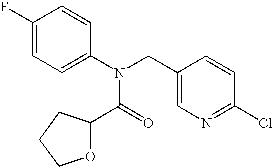

To a stirred solution of 2-tetrahydrofuroic acid (0.11 g, 0.95 mmol) in dichloromethane (2 mL) was added oxalyl chloride (0.85 mL, 9.7 mmol) and the resulting mixture was stirred overnight at room temperature. The mixture was then concentrated, redissolved in chloroform (3 mL) and reconcentrated. This crude material was dissolved in THF (2 mL) and to it was added triethylamine (0.3 mL, 2.1 mmol) and N-[(6-chloropyridin-3-yl)methyl]-4-fluoroaniline (0.15 g, 0.63 mmol). The resulting solution was stirred 1 hour, washed with water and concentrated. Flash column separation using 10-60% ethyl acetate/hexane gave N-[(6-chloropyridin-3-yl)methyl]-N-(4-fluorophenyl)tetrahydrofuran-2-carboxamide as a clear oil. (0.125 g, 59%)

### Example 37

### N-(4-FLUOROPHENYL)-N-({2-[3-(HYDROXYMETHYL)PIPERIDIN-1-YL]-1,3-THIAZOL-4-YL}METHYL)ACETAMIDE (COMPOUND NO. 225)

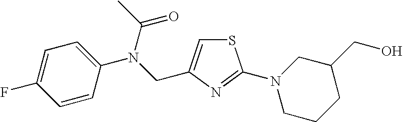

Preparation of N-(4-fluorophenyl)-N-({2-[3-(hydroxymethyl)piperidin-1-yl]-1,3-thiazol-4-yl}methyl)acetamide To a stirred solution of N-[(2-bromo-1,3-thiazol-4-yl)methyl]-N-(4-fluorophenyl)acetamide (0.10 g, 0.3 mmol) in THF (0.5 mL) was added 3-piperidinemethanol (0.29 mg, 2.5 mmol) and the resulting solution was microwave irradiated to 180° C. for 10 minutes. The crude mixture was concentrated and flash column separation using 0-6% methanol/methylene chloride gave N-(4-fluorophenyl)-N-({2-[3-(hydroxymethyl)piperidin-1-yl]-1,3-thiazol-4-yl}methyl)acetamide as a white solid. (0.07 g, 58%).

### Example 36

### N-(4-FLUOROPHENYL)-N-{[6-(4-METHYL-1,4-DIAZEPAN-1-YL)PYRIDIN-3-YL]METHYL}ACETAMIDE (COMPOUND NO. 226)

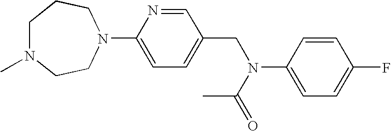

Preparation of N-(4-fluorophenyl)-N-{[6-(4-methyl-1,4-diazepan-1-yl)pyridin-3-yl]methyl}acetamide To a stirred solution of N-[(6-chloropyridin-3-yl)methyl]-N-(4-fluorophenyl)acetamide (0.10 g, 0.36 mmol) in THF (0.5 mL) was added 1-methylhomopiperizine (0.3 mL, 2.4 mmol) and the resulting solution was microwave irradiated to 180° C. for 60 minutes. The crude mixture was concentrated and flash column separation using 0-10% methanol/methylene chloride gave N-(4-fluorophenyl)-N-{[6-(4-methyl-1,4-diazepan-1-yl)pyridin-3-yl]methyl}acetamide as a white solid. (0.103 g, 67%).

TABLE 2A

| | | | | HPLC | | | |
|---|---|---|---|---|---|---|---|
| | | | | | Retention | | |
| Cpd | | Salt | | | Time | | Mol |
| no. | Name | Name | Scheme | Method | (min) | % purity | Ion |
| 116 | N-{[6-(diethylamino)pyridin-3- | | 14 | | | | 371.1 |
| | yl]methyl}-N-(4-fluorophenyl)-L- | | | | | | |
| | prolinamide | | | | | | |
| 117 | N-{4-[cyclopropyl(ethyl)amino]benzyl}- | | 15 | B | 3.8 | | 394.1 |
| | N-(4-methoxyphenyl)-L-prolinamide | | | | | | |
| 118 | N-{3-[cyclopropyl(ethyl)amino]benzyl}- | | 16 | B | 3.9 | | 382.1 |
| | N-(4-fluorophenyl)-L-prolinamide | | | | | | |
| 119 | N-(4-chlorophenyl)-N-{[6- | | 19 | B | 3.9 | | 387.1 |
| | (diethylamino)pyridin-2-yl]methyl}-L- | | | | | | |
| | prolinamide | | | | | | |
| 120 | N-(4-fluoro-2-methylphenyl)-N-[(2- | | 17 | B | 3.8 | | 398.1 |
| | piperidin-1-ylpyrimidin-5-yl)methyl]-L- | | | | | | |
| | prolinamide | | | | | | |
| 121 | N-(4-methylphenyl)-N-[(2-pyrrolidin-1- | | 18 | B | 3.7 | | 371.1 |
| | yl-1,3-thiazol-5-yl)methyl]-L- | | | | | | |
| | prolinamide | | | | | | |
| 122 | N-({2-[cyclopropyl(ethyl)amino]-1,3- | HCl | 20 | A | 9.5 | | 389.2 |
| | thiazol-4-yl}methyl)-N-(4- | | | | | | |
| | fluorophenyl)-L-prolinamide | | | | | | |
| 123 | N-({2-[cyclopropyl(ethyl)amino]-1,3- | HCl | 20 | A | 7.8 | | 389 |
| | thiazol-4-yl}methyl)-N-(4- | | | | | | |
| | fluorophenyl)-D-prolinamide | | | | | | |
| 124 | N-({2-[cyclopropyl(ethyl)amino]-1,3- | HCl | 20 | A | 8.7 | | 403 |
| | thiazol-4-yl}methyl)-N-(4- | | | | | | |
| | fluorophenyl)-5-oxo-L-prolinamide | | | | | | |
| 125 | N-(4-fluorophenyl)-N-{[6-(4- | HCl | | | | 100 | 411 |
| | methylpiperazin-1-yl)pyridin-3- | | | | | | |
| | yl]methyl}cyclohexanecarboxamide | | | | | | |
| 126 | N′-(tert-butyl)-N-(4-fluorophenyl)-N- | HCl | | | | 98 | 400 |
| | {[6-(4-methylpiperazin-1-yl)pyridin-3- | | | | | | |
| | yl]methyl}urea | | | | | | |
| 127 | 4,4,4-trifluoro-N-(4-fluorophenyl)-3- | HCl | | | | | |
| | methyl-N-({6-[methyl(2-pyridin-2- | | | | | | |
| | ylethyl)amino]pyridin-3- | | | | | | |
| | yl}methyl)butanamide | | | | | | |
| 128 | N-(4-fluorophenyl)-N-{[6-(4-pyridin-2- | HCl | | | | 98 | 474 |
| | ylpiperazin-1-yl)pyridin-3- | | | | | | |
| | yl]methyl}cyclohexanecarboxamide | | | | | | |
| 129 | N-{[6-(diethylamino)pyridin-3- | HCl | | | | 76 | 398 |
| | yl]methyl}-N-(4-fluorophenyl)-2-(2- | | | | | | |
| | thienyl)acetamide | | | | | | |
| 130 | N-(4-fluorophenyl)-N-({6-[(2- | HCl | | A | 6.7 | 99 | 360 |
| | hydroxyethyl)(methyl)amino]pyridin-3- | | | | | | |
| | yl}methyl)-2,2-dimethylpropanamide | | | | | | |
| 131 | N-(4-fluorophenyl)-2,2-dimethyl-N-{[6- | HCl | | | | 76 | 342 |
| | (methyl{2-[(5-methylpyridin-2- | | | | | | |
| | yl)oxy]ethyl}amino)pyridin-3- | | | | | | |
| | yl]methyl}propanamide | | | | | | |
| 132 | N-(4-fluorophenyl)-2,2-dimethyl-N-[(6- | HCl | | | | 99 | 356 |
| | pyrrolidin-1-ylpyridin-3- | | | | | | |
| | yl)methyl]propanamide | | | | | | |
| 133 | N-({6-[3-(diethylamino)pyrrolidin-1- | HCl | | | | 100 | 427 |
| | yl]pyridin-3-yl}methyl)-N-(4- | | | | | | |
| | fluorophenyl)-2,2- | | | | | | |
| | dimethylpropanamide | | | | | | |
| 134 | N-(4-fluorophenyl)-2,2-dimethyl-N-[(2- | HCl | 1 | A | 10.8 | 81 | 376.1 |
| | piperidin-1-yl-1,3-thiazol-4- | | | | | | |
| | yl)methyl]propanamide | | | | | | |
| 135 | N-(4-fluorophenyl)-2,2-dimethyl-N-({6- | HCl | | | | | |
| | [propionyl(2-pyridin-3- | | | | | | |
| | ylethyl)amino]pyridin-3- | | | | | | |
| | yl}methyl)propanamide | | | | | | |
| 136 | N-(4-fluorophenyl)-2,2-dimethyl-N-[(2- | HCl | 1 | A | 9.6 | 95 | 378.1 |
| | morpholin-4-yl-1,3-thiazol-4- | | | | | | |
| | yl)methyl]propanamide | | | | | | |
| 137 | N-(4-fluorophenyl)-N-[(2-morpholin-4- | HCl | | | | 97 | 404 |
| | yl-1,3-thiazol-4- | | | | | | |
| | yl)methyl]cyclohexanecarboxamide | | | | | | |
| 138 | N-(4-fluorophenyl)-2,2-dimethyl-N-[(2- | HCl | | | | 100 | 392 |
| | morpholin-4-yl-1,3-thiazol-4- | | | | | | |
| | yl)methyl]butanamide | | | | | | |
| 139 | N-({6-[cyclopropyl(ethyl)amino]pyridin- | HCl | | A | 8.7 | | |
| | 3-yl}methyl)-N-(4-fluorophenyl)-2,2- | | | | | | |
| | dimethylpropanamide | | | | | | |
| 140 | N-(4-fluorophenyl)-2,2-dimethyl-N-[(2- | HCl | 1 | A | 10.4 | 100 | 362.1 |
| | pyrrolidin-1-yl-1,3-thiazol-4- | | | | | | |
| | yl)methyl]propanamide | | | | | | |
| 141 | N-(4-fluorophenyl)-N-({2-[(4- | HCl | | | | 86 | 416 |
| | fluorophenyl)(methyl)amino]-1,3- | | | | | | |
| | thiazol-4-yl}methyl)-2,2- | | | | | | |
| | dimethylpropanamide | | | | | | |
| 142 | N-(4-fluorophenyl)-N-({2-[(4- | HCl | | | | 82 | 428 |
| | methoxyphenyl)(methyl)amino]-1,3- | | | | | | |
| | thiazol-4-yl}methyl)-2,2- | | | | | | |
| | dimethylpropanamide | | | | | | |
| 143 | N-({2-[cyclopropyl(ethyl)amino]-1,3- | HCl | 2 | A | 10.8 | | 375.7 |
| | thiazol-4-yl}methyl)-N-(4- | | | | | | |
| | fluorophenyl)-2,2- | | | | | | |
| | dimethylpropanamide | | | | | | |
| 144 | N-[(2-bromo-1,3-thiazol-4-yl)methyl]- | | 1 | A | 10.1 | | 370.7 |
| | N-(4-fluorophenyl)-2,2- | | | | | | 371.0 |
| | dimethylpropanamide | | | | | | |
| 145 | N-{[2-(3,5-dimethylpiperazin-1-yl)-1,3- | HCl | 1 | A | 9.7 | | 405.1 |
| | thiazol-4-yl]methyl}-N-(4- | | | | | | |
| | fluorophenyl)-2,2- | | | | | | |
| | dimethylpropanamide | | | | | | |
| 146 | tert-butyl 4-(4-{[(2,2- | | 1 | A | 10.9 | | 477.2 |
| | dimethylpropanoyl)(4- | | | | | | |
| | fluorophenyl)amino]methyl}--1,3- | | | | | | |
| | thiazol-2-yl)piperazine-1-carboxylate | | | | | | |
| 147 | N-(4-fluorophenyl)-2,2-dimethyl-N-{[2- | HCl | 1 | A | 11.3 | | 453.1 |
| | (4-phenylpiperazin-1-yl)-1,3-thiazol-4- | | | | | | |
| | yl]methyl}propanamide | | | | | | |
| 148 | N-(4-fluorophenyl)-N-{[2-(4- | HCl | 1 | A | 10.5 | | 419.2 |
| | isopropylpiperazin-1-yl)-1,3-thiazol-4- | | | | | | |
| | yl]methyl}-2,2-dimethylpropanamide | | | | | | |
| 149 | N-(4-fluorophenyl)-2,2-dimethyl-N-{[2- | HCl | 1 | A | 10.4 | | 454.1 |
| | (4-pyridin-2-ylpiperazin-1-yl)--1,3- | | | | | | |
| | thiazol-4-yl]methyl}propanamide | | | | | | |
| 150 | N-(4-fluorophenyl)-2,2-dimethyl-N-{[2- | HCl | 1 | A | 7.3 | | 391.1 |
| | (4-methylpiperazin-1-yl)-1,3-thiazol-4- | | | | | | |
| | yl]methyl}propanamide | | | | | | |
| 151 | N-(4-fluorophenyl)-2,2-dimethyl-N-{[2- | HCl | 1 | A | 10.6 | | 455.1 |
| | (4-pyrimidin-2-ylpiperazin-1-yl)--1,3- | | | | | | |
| | thiazol-4-yl]methyl}propanamide | | | | | | |
| 152 | N-(4-fluorophenyl)-2,2-dimethyl-N-[(2- | HCl | 1 | A | 7.3 | | 377.1 |
| | piperazin-1-yl-1,3-thiazol-4- | | | | | | |
| | yl)methyl]propanamide | | | | | | |
| 153 | N-[(2-{[2-(dimethylamino)ethyl]amino}- | HCl | 1 | A | 7.5 | | 379.2 |
| | 1,3-thiazol-4-yl)methyl]-N-(4- | | | | | | |
| | fluorophenyl)-2,2- | | | | | | |
| | dimethylpropanamide | | | | | | |
| 154 | N-[(2-{[3- | HCl | 1 | A | 7.6 | | 393.1 |
| | (dimethylamino)propyl]amino}-1,3- | | | | | | |
| | thiazol-4-yl)methyl]-N-(4- | | | | | | |
| | fluorophenyl)-2,2- | | | | | | |
| | dimethylpropanamide | | | | | | |
| 155 | N-[(2-{[4-(dimethylamino)butyl]amino}- | HCl | 1 | A | 7.4 | | 407.2 |
| | 1,3-thiazol-4-yl)methyl]-N-(4- | | | | | | |
| | fluorophenyl)-2,2- | | | | | | |
| | dimethylpropanamide | | | | | | |
| 156 | N-{[2-(cyclopentylamino)-1,3-thiazol-4- | HCl | 1 | A | 10.5 | | 376.1 |
| | yl]methyl}-N-(4-fluorophenyl)-2,2- | | | | | | |
| | dimethylpropanamide | | | | | | |
| 157 | N-{[2-(cyclohexylamino)-1,3-thiazol-4- | HCl | 1 | A | 10.9 | | 390.2 |
| | yl]methyl}-N-(4-fluorophenyl)-2,2- | | | | | | |
| | dimethylpropanamide | | | | | | |
| 158 | N-(4-fluorophenyl)-N-({2-[(4- | HCl | 1 | A | 11 | | 402.2 |
| | fluorophenyl)amino]-1,3-thiazol-4- | | | | | | |
| | yl}methyl)-2,2-dimethylpropanamide | | | | | | |
| 159 | N-(4-fluorophenyl)-N-({2-[(2- | HCl | 1 | A | 10.9 | | 402.2 |
| | fluorophenyl)amino]-1,3-thiazol-4- | | | | | | |
| | yl}methyl)-2,2-dimethylpropanamide | | | | | | |
| 160 | N-(4-fluorophenyl)-2,2-dimethyl-N-{[2- | | 1 | A | 8.7 | | 322.4 |
| | (methylamino)-1,3-thiazol-4- | | | | | | |
| | yl]methyl}propanamide | | | | | | |
| 161 | N-(4-fluorophenyl)-N-{[2- | | 1 | A | 10.3 | | 363.7 |
| | (isobutylamino)-1,3-thiazol-4- | | | | | | |
| | yl]methyl}-2,2-dimethylpropanamide | | | | | | |
| 162 | N-({2-[(1S,4S)-2,5- | HCl | 1 | A | 7.2 | | 389.2 |
| | diazabicyclo[2.2.1]hept-2-yl]-1,3- | | | | | | |
| | thiazol-4-yl}methyl)-N-(4- | | | | | | |
| | fluorophenyl)-2,2- | | | | | | |
| | dimethylpropanamide | | | | | | |
| 163 | N-(4-fluorophenyl)-2,2-dimethyl-N-{[2- | | 1 | A | 9.9 | | 349.5 |
| | (propylamino)-1,3-thiazol-4- | | | | | | |
| | yl]methyl}propanamide | | | | | | |
| 164 | N-{[2-(dimethylamino)-1,3-thiazol-4- | HCl | 1 | A | 9.6 | | 335.9 |
| | yl]methyl}-N-(4-fluorophenyl)-2,2- | | | | | | |
| | dimethylpropanamide | | | | | | |
| 165 | N-(4-fluorophenyl)-2,2-dimethyl-N-({2- | HCl | 1 | A | 11.3 | | 481.2 |
| | [4-(2-phenylethyl)piperazin-1-yl]--1,3- | | | | | | |
| | thiazol-4-yl}methyl)propanamide | | | | | | |
| 166 | N-[(2-{4-[3- | HCl | 1 | A | 9.9 | | 462.2 |
| | (dimethylamino)propyl]piperazin-1-yl}- | | | | | | |
| | 1,3-thiazol-4-yl)methyl]-N-(4- | | | | | | |
| | fluorophenyl)-2,2- | | | | | | |
| | dimethylpropanamide | | | | | | |
| 167 | N-({2-[4-(diphenylmethyl)piperazin-1- | HCl | 1 | A | 12.2 | | 543.2 |
| | yl]-1,3-thiazol-4-yl}methyl)-N-(4- | | | | | | |
| | fluorophenyl)-2,2- | | | | | | |
| | dimethylpropanamide | | | | | | |
| 168 | N-({2-[cyclopropyl(ethyl)amino]-1,3- | HCl | 3 | A | 10.9 | | 358.2 |
| | thiazol-4-yl}methyl)-2,2-dimethyl-N- | | | | | | |
| | phenylpropanamide | | | | | | |
| 169 | N-({2-[cyclopropyl(ethyl)amino]-1,3- | HCl | 3 | A | 10.9 | | 376.2 |
| | thiazol-4-yl}methyl)-N-(2- | | | | | | |
| | fluorophenyl)-2,2- | | | | | | |
| | dimethylpropanamide | | | | | | |
| 170 | N-({2-[cyclopropyl(ethyl)amino]-1,3- | HCl | 3 | A | 11 | | 376.2 |
| | thiazol-4-yl}methyl)-N-(3- | | | | | | |
| | fluorophenyl)-2,2- | | | | | | |
| | dimethylpropanamide | | | | | | |
| 171 | N-({2-[cyclopropyl(ethyl)amino]-1,3- | HCl | 3 | A | 11.1 | | 372.2 |
| | thiazol-4-yl}methyl)-2,2-dimethyl-N-(2- | | | | | | |
| | methylphenyl)propanamide | | | | | | |
| 172 | N-({2-[cyclopropyl(ethyl)amino]-1,3- | HCl | 3 | A | 11.2 | | 372.2 |
| | thiazol-4-yl}methyl)-2,2-dimethyl-N-(3- | | | | | | |
| | methylphenyl)propanamide | | | | | | |
| 173 | N-({2-[cyclopropyl(ethyl)amino]-1,3- | HCl | 3 | A | 11.2 | | 372.2 |
| | thiazol-4-yl}methyl)-2,2-dimethyl-N-(4- | | | | | | |
| | methylphenyl)propanamide | | | | | | |
| 174 | N-({2-[cyclopropyl(ethyl)amino]-1,3- | HCl | 3 | A | 10.8 | | 388.2 |
| | thiazol-4-yl}methyl)-N-(2- | | | | | | |
| | methoxyphenyl)-2,2- | | | | | | |
| | dimethylpropanamide | | | | | | |
| 175 | N-({2-[cyclopropyl(ethyl)amino]-1,3- | HCl | 3 | A | 10.8 | | 388.2 |
| | thiazol-4-yl}methyl)-N-(4- | | | | | | |
| | methoxyphenyl)-2,2- | | | | | | |
| | dimethylpropanamide | | | | | | |
| 176 | N-({2-[cyclopropyl(ethyl)amino]-1,3- | HCl | 3 | A | 11.9 | | 412.3 |
| | thiazol-4-yl}methyl)-2,2-dimethyl-N- | | | | | | |
| | (5,6,7,8-tetrahydronaphthalen-1- | | | | | | |
| | yl)propanamide | | | | | | |
| 177 | N-{[2-(4-benzylpiperazin-1-yl)-1,3- | HCl | 3 | A | 9.6 | | 467.2 |
| | thiazol-4-yl]methyl}-N-(4- | | | | | | |
| | fluorophenyl)-2,2- | | | | | | |
| | dimethylpropanamide | | | | | | |
| 178 | N-[(2-{4-[2- | HCl | 3 | A | 8.0 | | 448.2 |
| | (dimethylamino)ethyl]piperazin-1-yl}- | | | | | | |
| | 1,3-thiazol-4-yl)methyl]-N-(4- | | | | | | |
| | fluorophenyl)-2,2- | | | | | | |
| | dimethylpropanamide | | | | | | |
| 179 | N-{[2-(diphenylamino)-1,3-thiazol-4- | | 3 | A | 11.5 | | 460 |
| | yl]methyl}-N-(4-fluorophenyl)-2,2- | | | | | | |
| | dimethylpropanamide | | | | | | |
| 180 | N-(4-fluorophenyl)-2,2-dimethyl-N-[(2- | | 4 | A | 11 | | 369.1 |
| | phenyl-1,3-thiazol-4- | | | | | | |
| | yl)methyl]propanamide | | | | | | |
| 181 | N-{[2-(1,4-dioxa-8-azaspiro[4.5]dec-8- | | 1 | A | 10.2 | | 434 |
| | yl)-1,3-thiazol-4-yl]methyl}-N-(4- | | | | | | |
| | fluorophenyl)-2,2- | | | | | | |
| | dimethylpropanamide | | | | | | |
| 182 | N-(4-fluorophenyl)-N-{[2-(4- | HCl | 1 | A | 9.1 | | 392.2 |
| | hydroxypiperidin-1-yl)-1,3-thiazol-4- | | | | | | |
| | yl]methyl}-2,2-dimethylpropanamide | | | | | | |
| 183 | N-[(2-azepan-1-yl-1,3-thiazol-4- | HCl | 1 | A | 11 | | 390.8 |
| | yl)methyl]-N-(4-fluorophenyl)-2,2- | | | | | | |
| | dimethylpropanamide | | | | | | |
| 184 | N-[(2-azocan-1-yl-1,3-thiazol-4- | HCl | 1 | A | 11.4 | | 404.3 |
| | yl)methyl]-N-(4-fluorophenyl)-2,2- | | | | | | |
| | dimethylpropanamide | | | | | | |
| 185 | N-(4-fluorophenyl)-N-({2-[4- | | 1 | A | 9.4 | | 406.2 |
| | (hydroxymethyl)piperidin-1-yl]-1,3- | | | | | | |
| | thiazol-4-yl}methyl)-2,2- | | | | | | |
| | dimethylpropanamide | | | | | | |
| 186 | N-(4-fluorophenyl)-N-{[2-(3- | | 1 | A | 9.3 | | 392.1 |
| | hydroxypiperidin-1-yl)-1,3-thiazol-4- | | | | | | |
| | yl]methyl}-2,2-dimethylpropanamide | | | | | | |
| 187 | N-(4-fluorophenyl)-N-({2-[3- | | 1 | A | 9.6 | | 406.2 |
| | (hydroxymethyl)piperidin-1-yl]-1,3- | | | | | | |
| | thiazol-4-yl}methyl)-2,2- | | | | | | |
| | dimethylpropanamide | | | | | | |
| 188 | N-{[2-(4-cyanopiperidin-1-yl)-1,3- | | 1 | A | 9.8 | | 401.3 |
| | thiazol-4-yl]methyl}-N-(4- | | | | | | |
| | fluorophenyl)-2,2- | | | | | | |
| | dimethylpropanamide | | | | | | |
| 189 | N-(4-fluorophenyl)-2,2-dimethyl-N-[(2- | | 1 | A | 10.5 | | 394 |
| | thiomorpholin-4-yl-1,3-thiazol-4- | | | | | | |
| | yl)methyl]propanamide | | | | | | |
| 190 | N-{[2-(3,5-dimethylpiperidin-1-yl)-1,3- | HCl | 1 | A | 11.6 | | 404 |
| | thiazol-4-yl]methyl}-N-(4- | | | | | | |
| | fluorophenyl)-2,2- | | | | | | |
| | dimethylpropanamide | | | | | | |
| 191 | N-(4-fluorophenyl)-2,2-dimethyl-N-{[2- | HCl | 1 | A | 11.9 | | 430.2 |
| | (octahydroisoquinolin-2(1H)-yl)--1,3- | | | | | | |
| | thiazol-4-yl]methyl}propanamide | | | | | | |
| 192 | N-(4-fluorophenyl)-2,2-dimethyl-N-{[2- | HCl | 1 | A | 11.2 | | 389.6 |
| | (4-methylpiperidin-1-yl)-1,3-thiazol-4- | | | | | | 390.3 |
| | yl]methyl}propanamide | | | | | | |
| 193 | N-(4-fluorophenyl)-2,2-dimethyl-N-{[2- | HCl | 1 | A | 11.6 | | 452.0 |
| | (4-phenylpiperidin-1-yl)-1,3-thiazol-4- | | | | | | 452.1 |
| | yl]methyl}propanamide | | | | | | |
| 194 | N-(4-fluorophenyl)-2,2-dimethyl-N-[(2- | HCl | 1 | A | 10.3 | | 376 |
| | piperidin-1-yl-1,3-thiazol-5- | | | | | | |
| | yl)methyl]propanamide | | | | | | |
| 195 | N-[(6-chloropyridin-3-yl)methyl]-N-(4- | HCl | 5 | A | 9.7 | | 320.9 |
| | fluorophenyl)-2,2- | | | | | | |
| | dimethylpropanamide | | | | | | |
| 196 | N-(4-fluorophenyl)-2,2-dimethyl-N-[(6- | HCl | 5 | A | 9.2 | | 372.1 |
| | morpholin-4-ylpyridin-3- | | | | | | |
| | yl)methyl]propanamide | | | | | | |
| 197 | N-cyclohexyl-N-({2- | HCl | 6 | A | 9.7 | | 322.2 |
| | [cyclopropyl(ethyl)amino]-1,3-thiazol- | | | | | | |
| | 4-yl}methyl)acetamide | | | | | | |
| 198 | N-cyclohexyl-N-({2- | HCl | 6 | A | 11.6 | | 390.1 |
| | [cyclopropyl(ethyl)amino]-1,3-thiazol- | | | | | | |
| | 4-yl}methyl)cyclohexanecarboxamide | | | | | | |
| 199 | N-cyclohexyl-N-[(2-morpholin-4-yl-1,3- | HCl | 6 | A | 8.3 | | 324.4 |
| | thiazol-4-yl)methyl]acetamide | | | | | | |
| 200 | N-cyclohexyl-N-[(2-morpholin-4-yl-1,3- | HCl | 6 | A | 10.6 | | 392.1 |
| | thiazol-4- | | | | | | |
| | yl)methyl]cyclohexanecarboxamide | | | | | | |
| 201 | N-cyclohexyl-N-[(2-morpholin-4-yl-1,3- | HCl | 6 | A | 10.1 | | 400.4 |
| | thiazol-4-yl)methyl]-2- | | | | | | |
| | phenylacetamide | | | | | | |
| 202 | tert-butyl 3-(5-{[(2,2- | | 7 | A | 11.6 | | 462.9 |
| | dimethylpropanoyl)(4- | | | | | | |
| | fluorophenyl)amino]methyl}pyridin-2- | | | | | | |
| | yl)benzoate | | | | | | |
| 203 | 3-(5-{[(2,2-dimethylpropanoyl)(4- | | 7 | A | 9.9 | | 406.9 |
| | fluorophenyl)amino]methyl}pyridin-2- | | | | | | |
| | yl)benzoic acid | | | | | | |
| 204 | N-cyclohexyl-2,2-dimethyl-N-[(2- | HCl | 6 | A | 10.1 | | 366.4 |
| | morpholin-4-yl-1,3-thiazol-4- | | | | | | |
| | yl)methyl]propanamide | | | | | | |
| 205 | N-cyclohexyl-N-({2- | HCl | 6 | A | 11.2 | | 364 |
| | [cyclopropyl(ethyl)amino]-1,3-thiazol- | | | | | | |
| | 4-yl}methyl)-2,2-dimethylpropanamide | | | | | | |
| 206 | N-[(2-cyclohexyl-1,3-thiazol-4- | | 4 | A | 11.3 | | 375.3 |
| | yl)methyl]-N-(4-fluorophenyl)-2,2- | | | | | | |
| | dimethylpropanamide | | | | | | |
| 207 | N-(4-fluorophenyl)-N-[(2-morpholin-4- | HCl | 8 | A | 8.8 | | 362 |
| | yl-1,3-thiazol-4- | | | | | | |
| | yl)methyl]cyclopropanecarboxamide | | | | | | |
| 208 | N-(4-fluorophenyl)-2-methyl-N-[(2- | HCl | 8 | A | 9 | | 364 |
| | morpholin-4-yl-1,3-thiazol-4- | | | | | | |
| | yl)methyl]propanamide | | | | | | |
| 209 | tert-butyl 4-(4-{[(2,2- | | 4 | A | 10.9 | | 475.9 |
| | dimethylpropanoyl)(4- | | | | | | |
| | fluorophenyl)amino]methyl}--1,3- | | | | | | |
| | thiazol-2-yl)piperidine-1-carboxylate | | | | | | |
| 210 | N-({2-[cyclopropyl(ethyl)amino]-1,3- | HCl | 2 | A | 9.4 | | 334.4 |
| | thiazol-4-yl}methyl)-N-(4- | | | | | | |
| | fluorophenyl)acetamide | | | | | | |
| 211 | N-({2-[cyclopropyl(ethyl)amino]-1,3- | HCl | 2 | A | 11.1 | | 401.9 |
| | thiazol-4-yl}methyl)-N-(4- | | | | | | |
| | fluorophenyl)cyclohexanecarboxamide | | | | | | |
| 212 | N-(4-fluorophenyl)-2,2-dimethyl-N-[2- | HCl | 9 | A | 9.8 | | 392 |
| | (2-morpholin-4-yl-1,3-thiazol-4- | | | | | | |
| | yl)ethyl]propanamide | | | | | | |
| 213 | N-(4-fluorophenyl)-2,2-dimethyl-N-[3- | HCl | 9 | A | 10 | | 406.3 |
| | (2-morpholin-4-yl-1,3-thiazol-4- | | | | | | |
| | yl)propyl]propanamide | | | | | | |
| 214 | N-[(2-bromo-1,3-thiazol-4-yl)methyl]- | | 1 | A | 8.3 | | 328.8 |
| | N-(4-fluorophenyl)acetamide | | | | | | |
| 215 | N-[(6-chloropyridin-3-yl)methyl]-N-(4- | | 5 | A | 7.9 | | 278.9 |
| | fluorophenyl)acetamide | | | | | | |
| 216 | N-(4-fluorophenyl)-N-{[2-(4- | HCl | 1 | A | 5.1 | | 349 |
| | methylpiperazin-1-yl)-1,3-thiazol-4- | | | | | | |
| | yl]methyl}acetamide | | | | | | |
| 217 | N-(4-fluorophenyl)-N-{[2-(4-methyl- | HCl | 1 | A | 5.4 | | 363 |
| | 1,4-diazepan-1-yl)-1,3-thiazol-4- | | | | | | |
| | yl]methyl}acetamide | | | | | | |
| 218 | N-(4-fluorophenyl)-N-[(2-morpholin-4- | HCl | 1 | A | 7.8 | | 336.2 |
| | yl-1,3-thiazol-4-yl)methyl]acetamide | | | | | | |
| 219 | N-{[2-(diethylamino)-1,3-thiazol-4- | HCl | 1 | A | 9 | | 322 |
| | yl]methyl}-N-(4- | | | | | | |
| | fluorophenyl)acetamide | | | | | | |
| 220 | N-{[2-(4-cyanopiperidin-1-yl)-1,3- | HCl | 1 | A | 8.2 | | 359.2 |
| | thiazol-4-yl]methyl}-N-(4- | | | | | | |
| | fluorophenyl)acetamide | | | | | | |
| 221 | N-(4-fluorophenyl)-N-({2-[(2- | HCl | 1 | A | 6.7 | | 324.1 |
| | hydroxyethyl)(methyl)amino]-1,3- | | | | | | |
| | thiazol-4-yl}methyl)acetamide | | | | | | |
| 222 | N-(4-fluorophenyl)-N-{[2-(4- | HCl | 1 | A | 7.3 | | 350.1 |
| | hydroxypiperidin-1-yl)-1,3-thiazol-4- | | | | | | |
| | yl]methyl}acetamide | | | | | | |
| 223 | N-(4-fluorophenyl)-N-{[2-(3- | HCl | 1 | A | 7.5 | | 350.9 |
| | hydroxypiperidin-1-yl)-1,3-thiazol-4- | | | | | | |
| | yl]methyl}acetamide | | | | | | |
| 224 | N-(4-fluorophenyl)-N-({2-[4- | HCl | 1 | A | 7.7 | | 364.2 |
| | (hydroxymethyl)piperidin-1-yl]-1,3- | | | | | | |
| | thiazol-4-yl}methyl)acetamide | | | | | | |
| 225 | N-(4-fluorophenyl)-N-({2-[3- | HCl | 1 | A | 8 | | 364.1 |
| | (hydroxymethyl)piperidin-1-yl]-1,3- | | | | | | |
| | thiazol-4-yl}methyl)acetamide | | | | | | |
| 226 | N-(4-fluorophenyl)-N-{[6-(4-methyl- | HCl | 5 | A | 4.9 | | 357.1 |
| | 1,4-diazepan-1-yl)pyridin-3- | | | | | | |
| | yl]methyl}acetamide | | | | | | |
| 227 | N-(4-fluorophenyl)-N-[(6-morpholin-4- | HCl | 5 | A | 6.9 | | 330.2 |
| | ylpyridin-3-yl)methyl]acetamide | | | | | | |
| 228 | N-{[6-(diethylamino)pyridin-3- | HCl | 5 | A | 6 | | 316.2 |
| | yl]methyl}-N-(4- | | | | | | |
| | fluorophenyl)acetamide | | | | | | |
| 229 | N-{[6-(4-cyanopiperidin-1-yl)pyridin-3- | HCl | 5 | A | 7.4 | | 353.2 |
| | yl]methyl}-N-(4- | | | | | | |
| | fluorophenyl)acetamide | | | | | | |
| 230 | N-(4-fluorophenyl)-N-({6-[(2- | HCl | 5 | A | 4.5 | | 318.1 |
| | hydroxyethyl)(methyl)amino]pyridin-3- | | | | | | |
| | yl}methyl)acetamide | | | | | | |
| 231 | N-(4-fluorophenyl)-N-{[6-(4- | HCl | 5 | A | 5.3 | | 344.1 |
| | hydroxypiperidin-1-yl)pyridin-3- | | | | | | |
| | yl]methyl}acetamide | | | | | | |
| 232 | N-(4-fluorophenyl)-N-{[6-(3- | HCl | 5 | A | 5.5 | | 344.1 |
| | hydroxypiperidin-1-yl)pyridin-3- | | | | | | |
| | yl]methyl}acetamide | | | | | | |
| 233 | N-(4-fluorophenyl)-N-({6-[4- | HCl | 5 | A | 5.5 | | 358.1 |
| | (hydroxymethyl)piperidin-1-yl]pyridin- | | | | | | |
| | 3-yl}methyl)acetamide | | | | | | |
| 234 | N-(4-fluorophenyl)-N-({6-[3- | HCl | 5 | A | 5.8 | | 358.1 |
| | (hydroxymethyl)piperidin-1-yl]pyridin- | | | | | | |
| | 3-yl}methyl)acetamide | | | | | | |
| 235 | N-cyclopentyl-N-({6-[4- | HCl | 10 | A | 5 | | 332.2 |
| | (hydroxymethyl)piperidin-1-yl]pyridin- | | | | | | |
| | 3-yl}methyl)acetamide | | | | | | |
| 236 | N-cyclohexyl-N-{[6-(4- | HCl | 10 | A | 5.4 | | 331.1 |
| | methylpiperazin-1-yl)pyridin-3- | | | | | | |
| | yl]methyl}acetamide | | | | | | |
| 237 | N-cyclohexyl-N-({6-[4- | HCl | 10 | A | 5.7 | | 346.2 |
| | (hydroxymethyl)piperidin-1-yl]pyridin- | | | | | | |
| | 3-yl}methyl)acetamide | | | | | | |
| 238 | N-(4-tert-butylcyclohexyl)-N-({2- | HCl | 2 | A | 11.3 | | 378 |
| | [cyclopropyl(ethyl)amino]-1,3-thiazol- | | | | | | |
| | 4-yl}methyl)acetamide | | | | | | |
| 239 | 2,2-dimethylpropanamide | HCl | 1 | A | 10.4 | | 406.2 |
| | N-{[2-(2,6-dimethylmorpholin-4-yl)-1,3- | | | | | | |
| | thiazol-4-yl]methyl}-N-(4- | | | | | | |
| | fluorophenyl)- | | | | | | |
| 240 | N-(4-fluorophenyl)-2,2-dimethyl-N-[(2- | | 11 | A | 10.9 | | 420.2 |
| | quinolin-3-yl-1,3-thiazol-4- | | | | | | |
| | yl)methyl]propanamide | | | | | | |
| 241 | N-(4-fluorophenyl)-2,2-dimethyl-N-({2- | | 11 | A | 11.5 | | 437.1 |
| | [4-(trifluoromethyl)phenyl]-1,3-thiazol- | | | | | | |
| | 4-yl}methyl)propanamide | | | | | | |
| 242 | N-(4-fluorophenyl)-2,2-dimethyl-N-({2- | | 11 | A | 11.5 | | 437.1 |
| | [3-(trifluoromethyl)phenyl]-1,3-thiazol- | | | | | | |
| | 4-yl}methyl)propanamide | | | | | | |
| 243 | N-(4-fluorophenyl)-2,2-dimethyl-N-({2- | | 11 | A | 11.0 | | 437.1 |
| | [2-(trifluoromethyl)phenyl]-1,3-thiazol- | | | | | | |
| | 4-yl}methyl)propanamide | | | | | | |
| 244 | N-(4-fluorophenyl)-N-{[2-(4- | | 11 | A | 10.2 | | 385.1 |
| | hydroxyphenyl)-1,3-thiazol-4- | | | | | | 385.2 |
| | yl]methyl}-2,2-dimethylpropanamide | | | | | | |
| 245 | N-(4-fluorophenyl)-N-{[2-(3- | | 11 | A | 10.3 | | 385.1 |
| | hydroxyphenyl)-1,3-thiazol-4- | | | | | | |
| | yl]methyl}-2,2-dimethylpropanamide | | | | | | |
| 246 | N-(4-fluorophenyl)-N-{[2-(2- | | 11 | A | 11.0 | | 384.9 |
| | hydroxyphenyl)-1,3-thiazol-4- | | | | 11.1 | | 385.2 |
| | yl]methyl}-2,2-dimethylpropanamide | | | | | | 387.2 |
| 247 | N-(4-fluorophenyl)-N-{[2-(4- | | 11 | A | 11.0 | | 385.1 |
| | fluorophenyl)-1,3-thiazol-4-yl]methyl}- | | | | 11.1 | | 386.8 |
| | 2,2-dimethylpropanamide | | | | | | 387.1 |
| 248 | N-(4-fluorophenyl)-N-{[2-(3- | | 11 | A | 11.1 | | 387.1 |
| | fluorophenyl)-1,3-thiazol-4-yl]methyl}- | | | | | | 387.2 |
| | 2,2-dimethylpropanamide | | | | | | |
| 249 | N-(4-fluorophenyl)-N-{[2-(2- | | 11 | A | 11.1 | | 387.2 |
| | fluorophenyl)-1,3-thiazol-4-yl]methyl}- | | | | | | |
| | 2,2-dimethylpropanamide | | | | | | |
| 250 | N-(4-fluorophenyl)-N-{[6-(4- | HCl | | A | 5.5 | 399 | |
| | methylpiperazin-1-yl)pyridin-3- | TFA | | | | | |
| | yl]methyl}tetrahydrofuran-2- | | | | | | |
| | carboxamide | | | | | | |
| 251 | N-(4-fluorophenyl)-N-{[6-(4- | HCl | 12 | A | 5.4 | 399 | |
| | methylpiperazin-1-yl)pyridin-3- | TFA | | | | | |
| | yl]methyl}tetrahydrofuran-3- | | | | | | |
| | carboxamide | | | | | | |
| 252 | N-[(6-chloropyridin-3-yl)methyl]-N-(4- | | 12 | A | 8.2 | 335 | |
| | fluorophenyl)tetrahydrofuran-2- | | | | | | |
| | carboxamide | | | | | | |
| 253 | N-[(6-chloropyridin-3-yl)methyl]-N-(4- | | 12 | A | 8.1 | 334.9 | |
| | fluorophenyl)tetrahydrofuran-3- | | | | | | |
| | carboxamide | | | | | | |
| 254 | tert-butyl (3S)-3-{[(6-chloropyridin-3- | | 13 | A | 9.9 | 434 | |
| | yl)methyl](4- | | | | | | |
| | fluorophenyl)carbamoyl}pyrrolidine-1- | | | | | | |
| | carboxylate | | | | | | |
| 255 | tert-butyl (3R)-3-{[(6-chloropyridin-3- | | 13 | A | 9.9 | 434 | |
| | yl)methyl](4- | | | | | | |
| | fluorophenyl)carbamoyl}pyrrolidine-1- | | | | | | |
| | carboxylate | | | | | | |
| 256 | tert-butyl (3S)-3-[(4-fluorophenyl){[6- | | 13 | A | 7.8 | 498.2 | |
| | (4-methylpiperazin-1-yl)pyridin-3- | | | | | | |
| | yl]methyl}carbamoyl]pyrrolidine-1- | | | | | | |
| | carboxylate | | | | | | |
| 257 | tert-butyl (3R)-3-[(4-fluorophenyl){[6- | | 13 | A | 7.8 | 498.2 | |
| | (4-methylpiperazin-1-yl)pyridin-3- | | | | | | |
| | yl]methyl}carbamoyl]pyrrolidine-1- | | | | | | |
| | carboxylate | | | | | | |
| 258 | (3S)—N-(4-fluorophenyl)-N-{[6-(4- | HCl | 13 | A | 6.6 | 398 | |
| | methylpiperazin-1-yl)pyridin-3- | | | | | | |
| | yl]methyl}pyrrolidine-3-carboxamide | | | | | | |
| 259 | (3R)—N-(4-fluorophenyl)-N-{[6-(4- | HCl | 13 | A | 6.6 | 398 | |
| | methylpiperazin-1-yl)pyridin-3- | | | | | | |
| | yl]methyl}pyrrolidine-3-carboxamide | | | | | | |
| 260 | (3S)-1-(2,2-dimethylpropanoyl)-N-(4- | HCl | 13 | A | 6.8 | 482 | |
| | fluorophenyl)-N-{[6-(4- | | | | | | |
| | methylpiperazin-1-yl)pyridin-3- | | | | | | |
| | yl]methyl}pyrrolidine-3-carboxamide | | | | | | |
| 261 | (3R)-1-(2,2-dimethylpropanoyl)-N-(4- | HCl | 13 | A | 6.8 | 482 | |
| | fluorophenyl)-N-{[6-(4- | | | | | | |
| | methylpiperazin-1-yl)pyridin-3- | | | | | | |
| | yl]methyl}pyrrolidine-3-carboxamide | | | | | | |

TABLE 2B

| | | HPLC | | |
|---|---|---|---|---|
| | | | Retention | Observed Ion |
| | | | time (tᵣ, | m/e |
| No. | Chemical Name | Method | min.) | [M + H] |
| 22 | N-(4-Fluorophenyl)-2,2-dimethyl-N-[6- | C | 2.3 | 399 |
| | (4-methyl-[1,4]diazepan-1-yl)-pyridin-3- | | | |
| | ylmethyl]-propionamide | | | |
| 25 | N-(4-Fluorophenyl)-2,2-dimethyl-N-[6- | C | 3.3 | 448 |
| | (4-pyridin-2-yl-piperazin-1-yl)-pyridin-3- | | | |
| | ylmethyl]-propionamide | | | |
| 26 | N-(4-Fluorophenyl)-2,2-dimethyl-N-[6- | C | 3.4 | 462 |
| | (4-pyridin-2-yl-piperazin-1-yl)-pyridin-3- | | | |
| | ylmethyl]-butyramide | | | |
| 27 | N-(4-Fluorophenyl)-2,2-dimethyl-N-{6- | C | — | 421 |
| | [methyl-(2-pyridin-3-yl-ethyl)-amino]- | | | |
| | pyridin-3-ylmethyl}-propionamide | | | |
| 33 | N-(4-Fluorophenyl)-2,2-dimethyl-N-(4- | C | 3.8 | 455 |
| | morpholin-4-yl-3,4,5,6-tetrahydro-2H- | | | |
| | [1,2′]bipyridinyl-5′-ylmethyl)- | | | |
| | propionamide | | | |
| 34 | N-(4-Fluorophenyl)-2,2-dimethyl-N-(4- | C | 2.3 | 439 |
| | pyrrolidin-1-yl-3,4,5,6-tetrahydro-2H- | | | |
| | [1,2′]bipyridinyl-5′-ylmethyl)- | | | |
| | propionamide | | | |
| 38 | N-(4-Fluorophenyl)-N-{6-[(2-hydroxy- | C | 2.9 | 360 |
| | ethyl)-methyl-amino]-pyridin-3- | | | |
| | ylmethyl}-2,2-dimethyl-propionamide | | | |
| 40 | N-(4-Fluorophenyl)-2,2-dimethyl-N-(6- | C | 3.7 | 342 |
| | {methyl-[2-(6-methyl-pyridazin-3-yloxy)- | | | |
| | ethyl]-amino}-pyridin-3-ylmethyl)- | | | |
| | propionamide | | | |
| 41 | N-[6-(Ethyl-pyridin-4-ylmethyl-amino)- | C | 2.5 | 421 |
| | pyridin-3-ylmethyl]-N-(4-fluorophenyl)- | | | |
| | 2,2-dimethyl-propionamide | | | |
| 42 | N-(4-Fluorophenyl)-2,2-dimethyl-N-(6- | C | 3.2 | 356 |
| | pyrrolidin-1-yl-pyridin-3-ylmethyl)- | | | |
| | propionamide | | | |
| 47 | N-(4-Fluorophenyl)-2,2-dimethyl-N-(6- | C | 3.3 | 451 |
| | {methyl-[2-(pyridin-3-ylmethoxy)-ethyl]- | | | |
| | amino}-pyridin-3-ylmethyl)- | | | |
| | propionamide | | | |
| 48 | N-(4-Fluorophenyl)-N-[6-(2- | C | 3.1 | 386 |
| | hydroxymethyl-pyrrolidin-1-yl)-pyridin- | | | |
| | 3-ylmethyl]-2,2-dimethyl-propionamide | | | |
| 49 | N-(4-Fluorophenyl)-2,2-dimethyl-N-{6- | C | 3.5 | 463 |
| | [2-(pyridin-2-yloxymethyl)-pyrrolidin-1- | | | |
| | yl]-pyridin-3-ylmethyl}-propionamide | | | |
| 50 | N-(4-Fluorophenyl)-2,2-dimethyl-N-{6- | C | 3.4 | 449 |
| | [3-(pyridin-2-yloxy)-pyrrolidin-1-yl]- | | | |
| | pyridin-3-ylmethyl}-propionamide | | | |
| 51 | N-(4-Fluorophenyl)-N-[6-(3-hydroxy- | C | 2.9 | 372 |
| | pyrrolidin-1-yl)-pyridin-3-ylmethyl]-2,2- | | | |
| | dimethyl-propionamide | | | |
| 52 | N-[6-(3-Diethylamino-pyrrolidin-1-yl)- | C | 2.2 | 427 |
| | pyridin-3-ylmethyl]-N-(4-fluorophenyl)- | | | |
| | 2,2-dimethyl-propionamide | | | |
| 55 | N-(4-Fluorophenyl)-2,2-dimethyl-N-{6- | C | 3.4 | 449 |
| | [3-(pyridin-2-yloxy)-pyrrolidin-1-yl]- | | | |
| | pyridin-3-ylmethyl}-propionamide | | | |
| 58 | N-{6-[Ethyl-(2-pyridin-3-yl-ethyl)- | C | 3.4 | 435 |
| | amino]-pyridin-3-ylmethyl}-N-(4- | | | |
| | fluorophenyl)-2,2-dimethyl- | | | |
| | propionamide | | | |
| 59 | N-(4-Fluorophenyl)-2,2-dimethyl-N-[6- | C | 3.3 | 407 |
| | (2-pyridin-3-yl-ethylamino)-pyridin-3- | | | |
| | ylmethyl]-propionamide | | | |
| 62 | N-(6-Dimethylamino-pyridin-3- | C | 3.2 | 330 |
| | ylmethyl)-N-(4-fluorophenyl)-2,2- | | | |
| | dimethyl-propionamide | | | |
| 64 | N-(2-Diethylamino-thiazol-4-ylmethyl)- | C | 3.3 | 364 |
| | N-(4-fluorophenyl)-2,2-dimethyl- | | | |
| | propionamide | | | |
| 65 | N-(4-Fluorophenyl)-2,2-dimethyl-N-[2- | C | 3.3 | 398 |
| | (methyl-phenyl-amino)-thiazol-4- | | | |
| | ylmethyl]-propionamide | | | |
| 66 | N-(4-Fluorophenyl)-N-[6-(furan-2- | C | 3.3 | 396 |
| | ylmethyl-methyl-amino)-pyridin-3- | | | |
| | ylmethyl]-2,2-dimethyl-propionamide | | | |
| 68 | N-(4-Fluorophenyl)-N-[2-(4-methoxy- | C | 3.3 | 429 |
| | phenoxymethyl)-thiazol-4-ylmethyl]-2,2- | | | |
| | dimethyl-propionamide | | | |
| 69 | N-(4-Fluorophenyl)-2,2-dimethyl-N-(2- | C | 3.1 | 378 |
| | morpholin-4-yl-thiazol-4-ylmethyl)- | | | |
| | propionamide | | | |
| 70 | N-(4-Fluorophenyl)-2,2-dimethyl-N- | C | 3.4 | 370 |
| | (3,4,5,6-tetrahydro-2H-[1,2′]bipyridinyl- | | | |
| | 5′-ylmethyl)-propionamide | | | |
| 71 | N-(3-Isopropyl-phenyl)-2,2-dimethyl-N- | C | 3.7 | 400 |
| | (2-piperidin-1-yl-thiazol-4-ylmethyl)- | | | |
| | propionamide | | | |
| 72 | N-(4-Fluorophenyl)-2,2-dimethyl-N-[6- | C | 3.3 | 454 |
| | (4-thiazol-2-yl-piperazin-1-yl)-pyridin-3- | | | |
| | ylmethyl]-propionamide | | | |
| 73 | N-(4-Fluorophenyl)-2,2-dimethyl-N-(2- | C | 3.2 | 392 |
| | morpholin-4-yl-thiazol-4-ylmethyl)- | | | |
| | butyramide | | | |
| 77 | N-(4-Fluorophenyl)-2,2-dimethyl-N-(2- | C | 3.3 | 362 |
| | pyrrolidin-1-yl-thiazol-4-ylmethyl)- | | | |
| | propionamide | | | |
| 78 | N-(6-Cyclopropylamino-pyridin-3- | C | 3.2 | 342 |
| | ylmethyl)-N-(4-fluorophenyl)-2,2- | | | |
| | dimethyl-propionamide | | | |
| 79 | N-(4-Fluorophenyl)-N-{2-[(4- | C | 3.3 | 416 |
| | fluorophenyl)-methyl-amino]-thiazol-4- | | | |
| | ylmethyl}-2,2-dimethyl-propionamide | | | |
| 80 | N-(4-Fluorophenyl)-N-{2-[(4-methoxy- | C | 3.3 | 428 |
| | phenyl)-methyl-amino]-thiazol-4- | | | |
| | ylmethyl}-2,2-dimethyl-propionamide | | | |
| 82 | N-{2-[(4-Fluorophenyl)-methyl-amino]- | C | 3.3 | 417 |
| | thiazol-4-ylmethyl}-N-(5-fluoro-pyridin- | | | |
| | 2-yl)-2,2-dimethyl-propionamide | | | |
| 90 | N-(4-Diethylaminobenzyl)-N-(4- | C | 3.6 | 396 |
| | dimethylamino-phenyl)-2,2-dimethyl- | | | |
| | butyramide | | | |
| 91 | 4-Methyl-pentanoic acid (4- | C | 3.6 | 396 |
| | Diethylaminobenzyl)-(4-dimethylamino- | | | |
| | phenyl)-amide | | | |
| 94 | 4-Methyl-pent-2-enoic acid (4- | C | 3.5 | 394 |
| | Diethylaminobenzyl)-(4-dimethylamino- | | | |
| | phenyl)-amide | | | |
| 95 | 4-Methyl-pentanoic acid (2- | C | 3.5 | 403 |
| | diethylamino-thiazol-4-ylmethyl)-(4- | | | |
| | dimethylamino-phenyl)-amide | | | |
| 96 | N-(2-Diethylamino-thiazol-4-ylmethyl)- | C | 3.5 | 403 |
| | N-(4-dimethylamino-phenyl)-2,2- | | | |
| | dimethyl-butyramide | | | |
| 98 | 4-Methyl-pentanoic acid (4-amino- | C | 3.2 | 368 |
| | phenyl)-(4-Diethylaminobenzyl)-amide | | | |
| 99 | 4-Methyl-pentanoic acid (4- | C | 3.4 | 382 |
| | Diethylaminobenzyl)-(4-methylamino- | | | |
| | phenyl)-amide | | | |
| 100 | N-[6-(Cyclopropyl(ethyl)amino)-pyridin- | C | 3.5 | 395 |
| | 3-ylmethyl]-N-(4-dimethylamino- | | | |
| | phenyl)-2,2-dimethyl-propionamide | | | |
| 104 | Cyclohexanecarboxylic acid (4- | C | 2.5 | 411 |
| | fluorophenyl)-[6-(4-methyl-piperazin-1- | | | |
| | yl)-pyridin-3-ylmethyl]-amide | | | |
| 106 | Cyclohexanecarboxylic acid (4- | C | 3.4 | 474 |
| | fluorophenyl)-[6-(4-pyridin-2-yl- | | | |
| | piperazin-1-yl)-pyridin-3-ylmethyl]- | | | |
| | amide | | | |
| 109 | Tetrahydro-furan-2-carboxylic acid (4- | C | 3.1 | 462 |
| | fluorophenyl)-[6-(4-pyridin-2-yl- | | | |
| | piperazin-1-yl)-pyridin-3-ylmethyl]- | | | |
| | amide | | | |
| 110 | Tetrahydro-furan-3-carboxylic acid (4- | C | 3.5 | 399 |
| | fluorophenyl)-[6-(4-methyl-piperazin-1- | | | |
| | yl)-pyridin-3-ylmethyl]-amide | | | |
| 111 | Tetrahydro-furan-3-carboxylic acid (4- | C | 3.1 | 462 |
| | fluorophenyl)-[6-(4-pyridin-2-yl- | | | |
| | piperazin-1-yl)-pyridin-3-ylmethyl]- | | | |
| | amide | | | |
| 113 | Tetrahydro-pyran-4-carboxylic acid (6- | C | 3.1 | 386 |
| | diethylamino-pyridin-3-ylmethyl)-(4- | | | |
| | fluorophenyl)-amide | | | |
| 114 | 1-Methyl-pyrrolidine-2-carboxylic acid | C | 3.6 | 443 |
| | (4-fluorophenyl)-{2-[(4-fluorophenyl)- | | | |
| | methyl-amino]-thiazol-4-ylmethyl}- | | | |
| | amide | | | |
| 115 | 1-Benzyl-pyrrolidine-2-carboxylic acid | C | 3.7 | 473 |
| | [6-(Cyclopropyl(ethyl)amino)-pyridin-3- | | | |
| | ylmethyl]-(4-fluorophenyl)-amide | | | |
| 116 | N-{[6-(diethylamino)pyridin-3-yl]methyl}- | C | 3.6 | 371.1 |
| | N-(4-fluorophenyl)-L-prolinamide | | | |
| 117 | N-{4-[cyclopropyl(ethyl)amino]benzyl}-N- | C | 3.8 | 394.1 |
| | (4-methoxyphenyl)-L-prolinamide | | | |
| 118 | N-{3-[cyclopropyl(ethyl)amino]benzyl}-N- | C | 3.9 | 382.1 |
| | (4-fluorophenyl)-L-prolinamide | | | |
| 119 | N-(4-chlorophenyl)-N-{[6- | C | 3.9 | 387.1 |
| | (diethylamino)pyridin-2-yl]methyl}-L- | | | |
| | prolinamide | | | |
| 120 | N-(4-fluoro-2-methylphenyl)-N-[(2- | C | 3.8 | 398.1 |
| | piperidin-1-ylpyrimidin-5-yl)methyl]-L- | | | |
| | prolinamide | | | |
| 121 | N-(4-methylphenyl)-N-[(2-pyrrolidin-1-yl- | C | 3.7 | 371.1 |
| | 1,3-thiazol-5-yl)methyl]-L-prolinamide | | | |
| 122 | N-({2-[cyclopropyl(ethyl)amino]-1,3- | C | 9.5 | 389.2 |
| | thiazol-4-yl}methyl)-N-(4-fluorophenyl)-L- | | | |
| | prolinamide | | | |
| 123 | N-({2-[cyclopropyl(ethyl)amino]-1,3- | C | 7.8 | 389 |
| | thiazol-4-yl}methyl)-N-(4-fluorophenyl)-D- | | | |
| | prolinamide | | | |
| 124 | N-({2-[cyclopropyl(ethyl)amino]-1,3- | C | 8.7 | 403 |
| | thiazol-4-yl}methyl)-N-(4-fluorophenyl)-5- | | | |
| | oxo-L-prolinamide | | | |

### Example 39

### Pharmacological Testing

Representative compounds of formula (I) are screened for activity against calcium channel targets in several standard pharmacological test procedures. Based on the activity shown in the standard pharmacological test procedures, the compounds of the present teachings can be useful as ion channel modulators.

### Oocyte Assay

This assay was essentially performed as described in Lin et al. (1997), Neuron 18(11): 153-166; Pan J. and Lipsombe D. (2000), J. Neurosci., 20(13): 4768-75; and Xu W. and Lipscombe D. (2001), J. Neurosci., 21(16): 5944-5951, the entire disclosures of which are herein incorporated by reference, using Xenopus oocyte heterologous expression system. The assay was performed on various calcium channels (e.g., Caᵥ2.2 subfamily) whereby the modulation of the calcium channel was measured for each tested compound. For measuring compound potency on Caᵥ2.2, a train of five depolarizing pulses of 20-30 ms to about +10 mV was applied at a frequency of 5 Hz from a holding potential of −100 mV every 30 seconds. The 50% inhibitory concentration (IC₅₀) of the test compounds was calculated by measuring the current obtained at the fifth pulse (P₅).

### HEK Assay

HEK-293T/17 cells were transiently transfected in a similar manner as described in FuGENE 6 Package Insert Version 7, April 2002, Roche Applied Science, Indianapolis, Ind. The cells were plated at 2.5×10⁵ cells in 2 mL in a 6-well plate, incubated for one night, and achieved a ˜30-40% confluence. In a small sterile tube, sufficient serum-free medium was added as diluent for FuGENE Transfection Reagent (Roche Applied Science, Indianapolis, Ind.) to a total volume of 100 μL. To this medium was added 3 μL of FuGENE 6 Reagent. The mixture was tapped gently to mix. To the prediluted FuGENE 6 Reagent was added 2 μg of DNA solution (0.8-2.0 μg/μL). The DNA/Fugene 6 mixture was gently pipeted to mix the contents and incubated for about 15 minutes at room temperature. The complex mixture was then added to the HEK-293T/17 cells, distributed around the well, and swirled to ensure even dispersal. The cells were returned to the incubator for 24 hours. The transfected cells were then replated at density 2.5×10⁵ in a 35 mm dish with 5 glass coverslips and grew in low serum (1%) media for 24 hours. Coverslips with isolated cells were then transferred into a chamber, and calcium channel (e.g., L-type, N-type, etc.) current or other currents for counter screening were recorded from the transiently transfected HEK-293T/17 cells.

The whole-cell voltage clamp configuration of the patch clamp technique was employed to evaluate voltage-dependent calcium currents essentially as described by Thompson and Wong (1991), J. Physiol., 439: 671-689, the entire disclosure of which is herein incorporated by reference. To record calcium channel (e.g., L-type, N-type, etc.) currents for evaluation of inhibitory potency of compounds (steady-state concentration-response analysis), five pulses of 20-30 ms voltage steps to about +10 mV (the peak of the current voltage relationship) were delivered at five Hz every 30 second from a holding potential at −100 mV. In order to obtain an estimate of the degree of use-dependent block of the test compounds, IC₅₀ values were determined at the first and fifth pulse of the train (P₁ and P₅, respectively, in Table 2). Compound evaluations were carried out essentially as described by Sah D. W. and Bean B. P. (1994), Mol. Pharmacol., 45:84-92, the entire disclosure of which is herein incorporated by reference.

### FLIPR Assay

TSA201 cells stably transfected with human Caᵥ2.2 (composed of the subunits α1, β3 and α2δ) and human Kir2.3 to enhance the FLIPR Ca²⁺ signal were used. These cells were plated on 384-well collagen-coated plates (BD Bioscience, Franklin Lakes, N.J.) at a density of 2×10⁴ cells/well one day prior to the FLIPR assay. For each assay plate, FLUO-4 dye (Invitrogen, Carlsbad, Calif.) was diluted in 12 mL of Dulbecco's Modified Eagle's Medium (Invitrogen, Carlsbad, Calif.) to a final concentration of 4 μM in the presence of Pluronic F-127 (Invitrogen, Carlsbad, Calif.). Culture media is removed and replaced with 25 μl of the FLUO-4 dye solution and incubated for one hour at room temperature. The cell plate is then placed on the FLIPR where the dye is aspirated off and replaced with 25 μl of HBSS (Invitrogen, Carlsbad, Calif.). The HBSS is then aspirated and replaced with 25 l of compound which is diluted in HBSS with 1% DMSO. Compounds are incubated on the cells for 15 minutes. The cells are then depolarized with 25 μl of 140 mM KCl solution (also containing 2 mM CaCl₂ and 10 mM HEPES). The final concentration of KCl on the cells is 70 mM.

The results obtained for selected compounds are summarized in Table 3 and 4 below. Data presented represent the average value when one or more samples were tested.

TABLE 3

| | | Oocyte | HEK | HEK |
|---|---|---|---|---|
| | | Caᵥ2.2 | Caᵥ2.2 | Caᵥ2.2 |
| | | P₅ | P₁ | P₅ |
| | | IC₅₀ | IC₅₀ | IC₅₀ |
| No. | Name | (μM) | (μM) | (μM) |
| 1 | N-(4-Fluorophenyl)-2,2-dimethyl- | | 20.3 | 8.3 |
| | N-(2-pyrrolidin-1-yl-thiazol-5- | | | |
| | ylmethyl)-propionamide | | | |
| 2 | N-(4-Fluorophenyl)-2,2-dimethyl- | 16.9 | 16.8 | 2.0 |
| | N-(2-piperidin-1-yl-thiazol-4- | | | |
| | ylmethyl)-propionamide | | | |
| 3 | N-(6-Diethylamino-pyridin-3- | 12.1 | 4.3 | 1.3 |
| | ylmethyl)-N-(4-fluorophenyl)-2,2- | | | |
| | dimethyl-propionamide | | | |
| 4 | N-(6-Diethylamino-pyridin-2- | | 2.9 | 0.8 |
| | ylmethyl)-N-(4-fluorophenyl)-2,2- | | | |
| | dimethyl-propionamide | | | |
| 5 | N-(4-Diethylaminobenzyl)-N-(4- | | 2.9 | 1.1 |
| | fluorophenyl)-2,2-dimethyl- | | | |
| | propionamide | | | |
| 6 | N-[3-(Cyclopropyl(ethyl)amino)- | | 6.2 | 1.5 |
| | benzyl]-N-(4-fluorophenyl)-2,2- | | | |
| | dimethyl-propionamide | | | |
| 7 | N-[6-(Cyclopropyl(ethyl)amino)- | 8.0 | 12.9 | 1.7 |
| | pyridin-3-ylmethyl]-N-(4- | | | |
| | fluorophenyl)-2,2-dimethyl- | | | |
| | propionamide | | | |
| 8 | N-(4-Fluorophenyl)-2,2-dimethyl- | 8.7 | 5.1 | 1.6 |
| | N-(2-piperidin-1-yl-pyrimidin-5- | | | |
| | ylmethyl)-propionamide | | | |
| 11 | 3-tert-Butyl-1-(4-fluorophenyl)-1- | 44 | | |
| | [6-(4-methyl-piperazin-1-yl)- | | | |
| | pyridin-3-ylmethyl]-urea | | | |
| 12 | 1-Benzyl-piperidine-4-carboxylic | | 1.0 | 0.4 |
| | acid [6-(Cyclopropyl(ethyl)amino)- | | | |
| | pyridin-3-ylmethyl]-(4- | | | |
| | fluorophenyl)-amide | | | |
| 13 | 4-Methyl-pentanoic acid [6- | | | 1.8 |
| | (Cyclopropyl(ethyl)amino)-pyridin- | | | |
| | 3-ylmethyl]-(4-fluorophenyl)-amide | | | |
| 14 | 1,2,3,4-Tetrahydro-isoquinoline-3- | | 10.1 | 0.6 |
| | carboxylic acid [6- | | | |
| | (Cyclopropyl(ethyl)amino)-pyridin- | | | |
| | 3-ylmethyl]-(4-fluorophenyl)-amide | | | |
| 15 | 2-Methyl-1,2,3,4-tetrahydro- | | 2.1 | 0.4 |
| | isoquinoline-3-carboxylic acid [6- | | | |
| | (Cyclopropyl(ethyl)amino)-pyridin- | | | |
| | 3-ylmethyl]-(4-fluorophenyl)-amide | | | |
| 16 | Tetrahydro-furan-2-carboxylic acid | 86 | | |
| | (4-fluorophenyl)-[6-(4-methyl- | | | |
| | piperazin-1-yl)-pyridin-3-ylmethyl]- | | | |
| | amide | | | |
| 17 | N-(4-Fluorophenyl)-N-[6-(4- | 41.9 | | |
| | methyl-piperazin-1-yl)-pyridin-3- | | | |
| | ylmethyl)-2,2-dimethyl- | | | |
| | propionamide | | | |
| 18 | N-(4-Fluorophenyl)-2,2-dimethyl- | 7.1 | 4.0 | 1.7 |
| | N-{6-[methyl-(2-pyridin-2-yl-ethyl)- | | | |
| | amino]-pyridin-3-ylmethyl}- | | | |
| | propionamide | | | |
| 20 | N-(4-Fluorophenyl)-2,2-dimethyl- | 35.5 | 22.2 | 5.2 |
| | N-{6-[methyl-(2-morpholin-4-yl- | | | |
| | ethyl)-amino]-pyridin-3-ylmethyl}- | | | |
| | propionamide | | | |
| 21 | N-(4-Fluoro-2-methyl-phenyl)-2,2- | 18.0 | 10.5 | 2.7 |
| | dimethyl-N-[6-(4-methyl-piperazin- | | | |
| | 1-yl)-pyridin-3-ylmethyl]- | | | |
| | propionamide | | | |
| 22 | N-(4-Fluorophenyl)-2,2-dimethyl- | 18.3 | 27.8 | 5.7 |
| | N-[6-(4-methyl-[1,4]diazepan-1- | | | |
| | yl)-pyridin-3-ylmethyl]- | | | |
| | propionamide | | | |
| 23 | 4,4,4-Trifluoro-N-(4-fluorophenyl)- | | | 1.1 |
| | 3-methyl-N-{6-[methyl-(2-pyridin- | | | |
| | 2-yl-ethyl)-amino]-pyridin-3- | | | |
| | ylmethyl}-butyramide | | | |
| 24 | N-(4-Fluorophenyl)-2,2-dimethyl- | 3.8 | 2.9 | 0.6 |
| | N-{6-[methyl-(2-pyridin-2-yl-ethyl)- | | | |
| | amino]-pyridin-3-ylmethyl}- | | | |
| | butyramide | | | |
| 25 | N-(4-Fluorophenyl)-2,2-dimethyl- | 4.2 | 2.4 | 0.3 |
| | N-[6-(4-pyridin-2-yl-piperazin-1- | | | |
| | yl)-pyridin-3-ylmethyl]- | | | |
| | propionamide | | | |
| 26 | N-(4-Fluorophenyl)-2,2-dimethyl- | 12.2 | 1.7 | 0.4 |
| | N-[6-(4-pyridin-2-yl-piperazin-1- | | | |
| | yl)-pyridin-3-ylmethyl]-butyramide | | | |
| 27 | N-(4-Fluorophenyl)-2,2-dimethyl- | 5.2 | 4.6 | 1.1 |
| | N-{6-[methyl-(2-pyridin-3-yl-ethyl)- | | | |
| | amino]-pyridin-3-ylmethyl}- | | | |
| | propionamide | | | |
| 29 | N-(4-Fluorophenyl)-2,2-dimethyl- | 8.7 | 5.2 | 1.0 |
| | N-(6-{methyl-[2-(pyridin-2-yloxy)- | | | |
| | ethyl]-amino}-pyridin-3-ylmethyl)- | | | |
| | propionamide | | | |
| 30 | N-(2-Isopropyl-phenyl)-2,2- | 23.6 | 18.9 | 2.7 |
| | dimethyl-N-[6-(4-methyl-piperazin- | | | |
| | 1-yl)-pyridin-3-ylmethyl]- | | | |
| | propionamide | | | |
| 31 | N-(6-Diethylamino-pyridin-3- | 6.6 | 5.5 | 1.3 |
| | ylmethyl)-N-(4-fluorophenyl)-2,2- | | | |
| | dimethyl-butyramide | | | |
| 32 | N-(3,5-dimethylphenyl)-2,2- | 21.2 | 24.5 | 2.6 |
| | dimethyl-N-[6-(4-methyl-piperazin- | | | |
| | 1-yl)-pyridin-3-ylmethyl]- | | | |
| | propionamide | | | |
| 33 | N-(4-Fluorophenyl)-2,2-dimethyl- | 22.9 | 37.6 | 11.5 |
| | N-(4-morpholin-4-yl-3,4,5,6- | | | |
| | tetrahydro-2H-[1,2′]bipyridinyl-5′- | | | |
| | ylmethyl)-propionamide | | | |
| 34 | N-(4-Fluorophenyl)-2,2-dimethyl- | 19.6 | 5.5 | 2.4 |
| | N-(4-pyrrolidin-1-yl-3,4,5,6- | | | |
| | tetrahydro-2H-[1,2′]bipyridinyl-5′- | | | |
| | ylmethyl)-propionamide | | | |
| 35 | N-(2-Isopropyl-phenyl)-2,2- | 13.2 | 4.4 | 1.4 |
| | dimethyl-N-[6-(4-methyl-piperazin- | | | |
| | 1-yl)-pyridin-3-ylmethyl]- | | | |
| | butyramide | | | |
| 36 | N-(6-Diethylamino-pyridin-3- | | 3.5 | 1.3 |
| | ylmethyl)-N-(4-fluoro-2-methyl- | | | |
| | phenyl)-2,2-dimethyl- | | | |
| | propionamide | | | |
| 37 | N-(6-Diethylamino-pyridin-3- | | 1.8 | 0.7 |
| | ylmethyl)-N-(2-isopropyl-phenyl)- | | | |
| | 2,2-dimethyl-propionamide | | | |
| 38 | N-(4-Fluorophenyl)-N-{6-[(2- | | 17.1 | 7.6 |
| | hydroxy-ethyl)-methyl-amino]- | | | |
| | pyridin-3-ylmethyl}-2,2-dimethyl- | | | |
| | propionamide | | | |
| 39 | N-(4-Fluorophenyl)-2,2-dimethyl- | | 1.0 | 0.5 |
| | N-(6-{methyl-[2-(5-methyl-pyridin- | | | |
| | 2-yloxy)-ethyl]-amino}-pyridin-3- | | | |
| | ylmethyl)-propionamide | | | |
| 40 | N-(4-Fluorophenyl)-2,2-dimethyl- | | 21.0 | 11.2 |
| | N-(6-{methyl-[2-(6-methyl- | | | |
| | pyridazin-3-yloxy)-ethyl]-amino}- | | | |
| | pyridin-3-ylmethyl)-propionamide | | | |
| 41 | N-[6-(Ethyl-pyridin-4-ylmethyl- | | 6.3 | 2.0 |
| | amino)-pyridin-3-ylmethyl]-N-(4- | | | |
| | fluorophenyl)-2,2-dimethyl- | | | |
| | propionamide | | | |
| 42 | N-(4-Fluorophenyl)-2,2-dimethyl- | | 11.6 | 7.4 |
| | N-(6-pyrrolidin-1-yl-pyridin-3- | | | |
| | ylmethyl)-propionamide | | | |
| 43 | N-(4-Fluorophenyl)-N-[6-(4- | | 10.2 | 3.0 |
| | methanesulfonyl-piperazin-1-yl)- | | | |
| | pyridin-3-ylmethyl]-2,2-dimethyl- | | | |
| | butyramide | | | |
| 44 | N-[6-(4-Acetyl-piperazin-1-yl)- | | 18.4 | 7.5 |
| | pyridin-3-ylmethyl]-N-(4- | | | |
| | fluorophenyl)-2,2-dimethyl- | | | |
| | propionamide | | | |
| 45 | 1-{1-[(6-Diethylamino-pyridin-3- | | 10.6 | 2.5 |
| | ylmethyl)-(4-fluorophenyl)- | | | |
| | carbamoyl]-1-methyl-ethyl}-1H- | | | |
| | [1,2,3]triazole-4-carboxylic acid | | | |
| | ethyl ester | | | |
| 47 | N-(4-Fluorophenyl)-2,2-dimethyl- | | 9.5 | 1.6 |
| | N-(6-{methyl-[2-(pyridin-3- | | | |
| | ylmethoxy)-ethyl]-amino}-pyridin- | | | |
| | 3-ylmethyl)-propionamide | | | |
| 48 | N-(4-Fluorophenyl)-N-[6-(2- | | 19.8 | 4.2 |
| | hydroxymethyl-pyrrolidin-1-yl)- | | | |
| | pyridin-3-ylmethyl]-2,2-dimethyl- | | | |
| | propionamide | | | |
| 49 | N-(4-Fluorophenyl)-2,2-dimethyl- | | 2.3 | 0.6 |
| | N-{6-[2-(pyridin-2-yloxymethyl)- | | | |
| | pyrrolidin-1-yl]-pyridin-3-ylmethyl}- | | | |
| | propionamide | | | |
| 50 | N-(4-Fluorophenyl)-2,2-dimethyl- | | 3.7 | 0.8 |
| | N-{6-[3-(pyridin-2-yloxy)-pyrrolidin- | | | |
| | 1-yl]-pyridin-3-ylmethyl}- | | | |
| | propionamide | | | |
| 51 | N-(4-Fluorophenyl)-N-[6-(3- | | 34.0 | 12.8 |
| | hydroxy-pyrrolidin-1-yl)-pyridin-3- | | | |
| | ylmethyl]-2,2-dimethyl- | | | |
| | propionamide | | | |
| 52 | N-[6-(3-Diethylamino-pyrrolidin-1- | | 17.1 | 6.3 |
| | yl)-pyridin-3-ylmethyl]-N-(4- | | | |
| | fluorophenyl)-2,2-dimethyl- | | | |
| | propionamide | | | |
| 53 | N-[4-(1H-Benzoimidazol-2-yl)- | | 0.6 | 0.3 |
| | 3,4,5,6-tetrahydro-2H- | | | |
| | [1,2′]bipyridinyl-5′-ylmethyl]-N-(4- | | | |
| | fluorophenyl)-2,2-dimethyl- | | | |
| | propionamide | | | |
| 54 | N-(6-Diethylamino-pyridin-3- | | 8.9 | 5.3 |
| | ylmethyl)-2-(4- | | | |
| | dimethylaminomethyl- | | | |
| | [1,2,3]triazol-1-yl)-N-(4- | | | |
| | fluorophenyl)-isobutyramide | | | |
| 55 | N-(4-Fluorophenyl)-2,2-dimethyl- | | 1.7 | 0.6 |
| | N-{6-[3-(pyridin-2-yloxy)-pyrrolidin- | | | |
| | 1-yl]-pyridin-3-ylmethyl}- | | | |
| | propionamide | | | |
| 56 | N-(4-Fluorophenyl)-2,2-dimethyl- | | 6.0 | 2.8 |
| | N-[6-(3-methylamino-pyrrolidin-1- | | | |
| | yl)-pyridin-3-ylmethyl]- | | | |
| | propionamide | | | |
| 58 | N-{6-[Ethyl-(2-pyridin-3-yl-ethyl)- | | 4.9 | 0.9 |
| | amino]-pyridin-3-ylmethyl}-N-(4- | | | |
| | fluorophenyl)-2,2-dimethyl- | | | |
| | propionamide | | | |
| 59 | N-(4-Fluorophenyl)-2,2-dimethyl- | | 29.1 | 3.8 |
| | N-[6-(2-pyridin-3-yl-ethylamino)- | | | |
| | pyridin-3-ylmethyl]-propionamide | | | |
| 63 | 2-(4-Benzyl-[1,2,3]triazol-1-yl)-N- | | 2.7 | 0.7 |
| | (6-diethylamino-pyridin-3- | | | |
| | ylmethyl)-N-(4-fluorophenyl)- | | | |
| | isobutyramide | | | |
| 64 | N-(2-Diethylamino-thiazol-4- | 18.3 | 6.0 | 2.4 |
| | ylmethyl)-N-(4-fluorophenyl)-2,2- | | | |
| | dimethyl-propionamide | | | |
| 65 | N-(4-Fluorophenyl)-2,2-dimethyl- | | 1.8 | 0.6 |
| | N-[2-(methyl-phenyl-amino)- | | | |
| | thiazol-4-ylmethyl]-propionamide | | | |
| 66 | N-(4-Fluorophenyl)-N-[6-(furan-2- | | 3.5 | 1.1 |
| | ylmethyl-methyl-amino)-pyridin-3- | | | |
| | ylmethyl]-2,2-dimethyl- | | | |
| | propionamide | | | |
| 67 | N-(6-tert-Butylamino-pyridin-3- | | 9.3 | 3.7 |
| | ylmethyl)-N-(4-fluorophenyl)-2,2- | | | |
| | dimethyl-propionamide | | | |
| 68 | N-(4-Fluorophenyl)-N-[2-(4- | | 3.2 | 1.2 |
| | methoxy-phenoxymethyl)-thiazol- | | | |
| | 4-ylmethyl]-2,2-dimethyl- | | | |
| | propionamide | | | |
| 69 | N-(4-Fluorophenyl)-2,2-dimethyl- | | | 32% |
| | N-(2-morpholin-4-yl-thiazol-4- | | | |
| | ylmethyl)-propionamide | | | |
| 70 | N-(4-Fluorophenyl)-2,2-dimethyl- | 9.8 | 12.1 | 1.9 |
| | N-(3,4,5,6-tetrahydro-2H- | | | |
| | [1,2′]bipyridinyl-5′-ylmethyl)- | | | |
| | propionamide | | | |
| 71 | N-(3-Isopropyl-phenyl)-2,2- | | 2.4 | 0.5 |
| | dimethyl-N-(2-piperidin-1-yl- | | | |
| | thiazol-4-ylmethyl)-propionamide | | | |
| 73 | N-(4-Fluorophenyl)-2,2-dimethyl- | | 33.8 | 8.5 |
| | N-(2-morpholin-4-yl-thiazol-4- | | | |
| | ylmethyl)-butyramide | | | |
| 74 | N-[6-(Cyclopropyl-methyl-amino)- | | 12.8 | 2.8 |
| | pyridin-3-ylmethyl]-N-(4- | | | |
| | fluorophenyl)-2,2-dimethyl- | | | |
| | propionamide | | | |
| 75 | N-(4-Fluorophenyl)-2,2-dimethyl- | | 47.3 | 11.0 |
| | N-(2-pyridin-4-yl-thiazol-4- | | | |
| | ylmethyl)-propionamide | | | |
| 76 | N-(4-Fluorophenyl)-2,2-dimethyl- | | 29.0 | 5.2 |
| | N-[2-(4-methyl-piperazin-1-yl)- | | | |
| | thiazol-5-ylmethyl]-propionamide | | | |
| 77 | N-(4-Fluorophenyl)-2,2-dimethyl- | | 19.1 | 2.6 |
| | N-(2-pyrrolidin-1-yl-thiazol-4- | | | |
| | ylmethyl)-propionamide | | | |
| 78 | N-(6-Cyclopropylamino-pyridin-3- | | 14.2 | 7.2 |
| | ylmethyl)-N-(4-fluorophenyl)-2,2- | | | |
| | dimethyl-propionamide | | | |
| 79 | N-(4-Fluorophenyl)-N-{2-[(4- | | 0.3 | 0.1 |
| | fluorophenyl)-methyl-amino]- | | | |
| | thiazol-4-ylmethyl}-2,2-dimethyl- | | | |
| | propionamide | | | |
| 80 | N-(4-Fluorophenyl)-N-{2-[(4- | | 2.4 | 0.6 |
| | methoxy-phenyl)-methyl-amino]- | | | |
| | thiazol-4-ylmethyl}-2,2-dimethyl- | | | |
| | propionamide | | | |
| 81 | N-(4-Dimethylamino-phenyl)-N-{2- | 1.7 | 2.0 | 0.6 |
| | [(4-fluorophenyl)-methyl-amino]- | | | |
| | thiazol-4-ylmethyl}-2,2-dimethyl- | | | |
| | propionamide | | | |
| 82 | N-{2-[(4-Fluorophenyl)-methyl- | | 12.1 | 2.2 |
| | amino]-thiazol-4-ylmethyl}-N-(5- | | | |
| | fluoro-pyridin-2-yl)-2,2-dimethyl- | | | |
| | propionamide | | | |
| 83 | N-[2-(Cyclopropyl(ethyl)amino)- | 7.1 | 3.3 | 1.0 |
| | thiazol-4-ylmethyl]-N-(4- | | | |
| | fluorophenyl)-2,2-dimethyl- | | | |
| | propionamide | | | |
| 84 | N-(2-Diethylamino-pyrimidin-5- | 15.6 | 4.3 | 1.2 |
| | ylmethyl)-N-(4-fluorophenyl)-2,2- | | | |
| | dimethyl-propionamide | | | |
| 85 | N-(4-Dimethylamino-phenyl)-2,2- | 16.2 | 7.4 | 1.9 |
| | dimethyl-N-(2-piperidin-1-yl- | | | |
| | pyrimidin-5-ylmethyl)- | | | |
| | propionamide | | | |
| 86 | N-(4-Dimethylamino-phenyl)-2,2- | | 12.6 | 3.1 |
| | dimethyl-N-(2-piperidin-1-yl- | | | |
| | thiazol-4-ylmethyl)-propionamide | | | |
| 87 | N-[4-(Cyclopropyl(ethyl)amino)- | | 4.2 | 1.4 |
| | benzyl]-N-(4-fluorophenyl)-2,2- | | | |
| | dimethyl-propionamide | | | |
| 88 | N-(4-Diethylamino-phenyl)-N-{2- | | 0.4 | 0.1 |
| | [(4-fluorophenyl)-methyl-amino]- | | | |
| | thiazol-4-ylmethyl}-2,2-dimethyl- | | | |
| | propionamide | | | |
| 89 | N-(4-Dimethylamino-phenyl)-2,2- | | 1.1 | 0.3 |
| | dimethyl-N-{2-[methyl-(4- | | | |
| | trifluoromethyl-phenyl)-amino]- | | | |
| | thiazol-4-ylmethyl}-propionamide | | | |
| 90 | N-(4-Diethylaminobenzyl)-N-(4- | | 2.2 | 0.8 |
| | dimethylamino-phenyl)-2,2- | | | |
| | dimethyl-butyramide | | | |
| 91 | 4-Methyl-pentanoic acid (4- | | 1.0 | 0.6 |
| | Diethylaminobenzyl)-(4- | | | |
| | dimethylamino-phenyl)-amide | | | |
| 92 | N-[6-(Cyclopropyl(ethyl)amino)- | | 1.8 | 0.8 |
| | pyridin-2-ylmethyl]-N-(4- | | | |
| | fluorophenyl)-2,2-dimethyl- | | | |
| | propionamide | | | |
| 93 | N-(4-Fluorophenyl)-2,2-dimethyl- | 22.8 | 0.8 | 0.4 |
| | N-[2-(methyl-pyridin-2-yl-amino)- | | | |
| | thiazol-4-ylmethyl]-propionamide | | | |
| 94 | 4-Methyl-pent-2-enoic acid (4- | | 1.8 | 0.7 |
| | Diethylaminobenzyl)-(4- | | | |
| | dimethylamino-phenyl)-amide | | | |
| 95 | 4-Methyl-pentanoic acid (2- | | 7.9 | 2.3 |
| | diethylamino-thiazol-4-ylmethyl)- | | | |
| | (4-dimethylamino-phenyl)-amide | | | |
| 96 | N-(2-Diethylamino-thiazol-4- | | 4.5 | 1.1 |
| | ylmethyl)-N-(4-dimethylamino- | | | |
| | phenyl)-2,2-dimethyl-butyramide | | | |
| 97 | N-[3-(Cyclopropyl(ethyl)amino)- | | 1.1 | 0.4 |
| | benzyl]-N-(4-diethylamino- | | | |
| | phenyl)-2,2-dimethyl- | | | |
| | propionamide | | | |
| 98 | 4-Methyl-pentanoic acid (4-amino- | | 5.2 | 1.9 |
| | phenyl)-(4-Diethylaminobenzyl)- | | | |
| | amide | | | |
| 99 | 4-Methyl-pentanoic acid (4- | | 4.3 | <<0.1 |
| | Diethylaminobenzyl)-(4- | | | |
| | methylamino-phenyl)-amide | | | |
| 100 | N-[6-(Cyclopropyl(ethyl)amino)- | | 8.0 | 4.3 |
| | pyridin-3-ylmethyl]-N-(4- | | | |
| | dimethylamino-phenyl)-2,2- | | | |
| | dimethyl-propionamide | | | |
| 101 | (4-Fluorophenyl)-(2-pyridin-4-yl- | | | 8.4 |
| | thiazol-4-ylmethyl)-carbamic acid | | | |
| | tert-butyl ester | | | |
| 102 | (4-Fluorophenyl)-[2-(4-methyl- | | | 9.6 |
| | piperazin-1-yl)-thiazol-5-ylmethyl]- | | | |
| | carbamic acid tert-butyl ester | | | |
| 103 | Cyclopropanecarboxylic acid (4- | 128.7 | | |
| | fluorophenyl)-[6-(4-methyl- | | | |
| | piperazin-1-yl)-pyridin-3-ylmethyl]- | | | |
| | amide | | | |
| 104 | Cyclohexanecarboxylic acid (4- | 22.0 | 14.1 | 2.5 |
| | fluorophenyl)-[6-(4-methyl- | | | |
| | piperazin-1-yl)-pyridin-3-ylmethyl]- | | | |
| | amide | | | |
| 107 | Cyclopropanecarboxylic acid (4- | | 11.1 | 2.5 |
| | fluorophenyl)-(2-piperidin-1-yl- | | | |
| | thiazol-4-ylmethyl)-amide | | | |
| 108 | Cyclohexanecarboxylic acid (4- | | 14.1 | 3.9 |
| | fluorophenyl)-(2-morpholin-4-yl- | | | |
| | thiazol-4-ylmethyl)-amide | | | |
| 109 | Tetrahydro-furan-2-carboxylic acid | | 13.9 | 2.6 |
| | (4-fluorophenyl)-[6-(4-pyridin-2-yl- | | | |
| | piperazin-1-yl)-pyridin-3-ylmethyl]- | | | |
| | amide | | | |
| 110 | N-(4-fluorophenyl)-N-{[6-(4- | 65.0 | | |
| | methylpiperazin-1-yl)pyridin-3- | | | |
| | yl]methyl}tetrahydrofuran-2- | | | |
| | carboxamide | | | |
| 111 | Tetrahydro-furan-3-carboxylic acid | 23.2 | 18.6 | 2.1 |
| | (4-fluorophenyl)-[6-(4-pyridin-2-yl- | | | |
| | piperazin-1-yl)-pyridin-3-ylmethyl]- | | | |
| | amide | | | |
| 112 | Tetrahydro-furan-3-carboxylic acid | 37 | | |
| | (6-diethylamino-pyridin-3- | | | |
| | ylmethyl)-(4-fluorophenyl)-amide | | | |
| 113 | Tetrahydro-pyran-4-carboxylic | 30 | | |
| | acid (6-diethylamino-pyridin-3- | | | |
| | ylmethyl)-(4-fluorophenyl)-amide | | | |
| 114 | 1-Methyl-pyrrolidine-2-carboxylic | | 4.8 | 2.1 |
| | acid (4-fluorophenyl)-{2-[(4- | | | |
| | fluorophenyl)-methyl-amino]- | | | |
| | thiazol-4-ylmethyl}-amide | | | |
| 115 | 1-Benzyl-pyrrolidine-2-carboxylic | | 2.7 | 0.6 |
| | acid [6-(Cyclopropyl(ethyl)amino)- | | | |
| | pyridin-3-ylmethyl]-(4- | | | |
| | fluorophenyl)-amide | | | |

TABLE 4

| | | FLIPR |
|---|---|---|
| No. | Chemical Name | Caᵥ2.2 IC₅₀ (μM) |
| 116 | N-{[6-(diethylamino)pyridin-3-yl]methyl}-N-(4-fluorophenyl)-L-prolinamide | 34.8 |
| 117 | N-{4-[cyclopropyl(ethyl)amino]benzyl}-N-(4-methoxyphenyl)-L- | 9.7 |
| | prolinamide | |
| 118 | N-{3-[cyclopropyl(ethyl)amino]benzyl}-N-(4-fluorophenyl)-L-prolinamide | 3.4 |
| 119 | N-(4-chlorophenyl)-N-{[6-(diethylamino)pyridin-2-yl]methyl}-L-prolinamide | 12.2 |
| 120 | N-(4-fluoro-2-methylphenyl)-N-[(2-piperidin-1-ylpyrimidin-5-yl)methyl]-L- | 13.7 |
| | prolinamide | |
| 121 | N-(4-methylphenyl)-N-[(2-pyrrolidin-1-yl-1,3-thiazol-5-yl)methyl]-L- | 20.9 |
| | prolinamide | |
| 122 | N-({2-[cyclopropyl(ethyl)amino]-1,3-thiazol-4-yl}methyl)-N-(4- | 13.8 |
| | fluorophenyl)-L-prolinamide | |
| 123 | N-({2-[cyclopropyl(ethyl)amino]-1,3-thiazol-4-yl}methyl)-N-(4- | 10.2 |
| | fluorophenyl)-D-prolinamide | |
| 124 | N-({2-[cyclopropyl(ethyl)amino]-1,3-thiazol-4-yl}methyl)-N-(4- | 19.7 |
| | fluorophenyl)-5-oxo-L-prolinamide | |
| 125 | N-(4-fluorophenyl)-N-{[6-(4-methylpiperazin-1-yl)pyridin-3- | 9.5 |
| | yl]methyl}cyclohexanecarboxamide | |
| 126 | N′-(tert-butyl)-N-(4-fluorophenyl)-N-{[6-(4-methylpiperazin-1-yl)pyridin-3- | 30.05 |
| | yl]methyl}urea | |
| 127 | 4,4,4-trifluoro-N-(4-fluorophenyl)-3-methyl-N-({6-[methyl(2-pyridin-2- | 2.6 |
| | ylethyl)amino]pyridin-3-yl}methyl)butanamide | |
| 128 | N-(4-fluorophenyl)-N-{[6-(4-pyridin-2-ylpiperazin-1-yl)pyridin-3- | 9.1 |
| | yl]methyl}cyclohexanecarboxamide | |
| 129 | N-{[6-(diethylamino)pyridin-3-yl]methyl}-N-(4-fluorophenyl)-2-(2- | 1.5 |
| | thienyl)acetamide | |
| 130 | N-(4-fluorophenyl)-N-({6-[(2-hydroxyethyl)(methyl)amino]pyridin-3- | 14.8 |
| | yl}methyl)-2,2-dimethylpropanamide | |
| 131 | N-(4-fluorophenyl)-2,2-dimethyl-N-{[6-(methyl{2-[(5-methylpyridin-2- | 8.1 |
| | yl)oxy]ethyl}amino)pyridin-3-yl]methyl}propanamide | |
| 132 | N-(4-fluorophenyl)-2,2-dimethyl-N-[(6-pyrrolidin-1-ylpyridin-3- | 8.2 |
| | yl)methyl]propanamide | |
| 133 | N-({6-[3-(diethylamino)pyrrolidin-1-yl]pyridin-3-yl}methyl)-N-(4- | 11.5 |
| | fluorophenyl)-2,2-dimethylpropanamide | |
| 134 | N-(4-fluorophenyl)-2,2-dimethyl-N-[(2-piperidin-1-yl-1,3-thiazol-4- | 4.4 |
| | yl)methyl]propanamide | |
| 135 | N-(4-fluorophenyl)-2,2-dimethyl-N-({6-[propionyl(2-pyridin-3- | 1.35 |
| | ylethyl)amino]pyridin-3-yl}methyl)propanamide | |
| 136 | N-(4-fluorophenyl)-2,2-dimethyl-N-[(2-morpholin-4-yl-1,3-thiazol-4- | 5.6 |
| | yl)methyl]propanamide | |
| 137 | N-(4-fluorophenyl)-N-[(2-morpholin-4-yl-1,3-thiazol-4- | 11.2 |
| | yl)methyl]cyclohexanecarboxamide | |
| 138 | N-(4-fluorophenyl)-2,2-dimethyl-N-[(2-morpholin-4-yl-1,3-thiazol-4- | 14.55 |
| | yl)methyl]butanamide | |
| 139 | N-({6-[cyclopropyl(ethyl)amino]pyridin-3-yl}methyl)-N-(4-fluorophenyl)- | 1.3 |
| | 2,2-dimethylpropanamide | |
| 140 | N-(4-fluorophenyl)-2,2-dimethyl-N-[(2-pyrrolidin-1-yl-1,3-thiazol-4- | 4.4 |
| | yl)methyl]propanamide | |
| 141 | N-(4-fluorophenyl)-N-({2-[(4-fluorophenyl)(methyl)amino]-1,3-thiazol-4- | 7.6 |
| | yl}methyl)-2,2-dimethylpropanamide | |
| 142 | N-(4-fluorophenyl)-N-({2-[(4-methoxyphenyl)(methyl)amino]-1,3-thiazol-4- | 6.8 |
| | yl}methyl)-2,2-dimethylpropanamide | |
| 143 | N-({2-[cyclopropyl(ethyl)amino]-1,3-thiazol-4-yl}methyl)-N-(4- | 3.8 |
| | fluorophenyl)-2,2-dimethylpropanamide | |
| 144 | N-[(2-bromo-1,3-thiazol-4-yl)methyl]-N-(4-fluorophenyl)-2,2- | 3.2 |
| | dimethylpropanamide | |
| 145 | N-{[2-(3,5-dimethylpiperazin-1-yl)-1,3-thiazol-4-yl]methyl}-N-(4- | 18.45 |
| | fluorophenyl)-2,2-dimethylpropanamide | |
| 146 | tert-butyl 4-(4-{[(2,2-dimethylpropanoyl)(4-fluorophenyl)amino]methyl}-- | 35.4 |
| | 1,3-thiazol-2-yl)piperazine-1-carboxylate | |
| 147 | N-(4-fluorophenyl)-2,2-dimethyl-N-{[2-(4-phenylpiperazin-1-yl)-1,3- | 2.8 |
| | thiazol-4-yl]methyl}propanamide | |
| 148 | N-(4-fluorophenyl)-N-{[2-(4-isopropylpiperazin-1-yl)-1,3-thiazol-4- | 15.4 |
| | yl]methyl}-2,2-dimethylpropanamide | |
| 149 | N-(4-fluorophenyl)-2,2-dimethyl-N-{[2-(4-pyridin-2-ylpiperazin-1-yl)--1,3- | 8.1 |
| | thiazol-4-yl]methyl}propanamide | |
| 150 | N-(4-fluorophenyl)-2,2-dimethyl-N-{[2-(4-methylpiperazin-1-yl)-1,3- | 10.55 |
| | thiazol-4-yl]methyl}propanamide | |
| 151 | N-(4-fluorophenyl)-2,2-dimethyl-N-{[2-(4-pyrimidin-2-ylpiperazin-1-yl)-- | 4 |
| | 1,3-thiazol-4-yl]methyl}propanamide | |
| 152 | N-(4-fluorophenyl)-2,2-dimethyl-N-[(2-piperazin-1-yl-1,3-thiazol-4- | 8.3 |
| | yl)methyl]propanamide | |
| 153 | N-[(2-{[2-(dimethylamino)ethyl]amino}-1,3-thiazol-4-yl)methyl]-N-(4- | 12.9 |
| | fluorophenyl)-2,2-dimethylpropanamide | |
| 154 | N-[(2-{[3-(dimethylamino)propyl]amino}-1,3-thiazol-4-yl)methyl]-N-(4- | 9 |
| | fluorophenyl)-2,2-dimethylpropanamide | |
| 155 | N-[(2-{[4-(dimethylamino)butyl]amino}-1,3-thiazol-4-yl)methyl]-N-(4- | 8.5 |
| | fluorophenyl)-2,2-dimethylpropanamide | |
| 156 | N-{[2-(cyclopentylamino)-1,3-thiazol-4-yl]methyl}-N-(4-fluorophenyl)-2,2- | 3 |
| | dimethylpropanamide | |
| 157 | N-{[2-(cyclohexylamino)-1,3-thiazol-4-yl]methyl}-N-(4-fluorophenyl)-2,2- | 8.8 |
| | dimethylpropanamide | |
| 158 | N-(4-fluorophenyl)-N-({2-[(4-fluorophenyl)amino]-1,3-thiazol-4-yl}methyl)- | 9.4 |
| | 2,2-dimethylpropanamide | |
| 159 | N-(4-fluorophenyl)-N-({2-[(2-fluorophenyl)amino]-1,3-thiazol-4-yl}methyl)- | 10.2 |
| | 2,2-dimethylpropanamide | |
| 160 | N-(4-fluorophenyl)-2,2-dimethyl-N-{[2-(methylamino)-1,3-thiazol-4- | 6.1 |
| | yl]methyl}propanamide | |
| 161 | N-(4-fluorophenyl)-N-{[2-(isobutylamino)-1,3-thiazol-4-yl]methyl}-2,2- | 2.7 |
| | dimethylpropanamide | |
| 162 | N-({2-[(1S,4S)-2,5-diazabicyclo[2.2.1]hept-2-yl]-1,3-thiazol-4-yl}methyl)- | 11 |
| | N-(4-fluorophenyl)-2,2-dimethylpropanamide | |
| 163 | N-(4-fluorophenyl)-2,2-dimethyl-N-{[2-(propylamino)-1,3-thiazol-4- | 7.7 |
| | yl]methyl}propanamide | |
| 164 | N-{[2-(dimethylamino)-1,3-thiazol-4-yl]methyl}-N-(4-fluorophenyl)-2,2- | 4.75 |
| | dimethylpropanamide | |
| 165 | N-(4-fluorophenyl)-2,2-dimethyl-N-({2-[4-(2-phenylethyl)piperazin-1-yl]-- | 3.6 |
| | 1,3-thiazol-4-yl}methyl)propanamide | |
| 166 | N-[(2-{4-[3-(dimethylamino)propyl]piperazin-1-yl}-1,3-thiazol-4-yl)methyl]- | 19.8 |
| | N-(4-fluorophenyl)-2,2-dimethylpropanamide | |
| 167 | N-({2-[4-(diphenylmethyl)piperazin-1-yl]-1,3-thiazol-4-yl}methyl)-N-(4- | 13.35 |
| | fluorophenyl)-2,2-dimethylpropanamide | |
| 168 | N-({2-[cyclopropyl(ethyl)amino]-1,3-thiazol-4-yl}methyl)-2,2-dimethyl-N- | 5.1 |
| | phenylpropanamide | |
| 169 | N-({2-[cyclopropyl(ethyl)amino]-1,3-thiazol-4-yl}methyl)-N-(2- | 5.7 |
| | fluorophenyl)-2,2-dimethylpropanamide | |
| 170 | N-({2-[cyclopropyl(ethyl)amino]-1,3-thiazol-4-yl}methyl)-N-(3- | 3.9 |
| | fluorophenyl)-2,2-dimethylpropanamide | |
| 171 | N-({2-[cyclopropyl(ethyl)amino]-1,3-thiazol-4-yl}methyl)-2,2-dimethyl-N- | 6.5 |
| | (2-methylphenyl)propanamide | |
| 172 | N-({2-[cyclopropyl(ethyl)amino]-1,3-thiazol-4-yl}methyl)-2,2-dimethyl-N- | 4.3 |
| | (3-methylphenyl)propanamide | |
| 173 | N-({2-[cyclopropyl(ethyl)amino]-1,3-thiazol-4-yl}methyl)-2,2-dimethyl-N- | 3.8 |
| | (4-methylphenyl)propanamide | |
| 174 | N-({2-[cyclopropyl(ethyl)amino]-1,3-thiazol-4-yl}methyl)-N-(2- | 3.8 |
| | methoxyphenyl)-2,2-dimethylpropanamide | |
| 175 | N-({2-[cyclopropyl(ethyl)amino]-1,3-thiazol-4-yl}methyl)-N-(4- | 3.6 |
| | methoxyphenyl)-2,2-dimethylpropanamide | |
| 176 | N-({2-[cyclopropyl(ethyl)amino]-1,3-thiazol-4-yl}methyl)-2,2-dimethyl-N- | 7.1 |
| | (5,6,7,8-tetrahydronaphthalen-1-yl)propanamide | |
| 177 | N-{[2-(4-benzylpiperazin-1-yl)-1,3-thiazol-4-yl]methyl}-N-(4-fluorophenyl)- | 9.3 |
| | 2,2-dimethylpropanamide | |
| 178 | N-[(2-{4-[2-(dimethylamino)ethyl]piperazin-1-yl}-1,3-thiazol-4-yl)methyl]- | 19.7 |
| | N-(4-fluorophenyl)-2,2-dimethylpropanamide | |
| 179 | N-{[2-(diphenylamino)-1,3-thiazol-4-yl]methyl}-N-(4-fluorophenyl)-2,2- | 3.9 |
| | dimethylpropanamide | |
| 180 | N-(4-fluorophenyl)-2,2-dimethyl-N-[(2-phenyl-1,3-thiazol-4- | 7.9 |
| | yl)methyl]propanamide | |
| 181 | N-{[2-(1,4-dioxa-8-azaspiro[4.5]dec-8-yl)-1,3-thiazol-4-yl]methyl}-N-(4- | 2.7 |
| | fluorophenyl)-2,2-dimethylpropanamide | |
| 182 | N-(4-fluorophenyl)-N-{[2-(4-hydroxypiperidin-1-yl)-1,3-thiazol-4- | 8.1 |
| | yl]methyl}-2,2-dimethylpropanamide | |
| 183 | N-[(2-azepan-1-yl-1,3-thiazol-4-yl)methyl]-N-(4-fluorophenyl)-2,2- | 2.3 |
| | dimethylpropanamide | |
| 184 | N-[(2-azocan-1-yl-1,3-thiazol-4-yl)methyl]-N-(4-fluorophenyl)-2,2- | 1.6 |
| | dimethylpropanamide | |
| 185 | N-(4-fluorophenyl)-N-({2-[4-(hydroxymethyl)piperidin-1-yl]-1,3-thiazol-4- | 2.5 |
| | yl}methyl)-2,2-dimethylpropanamide | |
| 186 | N-(4-fluorophenyl)-N-{[2-(3-hydroxypiperidin-1-yl)-1,3-thiazol-4- | 5.6 |
| | yl]methyl}-2,2-dimethylpropanamide | |
| 187 | N-(4-fluorophenyl)-N-({2-[3-(hydroxymethyl)piperidin-1-yl]-1,3-thiazol-4- | 3 |
| | yl}methyl)-2,2-dimethylpropanamide | |
| 188 | N-{[2-(4-cyanopiperidin-1-yl)-1,3-thiazol-4-yl]methyl}-N-(4-fluorophenyl)- | 3.8 |
| | 2,2-dimethylpropanamide | |
| 189 | N-(4-fluorophenyl)-2,2-dimethyl-N-[(2-thiomorpholin-4-yl-1,3-thiazol-4- | 1.6 |
| | yl)methyl]propanamide | |
| 190 | N-{[2-(3,5-dimethylpiperidin-1-yl)-1,3-thiazol-4-yl]methyl}-N-(4- | 2.2 |
| | fluorophenyl)-2,2-dimethylpropanamide | |
| 191 | N-(4-fluorophenyl)-2,2-dimethyl-N-{[2-(octahydroisoquinolin-2(1H)-yl)-- | 2.7 |
| | 1,3-thiazol-4-yl]methyl}propanamide | |
| 192 | N-(4-fluorophenyl)-2,2-dimethyl-N-{[2-(4-methylpiperidin-1-yl)-1,3-thiazol- | 1.9 |
| | 4-yl]methyl}propanamide | |
| 193 | N-(4-fluorophenyl)-2,2-dimethyl-N-{[2-(4-phenylpiperidin-1-yl)-1,3-thiazol- | 3.1 |
| | 4-yl]methyl}propanamide | |
| 194 | N-(4-fluorophenyl)-2,2-dimethyl-N-[(2-piperidin-1-yl-1,3-thiazol-5- | 3.2 |
| | yl)methyl]propanamide | |
| 195 | N-[(6-chloropyridin-3-yl)methyl]-N-(4-fluorophenyl)-2,2- | 10.1 |
| | dimethylpropanamide | |
| 196 | N-(4-fluorophenyl)-2,2-dimethyl-N-[(6-morpholin-4-ylpyridin-3- | 13.8 |
| | yl)methyl]propanamide | |
| 197 | N-cyclohexyl-N-({2-[cyclopropyl(ethyl)amino]-1,3-thiazol-4- | 5.7 |
| | yl}methyl)acetamide | |
| 198 | N-cyclohexyl-N-({2-[cyclopropyl(ethyl)amino]-1,3-thiazol-4- | 3.3 |
| | yl}methyl)cyclohexanecarboxamide | |
| 199 | N-cyclohexyl-N-[(2-morpholin-4-yl-1,3-thiazol-4-yl)methyl]acetamide | 27.2 |
| 200 | N-cyclohexyl-N-[(2-morpholin-4-yl-1,3-thiazol-4- | 6.3 |
| | yl)methyl]cyclohexanecarboxamide | |
| 201 | N-cyclohexyl-N-[(2-morpholin-4-yl-1,3-thiazol-4-yl)methyl]-2- | 9.7 |
| | phenylacetamide | |
| 202 | tert-butyl 3-(5-{[(2,2-dimethylpropanoyl)(4- | 11.1 |
| | fluorophenyl)amino]methyl}pyridin-2-yl)benzoate | |
| 203 | 3-(5-{[(2,2-dimethylpropanoyl)(4-fluorophenyl)amino]methyl}pyridin-2- | 39 |
| | yl)benzoic acid | |
| 204 | N-cyclohexyl-2,2-dimethyl-N-[(2-morpholin-4-yl-1,3-thiazol-4- | 11.4 |
| | yl)methyl]propanamide | |
| 205 | N-cyclohexyl-N-({2-[cyclopropyl(ethyl)amino]-1,3-thiazol-4-yl}methyl)-2,2- | 2.1 |
| | dimethylpropanamide | |
| 206 | N-[(2-cyclohexyl-1,3-thiazol-4-yl)methyl]-N-(4-fluorophenyl)-2,2- | 6.1 |
| | dimethylpropanamide | |
| 207 | N-(4-fluorophenyl)-N-[(2-morpholin-4-yl-1,3-thiazol-4- | 23.9 |
| | yl)methyl]cyclopropanecarboxamide | |
| 208 | N-(4-fluorophenyl)-2-methyl-N-[(2-morpholin-4-yl-1,3-thiazol-4- | 22.2 |
| | yl)methyl]propanamide | |
| 209 | tert-butyl 4-(4-{[(2,2-dimethylpropanoyl)(4-fluorophenyl)amino]methyl}-- | 2.7 |
| | 1,3-thiazol-2-yl)piperidine-1-carboxylate | |
| 210 | N-({2-[cyclopropyl(ethyl)amino]-1,3-thiazol-4-yl}methyl)-N-(4- | 9.1 |
| | fluorophenyl)acetamide | |
| 211 | N-({2-[cyclopropyl(ethyl)amino]-1,3-thiazol-4-yl}methyl)-N-(4- | 2.4 |
| | fluorophenyl)cyclohexanecarboxamide | |
| 212 | N-(4-fluorophenyl)-2,2-dimethyl-N-[2-(2-morpholin-4-yl-1,3-thiazol-4- | 5.9 |
| | yl)ethyl]propanamide | |
| 213 | N-(4-fluorophenyl)-2,2-dimethyl-N-[3-(2-morpholin-4-yl-1,3-thiazol-4- | 3.8 |
| | yl)propyl]propanamide | |
| 214 | N-[(2-bromo-1,3-thiazol-4-yl)methyl]-N-(4-fluorophenyl)acetamide | 48.7 |
| 215 | N-[(6-chloropyridin-3-yl)methyl]-N-(4-fluorophenyl)acetamide | 21.4 |
| 216 | N-(4-fluorophenyl)-N-{[2-(4-methylpiperazin-1-yl)-1,3-thiazol-4- | 29.4 |
| | yl]methyl}acetamide | |
| 217 | N-(4-fluorophenyl)-N-{[2-(4-methyl-1,4-diazepan-1-yl)-1,3-thiazol-4- | 29.5 |
| | yl]methyl}acetamide | |
| 218 | N-(4-fluorophenyl)-N-[(2-morpholin-4-yl-1,3-thiazol-4-yl)methyl]acetamide | 23.4 |
| 219 | N-{[2-(diethylamino)-1,3-thiazol-4-yl]methyl}-N-(4-fluorophenyl)acetamide | 8.7 |
| 220 | N-{[2-(4-cyanopiperidin-1-yl)-1,3-thiazol-4-yl]methyl}-N-(4- | 16.6 |
| | fluorophenyl)acetamide | |
| 221 | N-(4-fluorophenyl)-N-({2-[(2-hydroxyethyl)(methyl)amino]-1,3-thiazol-4- | 37 |
| | yl}methyl)acetamide | |
| 222 | N-(4-fluorophenyl)-N-{[2-(4-hydroxypiperidin-1-yl)-1,3-thiazol-4- | 22.4 |
| | yl]methyl}acetamide | |
| 223 | N-(4-fluorophenyl)-N-{[2-(3-hydroxypiperidin-1-yl)-1,3-thiazol-4- | 31.4 |
| | yl]methyl}acetamide | |
| 224 | N-(4-fluorophenyl)-N-({2-[4-(hydroxymethyl)piperidin-1-yl]-1,3-thiazol-4- | 12.3 |
| | yl}methyl)acetamide | |
| 225 | N-(4-fluorophenyl)-N-({2-[3-(hydroxymethyl)piperidin-1-yl]-1,3-thiazol-4- | 16.4 |
| | yl}methyl)acetamide | |
| 226 | N-(4-fluorophenyl)-N-{[6-(4-methyl-1,4-diazepan-1-yl)pyridin-3- | 26.2 |
| | yl]methyl}acetamide | |
| 227 | N-(4-fluorophenyl)-N-[(6-morpholin-4-ylpyridin-3-yl)methyl]acetamide | 35 |
| 228 | N-{[6-(diethylamino)pyridin-3-yl]methyl}-N-(4-fluorophenyl)acetamide | 12.6 |
| 229 | N-{[6-(4-cyanopiperidin-1-yl)pyridin-3-yl]methyl}-N-(4- | 19.7 |
| | fluorophenyl)acetamide | |
| 230 | N-(4-fluorophenyl)-N-({6-[(2-hydroxyethyl)(methyl)amino]pyridin-3- | 45.3 |
| | yl}methyl)acetamide | |
| 231 | N-(4-fluorophenyl)-N-{[6-(4-hydroxypiperidin-1-yl)pyridin-3- | 49.7 |
| | yl]methyl}acetamide | |
| 232 | N-(4-fluorophenyl)-N-{[6-(3-hydroxypiperidin-1-yl)pyridin-3- | 30.2 |
| | yl]methyl}acetamide | |
| 233 | N-(4-fluorophenyl)-N-({6-[4-(hydroxymethyl)piperidin-1-yl]pyridin-3- | 22.2 |
| | yl}methyl)acetamide | |
| 234 | N-(4-fluorophenyl)-N-({6-[3-(hydroxymethyl)piperidin-1-yl]pyridin-3- | 13.9 |
| | yl}methyl)acetamide | |
| 235 | N-cyclopentyl-N-({6-[4-(hydroxymethyl)piperidin-1-yl]pyridin-3- | 36.4 |
| | yl}methyl)acetamide | |
| 236 | N-cyclohexyl-N-{[6-(4-methylpiperazin-1-yl)pyridin-3-yl]methyl}acetamide | 29.4 |
| 237 | N-cyclohexyl-N-({6-[4-(hydroxymethyl)piperidin-1-yl]pyridin-3- | 23.9 |
| | yl}methyl)acetamide | |
| 238 | N-(4-tert-butylcyclohexyl)-N-({2-[cyclopropyl(ethyl)amino]-1,3-thiazol-4- | 3.3 |
| | yl}methyl)acetamide | |
| 239 | 2,2-dimethylpropanamide | 3.4 |
| | N-{[2-(2,6-dimethylmorpholin-4-yl)-1,3-thiazol-4-yl]methyl}-N-(4- | |
| | fluorophenyl)- | |
| 240 | N-(4-fluorophenyl)-2,2-dimethyl-N-[(2-quinolin-3-yl-1,3-thiazol-4- | 10.1 |
| | yl)methyl]propanamide | |
| 241 | N-(4-fluorophenyl)-2,2-dimethyl-N-({2-[4-(trifluoromethyl)phenyl]-1,3- | 6.7 |
| | thiazol-4-yl}methyl)propanamide | |
| 242 | N-(4-fluorophenyl)-2,2-dimethyl-N-({2-[3-(trifluoromethyl)phenyl]-1,3- | 5.5 |
| | thiazol-4-yl}methyl)propanamide | |
| 243 | N-(4-fluorophenyl)-2,2-dimethyl-N-({2-[2-(trifluoromethyl)phenyl]- | 4.1 |
| | 1,3-thiazol-4-yl}methyl)propanamide | |
| 244 | N-(4-fluorophenyl)-N-{[2-(4-hydroxyphenyl)-1,3-thiazol-4-yl]methyl}-2,2- | 7.3 |
| | dimethylpropanamide | |
| 245 | N-(4-fluorophenyl)-N-{[2-(3-hydroxyphenyl)-1,3-thiazol-4-yl]methyl}-2,2- | 5.4 |
| | dimethylpropanamide | |
| 246 | N-(4-fluorophenyl)-N-{[2-(2-hydroxyphenyl)-1,3-thiazol-4-yl]methyl}-2,2- | 7.2 |
| | dimethylpropanamide | |
| 247 | N-(4-fluorophenyl)-N-{[2-(4-fluorophenyl)-1,3-thiazol-4-yl]methyl}-2,2- | 5 |
| | dimethylpropanamide | |
| 248 | N-(4-fluorophenyl)-N-{[2-(3-fluorophenyl)-1,3-thiazol-4-yl]methyl}-2,2- | 5.3 |
| | dimethylpropanamide | |
| 249 | N-(4-fluorophenyl)-N-{[2-(2-fluorophenyl)-1,3-thiazol-4-yl]methyl}-2,2- | 7.9 |
| | dimethylpropanamide | |
| 250 | N-(4-fluorophenyl)-N-{[6-(4-methylpiperazin-1-yl)pyridin-3- | 34.4 |
| | yl]methyl}tetrahydrofuran-2-carboxamide | |
| 251 | N-(4-fluorophenyl)-N-{[6-(4-methylpiperazin-1-yl)pyridin-3- | 45.6 |
| | yl]methyl}tetrahydrofuran-3-carboxamide | |
| 252 | N-[(6-chloropyridin-3-yl)methyl]-N-(4-fluorophenyl)tetrahydrofuran-2- | 13.4 |
| | carboxamide | |
| 253 | N-[(6-chloropyridin-3-yl)methyl]-N-(4-fluorophenyl)tetrahydrofuran-3- | 40.2 |
| | carboxamide | |
| 254 | tert-butyl (3S)-3-{[(6-chloropyridin-3-yl)methyl](4- | 2.9 |
| | fluorophenyl)carbamoyl}pyrrolidine-1-carboxylate | |
| 255 | tert-butyl (3R)-3-{[(6-chloropyridin-3-yl)methyl](4- | 2.8 |
| | fluorophenyl)carbamoyl}pyrrolidine-1-carboxylate | |
| 256 | tert-butyl (3S)-3-[(4-fluorophenyl){[6-(4-methylpiperazin-1-yl)pyridin-3- | 12.6 |
| | yl]methyl}carbamoyl]pyrrolidine-1-carboxylate | |
| 257 | tert-butyl (3R)-3-[(4-fluorophenyl){[6-(4-methylpiperazin-1-yl)pyridin-3- | 22.9 |
| | yl]methyl}carbamoyl]pyrrolidine-1-carboxylate | |
| 258 | (3S)—N-(4-fluorophenyl)-N-{[6-(4-methylpiperazin-1-yl)pyridin-3- | 57.7 |
| | yl]methyl}pyrrolidine-3-carboxamide | |
| 259 | (3R)—N-(4-fluorophenyl)-N-{[6-(4-methylpiperazin-1-yl)pyridin-3- | 59.5 |
| | yl]methyl}pyrrolidine-3-carboxamide | |
| 260 | (3S)-1-(2,2-dimethylpropanoyl)-N-(4-fluorophenyl)-N-{[6-(4- | 47.3 |
| | methylpiperazin-1-yl)pyridin-3-yl]methyl}pyrrolidine-3-carboxamide | |
| 261 | (3R)-1-(2,2-dimethylpropanoyl)-N-(4-fluorophenyl)-N-{[6-(4- | 55.6 |
| | methylpiperazin-1-yl)pyridin-3-yl]methyl}pyrrolidine-3-carboxamide | |

Variations, modifications, and other implementations of what is described herein will occur to those of ordinary skill in the art without departing from the spirit and the essential characteristics of the present teachings. It is not intended that the present invention be limited to the illustrated embodiments but rather by the following claims, and all changes that come within the meaning and range of equivalency of the claims are intended to be embraced therein.

What is claimed is:

1. A compound of formula (I) 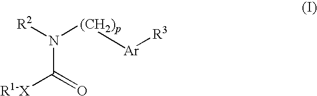
or a pharmaceutically acceptable salt, hydrate or ester thereof, wherein:
X is —NR_{c}—, —O—, or a covalent bond;
R¹ is C₁₋₁₀alkyl, a branched C₃₋₁₀alkyl group, a branched C₃₋₁₀alkenyl group, C₃₋₈cycloalkyl, or a 5-10 membered cycloheteroalkyl group, wherein:
the C₁₋₁₀alkyl, the branched C₃₋₁₀alkyl group and the branched C₃₋₁₀alkenyl group are optionally substituted with 1 to 3 substituents independently selected from a halogen and a 5-7 membered heteroaryl group, wherein the 5-7 membered heteroaryl group is optionally substituted with 1 to 3 substitutents independently selected from —C(O)OR_{c}, —Y—NR_{d}Rₑ, and —Y-phenyl; and
the 5-10 membered cycloheteroalkyl group is optionally substituted with 1 to 3 substituents independently selected from C₁₋₆alkyl, an oxo group, and —Y-phenyl, wherein the phenyl is optionally substituted with 1 to 3 substituted independently selected from halogen and C₁₋₆alkoxy;
the C₃₋₈cycloalkyl is optionally substituted with 1 to 3 substituents independently selected from C₁₋₆alkyl and —Y-phenyl, wherein —Y-phenyl is bonded to a carbon atom which is not bonded to X and is optionally substituted with 1 to 3 substituents independently selected from halogen and C₁₋₆alkoxy;
R² is C₃₋₆cycloalkyl, 5,6,7,8-tetrahydronaphthalen-1-yl, indole, benzyl, or phenyl, wherein
phenyl and benzyl are each s optionally substituted with 1 to 3 substituents independently selected from halogen, C₁₋₆alkyl, C₁₋₆alkoxy, C₁₋₆haloalkyl, C₁₋₆haloalkoxy, CN, —C(O)OR_{c}, and —NR_{d}Rₑ; and
the —C₃₋₆cycloalkyl is optionally substituted with 1 to 3 C₁₋₆alkyl groups;
Ar—R³ is selected from: 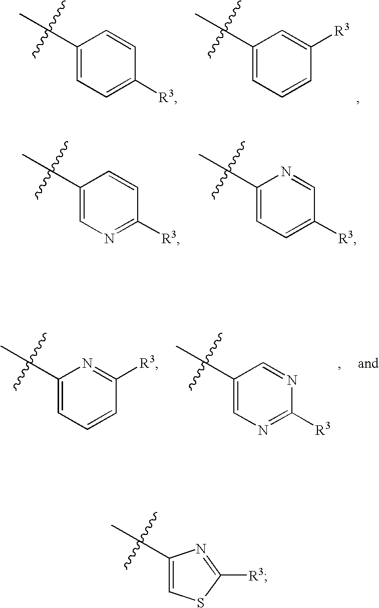
R³ is selected from halogen, C₁₋₁₀alkyl, C₁₋₁₀alkoxy, C₁₋₁₀haloalkyl, C₁₋₁₀haloalkoxy, C(O)R_{c}, piperidin-4-yl, C₃₋₆cycloalkyl, phenyl, 2-quinolin-3-yl, and —Y—NR_{f}R_{g}; wherein the phenyl is optionally substituted with 1 to 3 substituents independently selected from halogen, C₁₋₆haloalkyl, —OR_{c}, and —C(O)OR_{c}; and the C₁₋₁₀alkyl and the C₁₋₁₀alkoxy are optionally substituted with 1-3 substitutents selected from halogen, phenyl, and —OH;
Y, at each occurrence, is independently a divalent C₁₋₆alkyl group or a covalent bond;
R_{c}, R_{d} and Rₑ, at each occurrence, independently are independently H, C₁₋₆haloalkyl, or C₁₋₆alkyl; and
R_{f} and R_{g}, at each occurrence, are independently selected from the group consisting of —H, —C(O)R_{c}, C₂₋₆alkyl-OR_{c}, —C₂₋₆alkyl-NR_{d}Rₑ, C₁₋₁₀alkyl, C₃₋₆cycloalkyl, —Y-phenyl, —C(O)-phenyl, —Y-(5-7 membered cycloheteroalkyl), —Y-(5-7 membered heteroaryl), and —C₂₋₆alkyl-O—Y-(5-7 membered heteroaryl); or
alternatively, R_{f} and R_{g} taken together with the nitrogen atom to which they are bonded form a 5-7 membered cycloheteroalkyl group or a 5-7 membered heteroaryl group, the 5-7 membered cycloheteroalkyl group and the 5-7 membered heteroaryl group each containing up to two ring heteroatoms independently selected from nitrogen, oxygen, and sulfur, wherein
any sulfur atom in the ring is optionally substituted with 1 to 2 oxo groups,
one or more nitrogen atoms in the ring optionally are independently substituted with —C(O)R_{c}, —C₂₋₆alkyl-OR_{c}, —C₂₋₆alkyl-NR_{d}Rₑ, —Y—C(O)NR_{d}Rₑ, an —S(O)₂—C₁₋₆alkyl group, a —C₂₋₆alkyl-(5-7 membered cycloheteroalkyl) group, a C₁₋₆alkyl group, a —Y-(phenyl)_{q} group, a —C(O)O—C₁₋₆alkyl group, or a 5-7 membered heteroaryl group,
one or more carbon atoms in the ring optionally are independently substituted with CN, —C(O)—NR_{d}Rₑ, —Y—OR_{c}, —Y—NR_{d}Rₑ, a —Y-phenyl group, a —Y-(5-7 cycloheteroalkyl) group, a —Y-(5-9 membered heteroaryl) group, a C₁₋₆alkyl group, or a —Y—O-(5-7 membered heteroaryl) group; and wherein
each of the phenyl groups appearing anywhere in said R_{f} and R_{g} is optionally substituted with 1 to 3 substituents independently selected from halogen, C₁₋₆alkyl, C₁₋₆ haloalkyl, and C₁₋₆alkoxy, and
each of the 5-7 membered cycloheteroalkyl groups, the 5-7 membered heteroaryl groups, and the 5-9 membered heteroaryl groups appearing anywhere in said R_{f} and R_{g} is optionally substituted with 1 to 3 substituents independently selected from halogen and C₁₋₆alkyl;
p is 1, 2, 3, or 4;
n is 1, 2, or 3; and
q is 1, 2, or 3.

2. The compound of claim 1 or a pharmaceutically acceptable salt, hydrate or ester thereof, wherein X is a covalent bond.

3. The compound of claim 1 or a pharmaceutically acceptable salt, hydrate or ester thereof, wherein R¹ is methyl.

4. The compound of claim 1 or a pharmaceutically acceptable salt, hydrate or ester thereof, wherein R¹ is a branched lower alkyl group selected from: 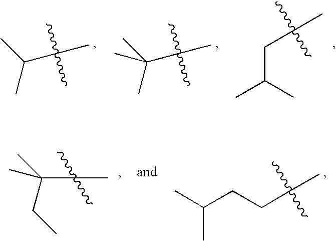
wherein each branched lower alkyl group is optionally substituted with 1 to 3 substituents independently selected from halogen and a 5-7 membered heteroaryl group, wherein the 5-7 membered heteroaryl group is optionally substituted with 1 to 3 substitutents independently selected from —C(O)OR_{c}, —Y—NR_{d}Rₑ, and —Y-phenyl, and Y, R_{c}, R_{d} and Rₑ are as defined in claim 1.

5. The compound of claim 1 or a pharmaceutically acceptable salt, hydrate or ester thereof, wherein R¹ is a tert-butyl group optionally substituted with a 5-7 membered heteroaryl group, the 5-7 membered heteroaryl group is optionally substituted with 1 to 3 substitutents independently selected from —C(O)OR^{c}, —Y—NR_{d}Rₑ, and —Y-phenyl, and Y, R_{c}, R_{d} and Rₑ are as defined in claim 1.

6. The compound of claim 1 or a pharmaceutically acceptable salt, hydrate or ester thereof, wherein R¹ is C₃₋₆cycloalkyl optionally substituted with —Y-phenyl bonded to a carbon atom which is not bonded to X, wherein the —Y-phenyl is optionally substituted with 1 to 3 substituents selected from halogen and C₁₋₆alkoxy.

7. The compound of claim 6 or a pharmaceutically acceptable salt, hydrate or ester thereof, wherein R¹ is cyclopropyl, cyclobutyl, cyclopentyl, or cyclohexyl.

8. The compound of claim 1 or a pharmaceutically acceptable salt, hydrate or ester thereof, wherein R¹ is a 5-10 membered cycloheteroalkyl group optionally substituted with C₁₋₆ alkyl or a benzyl group.

9. The compound of claim 8 or a pharmaceutically acceptable salt, hydrate or ester thereof, wherein R¹ is selected from the group consisting of tetrahydrofuran-2-yl, tetrahydrofuran-3-yl, tetrahydropyran-4-yl, piperidin-4-yl, pyrrolidin-2-yl, pyrrolidin-3-yl, and isoquinolin-3-yl.

10. The compound of claim 1 or a pharmaceutically acceptable salt, hydrate or ester thereof, wherein R² is phenyl optionally substituted with 1 to 2 substituents independently selected from halogen, C₁₋₆alkyl, —C(O)OR_{c} and —NR_{d}Rₑ, wherein R_{c}, R_{d} and Rₑ are as defined in claim 1.

11. The compound of claim 10 or a pharmaceutically acceptable salt, hydrate or ester thereof, wherein R² is selected from the group consisting of a 4-fluorophenyl group, a 4-chloro-phenyl group, a 4-methyl-phenyl group, a 3-methyl-phenyl group, a 2-methyl-phenyl group, a 4-fluoro-2-methyl-phenyl group, a 3,5-dimethylphenyl group, a 2,6-dimethylphenyl group, a 2-isopropyl-phenyl group, a 2-fluorophenyl group, a 3-fluorophenyl group, and a 3-isopropyl-phenyl group.

12. The compound of claim 1 or a pharmaceutically acceptable salt, hydrate or ester thereof, wherein R² is selected from cyclopropyl, cyclobutyl, and cyclopentyl.

13. The compound of claim 1 or a pharmaceutically acceptable salt, hydrate or ester thereof, wherein Ar—R³ is selected from: 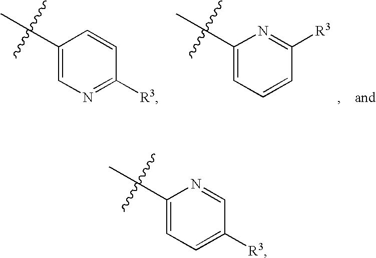
wherein R³ is as defined in claim 1.

14. The compound of claim 1 or a pharmaceutically acceptable salt, hydrate or ester thereof, wherein R³ is NR_{f}R_{g}, wherein R_{f} and R_{g} are as defined in claim 1.

15. The compound of claim 1 or a pharmaceutically acceptable salt, hydrate or ester thereof, wherein R³ is selected from NH₂, NH—C₁₋₆alkyl, N(C₁₋₆alkyl)₂, NH—C₃₋₆cycloalkyl, N(C₁₋₆alkyl)-C₃₋₆cycloalkyl, N(C₁₋₆alkyl)-C₂₋₆alkyl-OR_{c}, N(C₁₋₆alkyl)-Y-(5-7 membered cycloheteroalkyl), N(C₁₋₆alkyl)-phenyl, N(C₁₋₆alkyl)-Y-(5-7 membered heteroaryl), and N(C₁₋₆alkyl)-C₂₋₆alkyl-O—Y-(5-7 membered heteroaryl), wherein each of phenyl, the 5-7 membered cycloheteroalkyl, and the 5-7 heteroaryl is optionally substituted with 1 to 3 substituents independently selected from halogen and C₁₋₆alkyl, and Y and R_{c} are as defined in claim 1.

16. The compound of claim 1 or a pharmaceutically acceptable salt, hydrate or ester thereof, wherein R³ is diethylamino, diphenylamino or cyclopropyl(ethyl)amino.

17. The compound of claim 1 or a pharmaceutically acceptable salt, hydrate or ester thereof, wherein R³ is an optionally substituted 5-7 membered cycloheteroalkyl group or an optionally substituted 5-7 membered heteroaryl group as defined in claim 1.

18. The compound of claim 17 or a pharmaceutically acceptable salt, hydrate or ester thereof, wherein R³ is selected from a diazepanyl group, an imidazolyl group, a morpholinyl group, a piperidinyl group, a piperazinyl group, a pyridyl group, a pyrrolidyl group, and a thiomorpholinyl group, wherein each of these groups optionally includes a nitrogen ring atom substituted with —C(O)R_{c}, —C₂₋₆alkyl-OR_{c}, —C₂₋₆alkyl-NR_{d}Rₑ, —Y—C(O)NR_{d}Rₑ, —S(O)₂—C₁₋₆alkyl, —C₂₋₆alkyl-(5-7 membered cycloheteroalkyl), C₁₋₆alkyl, or 5-7 membered heteroaryl, a carbon ring atom substituted with —C(O)—NR_{d}Rₑ, —Y—NR_{d}Rₑ, —Y-phenyl, —Y-(5-7 cycloheteroalkyl), —Y-(5-9 membered heteroaryl), or —Y—O-(5-7 membered heteroaryl), and/or a sulfur ring atom substituted with 1 or 2 oxo groups, wherein each of the phenyl groups is optionally substituted with 1 to 3 substituents independently selected from halogen, C₁₋₆alkyl, C₁₋₆haloalkyl, and C₁₋₆alkoxy, and each of the 5-7 membered cycloheteroalkyl groups, the 5-7 membered heteroaryl groups, and the 5-9 membered heteroaryl groups is optionally substituted with 1 to 3 substituents independently selected from halogen and C₁₋₆alkyl, and wherein Y, R_{c}, R_{d} and Rₑ are as defined in claim 1.

19. The compound of claim 16 or a pharmaceutically acceptable salt, hydrate or ester thereof, wherein R³ is a 1-piperazinyl group having a nitrogen atom in the ring optionally substituted with —C(O)R_{c}, —C₂₋₆alkyl-OR_{c}, —C₂₋₆alkyl-NR_{d}Rₑ, —C₁₋₆alkyl-C(O)NR_{d}Rₑ, S(O)₂—C₁₋₆alkyl, —C₂₋₆alkyl-(5-7 membered cycloheteroalkyl), C₁₋₆alkyl, or a 5-7 membered heteroaryl group, wherein R_{c}, R_{d} and Rₑ are as defined in claim 1.

20. The compound of claim 16 or a pharmaceutically acceptable salt, hydrate or ester thereof, wherein R³ is an N-methyl 1-piperazinyl group.

21. The compound of claim 16 or a pharmaceutically acceptable salt, hydrate or ester thereof, wherein R³ is a 1-piperidinyl group having a carbon atom in the ring optionally substituted with —NR_{d}Rₑ, —C(O)—NR_{d}Rₑ, —Y—OR_{c}, a 5-7 cycloheteroalkyl group, a 5-9 membered heteroaryl group, or a —Y—O-(5-7 membered heteroaryl) group, wherein Y, R_{c}, R_{d} and Rₑ are as defined in claim 1.

22. The compound of claim 21 or a pharmaceutically acceptable salt, hydrate or ester thereof, wherein R³ is selected from a 1,4-dioxa-8-azaspiro[4.5]dec-8-yl group, a 4-(hydroxymethyl)piperidin-1-yl group, a 3-hydroxypiperidin-1-yl group, a 4-hydroxypiperidin-1-yl group, and a 3-(hydroxymethyl)piperidin-1-yl group.

23. A compound of claim 1 selected from:
N-(4-Fluorophenyl)-2,2-dimethyl-N-(2-piperidin-1-yl-thiazol-4-ylmethyl)-propionamide;
N-(6-Diethylamino-pyridin-3-ylmethyl)-N-(4-fluorophenyl)-2,2-dimethyl-propionamide;
N-(6-Diethylamino-pyridin-2-ylmethyl)-N-(4-fluorophenyl)-2,2-dimethyl-propionamide;
N-(4-Diethylaminobenzyl)-N-(4-fluorophenyl)-2,2-dimethyl-propionamide;
N-[3-(Cyclopropyl(ethyl)amino)-benzyl]-N-(4-fluorophenyl)-2,2-dimethyl-propionamide;
N-[6-(Cyclopropyl(ethyl)amino)-pyridin-3-ylmethyl]-N-(4-fluorophenyl)-2,2-dimethyl-propionamide;
N-(4-Fluorophenyl)-2,2-dimethyl-N-(2-piperidin-1-yl-pyrimidin-5-ylmethyl)-propionamide;
3-tert-Butyl-1-(4-fluorophenyl)-1-[6-(4-methyl-piperazin-1-yl)-pyridin-3-ylmethyl]-urea;
1-Benzyl-piperidine-4-carboxylic acid [6-(Cyclopropyl(ethyl)amino)-pyridin-3-ylmethyl]-(4-fluorophenyl)-amide;
4-Methyl-pentanoic acid [6-(Cyclopropyl(ethyl)amino)-pyridin-3-ylmethyl]-(4-fluorophenyl)-amide;
1,2,3,4-Tetrahydro-isoquinoline-3-carboxylic acid [6-(Cyclopropyl(ethyl)amino)-pyridin-3-ylmethyl]-(4-fluorophenyl)-amide;
2-Methyl-1,2,3,4-tetrahydro-isoquinoline-3-carboxylic acid [6-(Cyclopropyl(ethyl)amino)-pyridin-3-ylmethyl]-(4-fluorophenyl)-amide;
Tetrahydro-furan-2-carboxylic acid (4-fluorophenyl)-[6-(4-methyl-piperazin-1-yl)-pyridin-3-ylmethyl]-amide;
N-(4-Fluorophenyl)-N-[6-(4-methyl-piperazin-1-yl)-pyridin-3-ylmethyl)-2,2-dimethyl-propionamide;
N-(4-Fluorophenyl)-2,2-dimethyl-N-{6-[methyl-(2-pyridin-2-yl-ethyl)-amino]-pyridin-3-ylmethyl}-propionamide;
N-(4-Fluorophenyl)-2,2-dimethyl-N-{6-[methyl-(2-morpholin-4-yl-ethyl)-amino]-pyridin-3-ylmethyl}-propionamide;
N-(4-Fluoro-2-methyl-phenyl)-2,2-dimethyl-N-[6-(4-methyl-piperazin-1-yl)-pyridin-3-ylmethyl]-propionamide;
N-(4-Fluorophenyl)-2,2-dimethyl-N-[6-(4-methyl-[1,4]diazepan-1-yl)-pyridin-3-ylmethyl]-propionamide;
4,4,4-Trifluoro-N-(4-fluorophenyl)-3-methyl-N-{6-[methyl-(2-pyridin-2-yl-ethyl)-amino]-pyridin-3-ylmethyl}-butyramide;
N-(4-Fluorophenyl)-2,2-dimethyl-N-{6-[methyl-(2-pyridin-2-yl-ethyl)-amino]-pyridin-3-ylmethyl}-butyramide;
N-(4-Fluorophenyl)-2,2-dimethyl-N-[6-(4-pyridin-2-yl-piperazin-1-yl)-pyridin-3-ylmethyl]-propionamide;
N-(4-Fluorophenyl)-2,2-dimethyl-N-[6-(4-pyridin-2-yl-piperazin-1-yl)-pyridin-3-ylmethyl]-butyramide;
N-(4-Fluorophenyl)-2,2-dimethyl-N-{6-[methyl-(2-pyridin-3-yl-ethyl)-amino]-pyridin-3-ylmethyl}-propionamide;
N-(4-Fluorophenyl)-2,2-dimethyl-N-(6-{methyl-[2-(pyridin-2-yloxy)-ethyl]-amino}-pyridin-3-ylmethyl)-propionamide;
N-(2-Isopropyl-phenyl)-2,2-dimethyl-N-[6-(4-methyl-piperazin-1-yl)-pyridin-3-ylmethyl]-propionamide;
N-(6-Diethylamino-pyridin-3-ylmethyl)-N-(4-fluorophenyl)-2,2-dimethyl-butyramide;
N-(3,5-dimethylphenyl)-2,2-dimethyl-N-[6-(4-methyl-piperazin-1-yl)-pyridin-3-ylmethyl]-propionamide;
N-(4-Fluorophenyl)-2,2-dimethyl-N-(4-morpholin-4-yl-3,4,5,6-tetrahydro-2H-[1,2′]bipyridinyl-5′-ylmethyl)-propionamide;
N-(4-Fluorophenyl)-2,2-dimethyl-N-(4-pyrrolidin-1-yl-3,4,5,6-tetrahydro-2H-[1,2′]bipyridinyl-5′-ylmethyl)-propionamide;
N-(2-Isopropyl-phenyl)-2,2-dimethyl-N-[6-(4-methyl-piperazin-1-yl)-pyridin-3-ylmethyl]-butyramide;
N-(6-Diethylamino-pyridin-3-ylmethyl)-N-(4-fluoro-2-methyl-phenyl)-2,2-dimethyl-propionamide;
N-(6-Diethylamino-pyridin-3-ylmethyl)-N-(2-isopropyl-phenyl)-2,2-dimethyl-propionamide;
N-(4-Fluorophenyl)-N-{6-[(2-hydroxy-ethyl)-methyl-amino]-pyridin-3-ylmethyl}-2,2-dimethyl-propionamide;
N-(4-Fluorophenyl)-2,2-dimethyl-N-(6-{methyl-[2-(5-methyl-pyridin-2-yloxy)-ethyl]-amino}-pyridin-3-ylmethyl)-propionamide;
N-(4-Fluorophenyl)-2,2-dimethyl-N-(6-{methyl-[2-(6-methyl-pyridazin-3-yloxy)-ethyl]-amino}-pyridin-3-ylmethyl)-propionamide;
N-[6-(Ethyl-pyridin-4-ylmethyl-amino)-pyridin-3-ylmethyl]-N-(4-fluorophenyl)-2,2-dimethyl-propionamide;
N-(4-Fluorophenyl)-2,2-dimethyl-N-(6-pyrrolidin-1-yl-pyridin-3-ylmethyl)-propionamide;
N-(4-Fluorophenyl)-N-[6-(4-methanesulfonyl-piperazin-1-yl)-pyridin-3-ylmethyl]-2,2-dimethyl-butyramide;
N-[6-(4-Acetyl-piperazin-1-yl)-pyridin-3-ylmethyl]-N-(4-fluorophenyl)-2,2-dimethyl-propionamide;
1-{1-[(6-Diethylamino-pyridin-3-ylmethyl)-(4-fluorophenyl)-carbamoyl]-1-methyl-ethyl}-1H-[1,2,3]triazole-4-carboxylic acid ethyl ester;
N-(4-Fluorophenyl)-2,2-dimethyl-N-(6-{methyl-[2-(pyridin-3-ylmethoxy)-ethyl]-amino}-pyridin-3-ylmethyl)-propionamide;
N-(4-Fluorophenyl)-N-[6-(2-hydroxymethyl-pyrrolidin-1-yl)-pyridin-3-ylmethyl]-2,2-dimethyl-propionamide;
N-(4-Fluorophenyl)-2,2-dimethyl-N-{6-[2-(pyridin-2-yloxymethyl)-pyrrolidin-1-yl]-pyridin-3-ylmethyl}-propionamide;
N-(4-Fluorophenyl)-2,2-dimethyl-N-{6-[3-(pyridin-2-yloxy)-pyrrolidin-1-yl]-pyridin-3-ylmethyl}-propionamide;
N-(4-Fluorophenyl)-N-[6-(3-hydroxy-pyrrolidin-1-yl)-pyridin-3-ylmethyl]-2,2-dimethyl-propionamide;
N-[6-(3-Diethylamino-pyrrolidin-1-yl)-pyridin-3-ylmethyl]-N-(4-fluorophenyl)-2,2-dimethyl-propionamide;
N-[4-(1H-Benzoimidazol-2-yl)-3,4,5,6-tetrahydro-2H-[1,2′]-bipyridinyl-5′-ylmethyl]-N-(4-fluorophenyl)-2,2-dimethyl-propionamide;
N-(6-Diethylamino-pyridin-3-ylmethyl)-2-(4-dimethylaminomethyl-[1,2,3]triazol-1-yl)-N-(4-fluorophenyl)-isobutyramide;
N-(4-Fluorophenyl)-2,2-dimethyl-N-{6-[3-(pyridin-2-yloxy)-pyrrolidin-1-yl]-pyridin-3-ylmethyl}-propionamide;
N-(4-Fluorophenyl)-2,2-dimethyl-N-[6-(3-methylamino-pyrrolidin-1-yl)-pyridin-3-ylmethyl]-propionamide;
N-(6-Diethylaminomethyl-pyridin-3-ylmethyl)-N-(4-fluorophenyl)-2,2-dimethyl-propionamide;
N-{6-[Ethyl-(2-pyridin-3-yl-ethyl)-amino]-pyridin-3-ylmethyl}-N-(4-fluorophenyl)-2,2-dimethyl-propionamide;
N-(4-Fluorophenyl)-2,2-dimethyl-N-[6-(2-pyridin-3-yl-ethylamino)-pyridin-3-ylmethyl]-propionamide;
N-(4-Fluorophenyl)-N-[6-(3-imidazol-1-yl-propylamino)-pyridin-3-ylmethyl]-2,2-dimethyl-propionamide;
N-(4-Fluorophenyl)-2,2-dimethyl-N-{6-[propionyl-(2-pyridin-3-yl-ethyl)-amino]-pyridin-3-ylmethyl}-propionamide;
N-(6-Dimethylamino-pyridin-3-ylmethyl)-N-(4-fluorophenyl)-2,2-dimethyl-propionamide;
2-(4-Benzyl-[1,2,3]triazol-1-yl)-N-(6-diethylamino-pyridin-3-ylmethyl)-N-(4-fluorophenyl)-isobutyramide;
N-(2-Diethylamino-thiazol-4-ylmethyl)-N-(4-fluorophenyl)-2,2-dimethyl-propionamide;
N-(4-Fluorophenyl)-2,2-dimethyl-N-[2-(methyl-phenyl-amino)-thiazol-4-ylmethyl]-propionamide;
N-(4-Fluorophenyl)-N-[6-(furan-2-ylmethyl-methyl-amino)-pyridin-3-ylmethyl]-2,2-dimethyl-propionamide;
N-(6-tert-Butylamino-pyridin-3-ylmethyl)-N-(4-fluorophenyl)-2,2-dimethyl-propionamide;
N-(4-Fluorophenyl)-N-[2-(4-methoxy-phenoxymethyl)-thiazol-4-ylmethyl]-2,2-dimethyl-propionamide;
N-(4-Fluorophenyl)-2,2-dimethyl-N-(2-morpholin-4-yl-thiazol-4-ylmethyl)-propionamide;
N-(4-Fluorophenyl)-2,2-dimethyl-N-(3,4,5,6-tetrahydro-2H-[1,2′]bipyridinyl-5′-ylmethyl)-propionamide;
N-(3-Isopropyl-phenyl)-2,2-dimethyl-N-(2-piperidin-1-yl-thiazol-4-ylmethyl)-propionamide;
N-(4-Fluorophenyl)-2,2-dimethyl-N-[6-(4-thiazol-2-yl-piperazin-1-yl)-pyridin-3-ylmethyl]-propionamide;
N-(4-Fluorophenyl)-2,2-dimethyl-N-(2-morpholin-4-yl-thiazol-4-ylmethyl)-butyramide;
N-[6-(Cyclopropyl-methyl-amino)-pyridin-3-ylmethyl]-N-(4-fluorophenyl)-2,2-dimethyl-propionamide;
N-(4-Fluorophenyl)-2,2-dimethyl-N-(2-pyridin-4-yl-thiazol-4-ylmethyl)-propionamide;
N-(4-Fluorophenyl)-2,2-dimethyl-N-(2-pyrrolidin-1-yl-thiazol-4-ylmethyl)-propionamide;
N-(6-Cyclopropylamino-pyridin-3-ylmethyl)-N-(4-fluorophenyl)-2,2-dimethyl-propionamide;
N-(4-Fluorophenyl)-N-{2-[(4-fluorophenyl)-methyl-amino]-thiazol-4-ylmethyl}-2,2-dimethyl-propionamide;
N-(4-Fluorophenyl)-N-{2-[(4-methoxy-phenyl)-methyl-amino]-thiazol-4-ylmethyl}-2,2-dimethyl-propionamide;
N-(4-Dimethylamino-phenyl)-N-{2-[(4-fluorophenyl)-methyl-amino]-thiazol-4-ylmethyl}-2,2-dimethyl-propionamide;
N-{2-[(4-Fluorophenyl)-methyl-amino]-thiazol-4-ylmethyl}-N-(5-fluoro-pyridin-2-yl)-2,2-dimethyl-propionamide;
N-[2-(Cyclopropyl(ethyl)amino)-thiazol-4-ylmethyl]-N-(4-fluorophenyl)-2,2-dimethyl-propionamide;
N-(2-Diethylamino-pyrimidin-5-ylmethyl)-N-(4-fluorophenyl)-2,2-dimethyl-propionamide;
N-(4-Dimethylamino-phenyl)-2,2-dimethyl-N-(2-piperidin-1-yl-pyrimidin-5-ylmethyl)-propionamide;
N-(4-Dimethylamino-phenyl)-2,2-dimethyl-N-(2-piperidin-1-yl-thiazol-4-ylmethyl)-propionamide;
N-[4-(Cyclopropyl(ethyl)amino)-benzyl]-N-(4-fluorophenyl)-2,2-dimethyl-propionamide;
N-(4-Diethylamino-phenyl)-N-{2-[(4-fluorophenyl)-methyl-amino]-thiazol-4-ylmethyl}-2,2-dimethyl-propionamide;
N-(4-Dimethylamino-phenyl)-2,2-dimethyl-N-{2-[methyl-(4-trifluoromethyl-phenyl)-amino]-thiazol-4-ylmethyl}-propionamide;
N-(4-Diethylaminobenzyl)-N-(4-dimethylamino-phenyl)-2,2-dimethyl-butyramide;
4-Methyl-pentanoic acid (4-Diethylaminobenzyl)-(4-dimethylamino-phenyl)-amide;
N-[6-(Cyclopropyl(ethyl)amino)-pyridin-2-ylmethyl]-N-(4-fluorophenyl)-2,2-dimethyl-propionamide;
N-(4-Fluorophenyl)-2,2-dimethyl-N-[2-(methyl-pyridin-2-yl-amino)-thiazol-4-ylmethyl]-propionamide;
4-Methyl-pent-2-enoic acid (4-Diethylaminobenzyl)-(4-dimethylamino-phenyl)-amide;
4-Methyl-pentanoic acid (2-diethylamino-thiazol-4-ylmethyl)-(4-dimethylamino-phenyl)-amide;
N-(2-Diethylamino-thiazol-4-ylmethyl)-N-(4-dimethylamino-phenyl)-2,2-dimethyl-butyramide;
N-[3-(Cyclopropyl(ethyl)amino)-benzyl]-N-(4-diethylamino-phenyl)-2,2-dimethyl-propionamide;
4-Methyl-pentanoic acid (4-amino-phenyl)-(4-Diethylaminobenzyl)-amide;
4-Methyl-pentanoic acid (4-Diethylaminobenzyl)-(4-methylamino-phenyl)-amide;
N-[6-(Cyclopropyl(ethyl)amino)-pyridin-3-ylmethyl]-N-(4-dimethylamino-phenyl)-2,2-dimethyl-propionamide;
(4-Fluorophenyl)-(2-pyridin-4-yl-thiazol-4-ylmethyl)-carbamic acid tert-butyl ester;
Cyclopropanecarboxylic acid (4-fluorophenyl)-[6-(4-methyl-piperazin-1-yl)-pyridin-3-ylmethyl]-amide;
Cyclohexanecarboxylic acid (4-fluorophenyl)-[6-(4-methyl-piperazin-1-yl)-pyridin-3-ylmethyl]-amide;
Cyclohexanecarboxylic acid (4-fluorophenyl)-[6-(4-pyridin-2-yl-piperazin-1-yl)-pyridin-3-ylmethyl]-amide;
Cyclopropanecarboxylic acid (4-fluorophenyl)-(2-piperidin-1-yl-thiazol-4-ylmethyl)-amide;
Cyclohexanecarboxylic acid (4-fluorophenyl)-(2-morpholin-4-yl-thiazol-4-ylmethyl)-amide;
Tetrahydro-furan-2-carboxylic acid (4-fluorophenyl)-[6-(4-pyridin-2-yl-piperazin-1-yl)-pyridin-3-ylmethyl]-amide;
N-(4-fluorophenyl)-N-{[6-(4-methylpiperazin-1-yl)pyridin-3-yl]methyl}tetrahydrofuran-2-carboxamide;
Tetrahydro-furan-3-carboxylic acid (4-fluorophenyl)-[6-(4-pyridin-2-yl-piperazin-1-yl)-pyridin-3-ylmethyl]-amide;
Tetrahydro-furan-3-carboxylic acid (6-diethylamino-pyridin-3-ylmethyl)-(4-fluorophenyl)-amide;
Tetrahydro-pyran-4-carboxylic acid (6-diethylamino-pyridin-3-ylmethyl)-(4-fluorophenyl)-amide;
1-Methyl-pyrrolidine-2-carboxylic acid (4-fluorophenyl)-{2-[(4-fluorophenyl)-methyl-amino]-thiazol-4-ylmethyl}-amide;
1-Benzyl-pyrrolidine-2-carboxylic acid [6-(Cyclopropyl(ethyl)amino)-pyridin-3-ylmethyl]-(4-fluorophenyl)-amide;
N-{[6-(diethylamino)pyridin-3-yl]methyl}-N-(4-fluorophenyl)-L-prolinamide;
N-{4-[cyclopropyl(ethyl)amino]benzyl}-N-(4-methoxyphenyl)-L-prolinamide;
N-{3-[cyclopropyl(ethyl)amino]benzyl}-N-(4-fluorophenyl)-L-prolinamide;
N-(4-chlorophenyl)-N-{[6-(diethylamino)pyridin-2-yl]methyl}-L-prolinamide;
N-(4-fluoro-2-methylphenyl)-N-[(2-piperidin-1-ylpyrimidin-5-yl)methyl]-L-prolinamide;
N-({2-[cyclopropyl(ethyl)amino]-1,3-thiazol-4-yl}methyl)-N-(4-fluorophenyl)-D-prolinamide;
N-({2-[cyclopropyl(ethyl)amino]-1,3-thiazol-4-yl}methyl)-N-(4-fluorophenyl)-5-oxo-L-prolinamide;
N-(4-fluorophenyl)-N-{[6-(4-methylpiperazin-1-yl)pyridin-3-yl]methyl}cyclohexanecarboxamide;
N′-(tert-butyl)-N-(4-fluorophenyl)-N-{[6-(4-methylpiperazin-1-yl)pyridin-3-yl]methyl}urea;
4,4,4-trifluoro-N-(4-fluorophenyl)-3-methyl-N-({6-[methyl(2-pyridin-2-ylethyl)amino]pyridin-3-yl}methyl)butanamide;
N-(4-fluorophenyl)-N-{[6-(4-pyridin-2-ylpiperazin-1-yl)pyridin-3-yl]methyl}cyclohexanecarboxamide;
N-{[6-(diethylamino)pyridin-3-yl]methyl}-N-(4-fluorophenyl)-2-(2-thienyl)acetamide;
N-(4-fluorophenyl)-N-({6-[(2-hydroxyethyl)(methyl)amino]pyridin-3-yl}methyl)-2,2-dimethylpropanamide;
N-(4-fluorophenyl)-2,2-dimethyl-N-{[6-(methyl{2-[(5-methylpyridin-2-yl)oxy]ethyl}amino)pyridin-3-yl]methyl}propanamide;
N-(4-fluorophenyl)-2,2-dimethyl-N-[(6-pyrrolidin-1-ylpyridin-3-yl)methyl]propanamide;
N-({6-[3-(diethylamino)pyrrolidin-1-yl}pyridin-3-yl]methyl)-N-(4-fluorophenyl)-2,2-dimethylpropanamide;
N-(4-fluorophenyl)-2,2-dimethyl-N-[(2-piperidin-1-yl-1,3-thiazol-4-yl)methyl]propanamide;
N-(4-fluorophenyl)-2,2-dimethyl-N-({6-[propionyl(2-pyridin-3-ylethyl)amino]pyridin-3-yl}methyl)propanamide;
N-(4-fluorophenyl)-2,2-dimethyl-N-[(2-morpholin-4-yl-1,3-thiazol-4-yl)methyl]propanamide;
N-(4-fluorophenyl)-N-[(2-morpholin-4-yl-1,3-thiazol-4-yl)methyl]cyclohexanecarboxamide;
N-(4-fluorophenyl)-2,2-dimethyl-N-[(2-morpholin-4-yl-1,3-thiazol-4-yl)methyl]butanamide;
N-({6-[cyclopropyl(ethyl)amino]pyridin-3-yl}methyl)-N-(4-fluorophenyl)-2,2-dimethylpropanamide;
N-(4-fluorophenyl)-2,2-dimethyl-N-[(2-pyrrolidin-1-yl-1,3-thiazol-4-yl)methyl]propanamide;
N-(4-fluorophenyl)-N-({2-[(4-fluorophenyl)(methyl)amino]-1,3-thiazol-4-yl}methyl)-2,2-dimethylpropanamide;
N-(4-fluorophenyl)-N-({2-[(4-methoxyphenyl)(methyl)amino]-1,3-thiazol-4-yl}methyl)-2,2-dimethylpropanamide;
N-({2-[cyclopropyl(ethyl)amino]-1,3-thiazol-4-yl}methyl)-N-(4-fluorophenyl)-2,2-dimethylpropanamide;
N-[(2-bromo-1,3-thiazol-4-yl)methyl]-N-(4-fluorophenyl)-2,2-dimethylpropanamide;
N-{[2-(3,5-dimethylpiperazin-1-yl)-1,3-thiazol-4-yl]methyl}-N-(4-fluorophenyl)-2,2-dimethylpropanamide;
tert-butyl 4-(4-{[(2,2-dimethylpropanoyl)(4-fluorophenyl)amino]methyl}-1,3-thiazol-2-yl)piperazine-1-carboxylate;
N-(4-fluorophenyl)-2,2-dimethyl-N-{[2-(4-phenylpiperazin-1-yl)-1,3-thiazol-4-yl]methyl}propanamide;
N-(4-fluorophenyl)-N-{[2-(4-isopropylpiperazin-1-yl)-1,3-thiazol-4-yl]methyl}-2,2-dimethylpropanamideN-(4-fluorophenyl)-N-{[2-(4-isopropylpiperazin-1-yl)-1,3-thiazol-4-yl]methyl}-2,2-dimethylpropanamide;
N-(4-fluorophenyl)-2,2-dimethyl-N-{[2-(4-pyridin-2-ylpiperazin-1-yl)-1,3-thiazol-4-yl]methyl}propanamide;
N-(4-fluorophenyl)-2,2-dimethyl-N-{[2-(4-methylpiperazin-1-yl)-1,3-thiazol-4-yl]methyl}propanamide;
N-(4-fluorophenyl)-2,2-dimethyl-N-{[2-(4-pyrimidin-2-ylpiperazin-1-yl)-1,3-thiazol-4-yl]methyl}propanamide;
N-(4-fluorophenyl)-2,2-dimethyl-N-[(2-piperazin-1-yl-1,3-thiazol-4-yl)methyl]propanamide;
N-[(2-{[2-(dimethylamino)ethyl]amino}-1,3-thiazol-4-yl)methyl]-N-(4-fluorophenyl)-2,2-dimethylpropanamide;
N-[(2-{[3-(dimethylamino)propyl]amino}-1,3-thiazol-4-yl)methyl]-N-(4-fluorophenyl)-2,2-dimethylpropanamide;
N-{(2-[4-(dimethylamino)butyl]amino}-1,3-thiazol-4-yl)methyl]-N-(4-fluorophenyl)-2,2-dimethylpropanamide;
N-{[2-(cyclopentylamino)-1,3-thiazol-4-yl]methyl}-N-(4-fluorophenyl)-2,2-dimethylpropanamide;
N-{[2-(cyclohexylamino)-1,3-thiazol-4-yl]methyl}-N-(4-fluorophenyl)-2,2-dimethylpropanamide;
N-(4-fluorophenyl)-N-({2-[(4-fluorophenyl)amino]-1,3-thiazol-4-yl}methyl)-2,2-dimethylpropanamide;
N-(4-fluorophenyl)-N-({2-[(2-fluorophenyl)amino]-1,3-thiazol-4-yl}methyl)-2,2-dimethylpropanamide;
N-(4-fluorophenyl)-2,2-dimethyl-N-{[2-(methylamino)-1,3-thiazol-4-yl]methyl}propanamide;
N-(4-fluorophenyl)-N-{[2-(isobutylamino)-1,3-thiazol-4-yl]methyl}-2,2-dimethylpropanamide;
N-({2-[(1S,4S)-2,5-diazabicyclo[2.2.1]hept-2-yl]-1,3-thiazol-4-yl}methyl)-N-(4-fluorophenyl)-2,2-dimethylpropanamide;
N-(4-fluorophenyl)-2,2-dimethyl-N-{[2-(propylamino)-1,3-thiazol-4-yl]methyl}propanamide;
N-{[2-(dimethylamino)-1,3-thiazol-4-yl]methyl}-N-(4-fluorophenyl)-2,2-dimethylpropanamide;
N-(4-fluorophenyl)-2,2-dimethyl-N-({2-[4-(2-phenylethyl)piperazin-1-yl]-1,3-thiazol-4-yl}methyl)propanamide;
N-[(2-{4-[3-(dimethylamino)propyl]piperazin-1-yl}-1,3-thiazol-4-yl}methyl)-N-(4-fluorophenyl)-2,2-dimethylpropanamide;
N-({2-[4-(diphenylmethyl)piperazin-1-yl]-1,3-thiazol-4-yl}methyl)-N-(4-fluorophenyl)-2,2-dimethylpropanamide;
N-({2-[cyclopropyl(ethyl)amino]-1,3-thiazol-4-yl}methyl)-2,2-dimethyl-N-phenylpropanamide;
N-({2-[cyclopropyl(ethyl)amino]-1,3-thiazol-4-yl}methyl)-N-(2-fluorophenyl)-2,2-dimethylpropanamide;
N-({2-[cyclopropyl(ethyl)amino]-1,3-thiazol-4-yl}methyl)-N-(3-fluorophenyl)-2,2-dimethylpropanamide;
N-({2-[cyclopropyl(ethyl)amino]-1,3-thiazol-4-yl}methyl)-2,2-dimethyl-N-(2-methylphenyl)propanamide;
N-({2-[cyclopropyl(ethyl)amino]-1,3-thiazol-4-yl}methyl)-2,2-dimethyl-N-(3-methylphenyl)propanamide;
N-({2-[cyclopropyl(ethyl)amino]-1,3-thiazol-4-yl}methyl)-2,2-dimethyl-N-(4-methylphenyl)propanamide;
N-({2-[cyclopropyl(ethyl)amino]-1,3-thiazol-4-yl}methyl)-N-(2-methoxyphenyl)-2,2-dimethylpropanamide;
N-({2-[cyclopropyl(ethyl)amino]-1,3-thiazol-4-yl}methyl)-N-(4-methoxyphenyl)-2,2-dimethylpropanamide;
N-({2-[cyclopropyl(ethyl)amino]-1,3-thiazol-4-}methyl)-2,2-dimethyl-N-(5,6,7,8-tetrahydronaphthalen-1-yl)propanamide;
N-{[2-(4-benzylpiperazin-1-yl)-1,3-thiazol-4-yl]methyl}-N-(4-fluorophenyl)-2,2-dimethylpropanamide;
N-[(2-{4-[2-(dimethylamino)ethyl]piperazin-1-yl}-1,3-thiazol-4-yl)methyl]-N-(4-fluorophenyl)-2,2-dimethylpropanamide;
N-{[2-(diphenylamino)-1,3-thiazol-4-yl]methyl}-N-(4-fluorophenyl)-2,2-dimethylpropanamide;
N-(4-fluorophenyl)-2,2-dimethyl-N-[(2-phenyl-1,3-thiazol-4-yl)methyl]propanamide;
N-{[2-(1,4-dioxa-8-azaspiro[4.5]dec-8-yl)-1,3-thiazol-4-yl]methyl}-N-(4-fluorophenyl)-2,2-dimethylpropanamide;
N-(4-fluorophenyl)-N-{[2-(4-hydroxypiperidin-1-yl)-1,3-thiazol-4-yl]methyl}-2,2-dimethylpropanamide;
N-[(2-azepan-1-yl-1,3-thiazol-4-yl)methyl]-N-(4-fluorophenyl)-2,2-dimethylpropanamide;
N-[(2-azocan-1-yl-1,3-thiazol-4-yl)methyl]-N-(4-fluorophenyl)-2,2-dimethylpropanamide;
N-(4-fluorophenyl)-N-({2-[4-(hydroxymethyl)piperidin-1-yl]-1,3-thiazol-4-yl}methyl)-2,2-dimethylpropanamide;
N-(4-fluorophenyl)-N-{[2-(3-hydroxypiperidin-1-yl)-1,3-thiazol-4-yl]methyl}-2,2-dimethylpropanamide;
N-(4-fluorophenyl)-N-({2-[3-(hydroxymethyl)piperidin-1-yl]-1,3-thiazol-4-yl}methyl)-2,2-dimethylpropanamide;
N-{[2-(4-cyanopiperidin-1-yl)-1,3-thiazol-4-yl]methyl}-N-(4-fluorophenyl)-2,2-dimethylpropanamide;
N-(4-fluorophenyl)-2,2-dimethyl-N-[(2-thiomorpholin-4-yl-1,3-thiazol-4-yl)methyl]propanamide;
N-{[2-(3,5-dimethylpiperidin-1-yl)-1,3-thiazol-4-yl]methyl}-N-(4-fluorophenyl)-2,2-dimethylpropanamide;
N-(4-fluorophenyl)-2,2-dimethyl-N-{[2-(octahydroisoquinolin-2(1H)-yl)-1,3-thiazol-4-yl]methyl}propanamide;
N-(4-fluorophenyl)-2,2-dimethyl-N-{[2-(4-methylpiperidin-1-yl)-1,3-thiazol-4-yl]methyl}propanamide;
N-(4-fluorophenyl)-2,2-dimethyl-N-{[2-(4-phenylpiperidin-1-yl)-1,3-thiazol-4-yl]methyl}propanamide;
N-(4-fluorophenyl)-2,2-dimethyl-N-[(2-piperidin-1-yl-1,3-thiazol-5-yl)methyl]propanamide;
N-[(6-chloropyridin-3-yl)methyl]-N-(4-fluorophenyl)-2,2-dimethylpropanamide;
N-(4-fluorophenyl)-2,2-dimethyl-N-[(6-morpholin-4-ylpyridin-3-yl)methyl]propanamide;
N-cyclohexyl-N-({2-[cyclopropyl(ethyl)amino]-1,3-thiazol-4-yl}methyl)acetamide;
N-cyclohexyl-N-({2-[cyclopropyl(ethyl)amino]-1,3-thiazol-4-yl}methyl)cyclohexanecarboxamide;
N-cyclohexyl-N-[(2-morpholin-4-yl-1,3-thiazol-4-yl)methyl]acetamide;
N-cyclohexyl-N-[(2-morpholin-4-yl-1,3-thiazol-4-yl)methyl]cyclohexanecarboxamide;
N-cyclohexyl-N-[(2-morpholin-4-yl-1,3-thiazol-4-yl)methyl]-2-phenylacetamide;
tert-butyl 3-(5-{[(2,2-dimethylpropanoyl)(4-fluorophenyl)amino]methyl}pyridin-2-yl)benzoate;
3-(5-{[(2,2-dimethylpropanoyl)(4-fluorophenyl)amino]methyl}pyridin-2-yl)benzoic acid;
N-cyclohexyl-2,2-dimethyl-N-[(2-morpholin-4-yl-1,3-thiazol-4-yl)methyl]propanamide;
N-cyclohexyl-N-({2-[cyclopropyl(ethyl)amino]-1,3-thiazol-4-yl}methyl)-2,2-dimethylpropanamide;
N-[(2-cyclohexyl-1,3-thiazol-4-yl)methyl]-N-(4-fluorophenyl)-2,2-dimethylpropanamide;
N-(4-fluorophenyl)-N-[(2-morpholin-4-yl-1,3-thiazol-4-yl)methyl]cyclopropanecarboxamide;
N-(4-fluorophenyl)-2-methyl-N-[(2-morpholin-4-yl-1,3-thiazol-4-yl)methyl]propanamide;
tert-butyl 4-(4-{[(2,2-dimethylpropanoyl)(4-fluorophenyl)amino]methyl}-1,3-thiazol-2-yl)piperidine-1-carboxylate;
N-({2-[cyclopropyl(ethyl)amino]-1,3-thiazol-4-yl}methyl)-N-(4-fluorophenyl)acetamide;
N-({2-[cyclopropyl(ethyl)amino]-1,3-thiazol-4-yl}methyl)-N-(4-fluorophenyl)cyclohexanecarboxamide;
N-(4-fluorophenyl)-2,2-dimethyl-N-[2-(2-morpholin-4-yl-1,3-thiazol-4-yl)ethyl]propanamide;
N-(4-fluorophenyl)-2,2-dimethyl-N-[3-(2-morpholin-4-yl-1,3-thiazol-4-yl)propyl]propanamide;
N-[(2-bromo-1,3-thiazol-4-yl)methyl]-N-(4-fluorophenyl)acetamide;
N-[(6-chloropyridin-3-yl)methyl]-N-(4-fluorophenyl)acetamide;
N-(4-fluorophenyl)-N-{[2-(4-methylpiperazin-1-yl)-1,3-thiazol-4-yl]methyl}acetamide;
N-(4-fluorophenyl)-N-{[2-(4-methyl-1,4-diazepan-1-yl)-1,3-thiazol-4-yl]methyl}acetamide;
N-(4-fluorophenyl)-N-[(2-morpholin-4-yl-1,3-thiazol-4-yl)methyl]acetamide;
N-{[2-(diethylamino)-1,3-thiazol-4-yl]methyl}-N-(4-fluorophenyl)acetamide;
N-{[2-(4-cyanopiperidin-1-yl)-1,3-thiazol-4-yl]methyl}-N-(4-fluorophenyl)acetamide;
N-(4-fluorophenyl)-N-({2-[(2-hydroxyethyl)(methyl)amino]-1,3-thiazol-4-yl}methyl)acetamide;
N-(4-fluorophenyl)-N-{[2-(4-hydroxypiperidin-1-yl)-1,3-thiazol-4-yl]methyl}acetamide;
N-(4-fluorophenyl)-N-{[2-(3-hydroxypiperidin-1-yl)-1,3-thiazol-4-yl]methyl}acetamide;
N-(4-fluorophenyl)-N-({2-[4-(hydroxymethyl)piperidin-1-yl]-1,3-thiazol-4-yl}methyl)acetamide;
N-(4-fluorophenyl)-N-({2-[3-(hydroxymethyl)piperidin-1-yl]-1,3-thiazol-4-yl}methyl)acetamide;
N-(4-fluorophenyl)-N-{[6-(4-methyl-1,4-diazepan-1-yl)pyridin-3-yl]methyl}acetamide;
N-(4-fluorophenyl)-N-[(6-morpholin-4-ylpyridin-3-yl)methyl]acetamide;
N-{[6-(diethylamino)pyridin-3-yl]methyl}-N-(4-fluorophenyl)acetamide;
N-{[6-(4-cyanopiperidin-1-yl)pyridin-3-yl]methyl}-N-(4-fluorophenyl)acetamide;
N-(4-fluorophenyl)-N-({6-[(2-hydroxyethyl)(methyl)amino]pyridin-3-yl]methyl}acetamide;
N-(4-fluorophenyl)-N-{[6-(4-hydroxypiperidin-1-yl)pyridin-3-yl]methyl}acetamide;
N-(4-fluorophenyl)-N-{[6-(3-hydroxypiperidin-1-yl)pyridin-3-yl]methyl}acetamide;
N-(4-fluorophenyl)-N-({6-[4-(hydroxymethyl)piperidin-1-yl]pyridin-3-yl}methyl}acetamide;
N-(4-fluorophenyl)-N-({6-[3-(hydroxymethyl)piperidin-1-yl]pyridin-3-yl}methyl}acetamide;
N-cyclopentyl-N-({6-[4-(hydroxymethyl)piperidin-1-yl]pyridin-3-yl}methyl)acetamide;
N-cyclohexyl-N-{[6-(4-methylpiperazin-1-yl]pyridin-3-yl}methyl)acetamide;
N-cyclohexyl-N-({6-[4-(hydroxymethyl)piperidin-1-yl]pyridin-3-yl}methyl)acetamide;
N-(4-tert-butylcyclohexyl)-N-({2-[cyclopropyl(ethyl)amino]-1,3-thiazol-4-yl}methyl)acetamide;
N-{[2-(2,6-dimethylmorpholin-4-yl)-1,3-thiazol-4-yl]methyl}-N-(4-fluorophenyl)-2,2-dimethylpropanamide;
N-(4-fluorophenyl)-2,2-dimethyl-N-[(2-quinolin-3-yl-1,3-thiazol-4-yl)methyl]propanamide;
N-(4-fluorophenyl)-2,2-dimethyl-N-({2-[4-(trifluoromethyl)phenyl]-1,3-thiazol-4-yl}methyl)propanamide;
N-(4-fluorophenyl)-2,2-dimethyl-N-({2-[3-(trifluoromethyl)phenyl]-1,3-thiazol-4-yl}methyl)propanamide;
N-(4-fluorophenyl)-2,2-dimethyl-N-({2-[2-(trifluoromethyl)phenyl]-1,3-thiazol-4-yl}methyl)propanamide;
N-(4-fluorophenyl)-N-{[2-(4-hydroxyphenyl)-1,3-thiazol-4-yl]methyl}-2,2-dimethylpropanamide;
N-(4-fluorophenyl)-N-{[2-(3-hydroxyphenyl)-1,3-thiazol-4-yl]methyl}-2,2-dimethylpropanamide;
N-(4-fluorophenyl)-N-{[2-(2-hydroxyphenyl)-1,3-thiazol-4-yl]methyl}-2,2-dimethylpropanamide;
N-(4-fluorophenyl)-N-{[2-(4-fluorophenyl)-1,3-thiazol-4-yl]methyl}-2,2-dimethylpropanamide;
N-(4-fluorophenyl)-N-{[2-(3-fluorophenyl)-1,3-thiazol-4-yl]methyl}-2,2-dimethylpropanamide;
N-(4-fluorophenyl)-N-{[2-(2-fluorophenyl)-1,3-thiazol-4-yl]methyl}-2,2-dimethylpropanamide;
N-(4-fluorophenyl)-N-{[6-(4-methylpiperazin-1-yl)pyridin-3-yl]methyl}tetrahydrofuran-2-carboxamide;
N-(4-fluorophenyl)-N-{[6-(4-methylpiperazin-1-yl)pyridin-3-yl]methyl}tetrahydrofuran-3-carboxamide;
N-[(6-chloropyridin-3-yl)methyl]-N-(4-fluorophenyl)tetrahydrofuran-2-carboxamide;
N-[(6-chloropyridin-3-yl)methyl]-N-(4-fluorophenyl)tetrahydrofuran-3-carboxamide;
tert-butyl (3S)-3-{[(6-chloropyridin-3-yl)methyl](4-fluorophenyl)carbamoyl}pyrrolidine-1-carboxylate;
tert-butyl (3R)-3-{[(6-chloropyridin-3-yl)methyl](4-fluorophenyl)carbamoyl}pyrrolidine-1-carboxylate;
tert-butyl (3S)-3-[(4-fluorophenyl){[6-(4-methylpiperazin-1-yl)pyridin-3-yl]methyl}carbamoyl]pyrrolidine-1-carboxylate;
tert-butyl (3R)-3-[(4-fluorophenyl){[6-(4-methylpiperazin-1-yl)pyridin-3-yl]methyl}carbamoyl]pyrrolidine-1-carboxylate;
(3S)—N-(4-fluorophenyl)-N-{[6-(4-methylpiperazin-1-yl)pyridin-3-yl]methyl}pyrrolidine-3-carboxamide;
(3R)—N-(4-fluorophenyl)-N-{[6-(4-methylpiperazin-1-yl)pyridin-3-yl]methyl}pyrrolidine-3-carboxamide;
(3S)-1-(2,2-dimethylpropanoyl)-N-(4-fluorophenyl)-N-{[6-(4-methylpiperazin-1-yl)pyridin-3-yl]methyl}pyrrolidine-3-carboxamide;
(3R)-1-(2,2-dimethylpropanoyl)-N-(4-fluorophenyl)-N-{[6-(4-methylpiperazin-1-yl)pyridin-3-yl]methyl}pyrrolidine-3-carboxamide;
and pharmaceutically acceptable salts, hydrates, and esters thereof.

24. A pharmaceutical composition comprising the compound of claim 1 or a pharmaceutically acceptable salt, hydrate or ester thereof and a pharmaceutically acceptable carrier or excipient.

25. (canceled)

26. (canceled)

27. (canceled)

28. (canceled)

29. A method of treating pain in a subject, the method comprising administering to a subject a therapeutically effective amount of the compound of claim 1 or a pharmaceutically acceptable salt, hydrate, or ester thereof, wherein said pain is chronic pain, chronic back pain, neuropathic pain, pain associated with diabetic neuropathy, pain associated with post-herpetic neuropathy or pain associated with post-herpetic fibromyalgia.

30. A method of claim 29 wherein said pain is chronic pain, wherein said chronic pain is associated with diabetes, post traumatic pain of amputation, lower back pain, spinal cord damage, cancer, chemical injury, chemotherapy induced peripheral neuropathy, toxins, major surgery, peripheral nerve damage due to traumatic injury, post-herpetic neuralgia, trigeminal neuralgia, lumbar or cervical radiculopathies, fibromyalgia, glossopharyngeal neuralgia, reflex sympathetic dystrophy, causalgia, thalamic syndrome, nerve root avulsion, reflex sympathetic dystrophy or post thoracotomy pain, nutritional deficiencies, viral infection, bacterial infection, metastatic infiltration, adiposis dolorosa, burns, central pain conditions related to thalamic conditions, or a combination thereof.

31. (canceled)

32. (canceled)

33. (canceled)

34. (canceled)

35. (canceled)

36. (canceled)

37. A method of treating a disease or disease symptom modulated by calcium channel Caᵥ2.2, the method comprising administering to a subject a therapeutically effective amount of the compound of claim 1 or a pharmaceutically acceptable salt, hydrate, or ester thereof.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. The compound of claim 1 or a pharmaceutically acceptable salt thereof, wherein Ar—R³ is: 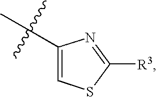
wherein R³ is as defined in claim 1.

62. The compound of claim 61, wherein the compound is:
N-(4-Fluorophenyl)-2,2-dimethyl-N-(2-pyrrolidin-1-yl-thiazol-5-ylmethyl)-propionamide;
N-(4-Fluorophenyl)-2,2-dimethyl-N-[2-(4-methyl-piperazin-1-yl)-thiazol-5-ylmethyl]-propionamide;
(4-Fluorophenyl)-[2-(4-methyl-piperazin-1-yl)-thiazol-5-ylmethyl]-carbamic acid tert-butyl ester;
N-(4-methylphenyl)-N-[(2-pyrrolidin-1-yl-1,3-thiazol-5-yl)methyl]-L-prolinamide;
N-({2-[cyclopropyl(ethyl)amino]-1,3-thiazol-4-yl}methyl)-N-(4-fluorophenyl)-L-prolinamide;
or a pharmaceutically acceptable salts thereof.
